(19) United States
(12) Patent Application Publication
(10) Pub. No.: US 2023/0128075 A1
(43) Pub. Date: Apr. 27, 2023

(54) MONOCLONAL ANTIBODIES TARGETING HSP70 AND THERAPEUTIC USES THEREOF

(71) Applicants: Board of Regents, The University of Texas System, Austin, TX (US); Asylia Therapeutics, Houston, TX (US)

(72) Inventors: Robert Z. ORLOWSKI, Houston, TX (US); Richard J. JONES, Houston, TX (US); Laura C. BOVER, Houston, TX (US); Jeno GYURIS, Houston, TX (US); John MILLER, Houston, TX (US); James Bernard McCLORY, Banbridge (GB); Darragh MAC CANN, Magherafelt (GB); William M. WINSTON, SR., West Newton, MA (US)

(73) Assignees: Board of Regents, The University of Texas System, Austin, TX (US); Asylia Therapeutics, Houston, TX (US)

(21) Appl. No.: 17/907,375

(22) PCT Filed: Mar. 26, 2021
(86) PCT No.: PCT/US2021/024473

§ 371 Date: Sep. 26, 2022

Related U.S. Application Data
(60) Provisional application No. 63/001,011, filed on Mar. 27, 2020.

(51) Int. Cl. C07K 16/18; A61K 45/06; A61K 39/395; C07K 14/705; C07K 14/725; G01N 33/68; A61P 35/00; A61P 1/00; A61P 17/00; A61K 47/68

(52) U.S. Cl. CPC C07K 16/18; A61K 45/06; A61K 39/3955; C07K 14/70517; C07K 14/70521; C07K 14/7051; G01N 33/68; A61P 35/00; A61P 1/00; A61P 17/00; A61K 47/6803; A61K 47/6843; C07K 2317/24; C07K 2317/31; C07K 2319/03; C07K 2319/02; C07K 2317/92; C07K 2317/76; A61K 2039/505

(57) ABSTRACT

Provided herein are agents, such as antibodies and chimeric antigen receptors, that target HSP70. Methods of treating cancer are provided, comprising administering to a patient in need thereof an effective amount of an HSP70-targeting agent. The HSP70-specific antibody may enhance uptake of HSP70 by antigen presenting cells.

### REFERENCE TO RELATED APPLICATIONS

The present application claims the priority benefit of U.S. provisional application No. 63/001,011, filed Mar. 27, 2020, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Mar. 25, 2021, is named UTFCP1469WO_ST25.txt and is 220 kilobytes in size.

### BACKGROUND

### 1. Field

The present invention relates generally to the fields of medicine, immunology, and cancer biology. More particularly, it concerns antibodies that target HSP70 and methods of their use.

### 2. Description of Related Art

The development of an immune response against cancerous cells is believed to depend upon a series of reinforcing events, which have been referred to as the Cancer

Immunity Cycle (Chen & Mellman, 2013), which starts with release of cancer cell antigens during cancer cell death. Dendritic cells (DCs) are believed to be key early components of this response by virtue of their ability to capture, process, and then present these tumor antigens to T cells via presentation through histocompatibility complex (MHC) class I and II molecules, which then results in the priming and activation of effector CD4+ and CD8+ T-cell responses. The crucial role of DCs is demonstrated, in part, by the many mechanisms leveraged by tumors to suppress DC activity, including hypoxia, adenosine, lactic acid, low pH, and expression of interleukin (IL)-10 and PD-L1, among others (Veglia & Gabrilovich, 2017).

Heat shock proteins (HSPs) in general, and HSP70 in particular, are believed to play a key role in this process because of their ability to link the innate and adaptive immune responses (Shevtsov & Multhoff, 2016). For example, extracellular HSP70 binds and chaperones tumor antigens and then targets antigen presenting cells, including DCs, through binding to distinct cell surface receptors, including CD91, oxidized low-density lipoprotein receptor 1 (OLR1), and scavenger receptor expressed by endothelial cells (SREC)-1, among others (McNulty et al., 2013), thereby delivering bound antigens to DCs for processing. Furthermore, extracellular HSP70 secreted from tumor cells induces inflammatory cytokines such as Interleukin (IL)-6 and Tumor necrosis factor (TNF)-α from macrophages (Vega et al., 2008), thereby enabling cross-presentation and T-cell activation, respectively. As such, HSP70 is considered to be an attractive target for cancer therapy because of its crucial intracellular role as a cytoprotective, anti-apoptotic factor that promotes cancer cell survival in the face of various stressors, including both radiation and a variety of chemotherapeutics (Boudesco et al., 2018). Furthermore, HSP70 is also considered to be an attractive target for cancer therapy because of its ability to stimulate immune responses through not just DCs, but possibly also macrophages, NK cells, and T cells (Shvetsov & Multhoff, 2016; Zininga et al., 2018).

Approaches that enhance DC uptake of HSP70-tumor antigen complexes hold the promise of enhancing anti-tumor immunity and breaking tolerance. Several pharmacologic inhibitors have been developed that target intracellular HSP70 directly, or some its co-chaperones (Boudesco et al., 2018), which may act as sensitizers to radiation or chemotherapy. Also, while membrane-bound HSP70 is usually either absent or found only at low levels on normal cells, it often shows enhanced expression on the surface of tumor cells, and in some malignancies has been associated with a more aggressive phenotype and inferior prognosis (Boudesco et al., 2018; Chatterjee & Burns, 2017). Moreover, HSP70^{-/-} tumors have been found to be less immunogenic and more aggressive (Dodd et al., 2015). This has led to the development and testing of a variety of approaches, including ferromagnetic and gold nanoparticle-based therapies, vaccine strategies (Shvetsov & Multhoff, 2016), and monoclonal antibodies such as cmHSP70.1 (Stangl et al., 2011), that rely on HSP70 cell surface expression for their activity.

Over recent years, immune checkpoint inhibitors (ICIs), including monoclonal antibodies to cytotoxic T-lymphocyte associated protein 4 (CTLA-4) and programmed cell death 1 (PD-1) and its ligand, PD-L1, have revolutionized immunotherapy through their ability to induce durable remissions in even advanced malignancies. In general, it is believed that tumors that respond to ICIs tend to have higher immune cell infiltration and/or an interferon gene signature, or a higher tumor mutation burden (TMB), and are sometimes referred to as “hot” tumors (Maleki Vareki, 2018). In contrast, so-called “cold” tumors with low immune cell infiltrates or low TMB tend not to respond to ICIs, and include pancreatic and prostate cancers (Maleki Vareki, 2018). Despite the advances in treating various malignancies, there is still a need for new approaches that convert cold tumors into more immunogenic tumors, which may provide alternative approaches for the treatment of these tumors.

### SUMMARY

The invention is based, in part, upon the discovery of anti-HSP70 monoclonal antibodies or antibody fragments. In certain circumstances, the anti-HSP70 monoclonal antibodies or antibody fragments may, for example, target extracellular or soluble HSP70 associated with tumor-derived antigens to immune cells (e.g., dendritic cells) and thereby treat cancer and/or enhance the efficacy of a cancer therapy (e.g., a cancer immunotherapy).

In one embodiment, the monoclonal antibody or antibody fragment is the 77A antibody, as described herein. For example, in some aspects, said antibodies or antibody fragments comprise a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

In some aspects, said antibodies or antibody fragments comprise a heavy chain variable sequence having at least 70%, 75%, 80%, 85%, or 90% identity to SEQ ID NO: 7 and a light chain variable sequence having at least 70%, 75%, 80%, 85%, or 90% identity to SEQ ID NO: 8. In some aspects, said antibodies or antibody fragments comprise a heavy chain variable sequence having at least 91%, 92%, 93%, 94%, 95%, 96%, 97%, 98%, or 99% identity to SEQ ID NO: 7 and a light chain variable sequence having at least 91%, 92%, 93%, 94%, 95%, 96%, 97%, 98%, or 99% identity to SEQ ID NO: 8. In some aspects, said antibodies or antibody fragments comprise a heavy chain variable sequence having a sequence according to SEQ ID NO: 7 and a light chain variable sequence having a sequence according to SEQ ID NO: 8.

In some aspects, said antibodies or antibody fragments are encoded by a heavy chain variable sequence having at least 70%, 75%, 80%, 85%, or 90% identity to SEQ ID NO: 9 and a light chain variable sequence having at least 70%, 75%, 80%, 85%, or 90% identity to SEQ ID NO: 10. In some aspects, said antibodies or antibody fragments are encoded by a heavy chain variable sequence having at least 91%, 92%, 93%, 94%, 95%, 96%, 97%, 98%, or 99% identity to SEQ ID NO: 9 and a light chain variable sequence having at least 91%, 92%, 93%, 94%, 95%, 96%, 97%, 98%, or 99% identity to SEQ ID NO: 10. In some aspects, said antibodies or antibody fragments are encoded by a heavy chain variable sequence according to SEQ ID NO: 9 and a light chain variable sequence according to SEQ ID NO: 10.

In another aspect, humanized variants of the 77A antibody are provided. For example, in some aspects, the monoclonal antibodies or antibody fragments comprise a heavy chain variable sequence having a sequence of

(SEQ ID NO: 18)
X₁X₂QLX₃X₄SGX₅X₆X₇X₈KPGX₉SX₁₀X₁₁X₁₂SCKX₁₃SGYTFTNYG
MNWVRQAPGX₁₄GLX₁₅WX₁₆GWINTYTGEPTYADDFKGRX₁₇TX₁₈X₁₉
X₂₀DX₂₁SX₂₂X₂₃TX₂₄YX₂₅X₂₆X₂₇X₂₈X₂₉LX₃₀X₃₁X₃₂DTAVYF
CARYDHAMDYWGQGTX₃₃VTVSS,

wherein X₁ is Q or E, X₂ is I or V, X₃ is V or Q, X₄ is Q or E, X₅ is A, P, or G, X₆ is E or G, X₇ is V or L, X₈ is V or K, X₉ is A, E, G, or S, X₁₀ is V or L, X₁₁ is K or R, X₁₂ is V, L, or I, X₁₃ is A or T, X₁₄ is K or Q, X₁₅ is E or K, X₁₆ is M or V, X₁₇ is F or V, X₁₈ is F, M, or I, X₁₉ is T or S, X₂₀ is T, R, or A, X₂₁ is T, D, or E, X₂₂ is T, A, or K, X₂₃ is S or N, X₂₄ is L or A, X₂₅ is M or L, X₂₆ is E or Q, X₂₇ is L or M, X₂₈ is R, S, T, or N, X₂₉ is S or G, X₃₀ is R, K, or M, X₃₁ is S or T, X₃₂ is D or E, and X₃₃ is L, S, or T; and/or a light chain variable sequence having a sequence of

(SEQ ID NO: 26)
X₁X₂X₃X₄TQSPX₅SLX₆X₇SX₈GX₉RX₁₀TIX₁₁CKSSQSLLNSGTRKN
YLAWYQQKX₁₂GX₁₃X₁₄PX₁₅LLIYWTSTRESGVPX₁₆RFSGSGSGTDF
TLTIX₁₇X₁₈LQX₁₉EDVAX₂₀YYCKQSYTLYTFGX₂₁GTKX₂₂EIK,

wherein X₁ is E or D, X₂ is I or V, X₃ is V or Q, X₄ is L or M, X₅ is D or S, X₆ is A or S, X₇ is V or A, X₈ is L or V, X₉ is E or D, X₁₀ is A or V, X₁₁ is N or T, X₁₂ is A or P, X₁₃ is Q or K, X₁₄ is S, V, or P, X₁₅ is K or R, X₁₆ is D or S, X₁₇ is S, D, or N, X₁₈ is S or T, X₁₉ is A or P, X₂₀ is V or T, X₂₁ is Q or G, and X₂₂ is L or V.

In certain aspects, the antibodies or antibody fragments comprise a heavy chain variable sequence having a sequence selected from the group consisting of SEQ ID NOs: 12-16, or a heavy chain variable sequence having at least 70%, 80%, 90%, 95%, 96%, 97%, 98%, or 99% identity to any one of SEQ ID NOs: 12-19; and/or a light chain variable sequence having a sequence selected from the group consisting of SEQ ID NOs: 19-23, or a light chain variable sequence having at least 70%, 80%, 90%, 95%, 96%, 97%, 98%, or 99% identity to any one of SEQ ID NOs: 19-23.

In addition, optimized humanized variants of the 77A antibody are provided. For example, in some aspects, the monoclonal antibodies or antibody fragments comprise a heavy chain variable region (VH) comprising a VHCDR1 amino acid sequence of GYX₁FTX₂YG (SEQ ID NO: 214), wherein X₁ is T, S, or I, and X₂ is N or K, a VHCDR2 amino acid sequence of INTYTGEX₁ (SEQ ID NO: 215), wherein X₁ is P, S, T, or A, and a VHCDR3 amino acid sequence of X₁RYDHX₂MDY (SEQ ID NO: 216), wherein X₁ is A, T, V, or G, and X₂ is A, R, F, T, P, V, S, D, N, H, L, Y, or G; and/or a light chain variable region (VL) comprising a VLCDR1 amino acid sequence of QSLX₁NSGTRKNY (SEQ ID NO: 212), wherein X₁ is L, F, or V, a VLCDR2 amino acid sequence of SEQ ID NO: 5, and a VLCDR3 amino acid sequence of KQSYX₁LYT (SEQ ID NO: 213), wherein X₁ is T, N, or S. In some aspects, the monoclonal antibodies or antibody fragments comprise VHCDR1 amino acid sequence selected from the group consisting of SEQ ID NOs: 1 and 164-166, a VHCDR2 amino acid sequence selected from the group consisting of SEQ ID NOs: 2 and 167-169, and a VHCDR3 amino acid sequence selected from the group consisting of SEQ ID NOs: 3 and 170-185; and/or a light chain variable region (VL) comprising a VLCDR1 amino acid sequence selected from the group consisting of SEQ ID NOs: 4 and 159-161, a VLCDR2 amino acid sequence of SEQ ID NO: 5, and a VLCDR3 amino acid sequence selected from the group consisting of SEQ ID NOs: 6, 162, and 163.

In some aspects, the monoclonal antibodies or antibody fragments comprise a heavy chain variable sequence having a sequence of

(SEQ ID NO: 104)
QIX₁LVQSGX₂EVKKPGASVKVSCKASGYX₃FTX₄YGMNWVRQAPGQGLE
WMGWINTYTGEX₅X₆YX₇DDFKGRFTFTTDTSTX₈TX₉YMX₁₀X₁₁RSLR
SDDTAVYFCX₁₂RYDHX₁₃MDYWGQGX₁₄LVTVSS,

wherein X₁ is Q or H, X₂ is A, D, T, V, S, or P, X₃ is T, S, or I, X₄ is N or K, X₅ is P, S, T, or A, X₆ is T, R, K, or I, X₇ is A, T, V, S, or G, X₈ is S, R, or T, X₉ is A, V, or G, X₁₀ is E or D, X₁₁ is L or V, X₁₂ is A, T, V, or G, X₁₃ is A, R, F, T, P, V, S, D, N, H, L, Y, or G, and X₁₄ is T or S;
and/or a light chain variable sequence having a sequence of

(SEQ ID NO: 158)
EIVLTQSPDSLX₁VSLGERATIX₂CKSSQSLX₃NSGTRKNYLX₄WYQX₅K
X₆GQSPX₇LX₈IYWTSTRESGVPDRFSX₉SGSGTDFTLX₁₀IDX₁₁LQ
X₁₂EDVAX₁₃YYCKQSYX₁₄LYTFGGGTKVEIK,

wherein X₁ is A, T, or S, X₂ is N or K, X₃ is L, F, or V, X₄ is A, S, or T, X₅ is Q or K, X₆ is A, P, or S, X₇ is K or N, X₈ is L, V, or I, X₉ is G or A, X₁₀ is T or S, X₁₁ is S or R, X₁₂ is A or T, X₁₃ is V, I, or L, and X₁₄ is T, N, or S.

In some aspects, said antibodies or antibody fragments comprise a heavy chain variable sequence having a sequence selected from the group consisting of SEQ ID NOs: 17 and 26-103, or a heavy chain variable sequence having at least 70%, 80%, 90%, 95%, 96%, 97%, 98%, or 99% identity to any one of SEQ ID NOs: 17 and 26-103; and/or a light chain variable sequence having a sequence selected from the group consisting of SEQ ID NOs: 24 and 105-157, or a light chain variable sequence having at least 70%, 80%, 90%, 95%, 96%, 97%, 98%, or 99% identity to any one of SEQ ID NOs: 24 and 105-157.

In some aspects, the antibodies bind, or are capable of binding, to HSP70. In some aspects, the antibodies bind to human HSP70 with a K_{D} less than about 10, 9, 8, 7, 6, 5, 4, 3, 2, 1, 0.9, 0.85, 0.8, 0.75, 0.7, 0.6, or 0.5 nM, as determined by Octet bio-layer interferometry (BLI) analysis.

Also provided herein are monoclonal antibodies or antibody fragments, which compete for binding to the same epitope as the monoclonal antibodies or antibody fragments according to any one of the present embodiments. For example, the present invention includes anti-HSP70 antibodies that compete for binding to HSP70 with the 77A antibody as defined herein. It is possible to determine whether an antibody binds to the same epitope as, or competes for binding with, the 77A antibody by using routine methods known in the art. For example, to determine if a test antibody binds to the same epitope as the 77A antibody, the 77A antibody is allowed to bind to an HSP70 protein or peptide under saturating conditions. Next, the ability of a test antibody to bind to the HSP70 protein or peptide is assessed. If the test antibody is able to bind to the HSP70 protein or peptide following saturation binding with the 77A antibody, it can be concluded that the test antibody binds to a different epitope than the 77A antibody. On the other hand, if the test antibody is not able to bind to the HSP70 protein or peptide following saturation binding with the 77A antibody, then the test antibody may bind to the same epitope as the epitope bound by the 77A antibody.

In one embodiment, provided herein are monoclonal antibodies or antibody fragments that binds, or is capable of binding, to an epitope on HSP70 recognized by an antibody or antibody fragment of any one of the present embodiments.

In one embodiment, provided herein are monoclonal antibodies or antibody fragments, wherein the monoclonal antibodies or antibody fragments bind to an epitope of HSP70 defined by a peptide corresponding to K573-Q601 of SEQ ID NO: 11. In certain aspects, when bound to HSP70, the monoclonal antibodies or antibody fragments bind to one or two of the following residues: H594, K595, and Q601 of SEQ ID NO:11. In some aspects, when bound to HSP70, the monoclonal antibodies or antibody fragments bind to all of the following residues: H594, K595, and Q601 of SEQ ID NO:11. In some aspects, when bound to HSP70, the monoclonal antibodies or antibody fragments additionally bind to at least one of the following residues: K573, E576, W580, R596, and E598 of SEQ ID NO:11. In some aspects, when bound to HSP70, the monoclonal antibodies or antibody fragments additionally bind to at least two, three, four, or five of the following residues: K573, E576, W580, R596, and E598 of SEQ ID NO:11. In some aspects, when bound to HSP70, the monoclonal antibodies or antibody fragments bind to all of the following residues: K573, E576, W580, H594, K595, R596, E598, and Q601 of SEQ ID NO:11.

In some aspects, provided herein are monoclonal antibodies or antibody fragments, wherein, when bound to HSP70, the monoclonal antibodies or antibody fragments enhance the uptake of tumor-derived ADP-HSP70-peptide antigen complexes by immune effector cells.

In some aspects of any of the present embodiments, the antibodies bind, or are capable of binding, to HSP70. In some aspects of any of the present embodiments, the antibodies bind, or are capable of binding, to HSP70 in its ADP-bound form. In some aspects of any of the present embodiments, the antibodies bind, or are capable of binding, to HSP70 in its peptide-bound form. In some aspects of any of the present embodiments, the antibodies bind, or are capable of binding, to HSP70 in its ADP-bound and peptide-bound form. In some aspects of any of the present embodiments, the antibody binds, or are capable of binding, to human HSP70 with a K_{D} less than about 10, 9, 8, 7, 6, 5, 4, 3, 2, 1, 0.9, 0.85, 0.8, 0.75, 0.7, 0.6, or 0.5 nM, as determined by Octet bio-layer interferometry (BLI) analysis. In some aspects of any of the present embodiments, the antibodies do not induce antibody-dependent cellular cytotoxicity. In some aspects of any of the present embodiments, the antibodies do not induce complement-dependent cellular cytotoxicity. In some aspects of any of the present embodiments, the antibodies enhance HSP70 uptake by immune effector cells, such as, for example, monocytes/macrophages and dendritic cells. In some aspects, the uptake is mediated by human FcγR2A and/or human FcγR2B.

In some aspects of any of the present embodiments, the antibody fragments are monovalent scFv (single chain fragment variable) antibodies, divalent scFvs, Fab fragments, F(ab′)₂ fragments, F(ab′)₃ fragments, Fv fragments, or single chain antibodies. In some aspects of any of the present embodiments, the antibodies are chimeric antibodies, bispecific antibodies, or BiTEs. In some aspects of any of the present embodiments, the antibodies are IgG antibodies or recombinant IgG antibodies or antibody fragments.

In some aspects of any of the present embodiments, the antibodies are conjugated or fused to an imaging agent or a cytotoxic agent. In some aspects of any of the present embodiments, the antibodies are labeled. In some aspects, the labels are fluorescent labels, enzymatic labels, or radioactive labels.

In some aspects, the antibodies or antibody fragments are IgG antibodies or antibody fragments. In some aspects, the antibodies or antibody fragments are IgG1, IgG2, IgG3, or IgG4 antibodies or antibody fragments, for example, the antibodies or antibody fragments comprise any one of SEQ ID NOs: 217-221.

In one embodiment, provided herein are isolated nucleic acids encoding the antibody heavy and/or light chain variable regions of the antibodies of any of the present embodiments. In some aspects, the isolated nucleic acids comprise a nucleotide sequence that is at least 85%, 90%, 91%, 92%, 93%, 94%, 95%, 96%, 97%, 98%, 99%, or 100% identical to SEQ ID NO: 9 and/or 10.

In one embodiment, provided herein are expression vectors comprising a nucleic acid of any one of the present embodiments.

In one embodiment, provided herein are hybridomas or engineered cells comprising a nucleic acid encoding an antibody or antibody fragment of any one of the present embodiments.

In one embodiment, provided herein are hybridomas or engineered cells comprising a nucleic acid of any one of the present embodiments.

In one embodiment, provided herein are methods of making a monoclonal antibody or antibody fragment of any one of the present embodiments, the methods comprising culturing a hybridoma or engineered cell of any one of the present embodiments under conditions that allow expression of the antibody and optionally isolating the antibody from the culture.

In one embodiment, provided herein are pharmaceutical formulations comprising one or more antibody or antibody fragment of any one of the present embodiments.

In one embodiment, provided herein are methods of treating a patient having a cancer, the methods comprising administering to the patient an effective amount of antibody or antibody fragment of any one of the present embodiments. In some aspects, the methods enhance uptake of HSP70 by antigen presenting cells. In some aspects, the uptake of HSP70 by antigen presenting cells is mediated by human FcγR2A and/or human FcγR2B.

In some aspects, the methods are further defined as methods for enhancing cytotoxic T cell-mediated antitumor immunity. In some aspects, the methods are further defined as methods for increasing sensitivity to immunotherapy. In some aspects, the methods are further defined as methods of enhancing uptake of tumor-derived ADP-HSP70-peptide antigen complexes by immune effector cells. In some aspects, the methods are further defined as methods of enhancing antigen presentation by dendritic cells. In some aspects, the methods are further defined as methods of enhancing CD4+ and CD8+ T-cell responses to tumor antigens.

In some aspects, the cancer is an immunologically cold cancer, e.g., a pancreatic cancer or a prostate cancer.

In some aspects, the methods further comprise administering at least a second anti-cancer therapy, e.g., a chemotherapy, immunotherapy, radiotherapy, gene therapy, surgery, hormonal therapy, anti-angiogenic therapy, or cytokine therapy.

In one embodiment, provided herein are chimeric antigen receptor (CAR) proteins comprising an antigen binding domain that binds to human HSP70.

In some aspects, the antigen binding domain comprises VHCDR1 amino acid sequence selected from the group consisting of SEQ ID NOs: 1 and 164-166, a VHCDR2 amino acid sequence selected from the group consisting of SEQ ID NOs: 2 and 167-169, and a VHCDR3 amino acid sequence selected from the group consisting of SEQ ID NOs: 3 and 170-185; and/or a light chain variable region (VL) comprising a VLCDR1 amino acid sequence selected from the group consisting of SEQ ID NOs: 4 and 159-161, a VLCDR2 amino acid sequence of SEQ ID NO: 5, and a VLCDR3 amino acid sequence selected from the group consisting of SEQ ID NOs: 6, 162, and 163. In some aspects, the antigen binding domain comprises a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6. In some aspects, the CAR proteins are capable of binding to HSP70.

In some aspects, the antigen binding domain comprises a heavy chain variable sequence having a sequence selected from the group consisting of SEQ ID NOs: 7, 12-17, and 26-103, or a heavy chain variable sequence having at least 70%, 80%, 90%, 95%, 96%, 97%, 98%, or 99% identity to any one of SEQ ID NOs: 7, 12-17, and 26-103; and/or a light chain variable sequence having a sequence selected from the group consisting of SEQ ID NOs: 8, 19-24, and 105-157, or a light chain variable sequence having at least 70%, 80%, 90%, 95%, 96%, 97%, 98%, or 99% identity to any one of SEQ ID NOs: 8, 19-24, and 105-157.

In some aspects, the antigen binding domain comprises a heavy chain variable sequence having at least 70%, 75%, 80%, 85%, or 90% identity to SEQ ID NO: 7 and a light chain variable sequence having at least 70%, 75%, 80%, 85%, or 90% identity to SEQ ID NO: 8. In some aspects, the antigen binding domain comprises a heavy chain variable sequence having at least 91%, 92%, 93%, 94%, 95%, 96%, 97%, 98%, or 99% identity to SEQ ID NO: 7 and a light chain variable sequence having at least 91%, 92%, 93%, 94%, 95%, 96%, 97%, 98%, or 99% identity to SEQ ID NO: 8. In some aspects, the antigen binding domain comprises a heavy chain variable sequence having a sequence according to SEQ ID NO: 7 and a light chain variable sequence having a sequence according to SEQ ID NO: 8.

In some aspects, the antigen binding domain is encoded by a heavy chain variable sequence having at least 70%, 75%, 80%, 85%, or 90% identity to SEQ ID NO: 9 and a light chain variable sequence having at least 70%, 75%, 80%, 85%, or 90% identity to SEQ ID NO: 10. In some aspects, the antigen binding domain is encoded by a heavy chain variable sequence having at least 91%, 92%, 93%, 94%, 95%, 96%, 97%, 98%, or 99% identity to SEQ ID NO: 9 and a light chain variable sequence having at least 91%, 92%, 93%, 94%, 95%, 96%, 97%, 98%, or 99% identity to SEQ ID NO: 10. In some aspects, the antigen binding domain is encoded by a heavy chain variable sequence according to SEQ ID NO: 9 and a light chain variable sequence according to SEQ ID NO: 10.

In some aspects, the antigen binding domain is a humanized antigen-binding domain. In some aspects, the CAR proteins further comprises a hinge domain, a transmembrane domain, and an intracellular signaling domain. The hinge domain can be a CD8a hinge domain or an IgG4 hinge domain. The transmembrane domain can be a CD8a transmembrane domain or a CD28 transmembrane domain. The intracellular signaling domain can comprise a CD3z intracellular signaling domain.

In one embodiment, provided herein are nucleic acid molecules encoding a CAR protein of any one of the present embodiments. In some aspects, the sequence encoding the CAR protein is operatively linked to expression control sequences. In some aspects, the nucleic acid molecules are further defined as expression vectors.

In one embodiment, provided herein are engineered cells comprising a nucleic acid molecule encoding a chimeric antigen receptor (CAR) protein comprising an antigen binding domain that binds, or is capable of binding, to human HSP70. In some aspects, the nucleic acid molecule encodes a CAR protein of any one of the present embodiments.

In some aspects, the cells are T cells or NK cells. In some aspects, the nucleic acid is integrated into the genome of the cells. In some aspects, the cells are human cells.

In one embodiment, provided herein are pharmaceutical compositions comprising a population of cells in accordance with any one of the present embodiments in a pharmaceutically acceptable carrier.

In one embodiment, provided herein are methods of treating cancer in a human patient in need thereof comprising administering to the patient an anti-tumor effective amount of a cell therapy comprising one or more cells in accordance with any one of the present embodiments. In some aspects, the cells are allogeneic cells or autologous cells. In some aspects, the cells are HLA matched to the subject. In some aspects, the cancer is a pancreatic cancer or a prostate cancer.

In some aspects, the methods further comprise administering at least a second anti-cancer therapy, e.g., a chemotherapy, immunotherapy, radiotherapy, gene therapy, surgery, hormonal therapy, anti-angiogenic therapy or cytokine therapy.

In one embodiment, provided herein are methods of detecting HSP70 in an in vitro sample, the methods comprising contacting the in vitro sample with an antibody or antibody fragment of any one of the present embodiments and detecting the binding of the antibody or antibody fragment to the sample. In some aspects, the detecting is by flow cytometry, mass spectrometry, western blot, immunohistochemistry, ELISA, or RIA.

In one embodiment, provided herein are antibody molecules, pharmaceutical compositions, cells, or pharmaceutical compositions of any one of the present embodiments for use in treating a cancer in a subject.

In one embodiment, provided herein are uses of antibody molecules, pharmaceutical compositions, cells, or pharmaceutical compositions of any one of the present embodiments, in the manufacture of a medicament for treating a cancer in a subject.

As used herein, the term “essentially free” in connection with a specified component, is used herein to mean that none of the specified component has been purposefully formulated into a composition and/or is present only as a contaminant or in trace amounts. The total amount of the specified component resulting from any unintended contamination of a composition is therefore below 0.5%, 0.1%, or 0.05%, and preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. BALB/c mice injected with MOPC315.BM-luc cells received 200 μg injections twice weekly in weeks 1 through 3 of the indicated HSP70 mAbs (squares) or their IgG2B isotype controls (circles). Disease burden was monitored using whole-animal in vivo imaging and confirmed by serum light chain levels.

FIGS. 2A-C. Octet analysis studying the affinity of 77A to murine HSP70 (top panel), human HSP70 made in E. coli (middle panel), and human HSP70 made in Sf9 insect cells (bottom panel) (A). 77A shows preferential binding to ADP-HSP70 complexes (B). 77A binding to HSP70-GFP shows greatest affinity when ADP and a peptide substrate (NRL) is present (C). Within each group of columns in (C), the columns represent, from left to right, Buffer, ATP, ADP, ATP NRL, and ADP NRL.

FIGS. 3A-F. Full-length (FL) HSP70 was expressed as an N-terminal GFP fusion protein in HSP70 KO 293T cells (A), along with deletion mutants of the indicated length removing progressively more C-terminal amino acids. IP of cell extracts with 77A was followed by detection of proteins by Western blotting (WB) with an anti-GFP antibody (B). Smaller deletions were then generated for finer mapping (C), and the indicated IPs were performed from cell lysates (CL) or culture media supernatants (CM). Loading was confirmed with an a-light chain (LC) antibody. The putative binding domain for 77A is indicated on a molecular model of HSP70 representing the relevant region of the protein (D). The exact amino acids that comprise the 77A epitope were determined using alanine scanning analysis, and the results are shown in (E), and the ribbon diagram showing the primary and secondary critical sites is shown in (F).

FIGS. 4A-B. Luc-labeled MM1.S human myeloma cells were injected into nude mice, and treatment was given twice weekly in weeks 2 through 5 with either an IgG2B isotype control mAb or 77A at the indicated doses. Whole animal live imaging data are shown at week 5 (A) with the dorsal (upper panels) and ventral (lower panels) views indicating significant tumor growth in the IgG2B-treated mice but not in 77A-treated mice, especially at the higher dose levels. The same experiment was then performed in NSG mice, and tumor growth was measured both by imaging (B) and by an ELISA for human light chains.

FIG. 5. Immature murine DC2.4 cells were incubated at 37° C. for 6 hours with either vehicle (left two panels) or 6x-His-tagged HSP70 (right two panels) in the presence of IgG2B or 77A as indicated. They were then stained either with control IgG (left panel) or an α-6x-His tag mAb (right three panels), and HSP70 uptake was determined by flow. Significant uptake of HSP70 is seen only in the presence of 77A (right most panel).

FIG. 6. DC2.4 cells exposed to SD-derived 6x-His-tagged human HSP70 in the presence of either an isotype control mAb or the 77A mAb were stained either with wheat germ agglutinin (WGA) conjugated to Alexa Fluor 594 to stain cell membranes, the Alexa Fluor 488-tagged α-6x-His-tag mAb to detect HSP70, or 4′,6-diamidino-2-phenylindole (DAPI) to stain nuclei, and individual as well as a merged images were obtained. Representative fields are shown at 200× magnification.

FIG. 7. Electron micrographs of DCs exposed to HSP70 and gold-labeled 77A. Magnification shown is 100,000×.

FIGS. 8A-C. Mouse DC2.4 cells were treated with either PBS, or 5 μg of ADP-HSP70 purified from A-375 melanoma cells, which are a good source of HSP70 since they express high levels of this protein, in combination with 10 μg of IgG2B or 77A for 48 hours. RNA was harvested and cDNA was hybridized to the qPCR array described in the text. Genes that were activated or repressed by ≥2-fold are shown for the comparison between IgG2B and PBS (A; top panel) as well as 77A and PBS (A; bottom panel). Data are shown from 3 biological replicates. Ingenuity Pathway Analysis was then performed (B) to identify biological processes which could be influenced by these changes. Cytokines released by the maturing DCs were then analyzed using the BioRad Bio-Plex™ Pro Mouse Cytokine Array. Notable changes induced in the HSP70 and 77A exposed cells versus the HSP70 and IgG2B exposed cells are shown in the bar graph (C). Within each group of columns in (C), the left column represents IgG CTRL and the right column represents HSP70 STIMULATED.

FIGS. 9A-G. BALB/c mice were injected into the 4th right mammary gland with 7,500 luc-4T1 cells. After 12 days, when all mice had palpable and measurable tumor, 200 μg of either IgG2B (filled box) or 77A (open box) were injected IV twice per week for 3 weeks. Tumor volumes were measured using both calipers and whole animal imaging for the primary (A), while imaging was used to assess pulmonary metastases (B). Peripheral blood was collected on day 32 and assessed for CD4+ and CD8+ T-cells (C), and also for dually CD11c+/MHC class II+ cells as well as total MHC class II+ cells (D). 77A induces uptake of HSP70 into human primary CD4+ and CD8+ T cells (E). 77A stimulates MHC-independent cytolytic CD4 T-cell activity (F). 77A activity against the A375 melanoma model in nude mice (G).

FIG. 10. HSP70 (represented in blue) bound to ATP in the nucleotide binding domain (NBD) has an open conformation to allow interactions with the substrate binding domain (SBD). Substrate peptide interaction with the SBD, in coordination with J-proteins and a nucleotide exchange factor (NEF), stimulates the HSP70 ATPase activity, resulting in closing of the lid, thereby stabilizing HSP70's interaction with the substrate. Adapted from (Craig & Marszalek, 2017). The approximate location of 77A binding on the HSP70 model is also shown in the right panel.

FIGS. 11A-C. Homogenate from a 10 mL pellet of 4T1 cells expressing HSP70-GFP was purified over an ADP-agarose column to isolate ADP-HSP70-peptide complexes, and 10 μg was injected intraperitoneally into BALB/c mice on day −24 and boosted subcutaneously on day −10. These were then injected on day 0 with 4T1-luc cells expressing HSP70-GFP as in the legend to FIGS. 9A-D, and tumor growth was monitored by whole animal imaging (A). At day 37, when the animals were euthanized, spleen cells were isolated and either analyzed by FACS on that day or placed into culture for 7 days in the presence of irradiated 4T1 cells expressing HSP70-GFP and then analyzed by FACS. Comparisons are provided for the CD4+ (B) and CD8+ (C) T-cell content at spleen isolation (day 0) and after 7 days of culture (left panels). Cytotoxic T-cell activity was also tested by exposing the indicated cell fractions to living wild-type 4T1 cells or 4T1 cells expressing HSP70-GFP followed by viability studies (right panels).

FIGS. 12A-B. Cytokine release assays were performed on CD4+ (A) and CD8+ (B) T cells isolated from murine spleens after exposure to either 4T1 cells or 4T1 cells expressing HSP70-GFP as detailed above. IFNγ secretion is shown in each top panel while IL-2 secretion is shown in the bottom panels.

FIG. 13. 77A induces uptake of HSP70 through FcγR2A and FcγR2B. The top row represents HSP70 knockout HEK 293T cells expressing human FcγR2A. The bottom row represents HSP70 knockout HEK 293T cells expressing the indicated human Fcγ receptor.

FIGS. 14A-B. The sequence of murine and human HSP70 at the proposed binding site for 77A is highlighted (A). Amino acid differences are boxed, while potential phosphorylation sites are in red and ubiquitination sites are in green. Mutation of the three amino acids in human HSP70 to mimic the murine version reduces the ability of 77A to recognize human HSP70 (B).

FIGS. 15A-B. Tumor targets for 77A. PLD with IgG2B or 77A was evaluated in BALB/c mice orthotopically injected with 4T1 cells (A) and in a CT26-based immune-competent colon carcinoma mode(B).

FIG. 16. The antibody 77A was tested in epitope binning experiments for competition in binding to the HSP70 protein with the indicated antibodies. Numbers in FIG. 16 reflect the percent of maximal binding in the presence of the potentially competing antibody.

FIG. 17. The antibody 77A binding to ADP-HSP70 (top) and ATP-HSP70 (bottom) as measured by biolayer interferometry (BLI).

FIG. 18. The binding of antibody 77A to ADP-HSP70 and ATP-HSP70 as measured by ELISA, where No. 1° and No. 2° represent negative controls where no primary antibody or secondary antibody were present, respectively.

FIG. 19. Tumor volume following treatment of mice bearing CT-26 tumors with the indicated antibodies. Boxed days (14, 17, 21) indicate days when mice received treatment. * p=0.0023 relative to isotype control (IgG2B) as determined by Dunnett's multiple comparison t test.

FIGS. 20A-B. A sequence alignment of humanized variants hVH-1 through hVH-5 (FIG. 20A) and hVL-1 through hVL-5 (FIG. 20B).

FIG. 21. HSP70 uptake following incubation of cells expressing the indicated human Fcγ receptor with HSP70GFP, GFP-Nanobody Alexa-488, and the indicated antibody, as measured by total MFI (left) and % GFP positive cells (right). 253-77A=77A; HC1 LC1=h77A-1; HC2 LC1=h77A-6; HC3 LC1=h77A-11.

FIG. 22. HSP70 uptake following incubation of cells expressing the indicated human Fcγ receptor with HSP70GFP, GFP-Nanobody Alexa-488, and the indicated IgG2 antibody, as measured by total MFI (left) and % GFP positive cells (right). 253-77A=77A; HC1 LC1=h77A-1; HC2 LC1=h77A-6; HC3 LC1=h77A-11.

FIG. 23. HSP70 uptake following incubation of cells expressing the indicated mouse Fcγ receptor with HSP70GFP, GFP-Nanobody Alexa-488, and the indicated antibody, as measured by total MFI (left) and % GFP positive cells (right). 253-77A=77A; HC1 LC1=h77A-1; HC2 LC1=h77A-6; HC3 LC1=h77A-11.

FIG. 24. HSP70 uptake following incubation of cells expressing the indicated mouse Fcγ receptor with HSP70GFP, GFP-Nanobody Alexa-488, and the indicated IgG2 antibody, as measured by total MFI (left) and % GFP positive cells (right). 253-77A=77A; HC1 LC1=h77A-1; HC2 LC1=h77A-6; HC3 LC1=h77A-11.

FIG. 25. HSP70 uptake following incubation of DCs with HSP70GFP, GFP-Nanobody Alexa-488, and the indicated antibody, as measured by MFI (left) and % GFP positive cells (right). Results are shown for total cells gated against HSP70GFP. 253-77A=77A; HC1 LC1=h77A-1; HC2 LC1=h77A-6; HC3 LC1=h77A-11.

FIG. 26. HSP70 uptake following incubation of DCs with HSP70GFP, GFP-Nanobody Alexa-488, and the indicated antibody, as measured by % GFP positive cells (left) and MFI (right). Results are shown for plasmacytoid DC, CD303+ve, CD1C-ve cells. 253-77A=77A; HC1 LC1=h77A-1; HC2 LC1=h77A-6; HC3 LC1=h77A-11.

FIG. 27. HSP70 uptake following incubation of DCs with HSP70GFP, GFP-Nanobody Alexa-488, and the indicated antibody, as measured by % GFP positive cells (left) and MFI (right). Results are shown for type 1 DC, CD141+ve, CD1c-ve cells. 253-77A=77A; HC1 LC1=h77A-1; HC2 LC1=h77A-6; HC3 LC1=h77A-11.

FIG. 28. HSP70 uptake following incubation of DCs with HSP70GFP, GFP-Nanobody Alexa-488, and the indicated antibody, as measured by % GFP positive cells (left) and MFI (right). Results are shown for type 2 DC, CD1C+ve, CD303−ve cells. 253-77A=77A; HC1 LC1=h77A-1; HC2 LC1=h77A-6; HC3 LC1=h77A-11.

FIGS. 29A-J. A sequence alignment of humanized variants hVH-1.1 through hVH-1.78 (FIGS. 29A-F) and hVL-1.1 through hVL-1.53 (FIGS. 29G-J).

### DETAILED DESCRIPTION

The invention is based, in part, upon the development of anti-HSP antibodies, or fragments thereof, that are useful in the treatment of certain indications, e.g., cancer. In certain circumstances, the anti-HSP70 monoclonal antibodies or antibody fragments may, for example, target extracellular or soluble HSP70 associated with tumor-derived antigens to immune cells (e.g., dendritic cells) and thereby treat cancer and/or enhance the efficacy of a cancer therapy (e.g., a cancer immunotherapy).

The data provided herein show that an anti-HSP70 mAb (denoted as clone 77A) shows activity independent of surface HSP70 expression and targets extracellular HSP70 with tumor-derived antigens to DCs. This antibody can be used to better understand the role of HSP70 in immunity, and also as a therapeutic to enhance the effectiveness of cancer immunotherapy. In particular, Clone 77A is a high affinity HSP70 mAb that shows anti-tumor efficacy in models of both hematologic malignancies and solid tumors in immune-competent and nude mice, but not in immune-deficient mice bearing the spontaneous Protein kinase, DNA-activated, catalytic subunit (PRKDCSCID) mutation, also known as SCID mice. The antibody enhances intracellular uptake of HSP70 by DCs in in vitro assays leading to up-regulation of genes associated with DC maturation. When tested against orthotopically implanted 4T1 cells, an immunologically cold model of murine triple-negative breast cancer that does not respond to ICIs, 77A reduced primary tumor growth and inhibited the development of pulmonary and hepatic metastases. In combination with pegylated liposomal doxorubicin (PLD), an agent that causes immunogenic cell death (ICD) and enhances release of HSP70-tumor peptide complexes, 77A cured some mice in both the 4T1 model and a model of colorectal cancer. Finally, when ADP-HSP70 complexes purified from 4T1 cells were used as a vaccine with 77A, tumor growth after subsequent challenge with live 4T1 cells was inhibited compared with a mAb isotype control, and the abundance of 4T1-specific cytolytic CD4+ and CD8+ T-cell activity was enhanced. As such, enhancing the uptake of HSP70 by immune cells using clone 77A mAb augments anti-tumor immunity both alone, and in a number of rationally designed combination regimens.

### I. DEFINITIONS

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

### II. ANTIBODIES AND MODIFICATIONS OF ANTIBODIES

Provided herein are monoclonal antibodies having clone-paired CDRs from the heavy and light chains as illustrated in Tables 1, 6, 9 and 10. Such antibodies may be produced using methods described herein.

The monoclonal antibodies of the present invention have several applications, include the production of diagnostic kits for use in detecting HSP70, as well as for treating diseases associated with increased levels of HSP70. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv, Fd, Fd′, single chain antibody (ScFv), diabody, linear antibody), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

An “isolated antibody” is an antibody that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular instances, the antibody is purified: (1) to greater than 95% by weight of antibody as determined by the Lowry method, and most particularly more than 99% by weight; or (2) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, an isolated antibody will be prepared by at least one purification step.

The basic four-chain antibody unit is a heterotetrameric glycoprotein composed of two identical light (L) chains and two identical heavy (H) chains. The term “heavy chain” as used herein refers to the larger immunoglobulin subunit which associates, through its amino terminal region, with the immunoglobulin light chain. The heavy chain comprises a variable region (V_{H}) and a constant region (C_{H}). The constant region further comprises the C_{H}1 hinge, C_{H}2, and C_{H}3 domains. In the case of IgE, IgM, and IgY, the heavy chain comprises a C_{H}4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4, α1-α2). It is the nature of this chain that determines the “class” of the antibody as IgG, IgM, IgA IgD, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (V_{L}) and a constant region (C_{L}). Light chains are classified as either kappa or lambda (κ, λ) based on the amino acid sequences of their constant domains (C_{L}). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

An IgM antibody, for example, consists of 5 basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

A “variable region” of an antibody refers to the variable region of the antibody light chain or the variable region of the antibody heavy chain, either alone or in combination. The term “variable” refers to the fact that certain segments of the variable regions differ extensively in sequence among antibodies. The variable regions of both the light (V_{L}) and heavy (V_{H}) chain portions mediate antigen binding and define the specificity of a particular antibody for its particular antigen. However, the variability is not evenly distributed across the entirety of the variable regions. Instead, the variable regions consist of relatively invariant stretches called framework regions (FRs) separated by shorter regions of extreme variability called complementarity determining regions (CDRs) or hypervariable regions. The variable regions of native heavy and light chains each comprise four FRs, largely adopting a beta-sheet configuration, connected by three CDRs, which form loops connecting, and in some cases forming part of, the beta-sheet structure. The CDRs complement an antigen's shape and determine the antibody's affinity and specificity for the antigen. There are six CDRs in both V_{L} and V_{H}. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)). As used herein, a CDR may refer to CDRs defined by any of these numbering approaches or by a combination of approaches or by other desirable approaches. In addition, a new definition of highly conserved core, boundary and hyper-variable regions can be used.

A “constant region” of an antibody refers to the constant region of the antibody light chain or the constant region of the antibody heavy chain, either alone or in combination. The constant regions of the light chain (C_{L} ) and the heavy chain (C_{H}1, C_{H}2 or C_{H}3, or C_{H}4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody. The constant regions are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The antibody may be an antibody fragment. “Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H}1 domains; (iv) the Fd′ fragment having VH and C_{H}1 domains and one or more cysteine residues at the C-terminus of the C_{H}1 domain; (v) the Fv fragment having the V_{H} and V_{H} domains of a single antibody; (vi) the dAb fragment which consists of a VH domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-C_{H}1-V_{H}-C_{H}1) which, together with complementary light chain polypeptides, form a pair of antigen binding regions.

The antibody may be a chimeric antibody. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. For example, a chimeric antibody may be an antibody comprising antigen binding sequences from a non-human donor grafted to a heterologous non-human, human, or humanized sequence (e.g., framework and/or constant domain sequences). Typically, in these chimeric antibodies, the variable region of both light and heavy chains mimics the variable regions of antibodies derived from one species of mammals, while the constant portions are homologous to the sequences in antibodies derived from another. For example, methods have been developed to replace light and heavy chain constant domains of a monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

### A. Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

Methods for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

### B. Single Chain Antibodies

A single chain variable fragment (scFv) is a fusion of the variable regions of the heavy and light chains of immunoglobulins, linked together with a short linker. This chimeric molecule retains the specificity of the original immunoglobulin, despite removal of the constant regions and the introduction of a linker peptide. This modification usually leaves the specificity unaltered. scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. For example, the linker may have a proline residue two residues after the V_{H} C terminus and an abundance of arginines and prolines at other positions.

A single-chain antibody may also be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

For example, SMPT is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site. The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Flexible linkers may also be used.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### C. Bispecific and Multispecific Antibodies

Antibodies may be bispecific or multispecific. “Bispecific antibodies” are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). Taki et al. (2015) describes a bispecific anti-HSP70/anti-CD3 antibody.

Methods for making bispecific antibodies are known in the art. Traditional production of full-length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

The bispecific antibodies may be composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1993)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

A bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400). Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). The antibodies may also involve sequences or moieties that permit dimerization or multimerization of the receptors. Such sequences include those derived from IgA, which permit formation of multimers in conjunction with the J-chain. Another multimerization domain is the Gal4 dimerization domain.

A multivalent antibody may be internalized (and/or catabolized) faster than a bivalent antibody by a cell expressing an antigen to which the antibody binds. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. Multivalent antibodies may comprise (or consist of) three to about eight, for example four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

### D. BiTES

A bi-specific T-cell engagers (BiTE®) is an artificial bispecific monoclonal antibody that directs a host's immune system, more specifically the T cells' cytotoxic activity, to target diseased cells. BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic activity on target cells by producing proteins like perforin and granzymes, independently of the presence of MHC I or co-stimulatory molecules. These proteins enter the target cells and initiate apoptosis. This action mimics physiological processes observed during T cell attacks against infected cells.

### E. Antibody Conjugates

Antibodies of the present disclosure may be linked to at least one agent to form an antibody conjugate. The conjugate can be, for example, an antibody conjugated to another proteinaceous, carbohydrate, lipid, or mixed moiety molecule(s). Such antibody conjugates include, but are not limited to, modifications that include linking the antibody to one or more polymers. For example, an antibody may be linked to one or more water-soluble polymers. Linkage to a water-soluble polymer reduces the likelihood that the antibody will precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric reaction), a substrate, a solid matrix, such as biotin. An antibody may comprise one, two, or more of any of these labels.

Antibody conjugates may be used to deliver cytotoxic agents to target cells. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, auristatins, a-amanitin, anthracyclines, calechaemicin, etc.), radioconjugates, enzyme conjugates (e.g., RNase conjugates, granzyme antibody-directed enzyme/prodrug therapy), and the like.

Antibody conjugates are also used as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III).

The radioactive isotopes contemplated for use as conjugated include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred. Technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA) .

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Derivatization of immunoglobulins by selectively introducing sulfhydryl groups in the Fc region of an immunoglobulin, using reaction conditions that do not alter the antibody combining site are also contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### F. Antibody Drug Conjugates

Antibody drug conjugates, or ADCs, are a new class of highly potent biopharmaceutical drugs designed as a targeted therapy for the treatment of people with disease. ADCs are complex molecules composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on diseased cells). Antibodies track these proteins down in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and non-cleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex—amino acid, linker and cytotoxic agent—now becomes the active drug. In contrast, cleavable linkers are catalyzed by enzymes in the host cell, thereby releasing the cytotoxic agent.

Another type of cleavable linker adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation. Future direction in the development of ADCs also include the development of site-specific conjugation (TDCs) to further improve stability and therapeutic index and a-emitting immunoconjugates and antibody-conjugated nanoparticles.

### G. Intrabodies

In a particular embodiment, the antibody is a recombinant antibody that is suitable for action inside of a cell—such antibodies are known as “intrabodies.” These antibodies may interfere with target function by a variety of mechanisms, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required. An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.).

The two major issues impacting the implementation of intrabody therapeutics are delivery, including cell/tissue targeting, and stability. With respect to delivery, a variety of approaches have been employed, such as tissue-directed delivery, use of cell-type specific promoters, viral-based delivery, use of cell-permeability/membrane translocating peptides, and delivery using exosomes. One means of delivery comprises the use of lipid-based nanoparticles, or exosomes, as taught in U.S. Pat. Appln. Publn. 2018/0177727, which is incorporated by reference here in its entirety. With respect to stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

### H. Production and Purification of Antibodies

The methods for generating monoclonal antibodies generally begin along the same lines as those for preparing polyclonal antibodies. The first step for both of these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxy succinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59, and combinations of immunostimulatory molecules (“Adjuvant Systems”, such as AS01 or AS03). Additional experimental forms of inoculation to induce antigen-specific B cells are possible, including nanoparticle vaccines, or gene-encoded antigens delivered as DNA or RNA genes in a physical delivery system (such as lipid nanoparticle or on a gold biolistic bead), and delivered with needle, gene gun, or transcutaneous electroporation device. The antigen gene also can be carried as encoded by a replication competent or defective viral vector such as adenovirus, adeno-associated virus, poxvirus, herpesvirus, or alphavirus replicon, or alternatively a virus-like particle.

Methods for generating hybrids of antibody-producing cells and myeloma cells usually comprise mixing somatic cells with myeloma cells in a 2:1 proportion, though the proportion may vary from about 20:1 to about 1:1, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus or polyethylene glycol (PEG) are also known. The use of electrically induced fusion methods is also appropriate. Fusion procedures usually produce viable hybrids at low frequencies, about 1×10⁻⁶ to 1×10⁻⁸, but with optimized procedures one can achieve fusion efficiencies close to 1 in 200. However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide monoclonal antibodies. The cell lines may be exploited for monoclonal antibody production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide monoclonal antibodies in high concentration. The individual cell lines could also be cultured in vitro, where the monoclonal antibodies are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

Alternatively, a molecular cloning approach may be used to generate monoclonal antibodies. Single B cells labeled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Monoclonal antibodies produced by any means may be purified, if desired, using filtration, centrifugation, and various chromatographic methods, such as FPLC or affinity chromatography. Fragments of the monoclonal antibodies of the disclosure can be obtained from the purified monoclonal antibodies by methods that include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

The antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxyapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

### I. Modification of Antibodies

The sequences of antibodies may be modified for a variety of reasons, such as improved expression, improved cross-reactivity, or diminished off-target binding. Modified antibodies may be made by any technique known to those of skill in the art, including expression through standard molecular biological techniques, or the chemical synthesis of polypeptides.

For example, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

An isolated monoclonal antibody, or antigen binding fragment thereof, may contain a substantially homogeneous glycan without sialic acid, galactose, or fucose. The aforementioned substantially homogeneous glycan may be covalently attached to the heavy chain constant region.

A monoclonal antibody may have a novel Fc glycosylation pattern. Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

The isolated monoclonal antibody, or antigen binding fragment thereof, may be present in a substantially homogenous composition represented by the GNGN or G1/G2 glycoform, which exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. Fc glycosylation plays a significant role in anti-viral and anti-cancer properties of therapeutic mAbs. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, may be expressed in cells that express beta (1,4)-N-acetylglucosaminyltransferase III (GnT III), such that GnT III adds GlcNAc to the antibody. Methods for producing antibodies in such a fashion are provided in WO/9954342 and WO/03011878. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene comprising the cDNA encoding the antibodies.

Antibodies can be engineered to enhance solubility. For example, some hydrophilic residues such as aspartic acid, glutamic acid, and serine contribute significantly more favorably to protein solubility than other hydrophilic residues, such as asparagine, glutamine, threonine, lysine, and arginine.

B cell repertoire deep sequencing of human B cells from blood donors has been performed on a wide scale. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, N.Y.), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, Calif.). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

### J. Characterization of Antibodies

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody binds may consist of a single contiguous sequence of 3 or more (e.g., 3, 4, 5, 6, 7, 8, 9, 10, 11,12, 13, 14, 15, 16, 17, 18, 19, 20) amino acids located within the antigen molecule (e.g., a linear epitope in a domain). Alternatively, the epitope may consist of a plurality of noncontiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce monoclonal antibodies having the desired characteristics. MAP may be used to sort the antibodies of the disclosure into groups of antibodies binding different epitopes.

The present disclosure includes antibodies that may bind to the same epitope, or a portion of the same epitope. One can easily determine whether an antibody binds to the same epitope as, or competes for binding with, a reference antibody by using routine methods known in the art. For example, to determine if a test antibody binds to the same epitope as a reference antibody, the reference antibody is allowed to bind to the target molecule under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with, e.g., the 77A antibody, the above-described binding methodology is performed in two orientations: In a first orientation, the 77A antibody is allowed to bind to an HSP70 protein under saturating conditions followed by assessment of binding of the test antibody to the HSP70 protein. In a second orientation, the test antibody is allowed to bind to an HSP70 protein under saturating conditions followed by assessment of binding of the 77A antibody to the HSP70 protein. If, in both orientations, only the first (saturating) antibody is capable of binding to the HSP70 molecule, then it is concluded that the test antibody and the 77A antibody compete for binding to HSP70. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90%, or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In another aspect, the antibodies may be defined by their variable sequence, which include additional “framework” regions. These are provided in Tables 2, 3, 6, 9, and 10, that represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the antibodies provided herein and their antigen-binding fragments. A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art.

One can determine the biophysical properties of antibodies. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection; however, it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

### K. Specific Embodiments

In one embodiment, provided herein are monoclonal antibodies or antibody fragments comprising a heavy chain variable region (VH) comprising a VHCDR1 amino acid sequence of GYX₁FTX₂YG (SEQ ID NO: 214), wherein X₁ is T, S, or I, and X₂ is N or K, a VHCDR2 amino acid sequence of INTYTGEX₁ (SEQ ID NO: 215), wherein X₁ is P, S, T, or A, and a VHCDR3 amino acid sequence of X₁RYDHX₂MDY (SEQ ID NO: 216), wherein X₁ is A, T, V, or G, and X₂ is A, R, F, T, P, V, S, D, N, H, L, Y, or G; and/or a light chain variable region (VL) comprising a VLCDR1 amino acid sequence of QSLX₁NSGTRKNY (SEQ ID NO: 212), wherein X₁ is L, F, or V, a VLCDR2 amino acid sequence of SEQ ID NO: 5, and a VLCDR3 amino acid sequence of KQSYX₁LYT (SEQ ID NO: 213), wherein X₁ is T, N, or S.

In one embodiment, provided herein are monoclonal antibodies or antibody fragments, wherein the antibodies or antibody fragments comprise a heavy chain variable region (VH) comprising a VHCDR1 amino acid sequence selected from the group consisting of SEQ ID NOs: 1 and 164-166, a VHCDR2 amino acid sequence selected from the group consisting of SEQ ID NOs: 2 and 167-169, and a VHCDR3 amino acid sequence selected from the group consisting of SEQ ID NOs: 3 and 170-185; and/or a light chain variable region (VL) comprising a VLCDR1 amino acid sequence selected from the group consisting of SEQ ID NOs: 4 and 159-161, a VLCDR2 amino acid sequence of SEQ ID NO: 5, and a VLCDR3 amino acid sequence selected from the group consisting of SEQ ID NOs: 6, 162, and 163.

In one embodiment, provided herein are monoclonal antibodies or antibody fragments, wherein the antibodies or antibody fragments comprise:

(i) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(ii) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(iii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(iv) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;

(v) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(vi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 172; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(vii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;

(viii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 173; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(ix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 174; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(x) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 175; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;

(xii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 176; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and/or a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;

(xiv) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 177; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xv) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 178; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xvi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and/or a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;

(xviii) a heavy chain variable region (VH) comprising a VHCDR1 amino acid sequence of SEQ ID NO: 164, a VHCDR2 amino acid sequence of SEQ ID NO: 167, and a VHCDR3 amino acid sequence of SEQ ID NO: 174; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 180; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;

(xxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 182; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxiv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 184; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 185; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxvi) a heavy chain variable region (VH) comprising a VHCDR1 amino acid sequence of SEQ ID NO: 166 a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xvii) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxviii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxix) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 174; and/or a light chain variable region (VL) comprising a VLCDR1 amino acid sequence of SEQ ID NO: 160, a VLCDR2 amino acid sequence of SEQ ID NO: 5, and a VLCDR3 amino acid sequence of SEQ ID NO: 6;

(xxx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 184; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 175; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxxii) a heavy chain variable region (VH) comprising a VHCDR1 amino acid sequence of SEQ ID NO: 1, a VHCDR2 amino acid sequence of SEQ ID NO: 167, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxxiii) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxxiv) a heavy chain variable region (VH) comprising a VHCDR1 amino acid sequence of SEQ ID NO: 166, a VHCDR2 amino acid sequence of SEQ ID NO: 167, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxxv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxxvi) a heavy chain variable region (VH) comprising a VHCDR1 amino acid sequence of SEQ ID NO: 166, a VHCDR2 amino acid sequence of SEQ ID NO: 168, and a VHCDR3 amino acid sequence of SEQ ID NO: 3; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xxxvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;

(xxxviii) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;

(xxxix) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 184; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xl) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 178; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;

(xli) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;

(xlii) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;

(xliii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xliv) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;

(xlv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and/or a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 162; or

(xlvi) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 183; and/or a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

In one embodiment, provided herein are monoclonal antibodies or antibody fragments, wherein the antibodies or antibody fragments comprise a heavy chain variable region (VH) comprising a VHCDR1 amino acid sequence selected from the group consisting of SEQ ID NOs: 192-195, a VHCDR2 amino acid sequence selected from the group consisting of SEQ ID NOs: 196-211, and a VHCDR3 amino acid sequence selected from the group consisting of SEQ ID NOs: 3 and 170-185; and/or a light chain variable region (VL) comprising a VLCDR1 amino acid sequence selected from the group consisting of SEQ ID NOs: 186-190, a VLCDR2 amino acid sequence of SEQ ID NO: 191, and a VLCDR3 amino acid sequence selected from the group consisting of SEQ ID NOs: 6, 162, and 163.

In one embodiment, provided herein are monoclonal antibodies or antibody fragments, wherein the antibodies or antibody fragments comprise:

(i) a heavy chain variable region (VH) comprising a VHCDR1 amino acid sequence of SEQ ID NO: 192, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(ii) a heavy chain variable region (VH) comprising a VHCDR1 amino acid sequence of SEQ ID NO: 193, a VHCDR2 amino acid sequence of SEQ ID NO: 197, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(iii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(iv) a heavy chain variable region (VH) comprising a VHCDR1 amino acid sequence of SEQ ID NO: 193, a VHCDR2 amino acid sequence of SEQ ID NO: 198, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(v) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;

(vi) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(vii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 171; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(viii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 172; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(ix) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;

(x) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 173; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xi) a heavy chain variable region (VH) comprising a VHCDR1 amino acid sequence of SEQ ID NO: 193, a VHCDR2 amino acid sequence of SEQ ID NO: 199, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 174; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xiii) a heavy chain variable region (VH) comprising a VHCDR1 amino acid sequence of SEQ ID NO: 192, a VHCDR2 amino acid sequence of SEQ ID NO: 196, and a VHCDR3 amino acid sequence of SEQ ID NO: 175; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xiv) a heavy chain variable region (VH) comprising a VHCDR1 amino acid sequence of SEQ ID NO: 193, a VHCDR2 amino acid sequence of SEQ ID NO: 200, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xv) a heavy chain variable region (VH) comprising a VHCDR1 amino acid sequence of SEQ ID NO: 193, a VHCDR2 amino acid sequence of SEQ ID NO: 201, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xvi) a heavy chain variable region (VH) comprising a VHCDR1 amino acid sequence of SEQ ID NO: 193, a VHCDR2 amino acid sequence of SEQ ID NO: 201, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;

(xvii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 163;

(xviii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 170; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;

(xix) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 176; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xx) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 171; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;

(xxi) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 173; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxii) a heavy chain variable region (VH) comprising a VHCDR1 amino acid sequence of SEQ ID NO: 193, a VHCDR2 amino acid sequence of SEQ ID NO: 197, and a VHCDR3 amino acid sequence of SEQ ID NO: 177; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxiii) a heavy chain variable region (VH) comprising a VHCDR1 amino acid sequence of SEQ ID NO: 192, a VHCDR2 amino acid sequence of SEQ ID NO: 196, and a VHCDR3 amino acid sequence of SEQ ID NO: 178; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxiv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 179; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 179; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;

(xxvi) a heavy chain variable region (VH) comprising a VHCDR1 amino acid sequence of SEQ ID NO: 192, a VHCDR2 amino acid sequence of SEQ ID NO: 202, and a VHCDR3 amino acid sequence of SEQ ID NO: 174; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxvii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 180; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxviii) a heavy chain variable region (VH) comprising a VHCDR1 amino acid sequence of SEQ ID NO: 193, a VHCDR2 amino acid sequence of SEQ ID NO: 201, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;

(xxix) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 181; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;

(xxx) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 182; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxxi) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxxii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 163;

(xxxiii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 181; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxxiv) a heavy chain variable region (VH) comprising a VHCDR1 amino acid sequence of SEQ ID NO: 193, a VHCDR2 amino acid sequence of SEQ ID NO: 203, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxxv) a heavy chain variable region (VH) comprising a VHCDR1 amino acid sequence of SEQ ID NO: 194, a VHCDR2 amino acid sequence of SEQ ID NO: 196, and a VHCDR3 amino acid sequence of SEQ ID NO: 185; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;

(xxxvi) a heavy chain variable region (VH) comprising a VHCDR1 amino acid sequence of SEQ ID NO: 195, a VHCDR2 amino acid sequence of SEQ ID NO: 197, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxxvii) a heavy chain variable region (VH) comprising a VHCDR1 amino acid sequence of SEQ ID NO: 193, a VHCDR2 amino acid sequence of SEQ ID NO: 204, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxxviii) a heavy chain variable region (VH) comprising a VHCDR1 amino acid sequence of SEQ ID NO: 192, a VHCDR2 amino acid sequence of SEQ ID NO: 205, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xxxix) a heavy chain variable region (VH) comprising a VHCDR1 amino acid sequence of SEQ ID NO: 193, a VHCDR2 amino acid sequence of SEQ ID NO: 206, and a VHCDR3 amino acid sequence of SEQ ID NO: 171; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xl) a heavy chain variable region (VH) comprising a VHCDR1 amino acid sequence of SEQ ID NO: 194, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xli) a heavy chain variable region (VH) comprising a VHCDR1 amino acid sequence of SEQ ID NO: 193, a VHCDR2 amino acid sequence of SEQ ID NO: 207, and a VHCDR3 amino acid sequence of SEQ ID NO: 174; and/or a light chain variable region (VL) comprising a VLCDR1 amino acid sequence of SEQ ID NO: 190, a VLCDR2 amino acid sequence of SEQ ID NO: 191, and a VLCDR3 amino acid sequence of SEQ ID NO: 6;

(xlii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xliii) a heavy chain variable region (VH) comprising a VHCDR1 amino acid sequence of SEQ ID NO: 193, a VHCDR2 amino acid sequence of SEQ ID NO: 206, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xliv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 175; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xlv) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 3; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;

(xlvi) a heavy chain variable region (VH) comprising a VHCDR1 amino acid sequence of SEQ ID NO: 193, a VHCDR2 amino acid sequence of SEQ ID NO: 202, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xlvii) a heavy chain variable region (VH) comprising a VHCDR1 amino acid sequence of SEQ ID NO: 193, a VHCDR2 amino acid sequence of SEQ ID NO: 207, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xlviii) a heavy chain variable region (VH) comprising a VHCDRI amino acid sequence of SEQ ID NO: 195, a VHCDR2 amino acid sequence of SEQ ID NO: 202, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(xlix) a heavy chain variable region (VH) comprising a VHCDRI amino acid sequence of SEQ ID NO: 193, a VHCDR2 amino acid sequence of SEQ ID NO: 208, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(l) a heavy chain variable region (VH) comprising a VHCDRI amino acid sequence of SEQ ID NO: 195, a VHCDR2 amino acid sequence of SEQ ID NO: 209, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(li) a heavy chain variable region (VH) comprising a VHCDRI amino acid sequence of SEQ ID NO: 193, a VHCDR2 amino acid sequence of SEQ ID NO: 209, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;

(lii) a heavy chain variable region (VH) comprising a VHCDRI amino acid sequence of SEQ ID NO: 195, a VHCDR2 amino acid sequence of SEQ ID NO: 206, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(liii) a heavy chain variable region (VH) comprising a VHCDRI amino acid sequence of SEQ ID NO: 193, a VHCDR2 amino acid sequence of SEQ ID NO: 210, and a VHCDR3 amino acid sequence of SEQ ID NO: 178; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 163;

(liv) a heavy chain variable region (VH) comprising a VHCDR1 amino acid sequence of SEQ ID NO: 192, a VHCDR2 amino acid sequence of SEQ ID NO: 197, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 162;

(lv) a heavy chain variable region (VH) comprising a VHCDR1 amino acid sequence of SEQ ID NO: 193, a VHCDR2 amino acid sequence of SEQ ID NO: 210, and a VHCDR3 amino acid sequence of SEQ ID NO: 174; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 6;

(lvi) a heavy chain variable region (VH) comprising a VHCDR1 amino acid sequence of SEQ ID NO: 195, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;

(lvii) a heavy chain variable region (VH) comprising a VHCDR1 amino acid sequence of SEQ ID NO: 193, a VHCDR2 amino acid sequence of SEQ ID NO: 209, and a VHCDR3 amino acid sequence of SEQ ID NO: 3; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(lviii) a heavy chain variable region (VH) comprising a VHCDR1 amino acid sequence of SEQ ID NO: 193, a VHCDR2 amino acid sequence of SEQ ID NO: 196, and a VHCDR3 amino acid sequence of SEQ ID NO: 184; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 6;

(lix) a heavy chain variable region (VH) comprising a VHCDR1 amino acid sequence of SEQ ID NO: 194, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(lx) a heavy chain variable region (VH) comprising a VHCDR1 amino acid sequence of SEQ ID NO: 195, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 188, a VLCDR2 amino acid sequence of SEQ ID NO: 191, and a VLCDR3 amino acid sequence of SEQ ID NO: 6;

(lxi) a heavy chain variable region (VH) comprising a VHCDR1 amino acid sequence of SEQ ID NO: 192, a VHCDR2 amino acid sequence of SEQ ID NO: 211, and a VHCDR3 amino acid sequence of SEQ ID NO: 170; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(lxii) a heavy chain variable region (VH) comprising a VHCDR1 amino acid sequence of SEQ ID NO: 193, a VHCDR2 amino acid sequence of SEQ ID NO: 210, and a VHCDR3 amino acid sequence of SEQ ID NO: 172; and/or a light chain variable region (VL) comprising a VLCDR1 amino acid sequence of SEQ ID NO: 189, a VLCDR2 amino acid sequence of SEQ ID NO: 191, and a VLCDR3 amino acid sequence of SEQ ID NO: 6;

(lxiii) a heavy chain variable region (VH) comprising a VHCDR1 amino acid sequence of SEQ ID NO: 195, a VHCDR2 amino acid sequence of SEQ ID NO: 196, and a VHCDR3 amino acid sequence of SEQ ID NO: 183; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6;

(lxiv) a heavy chain variable region (VH) comprising a VHCDR1 amino acid sequence of SEQ ID NO: 194, a VHCDR2 amino acid sequence of SEQ ID NO: 206, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 162;

(lxv) a heavy chain variable region (VH) comprising a VHCDR1 amino acid sequence of SEQ ID NO: 193, a VHCDR2 amino acid sequence of SEQ ID NO: 208, and a VHCDR3 amino acid sequence of SEQ ID NO: 183; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6;

(lxvi) a heavy chain variable region (VH) comprising a VHCDR1 amino acid sequence of SEQ ID NO: 193, a VHCDR2 amino acid sequence of SEQ ID NO: 199, and a VHCDR3 amino acid sequence of SEQ ID NO: 3; and/or a light chain variable region (VL) comprising a VLCDR1 amino acid sequence of SEQ ID NO: 187, a VLCDR2 amino acid sequence of SEQ ID NO: 191, and a VLCDR3 amino acid sequence of SEQ ID NO: 6; or

(lxvii) a heavy chain variable region (VH) comprising a VHCDR1 amino acid sequence of SEQ ID NO: 193, a VHCDR2 amino acid sequence of SEQ ID NO: 199, and a VHCDR3 amino acid sequence of SEQ ID NO: 184; and/or a light chain variable region (VL) comprising a VLCDR1 amino acid sequence of SEQ ID NO: 186, a VLCDR2 amino acid sequence of SEQ ID NO: 191, and a VLCDR3 amino acid sequence of SEQ ID NO: 6.

In some aspects, the antibodies or antibody fragments comprise a heavy chain variable sequence having a sequence of

(SEQ ID NO: 18)
X₁X₂QLX₃X₄SGX₅X₆X₇X₈KPGX₉SX₁₀X₁₁X₁₂SCKX₁₃SGYTFTNYG
MNWVRQAPGX₁₄GLX₁₅WX₁₆GWINTYTGEPTYADDFKGRX₁₇TX₁₈X₁₉
X₂₀DX₂₁SX₂₂X₂₃TX₂₄YX₂₅X₂₆X₂₇X₂₈X₂₉LX₃₀X₃₁X₃₂DTAVYF
CARYDHAMDYWGQGTX₃₃VTVSS,

wherein X₁ is Q or E, X₂ is I or V, X₃ is V or Q, X₄ is Q or E, X₅ is A, P, or G, X₆ is E or G, X₇ is V or L, X₈ is V or K, X₉ is A, E, G, or S, X₁₀ is V or L, X₁₁ is K or R, X₁₂ is V, L, or I, X₁₃ is A or T, X₁₄ is K or Q, X₁₅ is E or K, X₁₆ is M or V, X₁₇ is F or V, X₁₈ is F, M, or I, X₁₉ is T or S, X₂₀ is T, R, or A, X₂₁ is T, D, or E, X₂₂ is T, A, or K, X₂₃ is S or N, X₂₄ is L or A, X₂₅ is M or L, X₂₆ is E or Q, X₂₇ is L or M, X₂₈ is R, S, T, or N, X₂₉ is S or G, X₃₀ is R, K, or M, X₃₁ is S or T, X₃₂ is D or E, and X₃₃ is L, S, or T;
and/or a light chain variable sequence having a sequence of

(SEQ ID NO: 26)
X₁X₂X₃X₄TQSPX₅SLX₆X₇SX₈GX₉RX₁₀TIX₁₁CKSSQSLLNSGTRKN
YLAWYQQKX₁₂GX₁₃X₁₄PX₁₅LLIYWTSTRESGVPX₁₆RFSGSGSGTDF
TLTIX₁₇X₁₈LQX₁₉EDVAX₂₀YYCKQSYTLYTFGX₂₁GTKX₂₂EIK,

wherein
X₁ is E or D, X₂ is I or V, X₃ is V or Q, X₄ is L or M, X₅ is D or S, X₆ is A or S, X₇ is V or A, X₈ is L or V, X₉ is E or D, X₁₀ is A or V, X₁₁ is N or T, X₁₂ is A or P, X₁₃ is Q or K, X₁₄ is S, V, or P, X₁₅ is K or R, X₁₆ is D or S, X₁₇ is S, D, or N, X₁₈ is S or T, X₁₉ is A or P, X₂₀ is V or T, X₂₁ is Q or G, and X₂₂ is L or V.

In some aspects, said antibodies or antibody fragments comprise a heavy chain variable sequence having a sequence of

(SEQ ID NO: 104)
QIX₁LVQSGX₂EVKKPGASVKVSCKASGYX₃ETX₄YGMNWVRQAPGQGLE
WMGWINTYTGEX₅X₆YX₇DDFKGRFTFTTDTSTX₈TX₉YMX₁₀X₁₁RSLR
SDDTAVYFCX₁₂RYDHX₁₃MDYWGQGX₁₄LVTVSS,

wherein X₁ is Q or H, X₂ is A, D, T, V, S, or P, X₃ is T, S, or I, X₄ is N or K, X₅ is P, S, T, or A, X₆ is T, R, K, or I, X₇ is A, T, V, S, or G, X₈ is S, R, or T, X₉ is A, V, or G, X₁₀ is E or D, X₁₁ is L or V, X₁₂ is A, T, V, or G, X₁₃ is A, R, F, T, P, V, S, D, N, H, L, Y, or G, and X₁₄ is T or S;
and/or a light chain variable sequence having a sequence of

(SEQ ID NO: 158)
EIVLTQSPDSLX₁VSLGERATIX₂CKSSQSLX₃NSGTRKNYLX₄WYQX₅K
X₆GQSPX₇LX₈IYWTSTRESGVPDRFSX₉SGSGTDFTLX₁₀IDX₁₁LQ
X₁₂EDVAX₁₃YYCKQSYX₁₄LYTFGGGTKVEIK,

wherein X₁ is A, T, or S, X₂ is N or K, X₃ is L, F, or V, X₄ is A, S, or T, X₅ is Q or K, X₆ is A, P, or S, X₇ is K or N, X₈ is L, V, or I, X₉ is G or A, X₁₀ is T or S, X₁₁ is S or R, X₁₂ is A or T, X₁₃ is V, I, or L, and X₁₄ is T, N, or S.

In some aspects, said antibodies or antibody fragments comprise a heavy chain variable sequence having a sequence selected from the group consisting of SEQ ID NOs: 7, 12-17, 26-103, and 225-229, or a heavy chain variable sequence having at least 70%, 80%, 90%, 95%, 96%, 97%, 98%, or 99% identity to any one of SEQ ID NOs: 7, 12-17, 26-103, and 225-229; and/or a light chain variable sequence having a sequence selected from the group consisting of SEQ ID NOs: 8, 19-24, 105-157, and 230-234, or a light chain variable sequence having at least 70%, 80%, 90%, 95%, 96%, 97%, 98%, or 99% identity to any one of SEQ ID NOs: 8, 19-24, 105-157, and 230-234.

In some aspects, said antibodies or antibody fragments comprise:

(i) a heavy chain variable sequence having a sequence according to SEQ ID NO: 7, or a heavy chain variable sequence having at least 70%, 80%, 90%, 95%, 96%, 97%, 98%, or 99% identity to SEQ ID NO: 7; and/or a light chain variable sequence having a sequence according to SEQ ID NO: 8, or a light chain variable sequence having at least 70%, 80%, 90%, 95%, 96%, 97%, 98%, or 99% identity to SEQ ID NO: 8;

(ii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;

(iii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;

(iv) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;

(v) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;

(vi) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;

(vii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;

(viii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;

(ix) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;

(x) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;

(xi) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;

(xii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;

(xiii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;

(xiv) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;

(xv) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;

(xvi) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;

(xvii) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;

(xviii) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;

(xix) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;

(xx) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;

(xxi) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;

(xxii) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;

(xxiii) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;

(xxiv) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;

(xxv) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;

(xxvi) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;

(xxvii) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;

(xxviii) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;

(xxix) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;

(xxx) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;

(xxxi) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;

(xxxii) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 19, or a light chain variable sequence having at least 70%, 80%, 90%, 95%, 96%, 97%, 98%, or 99% identity to SEQ ID NO: 19;

(xxxiii) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 20, or a light chain variable sequence having at least 70%, 80%, 90%, 95%, 96%, 97%, 98%, or 99% identity to SEQ ID NO: 20;

(xxxiv) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 21, or a light chain variable sequence having at least 70%, 80%, 90%, 95%, 96%, 97%, 98%, or 99% identity to SEQ ID NO: 21;

(xxxv) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 22, or a light chain variable sequence having at least 70%, 80%, 90%, 95%, 96%, 97%, 98%, or 99% identity to SEQ ID NO: 22;

(xxxvi) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 23, or a light chain variable sequence having at least 70%, 80%, 90%, 95%, 96%, 97%, 98%, or 99% identity to SEQ ID NO: 23;

(xxxvii) a heavy chain variable sequence having a sequence according to SEQ ID NO: 17, or a heavy chain variable sequence having at least 70%, 80%, 90%, 95%, 96%, 97%, 98%, or 99% identity to SEQ ID NO: 17; and/or a light chain variable sequence having a sequence according to SEQ ID NO: 24, or a light chain variable sequence having at least 70%, 80%, 90%, 95%, 96%, 97%, 98%, or 99% identity to SEQ ID NO: 24;

(xxxviii) a heavy chain variable sequence having a sequence according to SEQ ID NO: 26, or a heavy chain variable sequence having at least 70%, 80%, 90%, 95%, 96%, 97%, 98%, or 99% identity to SEQ ID NO: 26; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xxxix) a heavy chain variable sequence having a sequence according to SEQ ID NO: 27, or a heavy chain variable sequence having at least 70%, 80%, 90%, 95%, 96%, 97%, 98%, or 99% identity to SEQ ID NO: 27; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xl) a heavy chain variable sequence having a sequence according to SEQ ID NO: 28, or a heavy chain variable sequence having at least 70%, 80%, 90%, 95%, 96%, 97%, 98%, or 99% identity to SEQ ID NO: 28; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xli) a heavy chain variable sequence having a sequence according to SEQ ID NO: 29, or a heavy chain variable sequence having at least 70%, 80%, 90%, 95%, 96%, 97%, 98%, or 99% identity to SEQ ID NO: 29; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xlii) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 106, or a light chain variable sequence having at least 70%, 80%, 90%, 95%, 96%, 97%, 98%, or 99% identity to SEQ ID NO: 106;

(xliii) a heavy chain variable sequence having a sequence according to SEQ ID NO: 31, or a heavy chain variable sequence having at least 70%, 80%, 90%, 95%, 96%, 97%, 98%, or 99% identity to SEQ ID NO: 31; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xliv) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xlv) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 107, or a light chain variable sequence having at least 70%, 80%, 90%, 95%, 96%, 97%, 98%, or 99% identity to SEQ ID NO: 107;

(xlvi) a heavy chain variable sequence having a sequence according to SEQ ID NO: 33, or a heavy chain variable sequence having at least 70%, 80%, 90%, 95%, 96%, 97%, 98%, or 99% identity to SEQ ID NO: 33; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xlvii) a heavy chain variable sequence having a sequence according to SEQ ID NO: 34, or a heavy chain variable sequence having at least 70%, 80%, 90%, 95%, 96%, 97%, 98%, or 99% identity to SEQ ID NO: 34; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xlviii) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 108, or a light chain variable sequence having at least 70%, 80%, 90%, 95%, 96%, 97%, 98%, or 99% identity to SEQ ID NO: 108;

(xlix) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;

(l) a heavy chain variable sequence having a sequence according to SEQ ID NO: 35, or a heavy chain variable sequence having at least 70%, 80%, 90%, 95%, 96%, 97%, 98%, or 99% identity to SEQ ID NO: 35; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(li) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lii) a heavy chain variable sequence having a sequence according to SEQ ID NO: 37, or a heavy chain variable sequence having at least 70%, 80%, 90%, 95%, 96%, 97%, 98%, or 99% identity to SEQ ID NO: 37; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(liii) a heavy chain variable sequence having a sequence according to SEQ ID NO: 26, or a heavy chain variable sequence having at least 70%, 80%, 90%, 95%, 96%, 97%, 98%, or 99% identity to SEQ ID NO: 26; and/or a light chain variable sequence having a sequence according to SEQ ID NO: 107, or a light chain variable sequence having at least 70%, 80%, 90%, 95%, 96%, 97%, 98%, or 99% identity to SEQ ID NO: 107;

(liv) a heavy chain variable sequence having a sequence according to SEQ ID NO: 38, or a heavy chain variable sequence having at least 70%, 80%, 90%, 95%, 96%, 97%, 98%, or 99% identity to SEQ ID NO: 38; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lv) a heavy chain variable sequence having a sequence according to SEQ ID NO: 31, or a heavy chain variable sequence having at least 70%, 80%, 90%, 95%, 96%, 97%, 98%, or 99% identity to SEQ ID NO: 31; and/or a light chain variable sequence having a sequence according to SEQ ID NO: 110, or a light chain variable sequence having at least 70%, 80%, 90%, 95%, 96%, 97%, 98%, or 99% identity to SEQ ID NO: 110;

(lvi) a heavy chain variable sequence having a sequence according to SEQ ID NO: 39, or a heavy chain variable sequence having at least 70%, 80%, 90%, 95%, 96%, 97%, 98%, or 99% identity to SEQ ID NO: 39; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lvii) a heavy chain variable sequence having a sequence according to SEQ ID NO: 40, or a heavy chain variable sequence having at least 70%, 80%, 90%, 95%, 96%, 97%, 98%, or 99% identity to SEQ ID NO: 40; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lviii) a heavy chain variable sequence having a sequence according to SEQ ID NO: 34, or a heavy chain variable sequence having at least 70%, 80%, 90%, 95%, 96%, 97%, 98%, or 99% identity to SEQ ID NO: 34; and/or a light chain variable sequence having a sequence according to SEQ ID NO: 111, or a light chain variable sequence having at least 70%, 80%, 90%, 95%, 96%, 97%, 98%, or 99% identity to SEQ ID NO: 111;

(lix) a heavy chain variable sequence having a sequence according to SEQ ID NO: 41, or a heavy chain variable sequence having at least 70%, 80%, 90%, 95%, 96%, 97%, 98%, or 99% identity to SEQ ID NO: 41; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;

(lx) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and/or a light chain variable sequence having a sequence according to SEQ ID NO: 112, or a light chain variable sequence having at least 70%, 80%, 90%, 95%, 96%, 97%, 98%, or 99% identity to SEQ ID NO: 112;

(lxi) a heavy chain variable sequence having a sequence according to SEQ ID NO: 28, or a heavy chain variable sequence having at least 70%, 80%, 90%, 95%, 96%, 97%, 98%, or 99% identity to SEQ ID NO: 28; and/or a light chain variable sequence having a sequence according to SEQ ID NO: 113, or a light chain variable sequence having at least 70%, 80%, 90%, 95%, 96%, 97%, 98%, or 99% identity to SEQ ID NO: 113;

(lxii) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and/or a light chain variable sequence having a sequence according to SEQ ID NO: 114, or a light chain variable sequence having at least 70%, 80%, 90%, 95%, 96%, 97%, 98%, or 99% identity to SEQ ID NO: 114;

(lxiii) a heavy chain variable sequence having a sequence according to SEQ ID NO: 42, or a heavy chain variable sequence having at least 70%, 80%, 90%, 95%, 96%, 97%, 98%, or 99% identity to SEQ ID NO: 42; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lxiv) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 115, or a light chain variable sequence having at least 70%, 80%, 90%, 95%, 96%, 97%, 98%, or 99% identity to SEQ ID NO: 115;

(lxv) a heavy chain variable sequence having a sequence according to SEQ ID NO: 43, or a heavy chain variable sequence having at least 70%, 80%, 90%, 95%, 96%, 97%, 98%, or 99% identity to SEQ ID NO: 43; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lxvi) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;

(lxvii) a heavy chain variable sequence having a sequence according to SEQ ID NO: 44, or a heavy chain variable sequence having at least 70%, 80%, 90%, 95%, 96%, 97%, 98%, or 99% identity to SEQ ID NO: 44; and/or a light chain variable sequence having a sequence according to SEQ ID NO: 116, or a light chain variable sequence having at least 70%, 80%, 90%, 95%, 96%, 97%, 98%, or 99% identity to SEQ ID NO: 116;

(lxviii) a heavy chain variable sequence having a sequence according to SEQ ID NO: 35, or a heavy chain variable sequence having at least 70%, 80%, 90%, 95%, 96%, 97%, 98%, or 99% identity to SEQ ID NO: 35; and/or a light chain variable sequence having a sequence according to SEQ ID NO: 117, or a light chain variable sequence having at least 70%, 80%, 90%, 95%, 96%, 97%, 98%, or 99% identity to SEQ ID NO: 117;

(lxix) a heavy chain variable sequence having a sequence according to SEQ ID NO: 45, or a heavy chain variable sequence having at least 70%, 80%, 90%, 95%, 96%, 97%, 98%, or 99% identity to SEQ ID NO: 45; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lxx) a heavy chain variable sequence having a sequence according to SEQ ID NO: 46, or a heavy chain variable sequence having at least 70%, 80%, 90%, 95%, 96%, 97%, 98%, or 99% identity to SEQ ID NO: 46; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lxxi) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 118, or a light chain variable sequence having at least 70%, 80%, 90%, 95%, 96%, 97%, 98%, or 99% identity to SEQ ID NO: 118;

(lxxii) a heavy chain variable sequence having a sequence according to SEQ ID NO: 47, or a heavy chain variable sequence having at least 70%, 80%, 90%, 95%, 96%, 97%, 98%, or 99% identity to SEQ ID NO: 47; and/or a light chain variable sequence having a sequence according to SEQ ID NO: 115, or a light chain variable sequence having at least 70%, 80%, 90%, 95%, 96%, 97%, 98%, or 99% identity to SEQ ID NO: 115;

(lxxiii) a heavy chain variable sequence having a sequence according to SEQ ID NO: 48, or a heavy chain variable sequence having at least 70%, 80%, 90%, 95%, 96%, 97%, 98%, or 99% identity to SEQ ID NO: 48; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;

(lxxiv) a heavy chain variable sequence having a sequence according to SEQ ID NO: 49, or a heavy chain variable sequence having at least 70%, 80%, 90%, 95%, 96%, 97%, 98%, or 99% identity to SEQ ID NO: 49; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lxxv) a heavy chain variable sequence having a sequence according to SEQ ID NO: 50, or a heavy chain variable sequence having at least 70%, 80%, 90%, 95%, 96%, 97%, 98%, or 99% identity to SEQ ID NO: 50; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lxxvi) a heavy chain variable sequence having a sequence according to SEQ ID NO: 51, or a heavy chain variable sequence having at least 70%, 80%, 90%, 95%, 96%, 97%, 98%, or 99% identity to SEQ ID NO: 51; and/or a light chain variable sequence having a sequence according to SEQ ID NO: 106, or a light chain variable sequence having at least 70%, 80%, 90%, 95%, 96%, 97%, 98%, or 99% identity to SEQ ID NO: 106;

(lxxvii) a heavy chain variable sequence having a sequence according to SEQ ID NO: 52, or a heavy chain variable sequence having at least 70%, 80%, 90%, 95%, 96%, 97%, 98%, or 99% identity to SEQ ID NO: 52; and/or a light chain variable sequence having a sequence according to SEQ ID NO: 119, or a light chain variable sequence having at least 70%, 80%, 90%, 95%, 96%, 97%, 98%, or 99% identity to SEQ ID NO: 119;

(lxxviii) a heavy chain variable sequence having a sequence according to SEQ ID NO: 53, or a heavy chain variable sequence having at least 70%, 80%, 90%, 95%, 96%, 97%, 98%, or 99% identity to SEQ ID NO: 53; and/or a light chain variable sequence having a sequence according to SEQ ID NO: 108, or a light chain variable sequence having at least 70%, 80%, 90%, 95%, 96%, 97%, 98%, or 99% identity to SEQ ID NO: 108;

(lxxix) a heavy chain variable sequence having a sequence according to SEQ ID NO: 54, or a heavy chain variable sequence having at least 70%, 80%, 90%, 95%, 96%, 97%, 98%, or 99% identity to SEQ ID NO: 54; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(lxxx) a heavy chain variable sequence having a sequence according to SEQ ID NO: 55, or a heavy chain variable sequence having at least 70%, 80%, 90%, 95%, 96%, 97%, 98%, or 99% identity to SEQ ID NO: 55; and/or a light chain variable sequence having a sequence according to SEQ ID NO: 116, or a light chain variable sequence having at least 70%, 80%, 90%, 95%, 96%, 97%, 98%, or 99% identity to SEQ ID NO: 116;

(lxxxi) a heavy chain variable sequence having a sequence according to SEQ ID NO: 56, or a heavy chain variable sequence having at least 70%, 80%, 90%, 95%, 96%, 97%, 98%, or 99% identity to SEQ ID NO: 56; and/or a light chain variable sequence having a sequence according to SEQ ID NO: 116, or a light chain variable sequence having at least 70%, 80%, 90%, 95%, 96%, 97%, 98%, or 99% identity to SEQ ID NO: 116;

(lxxxii) a heavy chain variable sequence having a sequence according to SEQ ID NO: 57, or a heavy chain variable sequence having at least 70%, 80%, 90%, 95%, 96%, 97%, 98%, or 99% identity to SEQ ID NO: 57; and/or a light chain variable sequence having a sequence according to SEQ ID NO: 120, or a light chain variable sequence having at least 70%, 80%, 90%, 95%, 96%, 97%, 98%, or 99% identity to SEQ ID NO: 120;

(lxxxiii) a heavy chain variable sequence having a sequence according to SEQ ID NO: 58, or a heavy chain variable sequence having at least 70%, 80%, 90%, 95%, 96%, 97%, 98%, or 99% identity to SEQ ID NO: 58; and/or a light chain variable sequence having a sequence according to SEQ ID NO: 121, or a light chain variable sequence having at least 70%, 80%, 90%, 95%, 96%, 97%, 98%, or 99% identity to SEQ ID NO: 121;

(lxxxiv) a heavy chain variable sequence having a sequence according to SEQ ID NO: 59, or a heavy chain variable sequence having at least 70%, 80%, 90%, 95%, 96%, 97%, 98%, or 99% identity to SEQ ID NO: 59; and/or a light chain variable sequence having a sequence according to SEQ ID NO: 122, or a light chain variable sequence having at least 70%, 80%, 90%, 95%, 96%, 97%, 98%, or 99% identity to SEQ ID NO: 122;

(lxxxv) a heavy chain variable sequence having a sequence according to SEQ ID NO: 60, or a heavy chain variable sequence having at least 70%, 80%, 90%, 95%, 96%, 97%, 98%, or 99% identity to SEQ ID NO: 60; and/or a light chain variable sequence having a sequence according to SEQ ID NO: 108, or a light chain variable sequence having at least 70%, 80%, 90%, 95%, 96%, 97%, 98%, or 99% identity to SEQ ID NO: 108;

(lxxxvi) a heavy chain variable sequence having a sequence according to SEQ ID NO: 61, or a heavy chain variable sequence having at least 70%, 80%, 90%, 95%, 96%, 97%, 98%, or 99% identity to SEQ ID NO: 61; and/or a light chain variable sequence having a sequence according to SEQ ID NO: 123, or a light chain variable sequence having at least 70%, 80%, 90%, 95%, 96%, 97%, 98%, or 99% identity to SEQ ID NO: 123;

(lxxxvii) a heavy chain variable sequence having a sequence according to SEQ ID NO: 62, or a heavy chain variable sequence having at least 70%, 80%, 90%, 95%, 96%, 97%, 98%, or 99% identity to SEQ ID NO: 62; and/or a light chain variable sequence having a sequence according to SEQ ID NO: 114, or a light chain variable sequence having at least 70%, 80%, 90%, 95%, 96%, 97%, 98%, or 99% identity to SEQ ID NO: 114;

(lxxxviii) a heavy chain variable sequence having a sequence according to SEQ ID NO: 63, or a heavy chain variable sequence having at least 70%, 80%, 90%, 95%, 96%, 97%, 98%, or 99% identity to SEQ ID NO: 63; and/or a light chain variable sequence having a sequence according to SEQ ID NO: 124, or a light chain variable sequence having at least 70%, 80%, 90%, 95%, 96%, 97%, 98%, or 99% identity to SEQ ID NO: 124;

(lxxxix) a heavy chain variable sequence having a sequence according to SEQ ID NO: 64, or a heavy chain variable sequence having at least 70%, 80%, 90%, 95%, 96%, 97%, 98%, or 99% identity to SEQ ID NO: 64; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xc) a heavy chain variable sequence having a sequence according to SEQ ID NO: 65, or a heavy chain variable sequence having at least 70%, 80%, 90%, 95%, 96%, 97%, 98%, or 99% identity to SEQ ID NO: 65; and/or a light chain variable sequence having a sequence according to SEQ ID NO: 125, or a light chain variable sequence having at least 70%, 80%, 90%, 95%, 96%, 97%, 98%, or 99% identity to SEQ ID NO: 125;

(xci) a heavy chain variable sequence having a sequence according to SEQ ID NO: 66, or a heavy chain variable sequence having at least 70%, 80%, 90%, 95%, 96%, 97%, 98%, or 99% identity to SEQ ID NO: 66; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(xcii) a heavy chain variable sequence having a sequence according to SEQ ID NO: 67, or a heavy chain variable sequence having at least 70%, 80%, 90%, 95%, 96%, 97%, 98%, or 99% identity to SEQ ID NO: 67; and/or a light chain variable sequence having a sequence according to SEQ ID NO: 125, or a light chain variable sequence having at least 70%, 80%, 90%, 95%, 96%, 97%, 98%, or 99% identity to SEQ ID NO: 125;

(xciii) a heavy chain variable sequence having a sequence according to SEQ ID NO: 68, or a heavy chain variable sequence having at least 70%, 80%, 90%, 95%, 96%, 97%, 98%, or 99% identity to SEQ ID NO: 68; and/or a light chain variable sequence having a sequence according to SEQ ID NO: 126, or a light chain variable sequence having at least 70%, 80%, 90%, 95%, 96%, 97%, 98%, or 99% identity to SEQ ID NO: 126;

(xciv) a heavy chain variable sequence having a sequence according to SEQ ID NO: 69, or a heavy chain variable sequence having at least 70%, 80%, 90%, 95%, 96%, 97%, 98%, or 99% identity to SEQ ID NO: 69; and/or a light chain variable sequence having a sequence according to SEQ ID NO: 127, or a light chain variable sequence having at least 70%, 80%, 90%, 95%, 96%, 97%, 98%, or 99% identity to SEQ ID NO: 127;

(xcv) a heavy chain variable sequence having a sequence according to SEQ ID NO: 70, or a heavy chain variable sequence having at least 70%, 80%, 90%, 95%, 96%, 97%, 98%, or 99% identity to SEQ ID NO: 70; and/or a light chain variable sequence having a sequence according to SEQ ID NO: 128, or a light chain variable sequence having at least 70%, 80%, 90%, 95%, 96%, 97%, 98%, or 99% identity to SEQ ID NO: 128;

(xcvi) a heavy chain variable sequence having a sequence according to SEQ ID NO: 71, or a heavy chain variable sequence having at least 70%, 80%, 90%, 95%, 96%, 97%, 98%, or 99% identity to SEQ ID NO: 71; and/or a light chain variable sequence having a sequence according to SEQ ID NO: 117, or a light chain variable sequence having at least 70%, 80%, 90%, 95%, 96%, 97%, 98%, or 99% identity to SEQ ID NO: 117;

(xcvii) a heavy chain variable sequence having a sequence according to SEQ ID NO: 72, or a heavy chain variable sequence having at least 70%, 80%, 90%, 95%, 96%, 97%, 98%, or 99% identity to SEQ ID NO: 72; and/or a light chain variable sequence having a sequence according to SEQ ID NO: 129, or a light chain variable sequence having at least 70%, 80%, 90%, 95%, 96%, 97%, 98%, or 99% identity to SEQ ID NO: 129;

(xcviii) a heavy chain variable sequence having a sequence according to SEQ ID NO: 73, or a heavy chain variable sequence having at least 70%, 80%, 90%, 95%, 96%, 97%, 98%, or 99% identity to SEQ ID NO: 73; and/or a light chain variable sequence having a sequence according to SEQ ID NO: 130, or a light chain variable sequence having at least 70%, 80%, 90%, 95%, 96%, 97%, 98%, or 99% identity to SEQ ID NO: 130;

(xcix) a heavy chain variable sequence having a sequence according to SEQ ID NO: 74, or a heavy chain variable sequence having at least 70%, 80%, 90%, 95%, 96%, 97%, 98%, or 99% identity to SEQ ID NO: 74; and/or a light chain variable sequence having a sequence according to SEQ ID NO: 131, or a light chain variable sequence having at least 70%, 80%, 90%, 95%, 96%, 97%, 98%, or 99% identity to SEQ ID NO: 131;

(c) a heavy chain variable sequence having a sequence according to SEQ ID NO: 73, or a heavy chain variable sequence having at least 70%, 80%, 90%, 95%, 96%, 97%, 98%, or 99% identity to SEQ ID NO: 73; and/or a light chain variable sequence having a sequence according to SEQ ID NO: 132, or a light chain variable sequence having at least 70%, 80%, 90%, 95%, 96%, 97%, 98%, or 99% identity to SEQ ID NO: 132;

(ci) a heavy chain variable sequence having a sequence according to SEQ ID NO: 75, or a heavy chain variable sequence having at least 70%, 80%, 90%, 95%, 96%, 97%, 98%, or 99% identity to SEQ ID NO: 75; and/or a light chain variable sequence having a sequence according to SEQ ID NO: 133, or a light chain variable sequence having at least 70%, 80%, 90%, 95%, 96%, 97%, 98%, or 99% identity to SEQ ID NO: 133;

(cii) a heavy chain variable sequence having a sequence according to SEQ ID NO: 76, or a heavy chain variable sequence having at least 70%, 80%, 90%, 95%, 96%, 97%, 98%, or 99% identity to SEQ ID NO: 76; and/or a light chain variable sequence having a sequence according to SEQ ID NO: 134, or a light chain variable sequence having at least 70%, 80%, 90%, 95%, 96%, 97%, 98%, or 99% identity to SEQ ID NO: 134;

(ciii) a heavy chain variable sequence having a sequence according to SEQ ID NO: 77, or a heavy chain variable sequence having at least 70%, 80%, 90%, 95%, 96%, 97%, 98%, or 99% identity to SEQ ID NO: 77; and/or a light chain variable sequence having a sequence according to SEQ ID NO: 107, or a light chain variable sequence having at least 70%, 80%, 90%, 95%, 96%, 97%, 98%, or 99% identity to SEQ ID NO: 107;

(civ) a heavy chain variable sequence having a sequence according to SEQ ID NO: 78, or a heavy chain variable sequence having at least 70%, 80%, 90%, 95%, 96%, 97%, 98%, or 99% identity to SEQ ID NO: 78; and/or a light chain variable sequence having a sequence according to SEQ ID NO: 135, or a light chain variable sequence having at least 70%, 80%, 90%, 95%, 96%, 97%, 98%, or 99% identity to SEQ ID NO: 135;

(cv) a heavy chain variable sequence having a sequence according to SEQ ID NO: 79, or a heavy chain variable sequence having at least 70%, 80%, 90%, 95%, 96%, 97%, 98%, or 99% identity to SEQ ID NO: 79; and/or a light chain variable sequence having a sequence according to SEQ ID NO: 136, or a light chain variable sequence having at least 70%, 80%, 90%, 95%, 96%, 97%, 98%, or 99% identity to SEQ ID NO: 136;

(cvi) a heavy chain variable sequence having a sequence according to SEQ ID NO: 80, or a heavy chain variable sequence having at least 70%, 80%, 90%, 95%, 96%, 97%, 98%, or 99% identity to SEQ ID NO: 80; and/or a light chain variable sequence having a sequence according to SEQ ID NO: 137, or a light chain variable sequence having at least 70%, 80%, 90%, 95%, 96%, 97%, 98%, or 99% identity to SEQ ID NO: 137;

(cvii) a heavy chain variable sequence having a sequence according to SEQ ID NO: 41, or a heavy chain variable sequence having at least 70%, 80%, 90%, 95%, 96%, 97%, 98%, or 99% identity to SEQ ID NO: 41; and/or a light chain variable sequence having a sequence according to SEQ ID NO: 138, or a light chain variable sequence having at least 70%, 80%, 90%, 95%, 96%, 97%, 98%, or 99% identity to SEQ ID NO: 138;

(cviii) a heavy chain variable sequence having a sequence according to SEQ ID NO: 81, or a heavy chain variable sequence having at least 70%, 80%, 90%, 95%, 96%, 97%, 98%, or 99% identity to SEQ ID NO: 31; and/or a light chain variable sequence having a sequence according to SEQ ID NO: 139, or a light chain variable sequence having at least 70%, 80%, 90%, 95%, 96%, 97%, 98%, or 99% identity to SEQ ID NO: 139;

(cix) a heavy chain variable sequence having a sequence according to SEQ ID NO: 82, or a heavy chain variable sequence having at least 70%, 80%, 90%, 95%, 96%, 97%, 98%, or 99% identity to SEQ ID NO: 82; and/or a light chain variable sequence having a sequence according to SEQ ID NO: 105, or a light chain variable sequence having at least 70%, 80%, 90%, 95%, 96%, 97%, 98%, or 99% identity to SEQ ID NO: 105;

(cx) a heavy chain variable sequence having a sequence according to SEQ ID NO: 83, or a heavy chain variable sequence having at least 70%, 80%, 90%, 95%, 96%, 97%, 98%, or 99% identity to SEQ ID NO: 83; and/or a light chain variable sequence having a sequence according to SEQ ID NO: 126, or a light chain variable sequence having at least 70%, 80%, 90%, 95%, 96%, 97%, 98%, or 99% identity to SEQ ID NO: 126;

(cxi) a heavy chain variable sequence having a sequence according to SEQ ID NO: 84, or a heavy chain variable sequence having at least 70%, 80%, 90%, 95%, 96%, 97%, 98%, or 99% identity to SEQ ID NO: 84; and/or a light chain variable sequence having a sequence according to SEQ ID NO: 140, or a light chain variable sequence having at least 70%, 80%, 90%, 95%, 96%, 97%, 98%, or 99% identity to SEQ ID NO: 140;

(cxii) a heavy chain variable sequence having a sequence according to SEQ ID NO: 85, or a heavy chain variable sequence having at least 70%, 80%, 90%, 95%, 96%, 97%, 98%, or 99% identity to SEQ ID NO: 85; and/or a light chain variable sequence having a sequence according to SEQ ID NO: 141, or a light chain variable sequence having at least 70%, 80%, 90%, 95%, 96%, 97%, 98%, or 99% identity to SEQ ID NO: 141;

(cxiii) a heavy chain variable sequence having a sequence according to SEQ ID NO: 86, or a heavy chain variable sequence having at least 70%, 80%, 90%, 95%, 96%, 97%, 98%, or 99% identity to SEQ ID NO: 86; and/or a light chain variable sequence having a sequence according to SEQ ID NO: 141, or a light chain variable sequence having at least 70%, 80%, 90%, 95%, 96%, 97%, 98%, or 99% identity to SEQ ID NO: 141;

(cxiv) a heavy chain variable sequence having a sequence according to SEQ ID NO: 87, or a heavy chain variable sequence having at least 70%, 80%, 90%, 95%, 96%, 97%, 98%, or 99% identity to SEQ ID NO: 87; and/or a light chain variable sequence having a sequence according to SEQ ID NO: 117, or a light chain variable sequence having at least 70%, 80%, 90%, 95%, 96%, 97%, 98%, or 99% identity to SEQ ID NO: 117;

(cxv) a heavy chain variable sequence having a sequence according to SEQ ID NO: 88, or a heavy chain variable sequence having at least 70%, 80%, 90%, 95%, 96%, 97%, 98%, or 99% identity to SEQ ID NO: 88; and/or a light chain variable sequence having a sequence according to SEQ ID NO: 142, or a light chain variable sequence having at least 70%, 80%, 90%, 95%, 96%, 97%, 98%, or 99% identity to SEQ ID NO: 142;

(cxvi) a heavy chain variable sequence having a sequence according to SEQ ID NO: 89, or a heavy chain variable sequence having at least 70%, 80%, 90%, 95%, 96%, 97%, 98%, or 99% identity to SEQ ID NO: 89; and/or a light chain variable sequence having a sequence according to SEQ ID NO: 143, or a light chain variable sequence having at least 70%, 80%, 90%, 95%, 96%, 97%, 98%, or 99% identity to SEQ ID NO: 143;

(cxvii) a heavy chain variable sequence having a sequence according to SEQ ID NO: 90, or a heavy chain variable sequence having at least 70%, 80%, 90%, 95%, 96%, 97%, 98%, or 99% identity to SEQ ID NO: 90; and/or a light chain variable sequence having a sequence according to SEQ ID NO: 144, or a light chain variable sequence having at least 70%, 80%, 90%, 95%, 96%, 97%, 98%, or 99% identity to SEQ ID NO: 144;

(cxviii) a heavy chain variable sequence having a sequence according to SEQ ID NO: 91, or a heavy chain variable sequence having at least 70%, 80%, 90%, 95%, 96%, 97%, 98%, or 99% identity to SEQ ID NO: 91; and/or a light chain variable sequence having a sequence according to SEQ ID NO: 109, or a light chain variable sequence having at least 70%, 80%, 90%, 95%, 96%, 97%, 98%, or 99% identity to SEQ ID NO: 109;

(cxix) a heavy chain variable sequence having a sequence according to SEQ ID NO: 92, or a heavy chain variable sequence having at least 70%, 80%, 90%, 95%, 96%, 97%, 98%, or 99% identity to SEQ ID NO: 92; and/or a light chain variable sequence having a sequence according to SEQ ID NO: 145, or a light chain variable sequence having at least 70%, 80%, 90%, 95%, 96%, 97%, 98%, or 99% identity to SEQ ID NO: 145;

(cxx) a heavy chain variable sequence having a sequence according to SEQ ID NO: 93, or a heavy chain variable sequence having at least 70%, 80%, 90%, 95%, 96%, 97%, 98%, or 99% identity to SEQ ID NO: 93; and/or a light chain variable sequence having a sequence according to SEQ ID NO: 146, or a light chain variable sequence having at least 70%, 80%, 90%, 95%, 96%, 97%, 98%, or 99% identity to SEQ ID NO: 146;

(cxxi) a heavy chain variable sequence having a sequence according to SEQ ID NO: 94, or a heavy chain variable sequence having at least 70%, 80%, 90%, 95%, 96%, 97%, 98%, or 99% identity to SEQ ID NO: 94; and/or a light chain variable sequence having a sequence according to SEQ ID NO: 147, or a light chain variable sequence having at least 70%, 80%, 90%, 95%, 96%, 97%, 98%, or 99% identity to SEQ ID NO: 147;

(cxxii) a heavy chain variable sequence having a sequence according to SEQ ID NO: 95, or a heavy chain variable sequence having at least 70%, 80%, 90%, 95%, 96%, 97%, 98%, or 99% identity to SEQ ID NO: 95; and/or a light chain variable sequence having a sequence according to SEQ ID NO: 148, or a light chain variable sequence having at least 70%, 80%, 90%, 95%, 96%, 97%, 98%, or 99% identity to SEQ ID NO: 148;

(cxxiii) a heavy chain variable sequence having a sequence according to SEQ ID NO: 96, or a heavy chain variable sequence having at least 70%, 80%, 90%, 95%, 96%, 97%, 98%, or 99% identity to SEQ ID NO: 96; and/or a light chain variable sequence having a sequence according to SEQ ID NO: 149, or a light chain variable sequence having at least 70%, 80%, 90%, 95%, 96%, 97%, 98%, or 99% identity to SEQ ID NO: 149;

(cxxiv) a heavy chain variable sequence having a sequence according to SEQ ID NO: 97, or a heavy chain variable sequence having at least 70%, 80%, 90%, 95%, 96%, 97%, 98%, or 99% identity to SEQ ID NO: 97; and/or a light chain variable sequence having a sequence according to SEQ ID NO: 150, or a light chain variable sequence having at least 70%, 80%, 90%, 95%, 96%, 97%, 98%, or 99% identity to SEQ ID NO: 150;

(cxxv) a heavy chain variable sequence having a sequence according to SEQ ID NO: 98, or a heavy chain variable sequence having at least 70%, 80%, 90%, 95%, 96%, 97%, 98%, or 99% identity to SEQ ID NO: 98; and/or a light chain variable sequence having a sequence according to SEQ ID NO: 151, or a light chain variable sequence having at least 70%, 80%, 90%, 95%, 96%, 97%, 98%, or 99% identity to SEQ ID NO: 151;

(cxxvi) a heavy chain variable sequence having a sequence according to SEQ ID NO: 99, or a heavy chain variable sequence having at least 70%, 80%, 90%, 95%, 96%, 97%, 98%, or 99% identity to SEQ ID NO: 99; and/or a light chain variable sequence having a sequence according to SEQ ID NO: 152, or a light chain variable sequence having at least 70%, 80%, 90%, 95%, 96%, 97%, 98%, or 99% identity to SEQ ID NO: 152;

(cxxvii) a heavy chain variable sequence having a sequence according to SEQ ID NO: 100, or a heavy chain variable sequence having at least 70%, 80%, 90%, 95%, 96%, 97%, 98%, or 99% identity to SEQ ID NO: 100; and/or a light chain variable sequence having a sequence according to SEQ ID NO: 136, or a light chain variable sequence having at least 70%, 80%, 90%, 95%, 96%, 97%, 98%, or 99% identity to SEQ ID NO: 136;

(cxxviii) a heavy chain variable sequence having a sequence according to SEQ ID NO: 91, or a heavy chain variable sequence having at least 70%, 80%, 90%, 95%, 96%, 97%, 98%, or 99% identity to SEQ ID NO: 91; and/or a light chain variable sequence having a sequence according to SEQ ID NO: 153, or a light chain variable sequence having at least 70%, 80%, 90%, 95%, 96%, 97%, 98%, or 99% identity to SEQ ID NO: 153;

(cxxix) a heavy chain variable sequence having a sequence according to SEQ ID NO: 101, or a heavy chain variable sequence having at least 70%, 80%, 90%, 95%, 96%, 97%, 98%, or 99% identity to SEQ ID NO: 101; and/or a light chain variable sequence having a sequence according to SEQ ID NO: 154, or a light chain variable sequence having at least 70%, 80%, 90%, 95%, 96%, 97%, 98%, or 99% identity to SEQ ID NO: 154;

(cxxx) a heavy chain variable sequence having a sequence according to SEQ ID NO: 102, or a heavy chain variable sequence having at least 70%, 80%, 90%, 95%, 96%, 97%, 98%, or 99% identity to SEQ ID NO: 102; and/or a light chain variable sequence having a sequence according to SEQ ID NO: 155, or a light chain variable sequence having at least 70%, 80%, 90%, 95%, 96%, 97%, 98%, or 99% identity to SEQ ID NO: 155;

(cxxxi) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and/or a light chain variable sequence having a sequence according to SEQ ID NO: 156, or a light chain variable sequence having at least 70%, 80%, 90%, 95%, 96%, 97%, 98%, or 99% identity to SEQ ID NO: 156; or

(cxxxii) a heavy chain variable sequence having a sequence according to SEQ ID NO: 103, or a heavy chain variable sequence having at least 70%, 80%, 90%, 95%, 96%, 97%, 98%, or 99% identity to SEQ ID NO: 103; and/or a light chain variable sequence having a sequence according to SEQ ID NO: 157, or a light chain variable sequence having at least 70%, 80%, 90%, 95%, 96%, 97%, 98%, or 99% identity to SEQ ID NO: 157.

In one embodiment, provided herein are monoclonal antibodies or antibody fragments, which compete for binding to the same epitope on HSP70 as the monoclonal antibodies or antibody fragments according to any one of the present embodiments. In one embodiment, provided herein are monoclonal antibodies or antibody fragments that binds, or is capable of binding, to an epitope on HSP70 recognized by an antibody or antibody fragment of any one of the present embodiments.

In one embodiment, provided herein are monoclonal antibodies or antibody fragments, wherein the monoclonal antibodies or antibody fragments bind to an epitope of HSP70 defined by a peptide corresponding to K573-Q601 of SEQ ID NO: 11. In some aspects, when bound to HSP70, the monoclonal antibodies or antibody fragments bind to one or two of the following residues: H594, K595, and Q601 of SEQ ID NO:11. In some aspects, when bound to HSP70, the monoclonal antibodies or antibody fragments bind to all of the following residues: H594, K595, and Q601 of SEQ ID NO:11. In some aspects, when bound to HSP70, the monoclonal antibodies or antibody fragments additionally bind to at least one of the following residues: K573, E576, W580, R596, and E598 of SEQ ID NO:11. In some aspects, when bound to HSP70, the monoclonal antibodies or antibody fragments additionally bind to at least two, three, four, or five of the following residues: K573, E576, W580, R596, and E598 of SEQ ID NO:11. In some aspects, when bound to HSP70, the monoclonal antibodies or antibody fragments bind to all of the following residues: K573, E576, W580, H594, K595, R596, E598, and Q601 of SEQ ID NO:11.

In some aspects of any of the present embodiments, the antibodies bind, or are capable of binding, to HSP70. In some aspects of any of the present embodiments, the antibodies bind, or are capable of binding, to HSP70 in its ADP-bound form. In some aspects of any of the present embodiments, the antibodies bind, or are capable of binding, to HSP70 in its peptide-bound form. In some aspects of any of the present embodiments, the antibodies bind, or are capable of binding, to HSP70 in its ADP-bound and peptide-bound form. In some aspects of any of the present embodiments, the antibodies do not induce antibody-dependent cellular cytotoxicity. In some aspects of any of the present embodiments, the antibodies do not induce complement-dependent cellular cytotoxicity. In some aspects of any of the present embodiments, the antibodies enhance HSP70 uptake by immune effector cells, such as, for example, monocytes/macrophages and dendritic cells. In some aspects, the uptake is mediated by human FcγR2A and/or human FcγR2B.

In some aspects, the antibodies bind, or are capable of binding, to HSP70. In some aspects, the antibodies bind to human HSP70 (e.g., HSP70 in its ADP-bound and/or peptide-bound form) with a K_{D} less than about 20, 10, 9, 8, 7, 6, 5, 4, 3, 2, 1, 0.9, 0.85, 0.8, 0.75, 0.7, 0.6, 0.5, 0.1, 0.05 nM, or 20, 10, 9, 8, 7, 6, 5, 4, 3, 2, 1, 0.9, 0.85, 0.8, 0.75, 0.7, 0.6, 0.5, 0.1, or 0.05 pM, as determined by Octet bio-layer interferometry (BLI) analysis. In some aspects, the antibodies bind to human HSP70 (e.g., HSP70 in its ADP-bound and/or peptide-bound form) with a K_{D} of from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, as determined by Octet bio-layer interferometry (BLI) analysis. In some aspects, the antibodies bind to human HSP70 (e.g., HSP70 in its ADP-bound and/or peptide-bound form) with a K_{D} of from about 20 pM to about 0.05 pM, from about 20 pM to about 0.075 pM, from about 20 pM to about 0.1 pM, from about 20 pM to about 0.5 pM, from about 20 pM to about 1 pM, from about 10 pM to about 0.05 pM, from about 10 pM to about 0.075 pM, from about 10 pM to about 0.1 pM, from about 10 pM to about 0.5 pM, from about 10 pM to about 1 pM, from about 5 pM to about 0.05 pM, from about 5 pM to about 0.075 pM, from about 5 pM to about 0.1 pM, from about 5 pM to about 0.5 pM, from about 5 pM to about 1 pM, from about 3 pM to about 0.05 pM, from about 3 pM to about 0.075 pM, from about 3 pM to about 0.1 pM, from about 3 pM to about 0.5 pM, from about 3 pM to about 1 pM, from about 3 pM to about 2 pM, from about 2 pM to about 0.05 pM, from about 2 pM to about 0.075 pM, from about 2 pM to about 0.1 pM, from about 2 pM to about 0.5 pM, from about 2 pM to about 1 pM, from about 1 pM to about 0.05 pM, from about 1 pM to about 0.075 pM, from about 1 pM to about 0.1 pM, from about 1 pM to about 0.5 pM, from about 0.5 pM to about 0.05 pM, from about 0.5 pM to about 0.075 pM, from about 0.5 pM to about 0.1 pM, from about 0.1 pM to about 0.05 pM, from about 0.1 pM to about 0.075 pM, or from about 0.075 pM to about 0.05 pM, as determined by Octet bio-layer interferometry (BLI) analysis.

In some aspects, it is contemplated that a heavy chain variable region sequence, for example, the VH sequence of any one of SEQ ID NOs: 7, 12-17, and 26-103, or any variants thereof, may be covalently linked to a variety of heavy chain constant region sequences known in the art. Similarly, it is contemplated that a light chain variable region sequence, for example, the VL of any one of SEQ ID NOs: 8, 19-24, and 105-157, or any variants thereof, may be covalently linked to a variety of light chain constant region sequences known in the art.

For example, an antibody or antibody fragment may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody or antibody fragment has a light chain constant region chosen from, e.g., the (e.g., human) light chain constant regions of kappa or lambda. The constant region can be altered, e.g., mutated, to modify the properties of the antibody or antibody fragment (e.g., to increase or decrease one or more of: Fc receptor binding, antibody glycosylation, the number of cysteine residues, effector cell function, and/or complement function). In one embodiment the antibody or antibody fragment has effector function and can fix complement. In other embodiments the antibody or antibody fragment does not recruit effector cells or fix complement. In another embodiment, the antibody or antibody fragment has reduced or no ability to bind an Fc receptor. For example, it is an isotype or subtype, fragment or other mutant, which does not support binding to an Fc receptor, e.g., it has a mutagenized or deleted Fc receptor binding region.

In some aspects, the constant region of the heavy chain of the antibody or antibody fragment is a human IgG1 isotype, having an amino acid sequence:

(SEQ ID NO: 217)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK

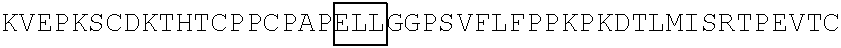

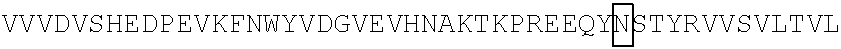

HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK.

In some aspects, the human IgG1 constant region is modified at amino acid Asn297 (boxed in SEQ ID NO: 217 in the preceding paragraph) to prevent to glycosylation of the antibody, for example Asn297Ala (N297A). In some aspects, the constant region of the antibody is modified at amino acid Leu235 (boxed in SEQ ID NO: 217 in the preceding paragraph) to alter Fc receptor interactions, for example Leu235Glu (L235E) or Leu235Ala (L235A). In some aspects, the constant region of the antibody is modified at amino acid Leu234 (boxed in SEQ ID NO: 217 in the preceding paragraph) to alter Fc receptor interactions, e.g., Leu234Ala (L234A). In some aspects, the constant region of the antibody is modified at amino acid Glu233 (boxed in SEQ ID NO: 217 in the preceding paragraph), e.g., Glu233Pro (E233P). In some aspects, the constant region of the antibody is altered at both amino acid 234 and 235, for example Leu234Ala and Leu235Ala (L234A/L235A). In some aspects, the constant region of the antibody is altered at amino acids 233, 234, and 234, for example, Glu233Pro, Leu234Ala, and Leu235Ala (E233P L234A/L235A) (Armour K L. et al. (1999) EUR. J. IMMUNOL. 29(8):2613-24). All residue numbers are according to EU numbering (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

In some aspects, the constant region of the heavy chain of the antibody is a human IgG1 isotype, having an amino acid sequence:

(SEQ ID NO: 218)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK

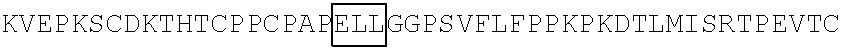

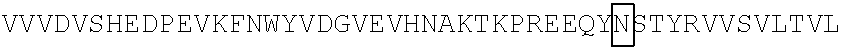

HQDWLNGKEYKCKVSNKALPAPIEKTISKAKGQPREPQVYTLPPSREE
MTKNQVSLTCLVKGFYPSDIAVEWESNGQPENNYKTTPPVLDSDGSFF
LYSKLTVDKSRWQQGNVFSCSVMHEALHNHYTQKSLSLSPGK.

In some aspects, the human IgG1 constant region is modified at amino acid Asn297 (boxed in SEQ ID NO: 218 in the preceding paragraph) to prevent to glycosylation of the antibody, for example Asn297Ala (N297A). In some aspects, the constant region of the antibody is modified at amino acid Leu235 (boxed in SEQ ID NO: 218 in the preceding paragraph) to alter Fc receptor interactions, for example Leu235Glu (L235E) or Leu235Ala (L235A). In some aspects, the constant region of the antibody is modified at amino acid Leu234 (boxed in SEQ ID NO: 218 in the preceding paragraph) to alter Fc receptor interactions, e.g., Leu234Ala (L234A). In some aspects, the constant region of the antibody is modified at amino acid Glu233 (boxed in SEQ ID NO: 218 in the preceding paragraph), e.g., Glu233Pro (E233P). In some aspects, the constant region of the antibody is altered at both amino acid 234 and 235, for example Leu234Ala and Leu235Ala (L234A/L235A). In some aspects, the constant region of the antibody is altered at amino acids 233, 234, and 234, for example, Glu233Pro, Leu234Ala, and Leu235Ala (E233P L234A/L235A) (Armour K L. et al. (1999) EUR. J. IMMUNOL. 29(8):2613-24). All residue numbers are according to EU numbering (Kabat, E. A., et al., supra).

In some aspects, the human IgG1 constant region is modified to comprise either a “knob” mutation, e.g., T366Y, or a “hole” mutation, e.g., Y407T, for heterodimerization with a second constant region (residue numbers according to EU numbering (Kabat, E. A., et al., supra)).

In some aspects, the constant region of the heavy chain of the antibody is a human IgG1 isotype, e.g., an allotype of the human IgG1 isotype, e.g., the IgG1 G1m3 allotype. Exemplary human IgG1 allotypes are described in Magdelaine-Beuzelin et al. (2009) PHARMACOGENET. GENOMICS 19(5):383-7.

In some aspects, the constant region of the heavy chain of the antibody is a human IgG2 isotype, having an amino acid sequence:

(SEQ ID NO: 219)
ASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDK
TVERKCCVECPPCPAPPVAGPSVFLFPPKPKDTLMISRTPEVTCVVVD

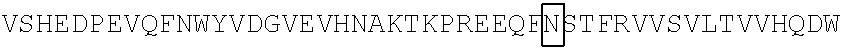

LNGKEYKCKVSNKGLPAPIEKTISKTKGQPREPQVYTLPPSREEMTKN
QVSLTCLVKGFYPSDISVEWESNGQPENNYKTTPPMLDSDGSFFLYSK
LTVDKSRWQQGNVFSCSVMHEALHNHYTQKSLSLSPGK.

In some aspects, the human IgG2 constant region is modified at amino acid Asn297 (boxed in SEQ ID NO: 219 in the preceding paragraph) to prevent to glycosylation of the antibody, e.g., Asn297Ala (N297A), where the residue numbers are according to EU numbering (Kabat, E. A., et al., supra).

In some aspects, the constant region of the heavy chain of the antibody is an human IgG3 isotype, having an amino acid sequence:

(SEQ ID NO: 220)
ASTKGPSVFPLAPCSRSTSGGTAALGCLVKDYFPEPVTVSWNSGALTS
GVHTFPAVLQSSGLYSLSSVVTVPSSSLGTQTYTCNVNHKPSNTKVDK
RVELKTPLGDTTHTCPRCPEPKSCDTPPPCPRCPEPKSCDTPPPCPRC
PEPKSCDTPPPCPRCPAPELLGGPSVFLFPPKPKDTLMISRTPEVTCV

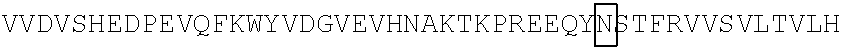

QDWLNGKEYKCKVSNKALPAPIEKTISKTKGQPREPQVYTLPPSREEM
TKNQVSLTCLVKGFYPSDIAVEWESSGQPENNYNTTPPMLDSDGSFFL

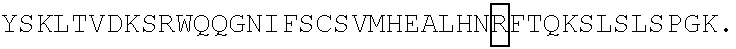

In some aspects, the human IgG3 constant region is modified at amino acid Asn297 (boxed in SEQ ID NO: 220 in the preceding paragraph) to prevent to glycosylation of the antibody, e.g., Asn297Ala (N297A). In some aspects, the human IgG3 constant region is modified at amino acid Arg435 (boxed in SEQ ID NO: 220 in the preceding paragraph) to extend the half-life, e.g., Arg435H (R435H). All residue numbers are according to EU numbering (Kabat, E. A., et al., supra).

In some aspects, the constant region of the heavy chain of the antibody is an human IgG4 isotype, having an amino acid sequence:

(SEQ ID NO: 221)
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK

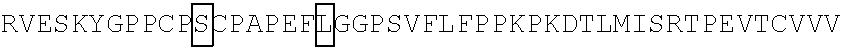

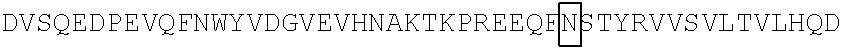

WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNHYTQKSLSLSLGK.

In some aspects, the human IgG4 constant region is modified within the hinge region to prevent or reduce strand exchange, e.g., in some aspects human IgG4 constant region is modified at Ser228 (boxed in SEQ ID NO: 221 in the preceding paragraph), e.g., Ser228Pro (S228P). In other embodiments, the human IgG4 constant region is modified at amino acid Leu235 (boxed in SEQ ID NO: 221 in the preceding paragraph) to alter Fc receptor interactions, e.g., Leu235Glu (L235E). In some aspects, the human IgG4 constant region is modified at both Ser228 and Leu335, e.g., Ser228Pro and Leu235Glu (S228P/L235E). In some aspects, the human IgG4 constant region is modified at amino acid Asn297 (boxed in SEQ ID NO: 221 in the preceding paragraph) to prevent to glycosylation of the antibody, e.g., Asn297Ala (N297A). All residue numbers are according to EU numbering (Kabat, E. A., et al., supra).

In some aspects, the human IgG constant region is modified to enhance FcRn binding. Examples of Fc mutations that enhance binding to FcRn are Met252Tyr, Ser254Thr, Thr256Glu (M252Y, S254T, T256E, respectively) (Dall'Acqua et al. (2006) J. BIOL. CHEM. 281(33): 23514-23524), or Met428Leu and Asn434Ser (M428L, N434S) (Zalevsky et al. (2010) NATURE BIOTECH. 28(2): 157-159). All residue numbers are according to EU numbering (Kabat, E. A., et al., supra).

In some aspects, the human IgG constant region is modified to alter antibody-dependent cellular cytotoxicity (ADCC) and/or complement-dependent cytotoxicity (CDC), e.g., the amino acid modifications described in Natsume et al. (2008) CANCER RES. 68(10): 3863-72; Idusogie et al. (2001) J. IMMUNOL. 166(4): 2571-5; Moore et al. (2010) MABs 2(2): 181-189; Lazar et al. (2006) PROC. NATL. ACAD. SCI. USA 103(11): 4005-4010, Shields et al. (2001) J. BIOL. CHEM. 276(9): 6591-6604; Stavenhagen et al. (2007) CANCER RES. 67(18): 8882-8890; Stavenhagen et al. (2008) ADVAN. ENZYME REGUL. 48: 152-164; Alegre et al. (1992) J. IMMUNOL. 148: 3461-3468.

In some aspects, the human IgG constant region is modified to induce heterodimerization. For example, a heavy chain having an amino acid modification within the CH3 domain at Thr366, e.g., a substitution with a more bulky amino acid, e.g., Tyr (T366W), is able to preferentially pair with a second heavy chain having a CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (see, Carter (2001) J. IMMUNOL. METHODS 248: 7-15).

In some aspects, the constant region of the light chain of the antibody is a human kappa constant region, e.g., a human kappa constant region having the amino acid sequence:

(SEQ ID NO: 222)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC,

In some aspects, the constant region of the light chain of the antibody is a human kappa constant region, e.g., a human kappa constant region having the amino acid sequence:

(SEQ ID NO: 223)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some aspects, the constant region of the light chain of the antibody is a human lambda constant region, e.g., a human lambda constant region having the amino acid sequence:

(SEQ ID NO: 224)
GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTEC.

### III. CHIMERIC ANTIGEN RECEPTORS

Chimeric antigen receptor (CAR) molecules are recombinant fusion protein and are distinguished by their ability to both bind antigen and transduce activation signals via immunoreceptor activation motifs (ITAMs) present in their cytoplasmic tails in order to activate genetically modified immune effector cells for killing, proliferation, and cytokine production. Receptor constructs utilizing an antigen-binding moiety (for example, generated from single chain antibodies (scFv)) afford the additional advantage of being “universal” in that they bind native antigen on the target cell surface in an HLA-independent fashion.

Embodiments of the CARs described herein include nucleic acids encoding an antigen-specific CAR polypeptide comprising an intracellular signaling domain, a transmembrane domain, and an extracellular domain comprising an antigen-binding domain. A CAR may recognize an epitope comprised of the shared space between one or more antigens. Optionally, a CAR can comprise a hinge domain positioned between the transmembrane domain and the antigen binding domain. A CAR may further comprise a signal peptide that directs expression of the CAR to the cell surface. For example, a CAR may comprise a signal peptide from GM-CSF. A CAR may also be co-expressed with a membrane-bound cytokine to improve persistence. For example, a CAR may be co-expressed with membrane-bound IL-15.

Depending on the arrangement of the domains of the CAR and the specific sequences used in the domains, immune effector cells expressing the CAR may have different levels activity against target cells. Different CAR sequences may be introduced into immune effector cells to generate engineered cells, the engineered cells selected for elevated SRC, and the selected cells tested for activity to identify the CAR constructs predicted to have the greatest therapeutic efficacy.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell or an NK cell.

The chimeric construct may be introduced into immune effector cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression. Alternatively, a viral vector (e.g., a retroviral vector, adenoviral vector, adeno-associated viral vector, or lentiviral vector) can be used to introduce the chimeric construct into immune effector cells. Suitable vectors for use in accordance with the method of the present invention are non-replicating in the immune effector cells. A large number of vectors are known that are based on viruses, where the copy number of the virus maintained in the cell is low enough to maintain the viability of the cell, such as, for example, vectors based on HIV, SV40, EBV, HSV, or BPV.

### A. Antigen Binding Domains

An antigen binding domain may comprise complementary determining regions of a monoclonal antibody, variable regions of a monoclonal antibody, and/or antigen binding fragments thereof. The antigen binding regions or domains may comprise a fragment of the VH and VL chains of a single-chain variable fragment (scFv) derived from a particular mouse, human, or humanized monoclonal antibody. The fragment can also be any number of different antigen binding domains of an antigen-specific antibody. The fragment may be an antigen-specific scFv encoded by a sequence that is optimized for human codon usage for expression in human cells. In certain aspects, VH and VL domains of a CAR are separated by a linker sequence, such as a Whitlow linker.

The prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to HSP70. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., conformations preferentially associated with cancer cells versus normal tissue).

Alternatively, a CAR may be engineered using VH and VL chains derived from different mAbs to generate a panel of CAR+ immune effector cells. The antigen binding domain of a CAR may contain any combination of the LCDR1-3 sequences of a first antibody and the HCDR1-3 sequences of a second antibody.

### B. Hinge Comains

A CAR polypeptide may include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-modified immune effector cells. The hinge domain may comprise a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor.

A CAR hinge domain may be derived from human immunoglobulin (Ig) constant region or a portion thereof including the Ig hinge, or from human CD8 α transmembrane domain and CD8a-hinge region. A CAR hinge domain may comprise a hinge-CH₂-CH₃ region of antibody isotype IgG4. The hinge domain (and/or the CAR) may not comprise a wild type human IgG4 CH2 and CH3 sequence. Point mutations may be introduced in antibody heavy chain CH₂ domain to reduce glycosylation and non-specific Fc gamma receptor binding of CAR-modified immune effector cells.

A CAR hinge domain may comprise an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. A CAR hinge domain may comprise an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. A CAR hinge domain may comprise an IgG4-Fc domain having an amino acid substitution at position L235 for an amino acid that is hydrophilic, such as R, H, K, D, E, S, T, N or Q, or that has similar properties to an “E,” such as D. A CAR hinge domain may comprise an IgG4-Fc domain having an amino acid substitution at position N297 for an amino acid that has similar properties to a “Q,” such as S or T.

The hinge domain may comprise a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain, or an engineered hinge domain.

### C. Transmembrane Domains

The antigen-specific extracellular domain and the intracellular signaling-domain may be linked by a transmembrane domain. Polypeptide sequences that can be used as part of transmembrane domain include, without limitation, the human CD4 transmembrane domain, the human CD28 transmembrane domain, the transmembrane human CD3ζ domain, a cysteine mutated human CD3ζ domain, or other transmembrane domains from other human transmembrane signaling proteins, such as CD16, CD8, and erythropoietin receptor. For example, the transmembrane domain may comprise a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8α transmembrane domain), both incorporated herein by reference. Transmembrane regions may be derived from (i.e. comprise at least the transmembrane region(s) of) the alpha, beta or zeta chain of the T-cell receptor, CD3 epsilon, CD45, CD4, CDS, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

### D. Intracellular Signaling Domains

The intracellular signaling domain of a CAR is responsible for activation of at least one of the normal effector functions of the immune cell engineered to express the CAR. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function. The intracellular signaling domain may be derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB/CD137, ICOS/CD278, IL-2Rβ/CD122, IL-2Rα/CD132, DAP10, DAP12, CD40, OX40/CD134, and combinations thereof, as well as other similar molecules and fragments. Intracellular signaling portions of other members of the families of activating proteins can be used.

While the entire intracellular signaling domain may be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. One or multiple cytoplasmic domains may be employed, as so-called third generation CARs have at least two or three signaling domains fused together for additive or synergistic effect, for example the CD28 and 4-1BB can be combined in a CAR construct. In certain specific aspects, the intracellular signaling domain comprises a sequence 85%, 90%, 91%, 92%, 93%, 94%, 95%, 96%, 97%, 98%, 99% or 100% identical to a CD3ζ intracellular domain, a CD28 intracellular domain, a CD137 intracellular domain, or a domain comprising a CD28 intracellular domain fused to the 4-1BB intracellular domain.

### E. Immune Effector Cells

Immune effectors cells may be T cells (e.g., regulatory T cells, CD4+ T cells, CD8+ T cells, or gamma-delta T cells), natural killer (NK) cells, invariant NK cells, or NKT cells. Also provided herein are methods of producing and engineering the immune effector cells as well as methods of using and administering the cells for adoptive cell therapy, in which case the cells may be autologous or allogeneic. Thus, the immune effector cells may be used as immunotherapy, such as to target cancer cells.

The immune effector cells may be isolated from subjects, particularly human subjects. The immune effector cells can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from a blood bank. Immune effector cells can be collected, enriched, and/or purified from any tissue or organ in which they reside in the subject including, but not limited to, blood, cord blood, spleen, thymus, lymph nodes, bone marrow, tissues removed and/or exposed during surgical procedures, and tissues obtained via biopsy procedures. The isolated immune effector cells may be used directly, or they can be stored for a period of time, such as by freezing.

Tissues/organs from which the immune effector cells are enriched, isolated, and/or purified may be isolated from both living and non-living subjects, wherein the non-living subjects are organ donors. Immune effector cells isolated from cord blood may have enhanced immunomodulation capacity, such as measured by CD4- or CD8-positive T cell suppression. The immune effector cells may be isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune effector cell activity. Thus, the cells will be autologous to the subject in need of therapy. Alternatively, the population of immune effector cells can be obtained from a donor, preferably an allogeneic donor. Allogeneic donor cells may or may not be human-leukocyte-antigen (HLA)-compatible. To be rendered subject-compatible, allogeneic cells can be treated to reduce immunogenicity.

### 1. T Cells

The immune effector cells may be T cells. The T cells may be derived from the blood, bone marrow, lymph, umbilical cord, or lymphoid organs. The T cells may be human T cells. The T cells typically are primary cells, such as those isolated directly from a subject and/or isolated from a subject and frozen. The cells may include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. For off-the-shelf technologies, the cells may be derived from pluripotent and/or multipotent cells, such as stem cells, such as induced pluripotent stem cells (iPSCs).

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

One or more of the T cell populations may be enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

T cells may be separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

CD8⁺ T cells may be further enriched for or depleted of naive, central memory, effector memory, and/or central memory stem cells, such as by positive or negative selection based on surface antigens associated with the respective subpopulation. Enrichment for central memory T (T_{CM}) cells may be carried out to increase efficacy, such as to improve long-term survival, expansion, and/or engraftment following administration, which in some aspects is particularly robust in such sub-populations.

The T cells may be autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days.

The cultured T cells can be pooled and rapidly expanded. Rapid expansion provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., MOLECULAR CLONING: A LABORATORY MANUAL, 3^{rd} ed., Cold Spring Harbor Press, Cold Spring Harbor, N.Y. 2001; and Ausubel et al., CURRENT PROTOCOLS IN MOLECULAR BIOLOGY, Greene Publishing Associates and John Wiley & Sons, NY, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

### 2. NK Cells

The immune effector cells may be natural killer (NK) cells. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of interleukin 2 (IL-2), strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD16, CD56, and CD8 in humans. NK cells do not express T-cell antigen receptors, the pan T marker CD3, or surface immunoglobulin B cell receptors.

Stimulation of NK cells is achieved through a cross-talk of signals derived from cell surface activating and inhibitory receptors. The activation status of NK cells is regulated by a balance of intracellular signals received from an array of germ-line-encoded activating and inhibitory receptors. When NK cells encounter an abnormal cell (e.g., tumor or virus-infected cell) and activating signals predominate, the NK cells can rapidly induce apoptosis of the target cell through directed secretion of cytolytic granules containing perform and granzymes or engagement of death domain-containing receptors. Activated NK cells can also secrete type I cytokines, such as interferon-γ, tumor necrosis factor-α and granulocyte-macrophage colony-stimulating factor (GM-CSF), which activate both innate and adaptive immune cells as well as other cytokines. Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

NK cells may be derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. In certain aspects, the NK cells are isolated and expanded ex vivo. For example, CB mononuclear cells may be isolated by ficoll density gradient centrifugation and cultured in a bioreactor with IL-2 and artificial antigen presenting cells (aAPCs). After 7 days, the cell culture may be depleted of any cells expressing CD3 and re-cultured for an additional 7 days. The cells may be again CD3-depleted and characterized to determine the percentage of CD56⁺/CD3⁻ cells or NK cells. In other methods, umbilical CB may be used to derive NK cells by the isolation of CD34⁺ cells and differentiation into CD56⁺/CD3⁻ cells by culturing in medium contain SCF, IL-7, IL-15, and IL-2.

### F. Engineering of Immune Effector Cells

The immune effectors cells (e.g., autologous or allogeneic T cells (e.g., regulatory T cells, CD4+ T cells, CD8+ T cells, or gamma-delta T cells), NK cells, invariant NK cells, or NKT cells) may be genetically engineered to express antigen receptors such as chimeric antigen receptors (CARs). For example, the host cells (e.g., autologous or allogeneic T-cells) may be modified to express a CAR having antigenic specificity for HSP70. In particular embodiments, NK cells are engineered to express a CAR. Multiple CARs, such as to different antigens, may be added to a single cell type, such as T cells or NK cells.

The cells may comprise one or more nucleic acids introduced via genetic engineering that encode one or more antigen receptors, and genetically engineered products of such nucleic acids. The nucleic acids may be heterologous, i.e., normally not present in a cell or sample obtained from the cell, such as one obtained from another organism or cell, which for example, is not ordinarily found in the cell being engineered and/or an organism from which such cell is derived. The nucleic acids may not be naturally occurring, such as a nucleic acid not found in nature (e.g., chimeric).

### IV. PHARMACEUTICAL FORMULATIONS

The present disclosure provides pharmaceutical compositions comprising antibodies that selectively target HSP70. Such compositions comprise a prophylactically or therapeutically effective amount of an antibody or a fragment thereof and a pharmaceutically acceptable carrier. Also provided herein are pharmaceutical compositions and formulations comprising immune cells (e.g., T cells or NK cells) expressing a CAR and a pharmaceutically acceptable carrier.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The active ingredients can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in Remington's Pharmaceutical Sciences. Such compositions will contain a prophylactically or therapeutically effective amount of the antibody or fragment thereof, preferably in purified form, together with a suitable amount of carrier so as to provide the form for proper administration to the patient.

Passive transfer of antibodies generally will involve the use of intravenous or intramuscular injections. The resulting immunity generally lasts for only a short period of time, and there is also a potential risk for hypersensitivity reactions, and serum sickness, especially from gamma globulin of non-human origin. The antibodies may be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, pharmaceutical compositions may comprise, for example, at least about 0.1% of an active ingredient. In other embodiments, an active ingredient may comprise between about 2% to about 75% of the weight of the unit, or between about 25% to about 60%, for example, and any range derivable therein.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

### V. METHODS OF TREATMENT

Certain aspects of the present embodiments can be used to prevent or treat a disease or disorder associated with elevated levels of HSP70, such as cancer, such as lung cancer, prostate cancer, stomach cancer, thyroid cancer, or breast cancer. Functioning of HSP70 may be reduced by any suitable drugs. Preferably, such substances would be an anti-HSP70 antibody, HSP70-specific CAR T cell, or HSP70-specific CAR NK cell.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that targets HSP70, either alone or in combination with administration of chemotherapy, immunotherapy, or radiotherapy, performance of surgery, or any combination thereof.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

In certain embodiments, the compositions and methods of the present embodiments involve an antibody or an antibody fragment against HSP70, in combination with a second or additional therapy, such as chemotherapy or immunotherapy. Such therapy can be applied in the treatment of any disease that is associated with elevated HSP70. For example, the disease may be a cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammaI1 and calicheamicin omegaI1); dynemicin, including dynemicin A; bisphosphonates, such as clodronate; an esperamicin; as well as neocarzinostatin chromophore and related chromoprotein enediyne antiobiotic chromophores, ad acinomysins, actinomycin, authrarnycin, azaserine, bleomycins, cactinomycin, carabicin, carminomycin, carzinophilin, chromomycini s, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine,plicomycin, gemcitabien, navelbine, farnesyl-protein transferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2,000 to 6,000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include B-cell maturation antigen, CD20, carcinoembryonic antigen, tyrosinase (p97), gp68, GPRC5D, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some aspects, a combination described herein includes an agent that decreases tumor immunosuppression, such as a chemokine (C-X-C motif) receptor 2 (CXCR2) inhibitor. In some embodiments, the CXCR2 inhibitor is danirixin (CAS Registry Number: 954126-98-8). Danirixin is also known as GSK1325756 or 1-(4-chloro-2-hydroxy-3-piperidin-3-yl sulfonylphenyl)-3-(3-fluoro-2-methylphenyl)urea. Danirixin is disclosed, e.g., in Miller et al. Eur J Drug Metab Pharmacokinet (2014) 39:173-181; and Miller et al. BMC Pharmacology and Toxicology (2015), 16:18. In some embodiments, the CXCR2 inhibitor is reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR1/2. Reparixin is disclosed, e.g., in Zarbock et al. British Journal of Pharmacology (2008), 1-8. In some embodiments, the CXCR2 inhibitor is navarixin. Navarixin is also known as MK-7123, SCH527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide Navarixin is disclosed, e.g., in Ning et al. Mol Cancer Ther. 2012; 11(6):1353-64. In some embodiments, the CXCR2 inhibitor is AZD5069, also known as N-[2-[[(2,3-difluoropheny)methyl]thio]-6-{[(1R,2S)-2,3-dihydroxy-1-methylpropyl]oxy}-4-pyrimidinyl]-1-azetidinesulfonamide. In some embodiments, the CXCR2 inhibitor is an anti-CXCR2 antibody, such as those disclosed in WO2020/028479.

In some aspects, a combination described herein includes an agent that activates dendritic cells, such as, for example, a TLR agonist. A “TLR agonist” as defined herein is any molecule which activates a toll-like receptor as described in Bauer et al., 2001, Proc. Natl. Acad. Sci. USA 98: 9237-9242. A TLR agonist may be a small molecule, a recombinant protein, an antibody or antibody fragment, a nucleic acid, or a protein. In certain embodiments, the TLR agonist is recombinant, a natural ligand, an immunostimulatory nucleotide sequence, a small molecule, a purified bacterial extract or an inactivated bacteria preparation.

Several agonists of TLR derived from microbes have been described, such as lipopolysaccharides, peptidoglycans, flagellin and lipoteichoic acid (Aderem et al., 2000, Nature 406:782-787; Akira et al., 2001, Nat. Immunol. 2: 675-680) Some of these ligands can activate different dendritic cell subsets, that express distinct patterns of TLRs (Kadowaki et al., 2001, J. Exp. Med. 194: 863-869). Therefore, a TLR agonist could be any preparation of a microbial agent that possesses TLR agonist properties. Certain types of untranslated DNA have been shown to stimulate immune responses by activating TLRs. In particular, immunostimulatory oligonucleotides containing CpG motifs have been widely disclosed and reported to activate lymphocytes (see, U.S. Pat. No. 6,194,388). A “CpG motif” as used herein is defined as an unmethylated cytosine-guanine (CpG) dinucleotide. Immunostimulatory oligonucleotides which contain CpG motifs can also be used as TLR agonists according to the methods of the present invention. The immunostimulatory nucleotide sequence may be stabilized by structure modification such as phosphorothioate modification or may be encapsulated in cationic liposomes to improve in vivo pharmacokinetics and tumor targeting.

In some embodiments, the immunotherapy may be an immune checkpoint inhibitor. Immune checkpoints either turn up a signal (e.g., co-stimulatory molecules) or turn down a signal. Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12(4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUIDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95(17): 10067-10071; Camacho et al. (2004) J Clin Oncology, 22(145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

In some embodiment, the immune therapy could be adoptive immunotherapy, which involves the transfer of autologous antigen- specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. METHODS OF DETECTION

In some aspects, the present disclosure concerns immunodetection methods for detecting expression of HSP70. A wide variety of assay formats are contemplated for detecting protein products, including immunohistochemistry, enzyme linked immunosorbent assay (ELISA), radioimmunoassay (RIA), immunoradiometric assay, fluoroimmunoassay, chemiluminescent assay, bioluminescent assay, dot blotting, FACS analyses, and Western blot to mention a few. The steps of various useful immunodetection methods have been described in the scientific literature. In general, the immunobinding methods include obtaining a sample, and contacting the sample with an antibody specific for the protein to be detected, as the case may be, under conditions effective to allow the formation of immunocomplexes. In general, the detection of immunocomplex formation is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

As used herein, the term “sample” refers to any sample suitable for the detection methods provided by the present invention. The sample may be any sample that includes material suitable for detection or isolation. Sources of samples include blood, pleural fluid, peritoneal fluid, urine, saliva, malignant ascites, broncho-alveolar lavage fluid, synovial fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer. In some aspects, the biological sample comprises a plurality of cells. In certain aspects, the biological sample comprises fresh or frozen tissue. In specific aspects, the biological sample comprises formalin fixed, paraffin embedded tissue. In some aspects, the biological sample is a tissue biopsy, fine needle aspirate, blood, serum, plasma, cerebral spinal fluid, urine, stool, saliva, circulating tumor cells, exosomes, or aspirates and bodily secretions, such as sweat. In some aspects, the biological sample contains cell-free DNA.

### VII. KITS

In various aspects of the embodiments, a kit is envisioned containing therapeutic agents and/or other therapeutic and delivery agents. In some embodiments, a kit is provided for preparing and/or administering a therapy of the embodiments. The kit may comprise one or more sealed vials containing any of the pharmaceutical compositions of the present embodiments. The kit may include, for example, at least one HSP70 antibody or HSP70-specific CAR construct, as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Generation of Therapeutic Antibodies Targeting HSP70

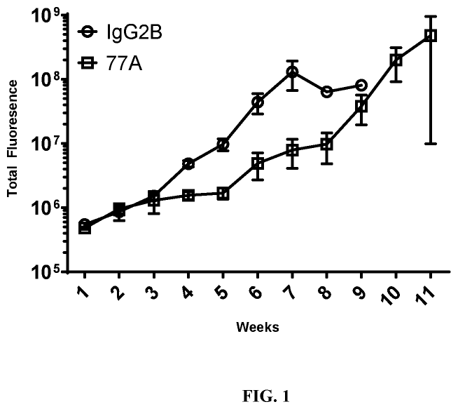

To develop an anti-HSP70 mAb, murine fibroblast L-cells (Willert et al., 2003) expressing human HSP70 fused to Green fluorescent protein (GFP) were generated and injected into the footpads of BALB/c mice in collaboration with the MD Anderson Monoclonal Antibody Core Facility. After an initial series of four injections every three days, and two boost injections on days 13 and 15, murine spleen cells were fused with Sp2/0-Ag14 murine myeloma cells (Shulman et al., 1978) to generate hybridomas. Single-cell cloning narrowed the initial output of 96 clones in mixed culture to 46 clones, followed by screening with a dry-cell ELISA to identify mAbs recognizing L-cells expressing HSP70-GFP but not vector-bearing wild-type, or GFP-expressing L-cells. The mAbs made by the remaining 28 hybridoma clones were next characterized by their ability to recognize HSP70 on intact MM1.S myeloma cells by flow, and in MM1.S cell extracts by Western blotting, which indicated that 17 clones had interesting properties. These were further screened for their binding to HSP70 versus to the closely homologous HSP70 family members (Daugaard et al., 2007) using RPMI 8226 human myeloma cells and 8226 cells in which HSP70 had been knocked out using CRISPR/Cas9-mediated genome editing, leading to the conclusion that six mAbs had the highest specificity for human HSP70. As a final screen, the antibody's anti-tumor activity was evaluated in immune-competent BALB/c mice injected with luciferase (luc)-expressing MOPC315.BM murine myeloma cells in a model that recapitulates much of the natural history of human myeloma, including the development of osteolytic bony disease (Hofgaard et al., 2012). This was possible because of the very close, 95% homology between murine and human HSP70 at the amino acid level (Hunt & Calderwood, 1990), and the finding that the mAbs bound both proteins. Clone 77A showed anti-tumor activity in pilot studies (FIG. 1), and clone 77A (hereafter referred to as 77A) was selected for further study because ⅖ treated mice showed complete myeloma resolution without recurrence at 100 days.

The complementarity-determining regions (CDR) and variable regions of the 77A antibody are provided in Tables 1-3.

TABLE 1
CDRs of heavy and light chain variable sequences
of the 77A antibody.

| | CDR1 | CDR2 | CDR3 |
|---|---|---|---|
| Chain | (SEQ ID NO:) | (SEQ ID NO:) | (SEQ ID NO:) |
| Heavy | GYTFTNYG | INTYTGEP | ARYDHAMDY |
| | (SEQ ID NO: 1) | (SEQ ID NO: 2) | (SEQ ID NO: 3) |
| Light | QSLLNSGTRKNY | WTS | KQSYTLYT |
| | (SEQ ID NO: 4) | (SEQ ID NO: 5) | (SEQ ID NO: 6) |

TABLE 2
Amino acid sequences encoding the 77A antibody
variable regions.

| | | SEQ |
|---|---|---|
| | | ID |
| Chain | Variable Sequence | NO: |
| Heavy | QIQLVQSGPELKKPGETVKISCKASGYTFTNYGMNWVKQA | 7 |
| | PGKGLKWMGWINTYTGEPTYADDFKGRFAFSLETSATTAY | |
| | LQINNLKNEDTATYFCARYDHAMDYWGQGTSVTVSS | |
| Light | DIVMSQSPSSLAVSAGEKVTMSCKSSQSLLNSGTRKNYLA | 8 |
| | WYQQKPGQSPKLLIYWTSTRESGVPDRFTGSGSGTDFTLT | |
| | INSVQAEDLAVYYCKQSYTLYTFGGGTKLEIK | |

TABLE 3
Nucleotide sequences encoding the 77A antibody variable regions.

| | | SEQ ID |
|---|---|---|
| Chain | Variable Sequence | NO: |
| Heavy | CAGATCCAGTTGGTGCAGTCTGGACCTGAGCTGAAGAAGCCTGGA | 9 |
| | GAGACAGTCAAGATCTCCTGCAAGGCTTCTGGGTATACCTTCACA | |
| | AACTATGGAATGAACTGGGTGAAGCAGGCTCCAGGAAAGGGTTTA | |
| | AAGTGGATGGGCTGGATAAACACCTACACTGGAGAGCCAACATAT | |
| | GCTGATGACTTCAAGGGACGGTTTGCCTTCTCTTTGGAAACCTCT | |
| | GCCACCACTGCCTATTTGCAGATCAACAACCTCAAAAATGAGGAC | |
| | ACGGCTACATATTTCTGTGCAAGGTACGACCATGCTATGGACTAC | |
| | TGGGGTCAAGGAACCTCAGTCACCGTCTCCTCAG | |
| Light | GACATTGTGATGTCACAGTCTCCATCCTCCCTGGCTGTGTCAGCT | 10 |
| | GGAGAGAAGGTGACTATGAGCTGCAAATCCAGTCAGAGTCTGCTC | |
| | AACAGTGGAACCCGAAAGAACTACTTGGCTTGGTACCAGCAGAAA | |
| | CCAGGGCAGTCTCCTAAACTGCTGATCTACTGGACATCCACTAGG | |
| | GAATCTGGGGTCCCTGATCGCTTCACAGGCAGTGGATCTGGGACA | |
| | GATTTCACTCTCACCATCAACAGTGTGCAGGCTGAAGACCTGGCA | |
| | GTTTATTACTGCAAGCAATCTTATACTCTGTACACGTTCGGAGGG | |
| | GGGACCAAGCTGGAAATAAAAC | |

### Example 2—mAb 77A Shows Strong Affinity for Human HSP70

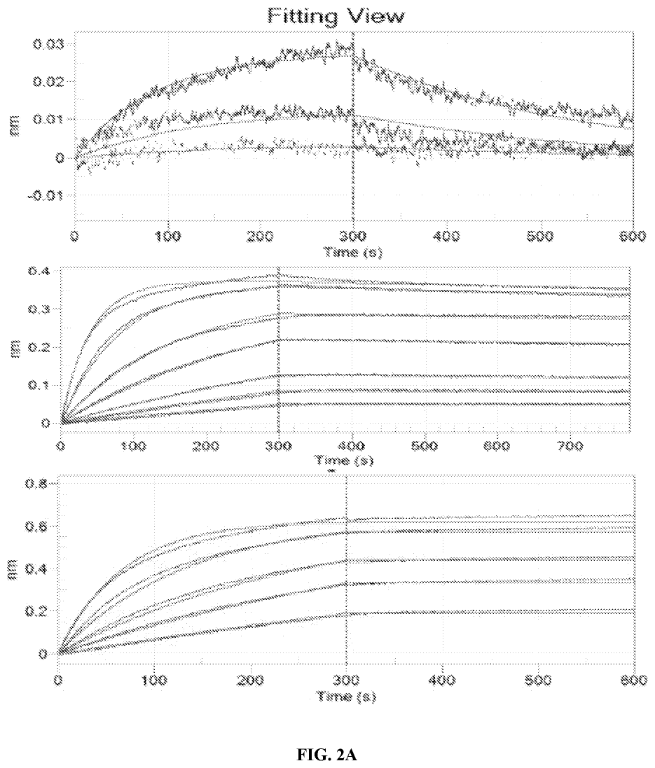

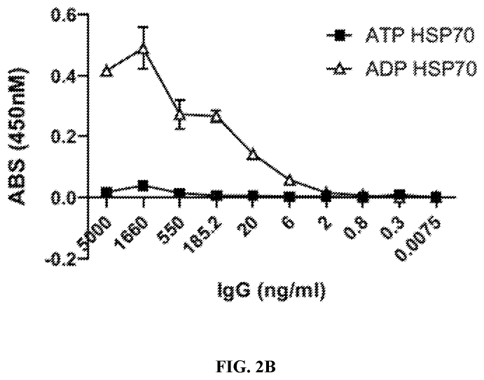

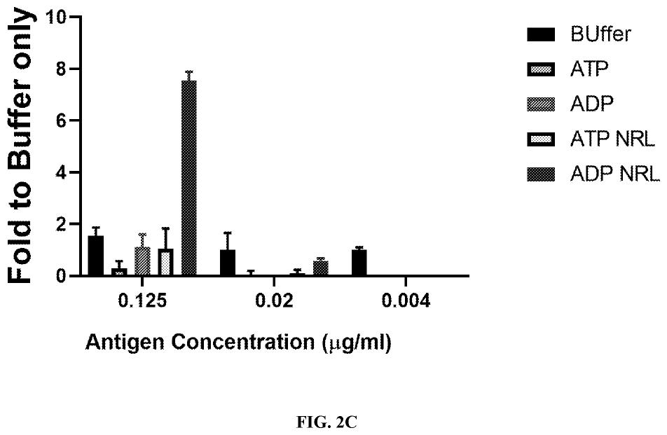

Octet analysis was pursued to study the affinity and kinetics of 77A binding (FIG. 2A). The dissociation constants (K_{D}) for murine HSP70 and human HSP70 made in E. coli were 9.88×10⁻⁷ and 8.50×10⁻¹⁰, respectively, while that for HSP70 made in eukaryotic insect Sf9 cells was below the limit of detection (1.0×10⁻¹²). To provide some context, the 0.85 nM affinity of 77A compares favorably to that of the anti-CD20 mAb rituximab (5.2 nM) (Malviay et al., 2012) and the anti-CD38 mAb daratumumab (4.36 nM) (van de Donk et al., 2016), which are in clinical use for B-cell lymphoma and myeloma, respectively. Therefore, 77A has strong affinity especially for human HSP70, and the difference between human HSP70 made in prokaryotic versus eukaryotic cells suggests that it may target an epitope that is either differently folded or post-translationally modified.

The studies to date have focused on ADP-HSP70 by purification of HSP70 over ADP-agarose as this is the form that has higher affinity for peptides and is more immunogenic (Greene et al., 2018; Peng et al., 1997). In fact, 77A shows preferential binding to ADP-HSP70 complexes, which would contain tumor-derived peptide antigens (FIG. 2B). Furthermore, an ELISA study of 77A binding to HSP70-GFP shows greatest affinity when ADP and a peptide substrate (NRL) is present (FIG. 2C). This preferential binding to HSP70 in its ADP-/peptide-bound form likely enhances delivery of tumor-associated antigens from the tumor microenvironment.

### Example 3—Mapping of the 77A Epitope Using HSP70 Deletion Mutants

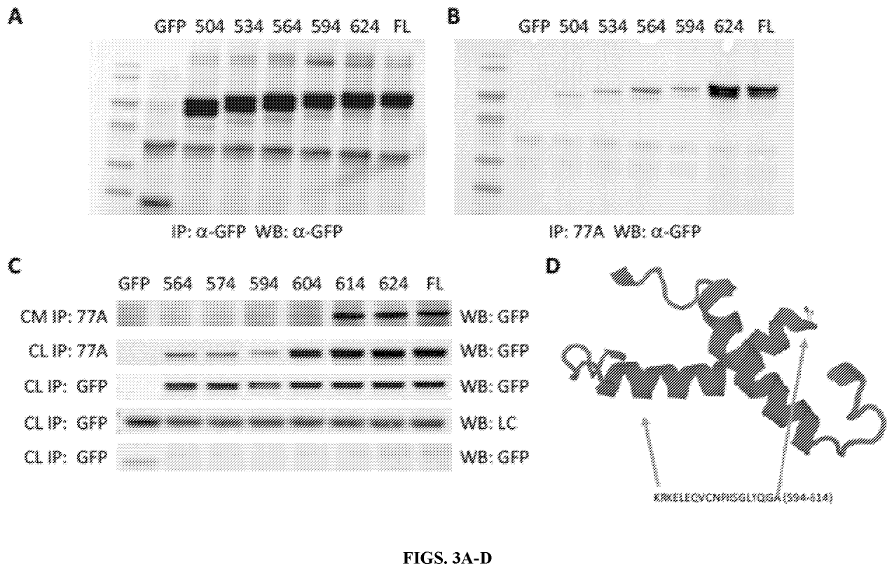

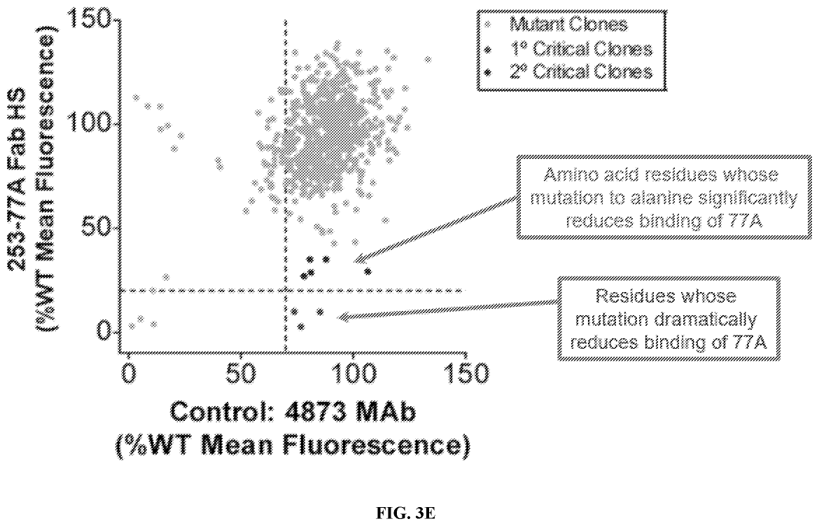

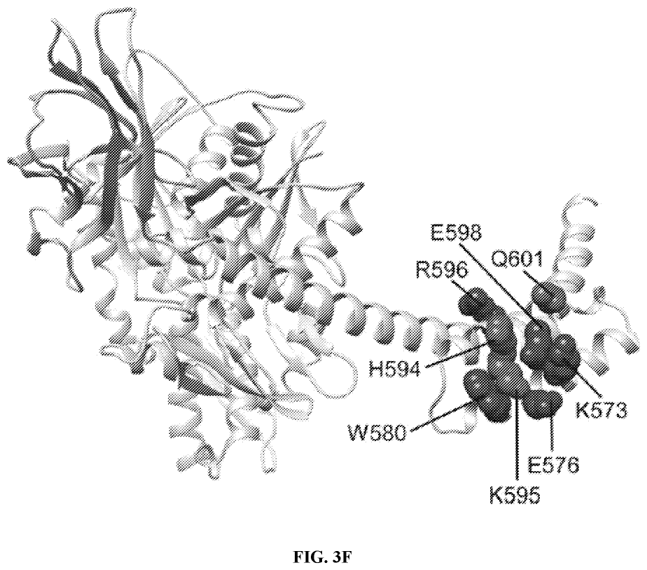

To better understand the binding of 77A to HSP70, HSP70 KO human embryonic kidney (HEK) 293T cells were generated using CRISPR/Cas9 editing, and then full-length HSP70 was expressed with an N-terminal GFP fusion, or various HSP70 deletion mutants (FIG. 3A). Immunoprecipitation (IP) of cell extracts with 77A, followed by Western blotting with an a-GFP antibody, indicated that 77A bound the 641 amino acid (aa) full-length HSP70, and the 624 aa protein with 17 amino acids (aas) deleted from the C-terminus, but binding decreased with an additional 30 aa deletion (FIG. 3B). These findings were confirmed through flow studies of 77A binding to 293T cells expressing these mutants. Smaller deletions showed a reduction in the ability of 77A to recognize secreted HSP70 from cell culture supernatants when aas 604-614 were deleted (FIG. 3C). Interestingly, when HSP70 from cell lysates was used as the target, deletion of 594-604 was associated with decreased recognition. These findings suggest that 77A recognizes an epitope in amino acids 594-614 (FIG. 3D), which is not a target for previously described mAbs, and that there may be differential post-translational modification of secreted HSP70 that impacts recognition by 77A or that 77A binds HSP70 in the context of co-chaperones. Alanine scanning analysis was performed to identify key amino acids in HSP70 with which 77A interacts (FIG. 3E). This analysis determined that 77A recognizes a conformational epitope of HSP70 that includes K573, E576, W580, R596, and E598 as secondary critical sites and H594, K595, and Q601 as primary critical sites (FIG. 3F), with the sites corresponding to the amino acids in SEQ ID NO: 11. These data support a model where 77A binds to a novel HSP70 domain when HSP70 is in its ADP- and antigenic peptide-bound form to lock in substrate binding and increase uptake.

### Example 4—Differential Activity of 77A in Immune-Deficient Myeloma Models

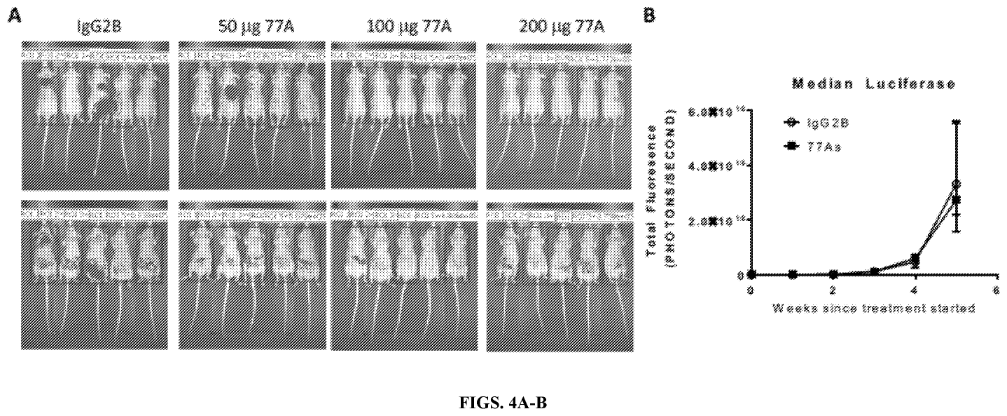

Since 77A bound strongly to human HSP70 from eukaryotic cells (FIG. 2A), its activity was studied in vivo against human myeloma cells. In a model of luc-labeled MM1.S cells in nude mice, 77A showed dose-dependent anti-tumor activity (FIG. 4A), with some tumor growth reduction when given as a 50 μg injection twice weekly, and stronger activity at 100 and 200 μg doses. This was accompanied by a reduction in human light chain levels as measured by an ELISA. Notably, when this study was repeated with MM1.S cells in SCID mice and in non-obese diabetic severe combined immunodeficient IL-2 receptor γ-null (NOD/SCID IL-2R^{γnull} (NSG)) mice, 77A did not show activity by in vivo imaging (FIG. 4B) or light chain quantitation. Together, these contrasting results suggested that 77A is dependent on an immune effector cell retained by nude but absent in NSG mice. Moreover, in vitro assays utilizing MM1.S cells and 77A did not show a direct cytotoxic activity, and 77A did not induce antibody- or complement-dependent cellular cytotoxicity (ADCC, CDC).

### Example 5—77A Enhances HSP70 Uptake by Dendritic Cells

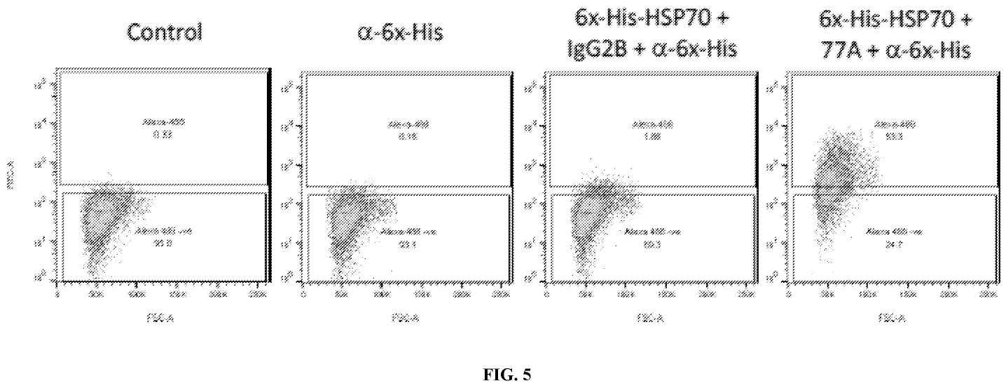

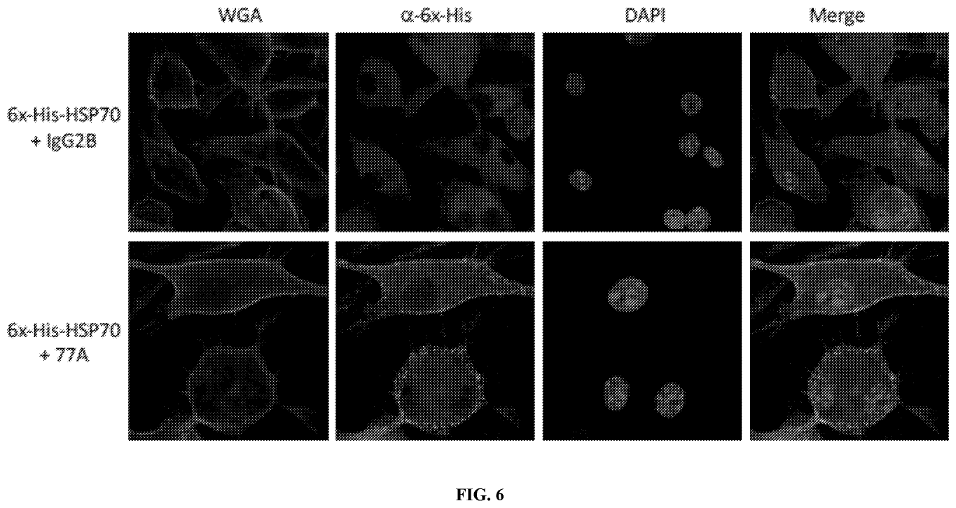

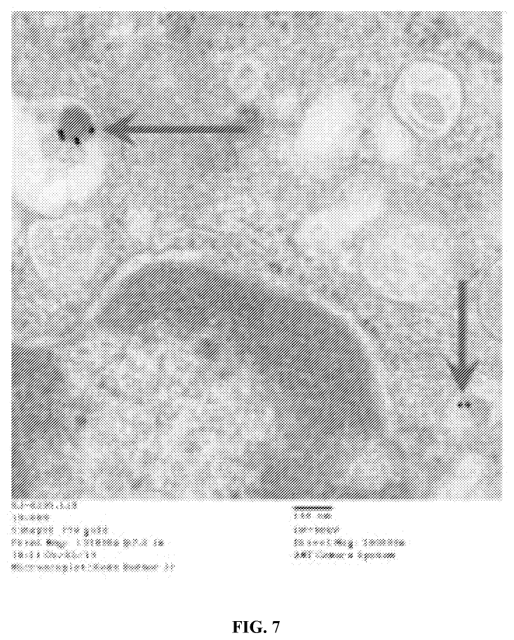

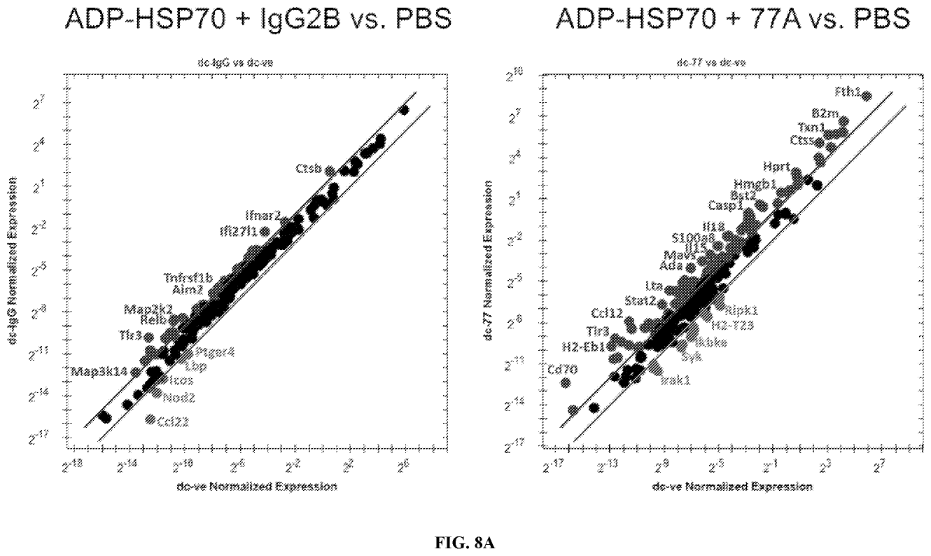

DCs, macrophages, and natural killer (NK) cells are defective in NSG mice (Shultz et al., 1995) whereas these cells are present and functional in nude mice. While there are other differences between NSG and nude mice, DCs were focused on first since HSP70 secreted from tumor cells is known to facilitate delivery of antigen to DCs (Shevtsov & Multhoff, 2016; Binder, 2008). Therefore, purified hexa-(6x)-histidine-tagged human HSP70 made in Sf9 cells was used and an Alexa Fluor 488-tagged α-6x-His-tag mAb was used to study uptake of 6x-His-HSP70 by DC2.4 immature DCs, an immortalized murine line created by transducing bone marrow isolates of C57BL/6 mice with retrovirus vectors expressing murine Granulocyte-macrophage CSF (GM-CSF) and the MYC and RAF oncogenes (Shen et al., 1997). 77A substantially increased HSP70 uptake at 4° C. and at 37° C. compared to a control isotype mAb (referred to as IgG2B) directed against hen egg lysozyme (FIG. 5), while other a-HSP70 mAbs did not have this activity. Enhanced HSP70 uptake in the presence of 77A could also be demonstrated by immunofluorescence staining of DC2.4 cells (FIG. 6). Finally, HSP70 uptake into DCs was examined using 77A conjugated to 15 nM gold particles by electron microscopy (EM). 77A enhanced HSP70 uptake versus IgG2B by EM as it had in prior assays (FIGS. 5 and 6), and HSP70 was found in intracellular cytoplasmic membrane-bound bodies resembling phagolysosomes (FIG. 7; red arrows).

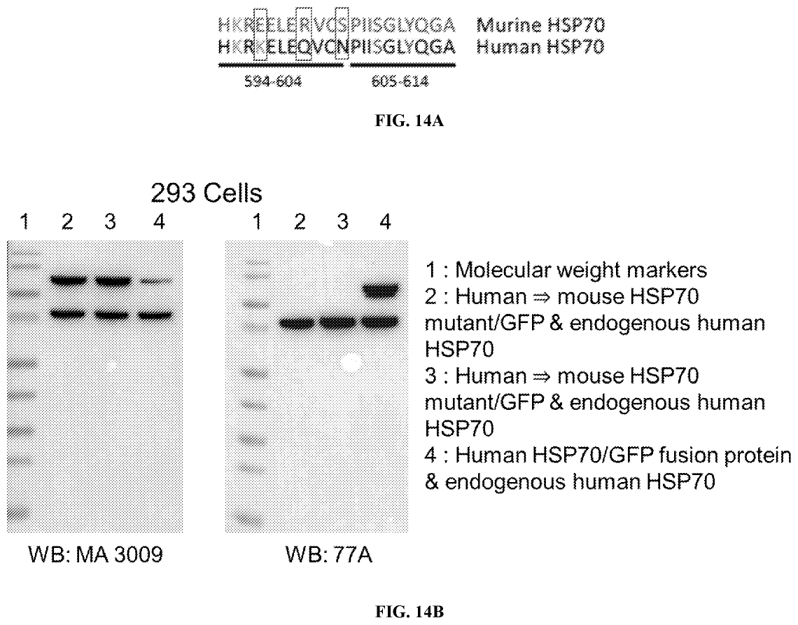

Given the substantial difference in affinity of 77A for murine and human HSP70 (FIG. 5), site-directed mutagenesis was used to express human HSP70 with single changes (K->E, Q->R, N->S) to each amino acid that is different between the two variants (FIG. 14A), each of the possible two amino acid changes, and one with all three. These were expressed in HSP70 KO 293T cells, and the ability of 77A to bind were examined in IP studies (as in FIG. 3). The ability of DC2.4 cells to take up these mutants in the presence of IgG2B and 77A will be studied by FACS (as in FIG. 5). Mutation of the human HSP70 sequence in the 594-614 region to match the murine sequence reduces the affinity of 77A for HSP70 and its ability to induce uptake by DC2.4 cells (FIG. 14B).

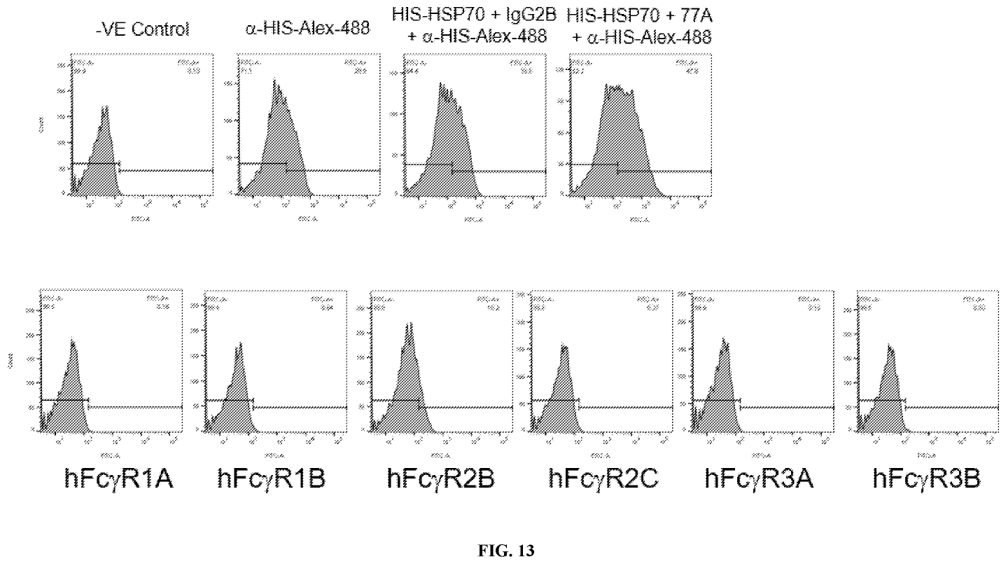

Determining the receptors through which 77A/HSP70 complexes are taken up by DCs could help identify other immune effectors that would be similarly influenced. HSP70 knock-out HEK 239T cells that express murine FcγR2B were found to take up 77A-bound HSP70. Likewise, HSP70 knock-out HEK 239T cells that express human FcγR2A or human FcγR2B were found to take up 77A-bound HSP70, while the expression of other Fc receptors did not produce this effect (FIG. 13). Human FcγR2A and FcγR2B are expressed in monocytes/macrophages and dendritic cells (Bruhns, 2012).

### Example 6—DC Uptake of HSP70 Enhances Maturation

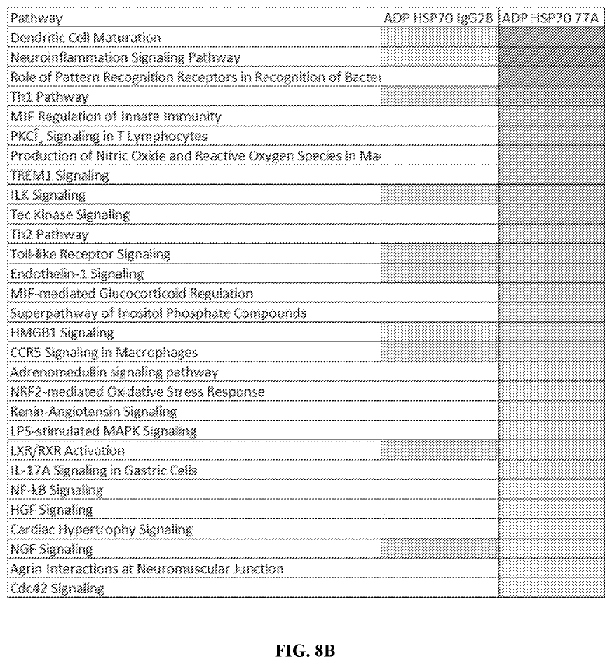

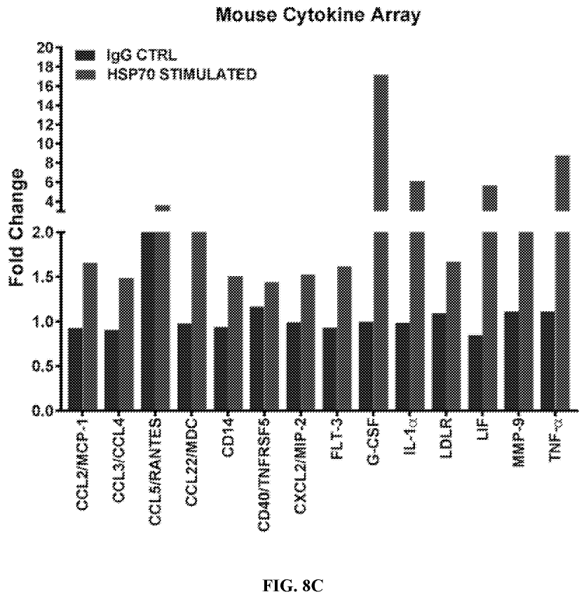

To begin to study the functional consequences of HSP70 uptake by DCs, DC2.4 cells were exposed to HSP70 in the presence of either IgG2B or 77A. RNA was harvested, converted to cDNA, and this was hybridized to the BioRad Immune Response Tier 1-4 qPCR array containing 384 genes. Using a threshold of a 2-fold or more change over controls, IgG2B activated relatively few genes compared to the negative control (FIG. 8A; top panel), while 77A activated transcription of a much larger gene set (FIG. 8A; bottom panel). Ingenuity Pathway Analysis noted that the top activated pathway was one representing dendritic cell maturation (FIG. 8B), reflected in part by the finding that some of the top genes play key roles in DC function, including CD70 (Bourque & Hawiger, 2018), Adenosine deaminase (Casanova et al., 2012), Cathepsin S (Kim et al., 2017), and CC chemokine ligand 5 (Wang et al., 2002). Other activated pathways included a Neuroinflammation signaling pathway, both Th1 and Th2 signaling, and Toll-like receptor (TLR) signaling. In addition, cell culture supernatants from these experiments were collected and analyzed using a cytokine array. Notable changes induced by HSP70 in the presence of 77A versus IgG2B as a control included increases in cytokines that play key roles in DC biology (Turner et al., 2014), including Tumor-necrosis factor (TNF)-α, Granulocyte colony stimulating factor (G-CSF), and Interleukin (IL)-1 (FIG. 8C), among others.

### Example 7—Activity of 77A in the Tumor Models

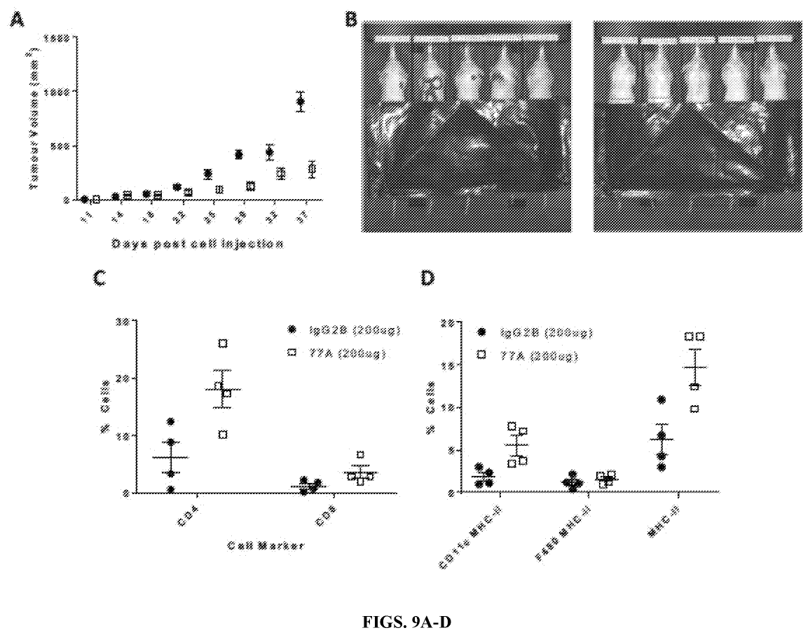

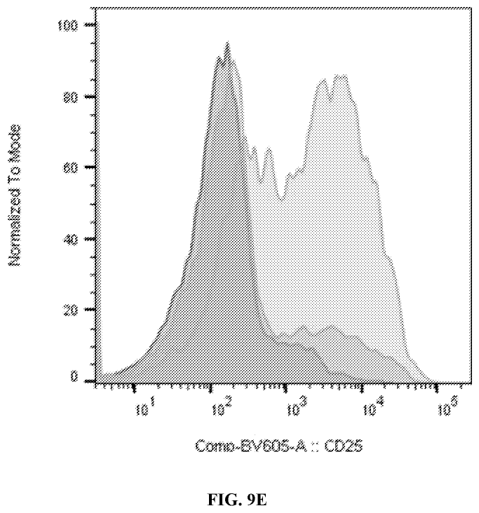

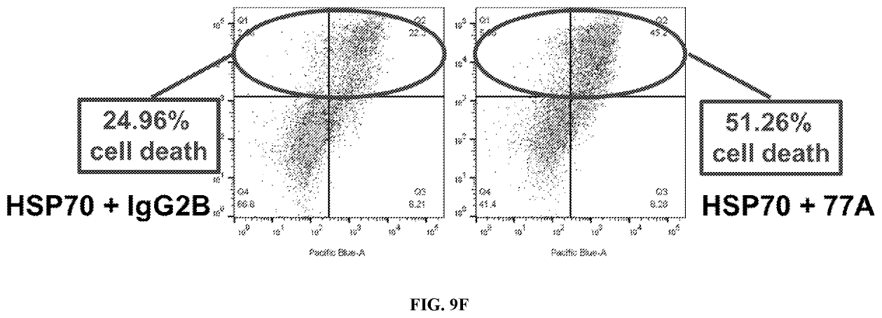

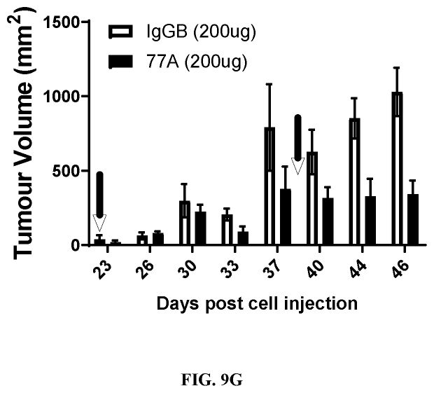

The potential that 77A influenced a general immunity mechanism relevant to many tumors prompted the examination of its activity in a melanoma model and in 4T1 cells, a murine triple-negative breast carcinoma (TNBC) (Chen et al., 2019) model that has been well characterized, is considered immunologically cold (Kim et al., 2014), and does not express surface HSP70. Immune-competent BALB/c mice injected with luc-labeled 4T1 cells into the mammary fat pad and treated with 77A showed slower primary tumor growth versus IgG2B (FIG. 9A). Notably, 77A strongly inhibited the development of metastatic disease to the lung (FIG. 9B) and also to the spleen and liver. Peripheral blood was collected on day 32 from surviving mice and analyzed by multi-parameter flow for T-cell subsets, which showed a significant increase in CD4+ T-cells and a more modest increase in CD8+ T-cells (FIG. 9C). Also, there was an increase noted for MHC class II+ cells in the peripheral blood (FIG. 9D), and cells that were both class II+ and CD11c+, consistent with an increase in murine DCs after 77A treatment. 77A was found to (a) induce uptake of HSP70 into human primary CD4+ and CD8+ T cells (FIG. 9E), (b) enhance T-cell proliferation and expression of maturation markers, including CD69 and HLA DR, and (c) stimulate MHC-independent cytolytic CD4 T-cell activity in a number of tumor models, including A549 human lung carcinoma cells (FIG. 9F).

For the melanoma model, the A375 melanoma model in nude mice was used. The first dose of 77A was given on day 23, and the last dose on day 39. Tumor volume was slower in 77A treated mice (FIG. 9G).

### Example 8—Utility of 77A to Enhance Efficacy of a 4T1-Based Vaccine Strategy

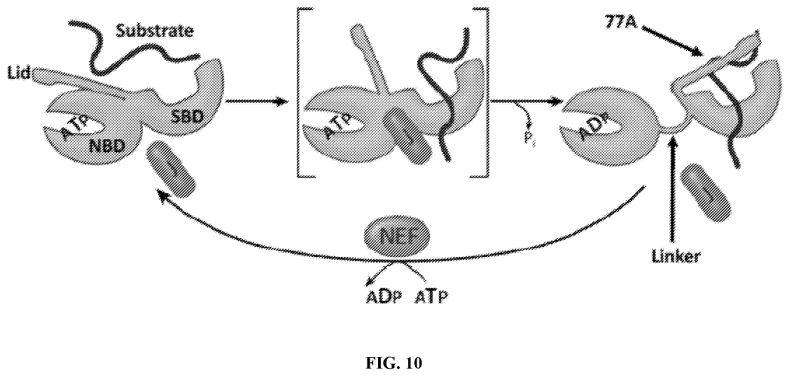

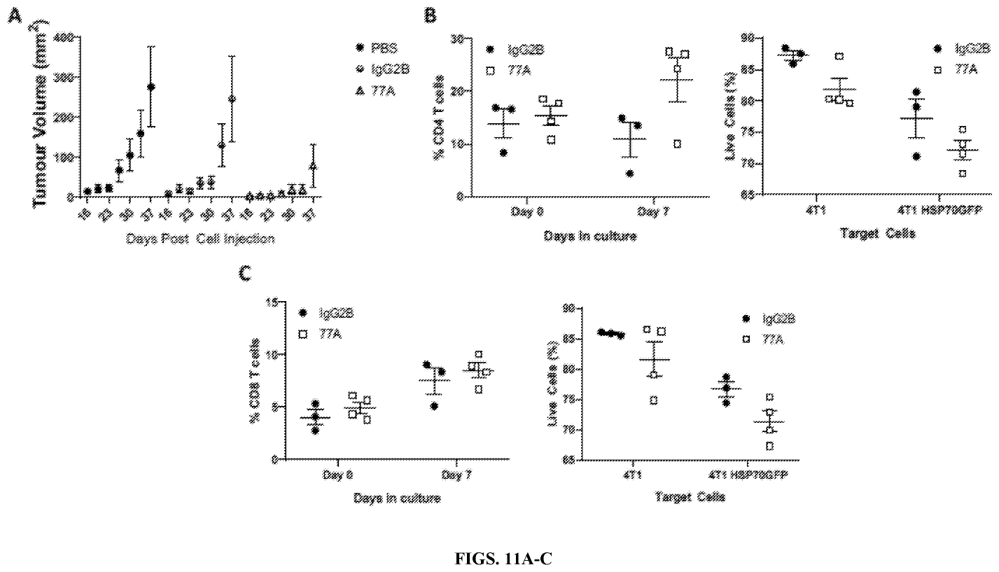

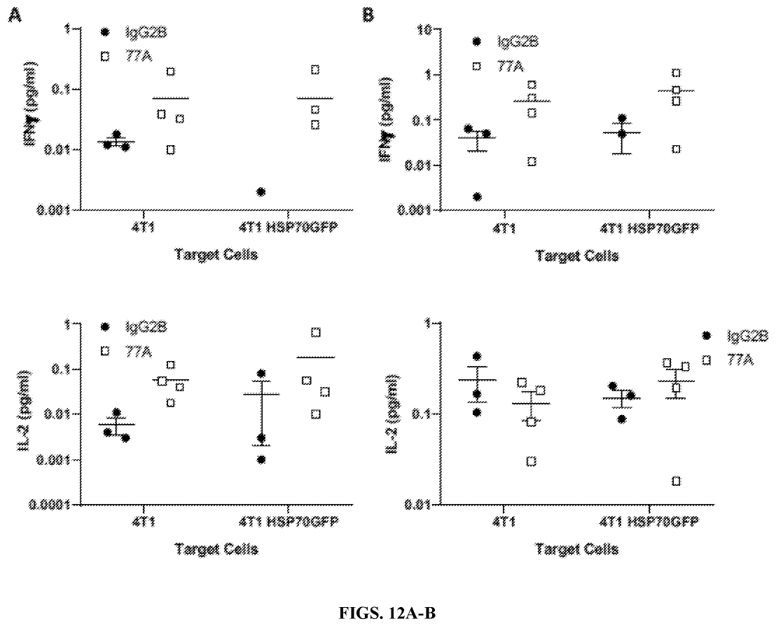

DCs have long been considered attractive targets for the development of vaccine strategies given their roles as professional antigen presenting cells (Gornati et al., 2018). Therefore, the possibility that 77A could increase vaccine efficacy through its ability to enhance HSP70-peptide antigen complex uptake was considered. To test this, 4T1 cells expressing HSP70-GFP were used to purify ADP-HSP70-peptide complexes over an ADP-agarose column, and BALB/c mice were injected with PBS, or with the ADP-HSP70-peptide vaccine with IgG2B or 77A. The focus was on ADP-HSP70 as, compared to ATP-HSP70, ADP-HSP70 is the form that has higher affinity for substrates, including tumor-derived peptides, and is therefore more immunogenic (Greene et al., 1995; Peng et al., 1997; Craig & Marszalek, 2017) (see FIG. 10 for an overview of the fundamentals of the HSP70 machinery). On day 0, mice were orthotopically injected with viable 4T1-luc labeled, HSP70-GFP-expressing cells, and the development of tumors was monitored by whole animal imaging. Vaccination with ADP-HSP-peptide complexes in the presence of IgG2B did not slow tumor growth in mice after challenge with live 4T1 cells compared to the PBS control (FIG. 11A), but vaccination with 77A did slow growth substantially. Splenocytes isolated from these mice at day 37 were then exposed to irradiated 4T1 cells for 7 days, and showed an increase in CD4+ and, to a lesser extent, CD8+ T-cells (FIGS. 11B & C; left panels). Also, these cells showed enhanced cytotoxicity after 7 days against 4T1 cells expressing HSP70-GFP, or 4T1 cells without HSP70-GFP (FIGS. 11B & C; right panels), supporting the possible development of cytotoxic T lymphocytes (CTLs) recognizing both HSP70-GFP-derived antigens and other tumor-derived antigens. This possibility was also tested by evaluating the ability of these T-cells to elaborate interferon (IFN)-γ and IL2. Putative CD4+ CTLs from 77A-vaccinated mice showed increased IFNγ and IL2 secretion when they were exposed to 4T1 or 4T1-HSP70-GFP cells compared with the IgG2B controls (FIG. 12A). In the case of CD8+ CTLs (FIG. 12B), these also showed increased IFNγ secretion when they were exposed to either 4T1 or 4T1-HSP70-GFP cells after vaccination with ADP-HSP70 complexes and 77A, but the results were less striking for IL-2 production. Together, these data suggest that 77A can enhance antigen uptake by DCs and produce more robust downstream CD4+ and CD8+ T-cell responses.

### Example 9—Identification of Tumor Targets for the Clone 77A HSP70 mAb

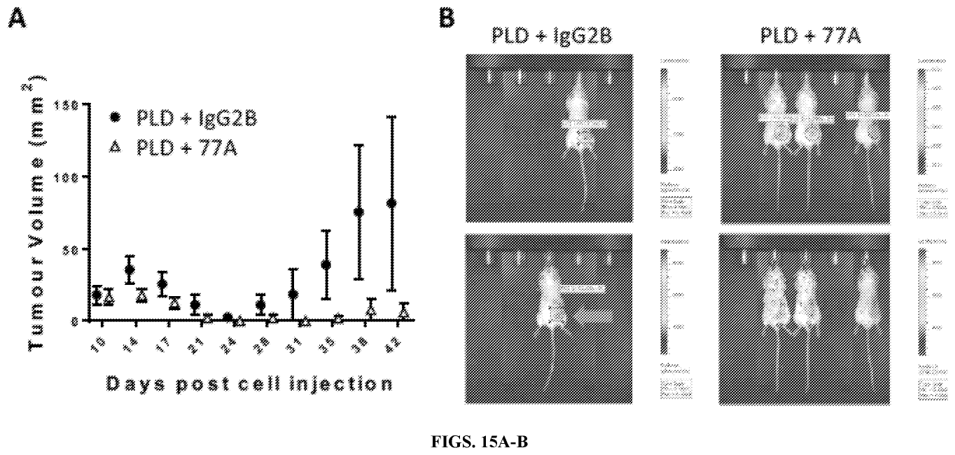

Pegylated liposomal doxorubicin (PLD; Doxil®) was chosen as the first agent to combine with 77A since it has regulatory approval for breast cancer therapy (Lao et al., 2013)], is active against other malignancies (Lyseng-Williamson et al., 2013), and causes ICD (Kroemer et al., 2013). PLD with IgG2B or 77A was evaluated in BALB/c mice orthotopically injected with 4T1 cells. Compared with PLD+IgG2B, PLD+77A induced a greater reduction and delay in tumor growth (FIG. 15A), and ⅘ mice were cured with PLD++77A (defined as lack of recurrence at day 100), while this was the case for only ⅕ PLD+IgG2B controls. Also, this combination was evaluated in a CT26-based immune-competent colon carcinoma model, where a greater reduction and delay in tumor growth was again seen. Notably, by day 81, ⅗ PLD+77A mice remained alive and without tumor recurrence, while only ⅕ IgG2B+PLD mice were alive, and that individual had measurable disease (FIG. 15B).

### Example 10—Anti-HSP70 Antibody Epitope Binning

Epitope binning experiments were conducted to determine the extent to which different, unrelated anti-HSP70 antibodies interfered or blocked the binding of 77A to HSP70.

To conduct binning experiments, antibodies were set up in binning pairs. The Octet platform was used in the sandwich configuration. Analysis was performed using the Data Analysis HT software. The first antibody for each pair was loaded onto the dip-and-read sensor surface, and then the sensor was dipped into the HSP70 solution to load the antigen. Finally, the sensor was dipped into the second antibody for each pair and the response was measured. Results for each pair were tabulated into a pairwise matrix.

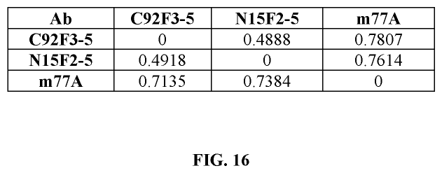

Results are depicted in FIG. 16. The numbers in FIG. 16 reflect the percent of maximal binding in the presence of the potentially competing antibody. As expected, all antibodies competed with themselves. As depicted, 77A does not compete with C92F3-5 (Fisher Scientific) or N15F2-5 (Enzo Life Sciences).

### Example 11—Binding Kinetics for ADP vs ATP-Bound HSP70

77A binding to either the ATP or ADP-bound forms of HSP70 was characterized using biolayer interferometry (BLI) instrumentation and ELISA.

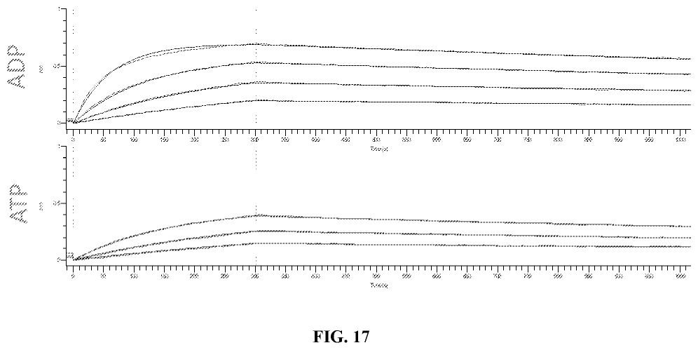

HSP70 was expressed in SF9 cells. Affinity chromatography using resins selective for ADP or ATP-binding proteins were used to enrich bulk preparations of the correspondingly bound HSP70 recombinant protein. Protein concentrations, determined after elution, were measured so that equal concentrations could be used in subsequent assays. For BLI-based assays, antibodies and reagents were diluted out onto microwell plates and loaded into the instrument. Antibodies being tested were immobilized onto dip-and-read sensors and then observed for kinetics during binding to ATP or ADP-enriched HSP70. For ELISA, the relevant enriched HSP70 proteins were directly coated onto ELISA plate wells overnight. Test antibodies were then serially diluted across the plate wells, and detected using an appropriate secondary antibody-HRP conjugate. The plates were developed using a TMB substrate, and the O.D. stabilized using an acid stop solution. BLI sensogram trace data is shown in FIG. 17. As depicted, there was a superior response (nm shift) during the antigen association step for ADP-enriched HSP70 in contrast to ATP-enriched HSP70. Binding kinetics are shown in Table 4. As depicted, 77A bound ADP-enriched HSP70 with a ≥4-fold greater affinity (KD) than ATP-enriched HSP70.

TABLE 4
77A Binding Kinetics for ADP- vs ATP-bound HSP70

| HSP70 | | KD | KD | ka | ka | kdis | kdis | | Full | Full |
|---|---|---|---|---|---|---|---|---|---|---|
| (Conc μM) | Resp. | (M) | Error | (1/Ms) | Error | (1/s) | Error | Rmax | X\hat{ }2 | R\hat{ }2 |
| ADP (300) | 0.6942 | 5.42E−09 | 3.37E−11 | 5.38E+04 | 1.85E+02 | 2.92E−04 | 1.51E−06 | 0.7056 | 0.114 | 0.9987 |
| ADP (150) | 0.5313 | 5.42E−09 | 3.37E−11 | 5.38E+04 | 1.85E+02 | 2.92E−04 | 1.51E−06 | 0.5955 | 0.114 | 0.9987 |
| ADP (75) | 0.3564 | 5.42E−09 | 3.37E−11 | 5.38E+04 | 1.85E+02 | 2.92E−04 | 1.51E−06 | 0.5216 | 0.114 | 0.9987 |
| ADP (37.5) | 0.1988 | 5.42E−09 | 3.37E−11 | 5.38E+04 | 1.85E+02 | 2.92E−04 | 1.51E−06 | 0.4538 | 0.114 | 0.9987 |
| ATP (300) | 0.3887 | 2.02E−08 | 1.61E−10 | 1.88E+04 | 1.17E+02 | 3.80E−04 | 1.89E−06 | 0.4937 | 0.0296 | 0.9985 |
| ATP (150) | 0.2458 | 2.02E−08 | 1.61E−10 | 1.88E+04 | 1.17E+02 | 3.80E−04 | 1.89E−06 | 0.4655 | 0.0296 | 0.9985 |
| ATP (75) | 0.1406 | 2.02E−08 | 1.61E−10 | 1.88E+04 | 1.17E+02 | 3.80E−04 | 1.89E−06 | 0.448 | 0.0296 | 0.9985 |

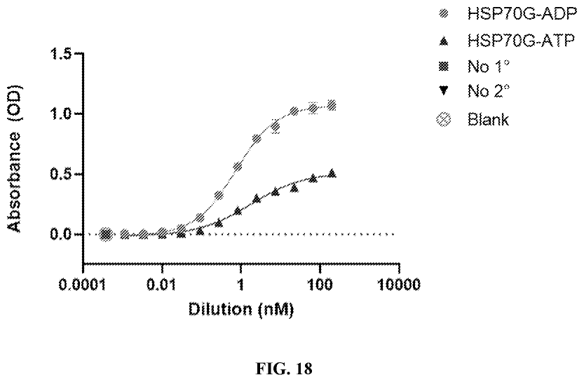

ELISA data, shown in FIG. 18, depicted the same trend, in which 77A had higher maximal absorbance with the ADP-enriched HSP70 than the ATP-enriched HSP70. 77A also had a lower EC50 value with the ADP-enriched HSP70 (0.7372 nM) than the ATP-enriched HSP70 (1.774 nM).

Together, these results show that 77A preferentially binds to the ADP-HSP70 protein relative to the ATP-HSP70 protein.

### Example 12—77A Activity in a Murine CT-26 Tumor Model

This Example describes the testing of the 77A antibody, alone and in combination with an anti-CTLA4 antibody, in a murine CT-26 colorectal adenocarcinoma cachexia model.

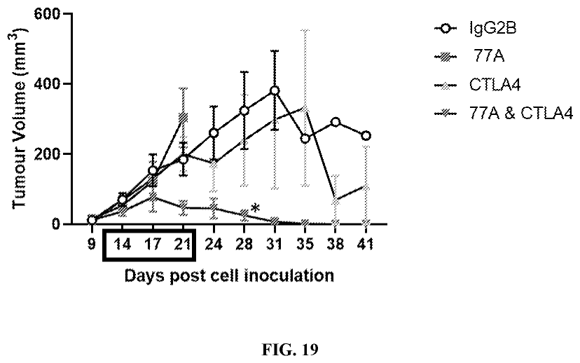

CT26 cells were inoculated subcutaneously in nude BALB/c mice. Treatment was started when the tumors reached ˜80 mm³ average volume. Mice were administered 10 mg/kg of 77A, isotype control (IgG2B), and/or anti-CTLA4 antibody on days 14, 17, and 21. Tumor volume is shown in FIG. 19. As depicted, the combination of 77A and anti-CTLA-4 antibody had the greatest effect on tumor volume, and the combination significantly reduced tumor volume relative to isotype control. The combination therapy completely eradicated the tumors in all 5 treated animals.

### Example 13—Humanization

This Example describes the generation of humanized variants of the 77A antibody.

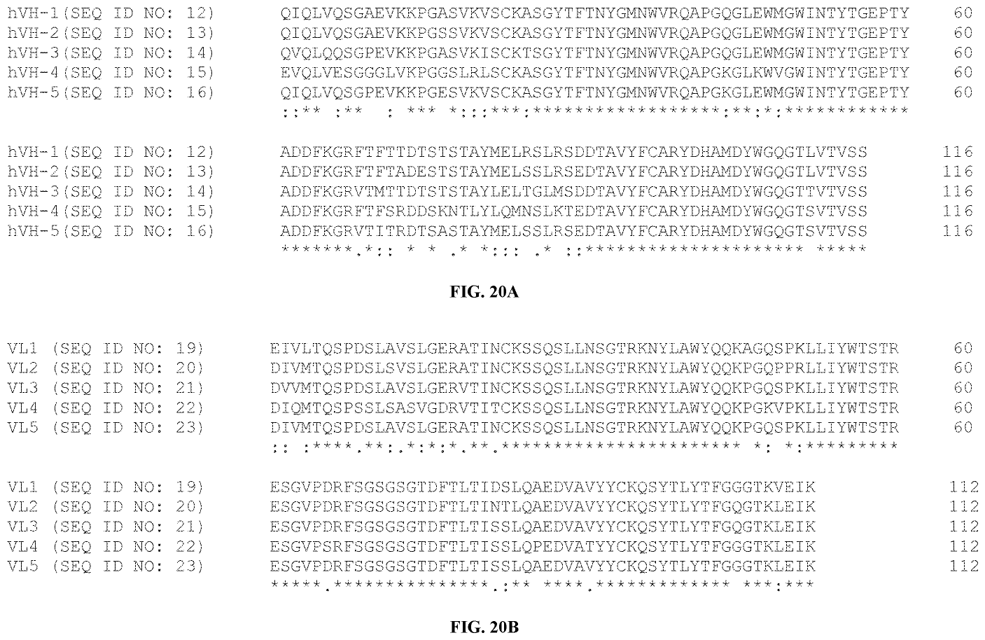

The murine immunoglobulin family subgroup for the 77A antibody was determined, and the antigen binding sequences were then modeled and grafted into compatible potential frameworks of relevant human immunoglobulin families for both the heavy and light chains. Backmutations were employed where necessary. Five humanized heavy chain variants (hVH-1 through hVH-5, as shown in Table 5), and five humanized light chain variants (hVL-1 through hVL-5, as shown in Table 5) were generated. A sequence alignment of hVH-1 through hVH-5 is depicted in FIG. 20A, and a sequence alignment of hVL-1 through hVL-5 is depicted in FIG. 20B.

TABLE 5
Humanized variable region sequences

| VH/VL | SEQ | |
|---|---|---|
| Name | ID NO | Amino Acid Sequence |
| hVH-1 | 12 | QIQLVQSGAEVKKPGASVKVSCKASGYTFTNYGMNWVRQAPGQGLEWM |
| | | GWINTYTGEPTYADDFKGRFTFTTDTSTSTAYMELRSLRSDDTAVYFC |
| | | ARYDHAMDYWGQGTLVTVSS |
| hVH-2 | 13 | QIQLVQSGAEVKKPGSSVKVSCKASGYTFTNYGMNWVRQAPGQGLEWM |
| | | GWINTYTGEPTYADDFKGRFTFTADESTSTAYMELSSLRSEDTAVYFC |
| | | ARYDHAMDYWGQGTLVTVSS |
| hVH-3 | 14 | QVQLQQSGPEVKKPGASVKISCKTSGYTFTNYGMNWVRQAPGQGLEWM |
| | | GWINTYTGEPTYADDFKGRVTMTTDTSTSTAYLELTGLMSDDTAVYFC |
| | | ARYDHAMDYWGQGTTVTVSS |
| hVH-4 | 15 | EVQLVESGGGLVKPGGSLRLSCKASGYTFTNYGMNWVRQAPGKGLKWV |
| | | GWINTYTGEPTYADDFKGRFTFSRDDSKNTLYLQMNSLKTEDTAVYFC |
| | | ARYDHAMDYWGQGTSVTVSS |
| hVH-5 | 16 | QIQLVQSGPEVKKPGESVKVSCKASGYTFTNYGMNWVRQAPGKGLEWM |
| | | GWINTYTGEPTYADDFKGRVTITRDTSASTAYMELSSLRSEDTAVYFC |
| | | ARYDHAMDYWGQGTSVTVSS |
| hVL-1 | 19 | EIVLTQSPDSLAVSLGERATINCKSSQSLLNSGTRKNYLAWYQQKAGQ |
| | | SPKLLIYWTSTRESGVPDRFSGSGSGTDFTLTIDSLQAEDVAVYYCKQ |
| | | SYTLYTFGGGTKVEIK |
| hVL-2 | 20 | DIVMTQSPDSLSVSLGERATINCKSSQSLLNSGTRKNYLAWYQQKPGQ |
| | | PPRLLIYWTSTRESGVPDRFSGSGSGTDFTLTINTLQAEDVAVYYCKQ |
| | | SYTLYTFGQGTKLEIK |
| hVL-3 | 21 | DVVMTQSPDSLAVSLGERVTINCKSSQSLLNSGTRKNYLAWYQQKPGQ |
| | | SPKLLIYWTSTRESGVPDRFSGSGSGTDFTLTISSLQAEDVAVYYCKQ |
| | | SYTLYTFGQGTKLEIK |
| hVL-4 | 22 | DIQMTQSPSSLSASVGDRVTITCKSSQSLLNSGTRKNYLAWYQQKPGK |
| | | VPKLLIYWTSTRESGVPSRFSGSGSGTDFTLTISSLQPEDVATYYCKQ |
| | | SYTLYTFGGGTKLEIK |
| hVL-5 | 23 | DIVMTQSPDSLAVSLGERATINCKSSQSLLNSGTRKNYLAWYQQKPGQ |
| | | SPKLLIYWTSTRESGVPDRFSGSGSGTDFTLTISSLQAEDVAVYYCKQ |
| | | SYTLYTFGGGTKLEIK |

Antibodies including combinations of each humanized heavy and light chain were made and characterized. These antibodies were referred to as h77A-1 through 5 h77A-25, and corresponding amino acid sequences are depicted in Table 6.

TABLE 6
Humanized 77A variants

| | | | SEQ ID NO | | | | | | | |
|---|---|---|---|---|---|---|---|---|---|---|
| | | | | VH | VH | VH | | VL | VL | VL |
| Name | | | | CDR | CDR | CDR | | CDR | CDR | CDR |
| Antibody | VH | VL | VH | 1 | 2 | 3 | VL | 1 | 2 | 3 |
| 77A | VH | VL | 7 | 1 | 2 | 3 | 8 | 4 | 5 | 6 |
| h77A-1 | hVH-1 | hVL-1 | 12 | 1 | 2 | 3 | 19 | 4 | 5 | 6 |
| h77A-2 | hVH-1 | hVL-2 | 12 | 1 | 2 | 3 | 20 | 4 | 5 | 6 |
| h77A-3 | hVH-1 | hVL-3 | 12 | 1 | 2 | 3 | 21 | 4 | 5 | 6 |
| h77A-4 | hVH-1 | hVL-4 | 12 | 1 | 2 | 3 | 22 | 4 | 5 | 6 |
| h77A-5 | hVH-1 | hVL-5 | 12 | 1 | 2 | 3 | 23 | 4 | 5 | 6 |
| h77A-6 | hVH-2 | hVL-1 | 13 | 1 | 2 | 3 | 19 | 4 | 5 | 6 |
| h77A-7 | hVH-2 | hVL-2 | 13 | 1 | 2 | 3 | 20 | 4 | 5 | 6 |
| h77A-8 | hVH-2 | hVL-3 | 13 | 1 | 2 | 3 | 21 | 4 | 5 | 6 |
| h77A-9 | hVH-2 | hVL-4 | 13 | 1 | 2 | 3 | 22 | 4 | 5 | 6 |
| h77A-10 | hVH-2 | hVL-5 | 13 | 1 | 2 | 3 | 23 | 4 | 5 | 6 |
| h77A-11 | hVH-3 | hVL-1 | 14 | 1 | 2 | 3 | 19 | 4 | 5 | 6 |
| h77A-12 | hVH-3 | hVL-2 | 14 | 1 | 2 | 3 | 20 | 4 | 5 | 6 |
| h77A-13 | hVH-3 | hVL-3 | 14 | 1 | 2 | 3 | 21 | 4 | 5 | 6 |
| h77A-14 | hVH-3 | hVL-4 | 14 | 1 | 2 | 3 | 22 | 4 | 5 | 6 |
| h77A-15 | hVH-3 | hVL-5 | 14 | 1 | 2 | 3 | 23 | 4 | 5 | 6 |
| h77A-16 | hVH-4 | hVL-1 | 15 | 1 | 2 | 3 | 19 | 4 | 5 | 6 |
| h77A-17 | hVH-4 | hVL-2 | 15 | 1 | 2 | 3 | 20 | 4 | 5 | 6 |
| h77A-18 | hVH-4 | hVL-3 | 15 | 1 | 2 | 3 | 21 | 4 | 5 | 6 |
| h77A-19 | hVH-4 | hVL-4 | 15 | 1 | 2 | 3 | 22 | 4 | 5 | 6 |
| h77A-20 | hVH-4 | hVL-5 | 15 | 1 | 2 | 3 | 23 | 4 | 5 | 6 |
| h77A-21 | hVH-5 | hVL-1 | 16 | 1 | 2 | 3 | 19 | 4 | 5 | 6 |
| h77A-22 | hVH-5 | hVL-2 | 16 | 1 | 2 | 3 | 20 | 4 | 5 | 6 |
| h77A-23 | hVH-5 | hVL-3 | 16 | 1 | 2 | 3 | 21 | 4 | 5 | 6 |
| h77A-24 | hVH-5 | hVL-4 | 16 | 1 | 2 | 3 | 22 | 4 | 5 | 6 |
| h77A-25 | hVH-5 | hVL-5 | 16 | 1 | 2 | 3 | 23 | 4 | 5 | 6 |

Humanized variants were characterized for binding using biolayer interferometry (BLI). Chimeric antibodies including the murine 77A antigen binding region and human Fc regions were also tested. Antibodies and reagents were diluted out onto microwell plates and loaded into the instrument. Diluted antibodies were first immobilized onto dip-and-read sensors, and then measurements for baselines were acquired. Subsequently, the sensors were dipped into wells containing the antigen in solution, or buffer alone. The change in number of molecules bound to the sensor was quantified by measurement of the shift in the interference pattern of light during each step of the protocol. Mathematical modeling was performed based on those values and the concentrations of reagents used in the assay and used to calculate kinetics values.

Results are shown in Table 7. As depicted, all of the tested humanized variants had affinity measurements within 2 to 5-fold of the parental murine antibody.

TABLE 7
Humanized 77A antibodies binding kinetics

| | Capture | | | | | | | |
|---|---|---|---|---|---|---|---|---|
| | level | kₐ | kₐ | K_{D} | K_{D} | | | Rₘₐₓ at |
| Antibody | (nm) | (m⁻¹s⁻¹) | (s⁻¹) | (M) | (nM) | R² | X² | 33.3 nM |
| 77A | 0.834 | 6.74E+04 | 8.08E−05 | 1.20E−09 | 1.2 | 0.9967 | 0.1175 | 0.146 |
| Chimera | 0.836 | 7.16E+04 | 9.94E−05 | 1.39E−09 | 1.39 | 0.9979 | 0.1813 | 0.2441 |
| IgG1 | | | | | | | | |
| Chimera | 0.786 | 7.13E+04 | 1.29E−04 | 1.81E−09 | 1.81 | 0.9973 | 0.2251 | 0.2397 |
| IgG4 | | | | | | | | |
| Chimera | 0.824 | 7.37E+04 | 1.12E−04 | 1.52E−09 | 1.52 | 0.9985 | 0.1527 | 0.2662 |
| IgG2 | | | | | | | | |
| h77A-1 | 0.819 | 6.62E+04 | 1.73E−04 | 2.61E−09 | 2.61 | 0.9972 | 0.2055 | 0.2409 |
| h77A-2 | 0.863 | 5.99E+04 | 3.08E−04 | 5.13E−09 | 5.13 | 0.9967 | 0.2669 | 0.2574 |
| h77A-3 | 0.806 | 6.23E+04 | 3.05E−04 | 4.90E−09 | 4.9 | 0.9978 | 0.1294 | 0.2158 |
| h77A-4 | 0.777 | 3.74E+04 | 2.87E−04 | 7.66E−09 | 7.66 | 0.9976 | 0.133 | 0.2462 |
| h77A-5 | 0.812 | 5.14E+04 | 3.53E−04 | 6.87E−09 | 6.87 | 0.9983 | 0.0856 | 0.239 |
| h77A-6 | 0.76 | 5.31E+04 | 1.40E−04 | 2.64E−09 | 2.64 | 0.9976 | 0.2202 | 0.2779 |
| h77A-7 | 0.789 | 4.87E+04 | 4.88E−04 | 1.00E−08 | 10 | 0.9956 | 0.1637 | 0.2465 |
| h77A-9 | 0.83 | 3.92E+04 | 3.48E−04 | 8.89E−09 | 8.89 | 0.9952 | 0.2576 | 0.2308 |
| h77A-10 | 0.854 | 5.77E+04 | 4.09E−04 | 7.10E−09 | 7.1 | 0.9943 | 0.3562 | 0.2578 |
| h77A-11 | 0.781 | 4.46E+04 | 1.89E−04 | 4.23E−09 | 4.23 | 0.9966 | 0.1939 | 0.2206 |
| h77A-12 | 0.78 | 4.45E+04 | 4.46E−04 | 1.00E−08 | 10 | 0.9966 | 0.1605 | 0.2252 |
| h77A-13 | 0.775 | 4.47E+04 | 3.96E−04 | 8.85E−09 | 8.85 | 0.9953 | 0.1987 | 0.1937 |
| h77A-14 | 0.81 | 4.11E+04 | 6.16E−04 | 1.50E−08 | 15 | 0.9933 | 0.1716 | 0.193 |
| h77A-15 | 0.777 | 4.76E+04 | 5.13E−04 | 1.08E−08 | 10.8 | 0.9977 | 0.1126 | 0.2158 |
| h77A-16 | 0.742 | 4.40E+04 | 4.60E−04 | 1.05E−08 | 10.5 | 0.9832 | 0.1968 | 0.0937 |
| h77A-21 | 0.77 | 4.17E+04 | 5.45E−04 | 1.31E−08 | 13.1 | 0.9926 | 0.1633 | 0.1492 |
| h77A-22 | 0.798 | 3.64E+04 | 3.02E−04 | 8.31E−09 | 8.31 | 0.9816 | 0.2804 | 0.1043 |
| h77A-23 | 0.799 | 4.99E+04 | 4.47E−04 | 8.96E−09 | 8.96 | 0.9759 | 0.2499 | 0.078 |
| h77A-24 | 0.815 | 3.18E+04 | 2.94E−04 | 9.26E−09 | 9.26 | 0.9801 | 0.9801 | 0.1012 |
| h77A-25 | 0.81 | 4.73E+04 | 5.03E−04 | 1.06E−08 | 10.6 | 0.9723 | 0.2785 | 0.0976 |

### Example 14—HSP70 Uptake Mediated by Humanized 77A Variants

This Example describes the evaluation of Fc receptor involvement in uptake of HSP70 mediated by humanized 77A variants.

293HSP70KO cells were transfected with vectors encoding a panel of mouse Fc receptors or human Fc Receptors for 24 hours. Transfections were performed using JetPrime in 10 cm dishes with 2.5 μg (for mouse Fc receptors) or 1.42 μg (for human Fc receptors) of each vector.

Next, HSP70GFP (BME Free; 5 μg/ml) and GFP-Nanobody Alexa-488 (1:1000) were added alone or in combination with antibody (1 μg/ml) for 1 hour at 37° C. Cells were then analyzed by FACs.

Antibodies tested were: IgG2 isotype control, 77A (murine), a chimeric 77A with a human IgG1 Fc domain, a chimeric 77A with a human IgG2 Fc domain, a chimeric 77a with a human IgG4 domain, and humanized variants h77A-1 (including hVH-1 and hVL-1), h77A-6 (including hVH-2 and hVL-1), and h77A-11 (including hVH-3 and hVL-1), each as described in Example 12. The humanized antibodies h77A-1, h77A-6, and h77A-11 were each tested with a human IgG2 Fc domain.

Mouse Fc receptors tested were: FcγR1 (Origene Catalog No. MR225268), FcγR2B (Origene Catalog No. MR204036), FcγR3 (Origene Catalog No. MR203404), and FcγR4 (Origene Catalog No. MR203178).

Human FC receptors tested were: FcγR1A (Origene Catalog No. RC207487), FcγR1B (Origene Catalog No. RC219204), FcγR2A (Origene Catalog No. RC205786), FcγR2B (Origene Catalog No. RC211982), FcγR2C (Origene Catalog No. RC213460), FcγR3A (Origene Catalog No. SC124061), and FcγR3B (Origene Catalog No. RC204749).

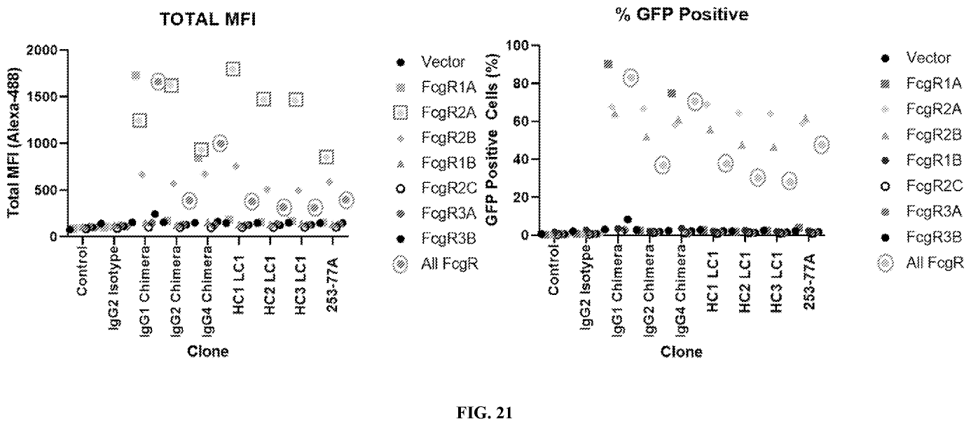

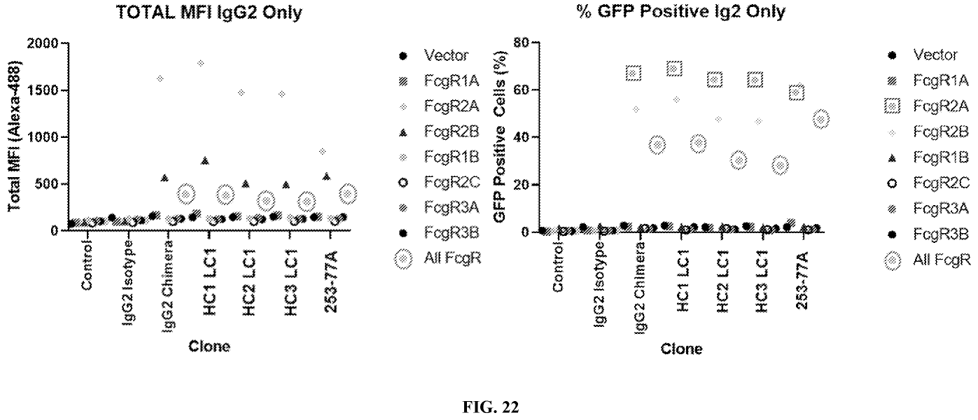

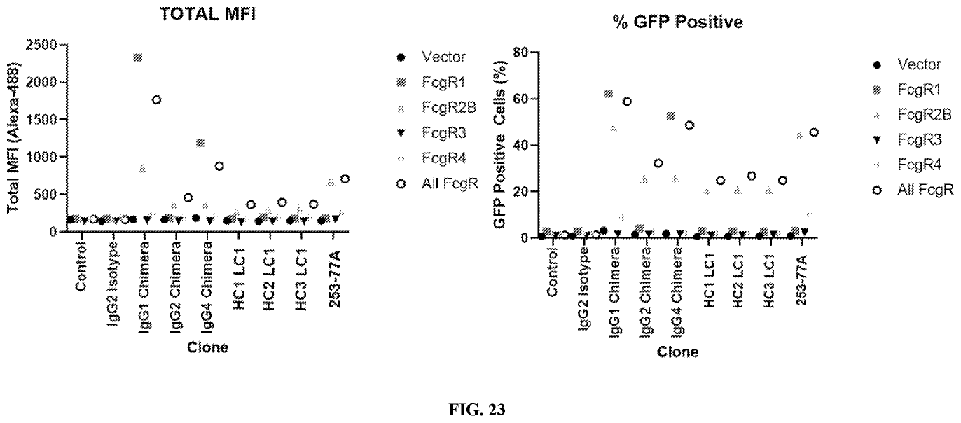

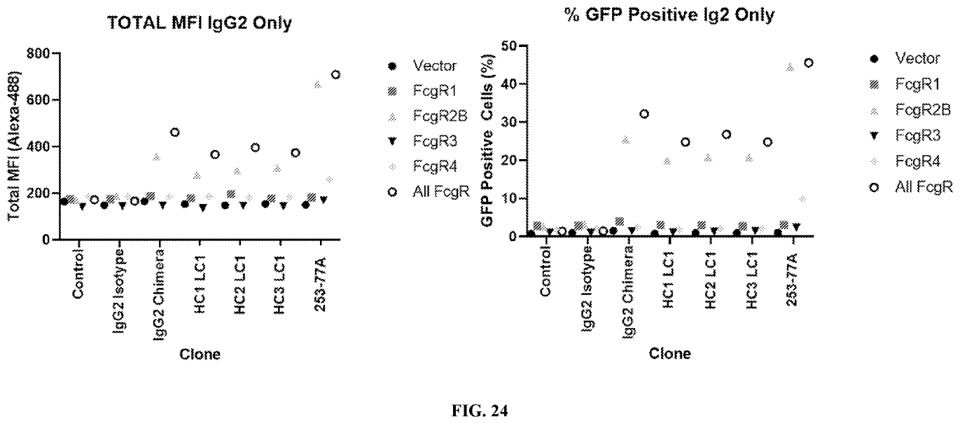

Results are depicted in FIGS. 21-24. A summary of the results with human FcγR is depicted in Table 8. The results demonstrated that all three humanized antibodies as well as the chimeric and parental murine 77A antibodies were able to mediate the uptake of HSP70 through the FcγR2A and FcγR2B receptors. The chimeric IgG1 and IgG4 antibodies also mediated HSP70 uptake through the FcγR1A receptor. A summary of the results with murine FcγR is depicted in Table 9. The experiments demonstrated that all three humanized antibodies were able to mediate the uptake of HSP70 through the murine FcγR2B receptor. The chimeric IgG1 antibody facilitated uptake through the FcγR1, FcγR2B, and FcγR4 receptors while the IgG4 chimera utilized the FcγR1 and FcγR2B receptors for HSP70 uptake. The parental murine antibody promoted the uptake of through receptors FcγR2B and FcγR4.

TABLE 8
Summary of uptake experiments with Human FcγR

| Antibody | FcγR1A | FcγR1B | FcγR2A | FcγR2B | FcγR2C | FcγR3A | FcγR3B |
|---|---|---|---|---|---|---|---|
| IgG1 Chimera | + | − | + | + | − | − | − |
| IgG2 Chimera | − | − | + | + | − | − | − |
| IgG4 Chimera | + | − | + | + | − | − | − |
| h77A-1 | − | − | + | + | − | − | − |
| h77A-6 | − | − | + | + | − | − | − |
| h77A-11 | − | − | + | + | − | − | − |
| 77A | − | − | + | + | − | − | − |

TABLE 9
Summary of uptake experiments with Mouse FcγR

| Antibody | FcγR1 | FcγR2B | FcγR3 | FcγR4 |
|---|---|---|---|---|
| IgG1 | + | + | − | + |
| Chimera | | | | |
| IgG2 | − | + | − | − |
| Chimera | | | | |
| IgG4 | + | + | − | − |
| Chimera | | | | |
| h77A-1 | − | + | − | − |
| h77A-6 | − | + | − | − |
| h77A-11 | − | + | − | − |
| 77A | − | + | − | + |

### Example 15—HSP70 Dendritic Cell Uptake Mediated by Humanized 77A Variants

This Example describes the uptake of HSP70 by dendritic cells (DCs) mediated by humanized 77A variants.

The Blood Dendritic Cell Isolation Kit II (Miltenyi Biotec) was used to isolate dendritic cells from human buffy coat. Briefly, B cells and monocytes were magnetically labeled and depleted using a cocktail of CD19 and CD14 MicroBeads. Subsequently, pre-enriched dendritic cells in the non-magnetic flow-through fraction were magnetically labeled and enriched using a cocktail of antibodies against the dendritic cell markers CD304 (BDCA-4/Neuropilin-1), CD141 (BDCA-3), and CD1c (BDCA-1). Fractions collected included plasmacytoid dendritic cells, type-1 myeloid dendritic cells (MDC1s), type-2 myeloid dendritic cells (MDC2s), and the unlabeled flow through fraction representing the non-DC fraction. The highly pure enriched cell fraction includes: plasmacytoid dendritic cells, type-1 myeloid dendritic cells (MDC1s), and type-2 myeloid dendritic cells (MDC2s).

Cells were incubated with HSP70GFP (BME Free; 5 μg/ml) and GFP-Nanobody Alexa-488 (1:1000) were added alone or in combination with antibody (1 μg/ml) for 1 hour at 4° C. Cells were then stained with or for Ghost Violet 450, CD11C, CD19, CD14, CD80, CD86, CD141, CD1C, and CD303.

Antibodies tested were: IgG2 isotype control, 77A (murine), a chimeric 77A with a human IgG1 Fc domain, a chimeric 77A with a human IgG2 Fc domain, a chimeric 77a with a human IgG4 domain, and humanized variants h77A-1 (including hVH-1 and hVL-1), h77A-6 (including hVH-2 and hVL-1), and h77A-11 (including hVH-3 and hVL-1), each as described in Example 12. The humanized antibodies h77A-1, h77A-6, and h77A-11 were each tested with a human IgG2 Fc domain.

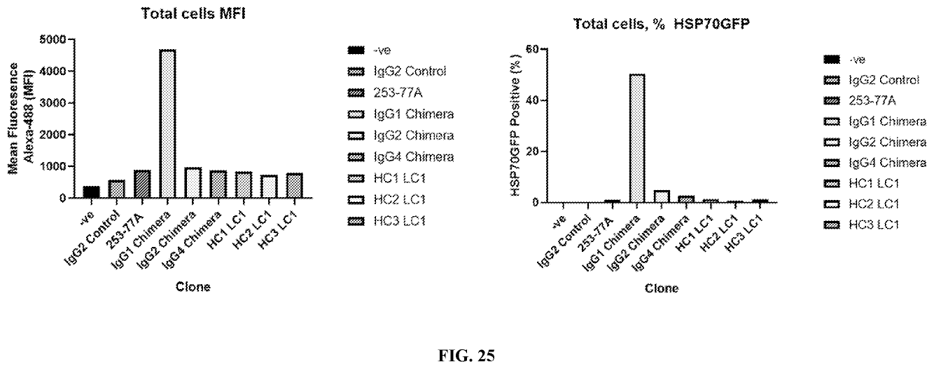

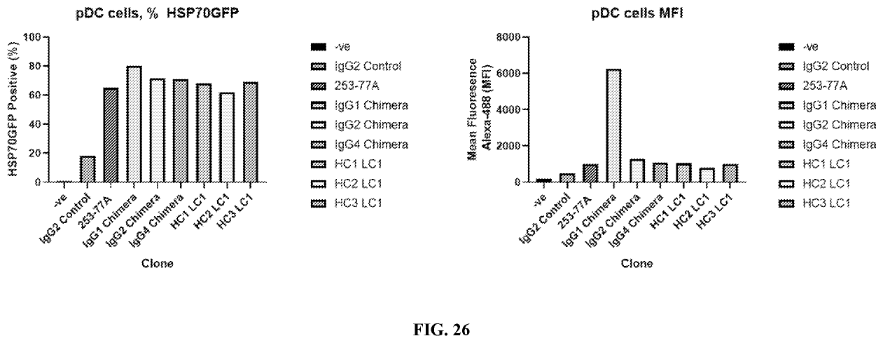

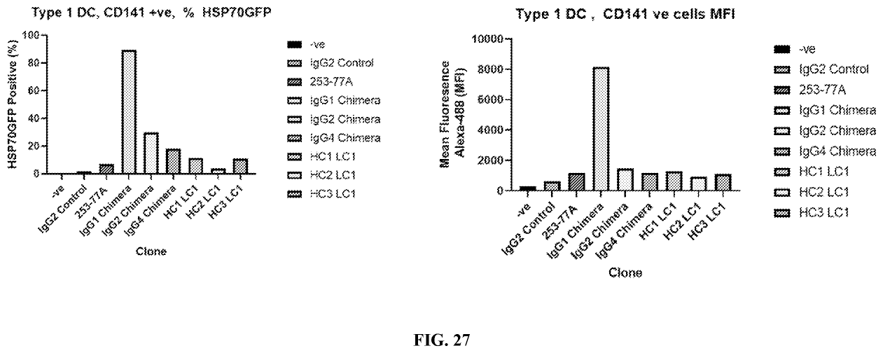

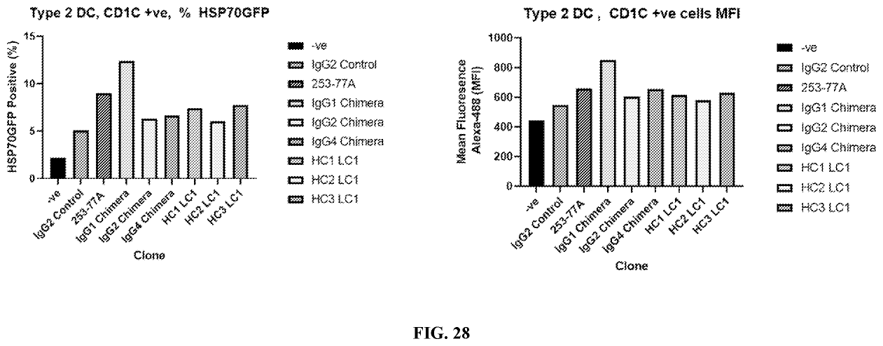

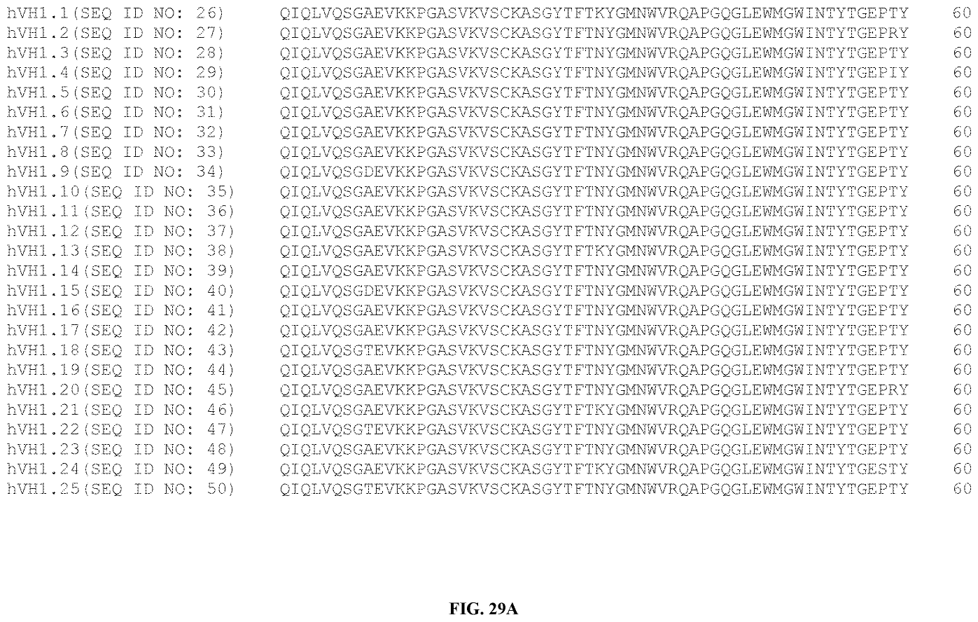

Results are depicted in FIGS. 25-28. Together, the results showed that human IgG2 isoforms of 77A mediated the uptake of HSP70 into primary human DCs, preferentially targeting plasmacytoid and type 2 peripheral blood DCs. Lesser uptake was observed for type 1 peripheral blood DCs. In addition, the IgG1 chimera only induced uptake into the unlabeled flow through fraction (FIG. 25).

### Example 16—Further 77A Variants

h77A-1, including the combination of hVH-1 and hVL-1 (as described in Example 12) was selected for further optimization.

To optimize these sequences, a large library including variants of hVH-1 and hVL-1 was generated. Subsequently, an in silico model of antibody-antigen binding was generated and antibody-antigen binding for the full library was computationally simulated. The top 95 antibodies are referred to as h77A-1.1 through h77A-1.95, and are described in Table 10 (Kabat CDR Sequences) and Table 11 (IMGT CDR sequences). Amino acid sequences of CDR variants included in h77A-1.1 through h77A-1.95 are provided in Table 12, and amino acid sequences of variable region variants included in h77A-1.1 through h77A-1.95 are provided in Table 13. Additional antibody variants h77A-1.96, h77A-1.97, h77A-1.98 (also described in Tables 10 and 11) were also made and tested.

TABLE 10
h77A-1 Variants (Kabat CDR Sequences)

| | | | SEQ ID NO | | | | | | | |
|---|---|---|---|---|---|---|---|---|---|---|
| | | | | VH | VH | VH | | VL | VL | VL |
| Name | | | | CDR | CDR | CDR | | CDR | CDR | CDR |
| Antibody | VH | VL | VH | 1 | 2 | 3 | VL | 1 | 2 | 3 |
| h77A-1.1 | hVH-1.1 | hVL-1.1 | 26 | 164 | 2 | 3 | 105 | 4 | 5 | 6 |
| h77A-1.2 | hVH-1.2 | hVL-1.1 | 27 | 1 | 2 | 3 | 105 | 4 | 5 | 6 |
| h77A-1.3 | hVH-1.3 | hVL-1.1 | 28 | 1 | 2 | 170 | 105 | 4 | 5 | 6 |
| h77A-1.4 | hVH-1.4 | hVL-1.1 | 29 | 1 | 2 | 3 | 105 | 4 | 5 | 6 |
| h77A-1.5 | hVH-1.5 | hVL-1.2 | 30 | 1 | 2 | 3 | 106 | 4 | 5 | 162 |
| h77A-1.6 | hVH-1.6 | hVL-1.1 | 31 | 1 | 2 | 3 | 105 | 4 | 5 | 6 |
| h77A-1.7 | hVH-1.7 | hVL-1.1 | 32 | 1 | 2 | 171 | 105 | 4 | 5 | 6 |
| h77A-1.8 | hVH-1.5 | hVL-1.3 | 30 | 1 | 2 | 3 | 107 | 4 | 5 | 6 |
| h77A-1.9 | hVH-1.8 | hVL-1.1 | 33 | 1 | 2 | 172 | 105 | 4 | 5 | 6 |
| h77A-1.10 | hVH-1.9 | hVL-1.1 | 34 | 1 | 2 | 3 | 105 | 4 | 5 | 6 |
| h77A-1.11 | hVH-1.5 | hVL-1.4 | 30 | 1 | 2 | 3 | 108 | 4 | 5 | 6 |
| h77A-1.12 | hVH-1.5 | hVL-1.5 | 30 | 1 | 2 | 3 | 109 | 159 | 5 | 6 |
| h77A-1.13 | hVH-1.10 | hVL-1.1 | 35 | 1 | 2 | 173 | 105 | 4 | 5 | 6 |
| h77A-1.14 | hVH-1.11 | hVL-1.1 | 36 | 1 | 2 | 3 | 105 | 4 | 5 | 6 |
| h77A-1.15 | hVH-1.12 | hVL-1.1 | 37 | 1 | 2 | 174 | 105 | 4 | 5 | 6 |
| h77A-1.16 | hVH-1.1 | hVL-1.3 | 26 | 164 | 2 | 3 | 107 | 4 | 5 | 6 |
| h77A-1.17 | hVH-1.13 | hVL-1.1 | 38 | 164 | 2 | 175 | 105 | 4 | 5 | 6 |
| h77A-1.18 | hVH-1.6 | hVL-1.6 | 31 | 1 | 2 | 3 | 110 | 4 | 5 | 6 |
| h77A-1.19 | hVH-1.14 | hVL-1.1 | 39 | 1 | 2 | 3 | 105 | 4 | 5 | 6 |
| h77A-1.20 | hVH-1.15 | hVL-1.1 | 40 | 1 | 2 | 3 | 105 | 4 | 5 | 6 |
| h77A-1.21 | hVH-1.9 | hVL-1.7 | 34 | 1 | 2 | 3 | 111 | 4 | 5 | 6 |
| h77A-1.22 | hVH-1.16 | hVL-1.5 | 41 | 1 | 2 | 3 | 109 | 159 | 5 | 6 |
| h77A-1.23 | hVH-1.5 | hVL-1.8 | 30 | 1 | 2 | 3 | 112 | 4 | 5 | 163 |
| h77A-1.24 | hVH-1.3 | hVL-1.9 | 28 | 1 | 2 | 170 | 113 | 4 | 5 | 6 |
| h77A-1.25 | hVH-1.7 | hVL-1.10 | 32 | 1 | 2 | 171 | 114 | 4 | 5 | 6 |
| h77A-1.26 | hVH-1.17 | hVL-1.1 | 42 | 1 | 2 | 176 | 105 | 4 | 5 | 6 |
| h77A-1.27 | hVH-1.11 | hVL-1.11 | 36 | 1 | 2 | 3 | 115 | 4 | 5 | 6 |
| h77A-1.28 | hVH-1.18 | hVL-1.1 | 43 | 1 | 2 | 172 | 105 | 4 | 5 | 6 |
| h77A-1.29 | hVH-1.7 | hVL-1.5 | 32 | 1 | 2 | 171 | 109 | 159 | 5 | 6 |
| h77A-1.30 | hVH-1.19 | hVL-1.12 | 44 | 1 | 2 | 3 | 116 | 4 | 5 | 6 |
| h77A-1.31 | hVH-1.10 | hVL-1.13 | 35 | 1 | 2 | 173 | 117 | 4 | 5 | 6 |
| h77A-1.32 | hVH-1.20 | hVL-1.1 | 45 | 1 | 2 | 177 | 105 | 4 | 5 | 6 |
| h77A-1.33 | hVH-1.21 | hVL-1.1 | 46 | 164 | 2 | 178 | 105 | 4 | 5 | 6 |
| h77A-1.34 | hVH-1.11 | hVL-1.14 | 36 | 1 | 2 | 3 | 118 | 4 | 5 | 6 |
| h77A-1.35 | hVH-1.22 | hVL-1.11 | 47 | 1 | 2 | 179 | 115 | 4 | 5 | 6 |
| h77A-1.36 | hVH-1.23 | hVL-1.5 | 48 | 1 | 2 | 179 | 109 | 159 | 5 | 6 |
| h77A-1.37 | hVH-1.24 | hVL-1.1 | 49 | 164 | 167 | 174 | 105 | 4 | 5 | 6 |
| h77A-1.38 | hVH-1.25 | hVL-1.1 | 50 | 1 | 2 | 180 | 105 | 4 | 5 | 6 |
| h77A-1.39 | hVH-1.26 | hVL-1.2 | 51 | 1 | 2 | 3 | 106 | 4 | 5 | 162 |
| h77A-1.40 | hVH-1.27 | hVL-1.15 | 52 | 1 | 2 | 181 | 119 | 4 | 5 | 162 |
| h77A-1.41 | hVH-1.28 | hVL-1.4 | 53 | 1 | 2 | 174 | 108 | 4 | 5 | 6 |
| h77A-1.42 | hVH-1.29 | hVL-1.1 | 54 | 1 | 2 | 182 | 105 | 4 | 5 | 6 |
| h77A-1.43 | hVH-1.30 | hVL-1.12 | 55 | 1 | 2 | 3 | 116 | 4 | 5 | 6 |
| h77A-1.44 | hVH-1.31 | hVL-1.12 | 56 | 1 | 2 | 183 | 116 | 4 | 5 | 6 |
| h77A-1.45 | hVH-1.32 | hVL-1.16 | 57 | 1 | 2 | 3 | 120 | 4 | 5 | 163 |
| h77A-1.46 | hVH-1.33 | hVL-1.17 | 58 | 1 | 2 | 3 | 121 | 4 | 5 | 6 |
| h77A-1.47 | hVH-1.34 | hVL-1.18 | 59 | 1 | 2 | 180 | 122 | 4 | 5 | 6 |
| h77A-1.48 | hVH-1.35 | hVL-1.4 | 60 | 1 | 2 | 181 | 108 | 4 | 5 | 6 |
| h77A-1.49 | hVH-1.36 | hVL-1.19 | 61 | 1 | 168 | 184 | 123 | 4 | 5 | 6 |
| h77A-1.50 | hVH-1.37 | hVL-1.10 | 62 | 1 | 2 | 3 | 114 | 4 | 5 | 6 |
| h77A-1.51 | hVH-1.38 | hVL-1.20 | 63 | 165 | 2 | 185 | 124 | 4 | 5 | 6 |
| h77A-1.52 | hVH-1.39 | hVL-1.1 | 64 | 166 | 2 | 170 | 105 | 4 | 5 | 6 |
| h77A-1.53 | hVH-1.40 | hVL-1.21 | 65 | 1 | 2 | 3 | 125 | 4 | 5 | 6 |
| h77A-1.54 | hVH-1.41 | hVL-1.1 | 66 | 164 | 2 | 170 | 105 | 4 | 5 | 6 |
| h77A-1.55 | hVH-1.42 | hVL-1.21 | 67 | 1 | 2 | 171 | 125 | 4 | 5 | 6 |
| h77A-1.56 | hVH-1.43 | hVL-1.22 | 68 | 165 | 2 | 3 | 126 | 4 | 5 | 6 |
| h77A-1.57 | hVH-1.44 | hVL-1.23 | 69 | 1 | 169 | 174 | 127 | 160 | 5 | 6 |
| h77A-1.58 | hVH-1.45 | hVL-1.24 | 70 | 1 | 2 | 184 | 128 | 4 | 5 | 6 |
| h77A-1.59 | hVH-1.46 | hVL-1.13 | 71 | 1 | 2 | 184 | 117 | 4 | 5 | 6 |
| h77A-1.60 | hVH-1.47 | hVL-1.25 | 72 | 1 | 2 | 175 | 129 | 4 | 5 | 6 |
| h77A-1.61 | hVH-1.48 | hVL-1.26 | 73 | 1 | 2 | 3 | 130 | 4 | 5 | 6 |
| h77A-1.62 | hVH-1.49 | hVL-1.27 | 74 | 1 | 2 | 3 | 131 | 4 | 5 | 6 |
| h77A-1.63 | hVH-1.48 | hVL-1.28 | 73 | 1 | 2 | 3 | 132 | 4 | 5 | 6 |
| h77A-1.64 | hVH-1.50 | hVL-1.29 | 75 | 1 | 167 | 3 | 133 | 4 | 5 | 6 |
| h77A-1.65 | hVH-1.51 | hVL-1.30 | 76 | 1 | 169 | 3 | 134 | 4 | 5 | 6 |
| h77A-1.66 | hVH-1.52 | hVL-1.3 | 77 | 166 | 167 | 3 | 107 | 4 | 5 | 6 |
| h77A-1.67 | hVH-1.53 | hVL-1.31 | 78 | 1 | 2 | 3 | 135 | 4 | 5 | 6 |
| h77A-1.68 | hVH-1.54 | hVL-1.32 | 79 | 1 | 2 | 3 | 136 | 4 | 5 | 6 |
| h77A-1.69 | hVH-1.55 | hVL-1.33 | 80 | 1 | 168 | 3 | 137 | 4 | 5 | 6 |
| h77A-1.70 | hVH-1.16 | hVL-1.34 | 41 | 1 | 2 | 3 | 138 | 4 | 5 | 6 |
| h77A-1.71 | hVH-1.56 | hVL-1.35 | 81 | 1 | 2 | 3 | 139 | 4 | 5 | 6 |
| h77A-1.72 | hVH-1.57 | hVL-1.1 | 82 | 166 | 168 | 3 | 105 | 4 | 5 | 6 |
| h77A-1.73 | hVH-1.58 | hVL-1.22 | 83 | 1 | 2 | 3 | 126 | 4 | 5 | 6 |
| h77A-1.74 | hVH-1.59 | hVL-1.36 | 84 | 1 | 2 | 3 | 140 | 4 | 5 | 6 |
| h77A-1.75 | hVH-1.60 | hVL-1.37 | 85 | 1 | 2 | 3 | 141 | 161 | 5 | 6 |
| h77A-1.76 | hVH-1.61 | hVL-1.37 | 86 | 1 | 168 | 3 | 141 | 161 | 5 | 6 |
| h77A-1.77 | hVH-1.62 | hVL-1.13 | 87 | 166 | 2 | 184 | 117 | 4 | 5 | 6 |
| h77A-1.78 | hVH-1.63 | hVL-1.38 | 88 | 1 | 2 | 178 | 142 | 4 | 5 | 163 |
| h77A-1.79 | hVH-1.64 | hVL-1.39 | 89 | 164 | 2 | 170 | 143 | 4 | 5 | 162 |
| h77A-1.80 | hVH-1.65 | hVL-1.40 | 90 | 1 | 2 | 174 | 144 | 4 | 5 | 6 |
| h77A-1.81 | hVH-1.66 | hVL-1.5 | 91 | 166 | 2 | 183 | 109 | 161 | 5 | 6 |
| h77A-1.82 | hVH-1.67 | hVL-1.41 | 92 | 1 | 168 | 3 | 145 | 4 | 5 | 6 |
| h77A-1.83 | hVH-1.68 | hVL-1.42 | 93 | 1 | 2 | 184 | 146 | 4 | 5 | 6 |
| h77A-1.84 | hVH-1.69 | hVL-1.43 | 94 | 165 | 2 | 183 | 147 | 4 | 5 | 6 |
| h77A-1.85 | hVH-1.70 | hVL-1.44 | 95 | 166 | 2 | 183 | 148 | 4 | 5 | 6 |
| h77A-1.86 | hVH-1.71 | hVL-1.45 | 96 | 1 | 168 | 3 | 149 | 161 | 5 | 6 |
| h77A-1.87 | hVH-1.72 | hVL-1.46 | 97 | 164 | 2 | 170 | 150 | 4 | 5 | 6 |
| h77A-1.88 | hVH-1.73 | hVL-1.47 | 98 | 1 | 2 | 172 | 151 | 4 | 5 | 6 |
| h77A-1.89 | hVH-1.74 | hVL-1.48 | 99 | 1 | 167 | 3 | 152 | 4 | 5 | 6 |
| h77A-1.90 | hVH-1.75 | hVL-1.32 | 100 | 166 | 167 | 3 | 136 | 4 | 5 | 6 |
| h77A-1.91 | hVH-1.66 | hVL-1.49 | 91 | 166 | 2 | 183 | 153 | 161 | 5 | 6 |
| h77A-1.92 | hVH-1.76 | hVL-1.50 | 101 | 165 | 2 | 3 | 154 | 161 | 5 | 162 |
| h77A-1.93 | hVH-1.77 | hVL-1.51 | 102 | 1 | 168 | 183 | 155 | 4 | 5 | 6 |
| h77A-1.94 | hVH-1.11 | hVL-1.52 | 36 | 1 | 2 | 3 | 156 | 161 | 5 | 6 |
| h77A-1.95 | hVH-1.78 | hVL-1.53 | 103 | 1 | 2 | 184 | 157 | 4 | 5 | 6 |
| h77A-1.96 | hVH-1 | hVL-1.54 | 12 | 1 | 2 | 3 | 24 | 4 | 5 | 6 |
| h77A-1.97 | hVH-1.79 | hVL-1.54 | 17 | 1 | 2 | 3 | 24 | 4 | 5 | 6 |
| h77A-1.98 | hVH-1.79 | hVL-1 | 17 | 1 | 2 | 3 | 19 | 4 | 5 | 6 |

TABLE 11
h77A-1 Variants (IMGT CDR Sequences)

| | | | SEQ ID NO | | | | | | | |
|---|---|---|---|---|---|---|---|---|---|---|
| | | | | VH | VH | VH | | VL | VL | VL |
| Name | | | | CDR | CDR | CDR | | CDR | CDR | CDR |
| Antibody | VH | VL | VH | 1 | 2 | 3 | VL | 1 | 2 | 3 |
| h77A-1.1 | hVH-1.1 | hVL-1.1 | 26 | 192 | 196 | 3 | 105 | 186 | 191 | 6 |
| h77A-1.2 | hVH-1.2 | hVL-1.1 | 27 | 193 | 197 | 3 | 105 | 186 | 191 | 6 |
| h77A-1.3 | hVH-1.3 | hVL-1.1 | 28 | 193 | 196 | 170 | 105 | 186 | 191 | 6 |
| h77A-1.4 | hVH-1.4 | hVL-1.1 | 29 | 193 | 198 | 3 | 105 | 186 | 191 | 6 |
| h77A-1.5 | hVH-1.5 | hVL-1.2 | 30 | 193 | 196 | 3 | 106 | 186 | 191 | 162 |
| h77A-1.6 | hVH-1.6 | hVL-1.1 | 31 | 193 | 196 | 3 | 105 | 186 | 191 | 6 |
| h77A-1.7 | hVH-1.7 | hVL-1.1 | 32 | 193 | 196 | 171 | 105 | 186 | 191 | 6 |
| h77A-1.8 | hVH-1.5 | hVL-1.3 | 30 | 193 | 196 | 3 | 107 | 186 | 191 | 6 |
| h77A-1.9 | hVH-1.8 | hVL-1.1 | 33 | 193 | 196 | 172 | 105 | 186 | 191 | 6 |
| h77A-1.10 | hVH-1.9 | hVL-1.1 | 34 | 193 | 196 | 3 | 105 | 186 | 191 | 6 |
| h77A-1.11 | hVH-1.5 | hVL-1.4 | 30 | 193 | 196 | 3 | 108 | 186 | 191 | 6 |
| h77A-1.12 | hVH-1.5 | hVL-1.5 | 30 | 193 | 196 | 3 | 109 | 187 | 191 | 6 |
| h77A-1.13 | hVH-1.10 | hVL-1.1 | 35 | 193 | 196 | 173 | 105 | 186 | 191 | 6 |
| h77A-1.14 | hVH-1.11 | hVL-1.1 | 36 | 193 | 199 | 3 | 105 | 186 | 191 | 6 |
| h77A-1.15 | hVH-1.12 | hVL-1.1 | 37 | 193 | 196 | 174 | 105 | 186 | 191 | 6 |
| h77A-1.16 | hVH-1.1 | hVL-1.3 | 26 | 192 | 196 | 3 | 107 | 186 | 191 | 6 |
| h77A-1.17 | hVH-1.13 | hVL-1.1 | 38 | 192 | 196 | 175 | 105 | 186 | 191 | 6 |
| h77A-1.18 | hVH-1.6 | hVL-1.6 | 31 | 193 | 196 | 3 | 110 | 186 | 191 | 6 |
| h77A-1.19 | hVH-1.14 | hVL-1.1 | 39 | 193 | 200 | 3 | 105 | 186 | 191 | 6 |
| h77A-1.20 | hVH-1.15 | hVL-1.1 | 40 | 193 | 201 | 3 | 105 | 186 | 191 | 6 |
| h77A-1.21 | hVH-1.9 | hVL-1.7 | 34 | 193 | 196 | 3 | 111 | 186 | 191 | 6 |
| h77A-1.22 | hVH-1.16 | hVL-1.5 | 41 | 193 | 201 | 3 | 109 | 187 | 191 | 6 |
| h77A-1.23 | hVH-1.5 | hVL-1.8 | 30 | 193 | 196 | 3 | 112 | 188 | 191 | 163 |
| h77A-1.24 | hVH-1.3 | hVL-1.9 | 28 | 193 | 196 | 170 | 113 | 189 | 191 | 6 |
| h77A-1.25 | hVH-1.7 | hVL-1.10 | 32 | 193 | 196 | 171 | 114 | 186 | 191 | 6 |
| h77A-1.26 | hVH-1.17 | hVL-1.1 | 42 | 193 | 196 | 176 | 105 | 186 | 191 | 6 |
| h77A-1.27 | hVH-1.11 | hVL-1.11 | 36 | 193 | 199 | 3 | 115 | 186 | 191 | 6 |
| h77A-1.28 | hVH-1.18 | hVL-1.1 | 43 | 193 | 196 | 172 | 105 | 186 | 191 | 6 |
| h77A-1.29 | hVH-1.7 | hVL-1.5 | 32 | 193 | 196 | 171 | 109 | 187 | 191 | 6 |
| h77A-1.30 | hVH-1.19 | hVL-1.12 | 44 | 193 | 196 | 3 | 116 | 186 | 191 | 6 |
| h77A-1.31 | hVH-1.10 | hVL-1.13 | 35 | 193 | 196 | 173 | 117 | 186 | 191 | 6 |
| h77A-1.32 | hVH-1.20 | hVL-1.1 | 45 | 193 | 197 | 177 | 105 | 186 | 191 | 6 |
| h77A-1.33 | hVH-1.21 | hVL-1.1 | 46 | 192 | 196 | 178 | 105 | 186 | 191 | 6 |
| h77A-1.34 | hVH-1.11 | hVL-1.14 | 36 | 193 | 199 | 3 | 118 | 186 | 191 | 6 |
| h77A-1.35 | hVH-1.22 | hVL-1.11 | 47 | 193 | 196 | 179 | 115 | 186 | 191 | 6 |
| h77A-1.36 | hVH-1.23 | hVL-1.5 | 48 | 193 | 196 | 179 | 109 | 187 | 191 | 6 |
| h77A-1.37 | hVH-1.24 | hVL-1.1 | 49 | 192 | 202 | 174 | 105 | 186 | 191 | 6 |
| h77A-1.38 | hVH-1.25 | hVL-1.1 | 50 | 193 | 196 | 180 | 105 | 186 | 191 | 6 |
| h77A-1.39 | hVH-1.26 | hVL-1.2 | 51 | 193 | 201 | 3 | 106 | 186 | 191 | 162 |
| h77A-1.40 | hVH-1.27 | hVL-1.15 | 52 | 193 | 196 | 181 | 119 | 186 | 191 | 162 |
| h77A-1.41 | hVH-1.28 | hVL-1.4 | 53 | 193 | 196 | 174 | 108 | 186 | 191 | 6 |
| h77A-1.42 | hVH-1.29 | hVL-1.1 | 54 | 193 | 196 | 182 | 105 | 186 | 191 | 6 |
| h77A-1.43 | hVH-1.30 | hVL-1.12 | 55 | 193 | 196 | 3 | 116 | 186 | 191 | 6 |
| h77A-1.44 | hVH-1.31 | hVL-1.12 | 56 | 193 | 196 | 183 | 116 | 186 | 191 | 6 |
| h77A-1.45 | hVH-1.32 | hVL-1.16 | 57 | 193 | 196 | 3 | 120 | 186 | 191 | 163 |
| h77A-1.46 | hVH-1.33 | hVL-1.17 | 58 | 193 | 196 | 3 | 121 | 186 | 191 | 6 |
| h77A-1.47 | hVH-1.34 | hVL-1.18 | 59 | 193 | 196 | 180 | 122 | 186 | 191 | 6 |
| h77A-1.48 | hVH-1.35 | hVL-1.4 | 60 | 193 | 196 | 181 | 108 | 186 | 191 | 6 |
| h77A-1.49 | hVH-1.36 | hVL-1.19 | 61 | 193 | 203 | 184 | 123 | 186 | 191 | 6 |
| h77A-1.50 | hVH-1.37 | hVL-1.10 | 62 | 193 | 199 | 3 | 114 | 186 | 191 | 6 |
| h77A-1.51 | hVH-1.38 | hVL-1.20 | 63 | 194 | 196 | 185 | 124 | 189 | 191 | 6 |
| h77A-1.52 | hVH-1.39 | hVL-1.1 | 64 | 195 | 197 | 170 | 105 | 186 | 191 | 6 |
| h77A-1.53 | hVH-1.40 | hVL-1.21 | 65 | 193 | 204 | 3 | 125 | 186 | 191 | 6 |
| h77A-1.54 | hVH-1.41 | hVL-1.1 | 66 | 192 | 205 | 170 | 105 | 186 | 191 | 6 |
| h77A-1.55 | hVH-1.42 | hVL-1.21 | 67 | 193 | 206 | 171 | 125 | 186 | 191 | 6 |
| h77A-1.56 | hVH-1.43 | hVL-1.22 | 68 | 194 | 196 | 3 | 126 | 186 | 191 | 6 |
| h77A-1.57 | hVH-1.44 | hVL-1.23 | 69 | 193 | 207 | 174 | 127 | 190 | 191 | 6 |
| h77A-1.58 | hVH-1.45 | hVL-1.24 | 70 | 193 | 196 | 184 | 128 | 186 | 191 | 6 |
| h77A-1.59 | hVH-1.46 | hVL-1.13 | 71 | 193 | 206 | 184 | 117 | 186 | 191 | 6 |
| h77A-1.60 | hVH-1.47 | hVL-1.25 | 72 | 193 | 196 | 175 | 129 | 186 | 191 | 6 |
| h77A-1.61 | hVH-1.48 | hVL-1.26 | 73 | 193 | 196 | 3 | 130 | 186 | 191 | 6 |
| h77A-1.62 | hVH-1.49 | hVL-1.27 | 74 | 193 | 196 | 3 | 131 | 186 | 191 | 6 |
| h77A-1.63 | hVH-1.48 | hVL-1.28 | 73 | 193 | 196 | 3 | 132 | 189 | 191 | 6 |
| h77A-1.64 | hVH-1.50 | hVL-1.29 | 75 | 193 | 202 | 3 | 133 | 186 | 191 | 6 |
| h77A-1.65 | hVH-1.51 | hVL-1.30 | 76 | 193 | 207 | 3 | 134 | 186 | 191 | 6 |
| h77A-1.66 | hVH-1.52 | hVL-1.3 | 77 | 195 | 202 | 3 | 107 | 186 | 191 | 6 |
| h77A-1.67 | hVH-1.53 | hVL-1.31 | 78 | 193 | 196 | 3 | 135 | 186 | 191 | 6 |
| h77A-1.68 | hVH-1.54 | hVL-1.32 | 79 | 193 | 199 | 3 | 136 | 186 | 191 | 6 |
| h77A-1.69 | hVH-1.55 | hVL-1.33 | 80 | 193 | 208 | 3 | 137 | 186 | 191 | 6 |
| h77A-1.70 | hVH-1.16 | hVL-1.34 | 41 | 193 | 201 | 3 | 138 | 186 | 191 | 6 |
| h77A-1.71 | hVH-1.56 | hVL-1.35 | 81 | 193 | 196 | 3 | 139 | 186 | 191 | 6 |
| h77A-1.72 | hVH-1.57 | hVL-1.1 | 82 | 195 | 209 | 3 | 105 | 186 | 191 | 6 |
| h77A-1.73 | hVH-1.58 | hVL-1.22 | 83 | 193 | 196 | 3 | 126 | 186 | 191 | 6 |
| h77A-1.74 | hVH-1.59 | hVL-1.36 | 84 | 193 | 196 | 3 | 140 | 186 | 191 | 6 |
| h77A-1.75 | hVH-1.60 | hVL-1.37 | 85 | 193 | 201 | 3 | 141 | 187 | 191 | 6 |
| h77A-1.76 | hVH-1.61 | hVL-1.37 | 86 | 193 | 209 | 3 | 141 | 187 | 191 | 6 |
| h77A-1.77 | hVH-1.62 | hVL-1.13 | 87 | 195 | 206 | 184 | 117 | 186 | 191 | 6 |
| h77A-1.78 | hVH-1.63 | hVL-1.38 | 88 | 193 | 210 | 178 | 142 | 186 | 191 | 163 |
| h77A-1.79 | hVH-1.64 | hVL-1.39 | 89 | 192 | 197 | 170 | 143 | 186 | 191 | 162 |
| h77A-1.80 | hVH-1.65 | hVL-1.40 | 90 | 193 | 210 | 174 | 144 | 188 | 191 | 6 |
| h77A-1.81 | hVH-1.66 | hVL-1.5 | 91 | 195 | 196 | 183 | 109 | 187 | 191 | 6 |
| h77A-1.82 | hVH-1.67 | hVL-1.41 | 92 | 193 | 209 | 3 | 145 | 186 | 191 | 6 |
| h77A-1.83 | hVH-1.68 | hVL-1.42 | 93 | 193 | 196 | 184 | 146 | 188 | 191 | 6 |
| h77A-1.84 | hVH-1.69 | hVL-1.43 | 94 | 194 | 196 | 183 | 147 | 186 | 191 | 6 |
| h77A-1.85 | hVH-1.70 | hVL-1.44 | 95 | 195 | 196 | 183 | 148 | 188 | 191 | 6 |
| h77A-1.86 | hVH-1.71 | hVL-1.45 | 96 | 193 | 209 | 3 | 149 | 187 | 191 | 6 |
| h77A-1.87 | hVH-1.72 | hVL-1.46 | 97 | 192 | 211 | 170 | 150 | 186 | 191 | 6 |
| h77A-1.88 | hVH-1.73 | hVL-1.47 | 98 | 193 | 210 | 172 | 151 | 189 | 191 | 6 |
| h77A-1.89 | hVH-1.74 | hVL-1.48 | 99 | 193 | 202 | 3 | 152 | 186 | 191 | 6 |
| h77A-1.90 | hVH-1.75 | hVL-1.32 | 100 | 195 | 202 | 3 | 136 | 186 | 191 | 6 |
| h77A-1.91 | hVH-1.66 | hVL-1.49 | 91 | 195 | 196 | 183 | 153 | 187 | 191 | 6 |
| h77A-1.92 | hVH-1.76 | hVL-1.50 | 101 | 194 | 206 | 3 | 154 | 187 | 191 | 162 |
| h77A-1.93 | hVH-1.77 | hVL-1.51 | 102 | 193 | 208 | 183 | 155 | 186 | 191 | 6 |
| h77A-1.94 | hVH-1.11 | hVL-1.52 | 36 | 193 | 199 | 3 | 156 | 187 | 191 | 6 |
| h77A-1.95 | hVH-1.78 | hVL-1.53 | 103 | 193 | 199 | 184 | 157 | 186 | 191 | 6 |
| h77A-1.96 | hVH-1 | hVL-1.54 | 193 | 196 | 3 | 193 | 24 | 186 | 191 | 6 |
| h77A-1.97 | hVH-1.79 | hVL-1.54 | 193 | 196 | 3 | 193 | 24 | 186 | 191 | 6 |
| h77A-1.98 | hVH-1.79 | hVL-1 | 193 | 196 | 3 | 193 | 19 | 186 | 191 | 6 |

TABLE 12
CDR Amino Acid Sequences

| CDR | SEQ ID NO | Amino Acid Sequence |
|---|---|---|
| VLCDR1 | 159 | QSLFNSGTRKNY |
| VLCDR1 | 160 | QSLVNSGTRKNY |
| VLCDR1 | 161 | QSLFNSGTRKNY |
| VLCDR3 | 162 | KQSYNLYT |
| VLCDR3 | 163 | KQSYSLYT |
| VHCDR1 | 164 | GYTFTKYG |
| VHCDR1 | 165 | GYSFTNYG |
| VHCDR1 | 166 | GYIFTNYG |
| VHCDR2 | 167 | INTYTGES |
| VHCDR2 | 168 | INTYTGET |
| VHCDR2 | 169 | INTYTGEA |
| VHCDR3 | 170 | ARYDHRMDY |
| VHCDR3 | 171 | ARYDHFMDY |
| VHCDR3 | 172 | ARYDHTMDY |
| VHCDR3 | 173 | ARYDHPMDY |
| VHCDR3 | 174 | ARYDHVMDY |
| VHCDR3 | 175 | ARYDHSMDY |
| VHCDR3 | 176 | TRYDHAMDY |
| VHCDR3 | 177 | ARYDHDMDY |
| VHCDR3 | 178 | ARYDHNMDY |
| VHCDR3 | 179 | ARYDHHMDY |
| VHCDR3 | 180 | ARYDHLMDY |
| VHCDR3 | 181 | ARYDHYMDY |
| VHCDR3 | 182 | TRYDHRMDY |
| VHCDR3 | 183 | VRYDHAMDY |
| VHCDR3 | 184 | GRYDHAMDY |
| VHCDR3 | 185 | ARYDHGMDY |
| VLCDR1 | 186 | KSSQSLLNSGTRKNYLA |
| VLCDR1 | 187 | KSSQSLFNSGTRKNYLA |
| VLCDR1 | 188 | KSSQSLLNSGTRKNYLS |
| VLCDR1 | 189 | KSSQSLLNSGTRKNYLT |
| VLCDR1 | 190 | KSSQSLVNSGTRKNYLA |
| VLCDR2 | 191 | WTSTRES |
| VHCDR1 | 192 | GYTFTKYGMN |
| VHCDR1 | 193 | GYTFTNYGMN |
| VHCDR1 | 194 | GYSFTNYGMN |
| VHCDR1 | 195 | GYIFTNYGMN |
| VHCDR2 | 196 | WINTYTGEPTYADDFKG |
| VHCDR2 | 197 | WINTYTGEPRYADDFKG |
| VHCDR2 | 198 | WINTYTGEPIYADDFKG |
| VHCDR2 | 199 | WINTYTGEPTYTDDFKG |
| VHCDR2 | 200 | WINTYTGEPTYVDDFKG |
| VHCDR2 | 201 | WINTYTGEPTYSDDFKG |
| VHCDR2 | 202 | WINTYTGESTYADDFKG |
| VHCDR2 | 203 | WINTYTGETTYGDDFKG |
| VHCDR2 | 204 | WINTYTGEPKYTDDFKG |
| VHCDR2 | 205 | WINTYTGEPKYGDDFKG |
| VHCDR2 | 206 | WINTYTGEPTYGDDFKG |
| VHCDR2 | 207 | WINTYTGEATYADDFKG |
| VHCDR2 | 208 | WINTYTGETTYTDDFKG |
| VHCDR2 | 209 | WINTYTGETTYADDFKG |
| VHCDR2 | 210 | WINTYTGEPKYADDFKG |
| VHCDR2 | 211 | WINTYTGEPRYVDDFKG |

TABLE 13
Variable Region Amino Acid Sequences

| VH/VL | SEQ | |
|---|---|---|
| Name | ID NO | Amino Acid Sequence |
| hVH-1.1 | 26 | QIQLVQSGAEVKKPGASVKVSCKASGYTFTKYGMNWVRQAPGQGLEWMGWIN |
| | | TYTGEPTYADDFKGRFTFTTDTSTSTAYMELRSLRSDDTAVYFCARYDHAMD |
| | | YWGQGTLVTVSS |
| hVH-1.2 | 27 | QIQLVQSGAEVKKPGASVKVSCKASGYTFTNYGMNWVRQAPGQGLEWMGWIN |
| | | TYTGEPRYADDFKGRFTFTTDTSTSTAYMELRSLRSDDTAVYFCARYDHAMD |
| | | YWGQGTLVTVSS |
| hVH-1.3 | 28 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHRMD |
| | | YWGQGTLVTVSS |
| hVH-1.4 | 29 | QIQLVQSGAEVKKPGASVKVSCKASGYTFTNYGMNWVRQAPGQGLEWMGWIN |
| | | TYTGEPIYADDFKGRFTFTTDTSTSTAYMELRSLRSDDTAVYFCARYDHAMD |
| | | YWGQGTLVTVSS |
| hVH-1.5 | 30 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHAMD |
| | | YWGQGTLVTVSS |
| hVH-1.6 | 31 | QIQLVQSGAEVKKPGASVKVSCKASGYTFTNYGMNWVRQAPGQGLEWMGWIN |
| | | TYTGEPTYADDFKGRFTFTTDTSTRTAYMELRSLRSDDTAVYFCARYDHAMD |
| | | YWGQGTLVTVSS |
| hVH-1.7 | 32 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHFMD |
| | | YWGQGTLVTVSS |
| hVH-1.8 | 33 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHTMD |
| | | YWGQGTLVTVSS |
| hVH-1.9 | 34 | QIQLVQSGDEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHAMD |
| | | YWGQGTLVTVSS |
| hVH-1.10 | 35 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHPMD |
| | | YWGQGTLVTVSS |
| hVH-1.11 | 36 | QIQLVQSGAEVKKPGASVKVSCKASGYTFTNYGMNWVRQAPGQGLEWMGWIN |
| | | TYTGEPTYTDDFKGRFTFTTDTSTSTAYMELRSLRSDDTAVYFCARYDHAMD |
| | | YWGQGTLVTVSS |
| hVH-1.12 | 37 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHVMD |
| | | YWGQGTLVTVSS |
| hVH-1.13 | 38 | QIQLVQSGAEVKKPGASVKVSCKASGYTFTKYGMNWVRQAPGQGLEWMGWIN |
| | | TYTGEPTYADDFKGRFTFTTDTSTSTAYMELRSLRSDDTAVYFCARYDHSMD |
| | | YWGQGTLVTVSS |
| hVH-1.14 | 39 | QIQLVQSGAEVKKPGASVKVSCKASGYTFTNYGMNWVRQAPGQGLEWMGWIN |
| | | TYTGEPTYVDDFKGRFTFTTDTSTSTAYMDLRSLRSDDTAVYFCARYDHAMD |
| | | YWGQGTLVTVSS |
| hVH-1.15 | 40 | QIQLVQSGDEVKKPGASVKVSCKASGYTFTNYGMNWVRQAPGQGLEWMGWIN |
| | | TYTGEPTYSDDFKGRFTFTTDTSTSTAYMELRSLRSDDTAVYFCARYDHAMD |
| | | YWGQGTLVTVSS |
| hVH-1.16 | 41 | QIQLVQSGAEVKKPGASVKVSCKASGYTFTNYGMNWVRQAPGQGLEWMGWIN |
| | | TYTGEPTYSDDFKGRFTFTTDTSTSTAYMELRSLRSDDTAVYFCARYDHAMD |
| | | YWGQGTLVTVSS |
| hVH-1.17 | 42 | QIQLVQSGAEVKKPGASVKVSCKASGYTFTNYGMNWVRQAPGQGLEWMGWIN |
| | | TYTGEPTYADDFKGRFTFTTDTSTRTAYMELRSLRSDDTAVYFCTRYDHAMD |
| | | YWGQGTLVTVSS |
| hVH-1.18 | 43 | QIQLVQSGTEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHTMD |
| | | YWGQGTLVTVSS |
| hVH-1.19 | 44 | QIQLVQSGAEVKKPGASVKVSCKASGYTFTNYGMNWVRQAPGQGLEWMGWIN |
| | | TYTGEPTYADDFKGRFTFTTDTSTSTAYMEVRSLRSDDTAVYFCARYDHAMD |
| | | YWGQGTLVTVSS |
| hVH-1.20 | 45 | QIQLVQSGAEVKKPGASVKVSCKASGYTFTNYGMNWVRQAPGQGLEWMGWIN |
| | | TYTGEPRYADDFKGRFTFTTDTSTSTAYMELRSLRSDDTAVYFCARYDHDMD |
| | | YWGQGTLVTVSS |
| hVH-1.21 | 46 | QIQLVQSGAEVKKPGASVKVSCKASGYTFTKYGMNWVRQAPGQGLEWMGWIN |
| | | TYTGEPTYADDFKGRFTFTTDTSTSTAYMELRSLRSDDTAVYFCARYDHNMD |
| | | YWGQGTLVTVSS |
| hVH-1.22 | 47 | QIQLVQSGTEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHHMD |
| | | YWGQGTLVTVSS |
| hVH-1.23 | 48 | QIQLVQSGAEVKKPGASVKVSCKASGYTFTNYGMNWVRQAPGQGLEWMGWIN |
| | | TYTGEPTYADDFKGRFTFTTDTSTTTAYMELRSLRSDDTAVYFCARYDHHMD |
| | | YWGQGTLVTVSS |
| hVH-1.24 | 49 | QIQLVQSGAEVKKPGASVKVSCKASGYTFTKYGMNWVRQAPGQGLEWMGWIN |
| | | TYTGESTYADDFKGRFTFTTDTSTSTAYMELRSLRSDDTAVYFCARYDHVMD |
| | | YWGQGTLVTVSS |
| hVH-1.25 | 50 | QIQLVQSGTEVKKPGASVKVSCKASGYTFTNYGMNWVRQAPGQGLEWMGWIN |
| | | TYTGEPTYADDFKGRFTFTTDTSTRTAYMELRSLRSDDTAVYFCARYDHLMD |
| | | YWGQGTLVTVSS |
| hVH-1.26 | 51 | QIQLVQSGAEVKKPGASVKVSCKASGYTFTNYGMNWVRQAPGQGLEWMGWIN |
| | | TYTGEPTYSDDFKGRFTFTTDTSTRTAYMELRSLRSDDTAVYFCARYDHAMD |
| | | YWGQGTLVTVSS |
| hVH-1.27 | 52 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHYMD |
| | | YWGQGTLVTVSS |
| hVH-1.28 | 53 | QIQLVQSGAEVKKPGASVKVSCKASGYTFTNYGMNWVRQAPGQGLEWMGWIN |
| | | TYTGEPTYADDFKGRFTFTTDTSTTTAYMELRSLRSDDTAVYFCARYDHVMD |
| | | YWGQGTLVTVSS |
| hVH-1.29 | 54 | QIHLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCTRYDHRMD |
| | | YWGQGTLVTVSS |
| hVH-1.30 | 55 | QIHLVQSGAEVKKPGASVKVSCKASGYTFTNYGMNWVRQAPGQGLEWMGWIN |
| | | TYTGEPTYADDFKGRFTFTTDTSTSTVYMELRSLRSDDTAVYFCARYDHAMD |
| | | YWGQGTLVTVSS |
| hVH-1.31 | 56 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCVRYDHAMD |
| | | YWGQGSLVTVSS |
| hVH-1.32 | 57 | QIQLVQSGVEVKKPGASVKVSCKASGYTFTNYGMNWVRQAPGQGLEWMGWIN |
| | | TYTGEPTYADDFKGRFTFTTDTSTSTVYMELRSLRSDDTAVYFCARYDHAMD |
| | | YWGQGTLVTVSS |
| hVH-1.33 | 58 | QIQLVQSGVEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHAMD |
| | | YWGQGTLVTVSS |
| hVH-1.34 | 59 | QIQLVQSGTEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHLMD |
| | | YWGQGTLVTVSS |
| hVH-1.35 | 60 | QIQLVQSGAEVKKPGASVKVSCKASGYTFTNYGMNWVRQAPGQGLEWMGWIN |
| | | TYTGEPTYADDFKGRFTFTTDTSTSTAYMDLRSLRSDDTAVYFCARYDHYMD |
| | | YWGQGTLVTVSS |
| hVH-1.36 | 61 | QIQLVQSGAEVKKPGASVKVSCKASGYTFTNYGMNWVRQAPGQGLEWMGWIN |
| | | TYTGETTYGDDFKGRFTFTTDTSTSTAYMELRSLRSDDTAVYFCGRYDHAMD |
| | | YWGQGTLVTVSS |
| hVH-1.37 | 62 | QIHLVQSGVEVKKPGASVKVSCKASGYTFTNYGMNWVRQAPGQGLEWMGWIN |
| | | TYTGEPTYTDDFKGRFTFTTDTSTSTAYMELRSLRSDDTAVYFCARYDHAMD |
| | | YWGQGTLVTVSS |
| hVH-1.38 | 63 | QIQLVQSGAEVKKPGASVKVSCKASGYSFTNYGMNWVRQAPGQGLEWMGWIN |
| | | TYTGEPTYADDFKGRFTFTTDTSTSTAYMELRSLRSDDTAVYFCARYDHGMD |
| | | YWGQGTLVTVSS |
| hVH-1.39 | 64 | QIQLVQSGVEVKKPGASVKVSCKASGYIFTNYGMNWVRQAPGQGLEWMGWIN |
| | | TYTGEPRYADDFKGRFTFTTDTSTSTAYMELRSLRSDDTAVYFCARYDHRMD |
| | | YWGQGTLVTVSS |
| hVH-1.40 | 65 | QIQLVQSGAEVKKPGASVKVSCKASGYTFTNYGMNWVRQAPGQGLEWMGWIN |
| | | TYTGEPKYTDDFKGRFTFTTDTSTRTAYMELRSLRSDDTAVYFCARYDHAMD |
| | | YWGQGTLVTVSS |
| hVH-1.41 | 66 | QIQLVQSGAEVKKPGASVKVSCKASGYTFTKYGMNWVRQAPGQGLEWMGWIN |
| | | TYTGEPKYGDDFKGRFTFTTDTSTSTAYMELRSLRSDDTAVYFCARYDHRMD |
| | | YWGQGTLVTVSS |
| hVH-1.42 | 67 | QIQLVQSGSEVKKPGASVKVSCKASGYTFTNYGMNWVRQAPGQGLEWMGWIN |
| | | TYTGEPTYGDDFKGRFTFTTDTSTSTAYMELRSLRSDDTAVYFCARYDHFMD |
| | | YWGQGTLVTVSS |
| hVH-1.43 | 68 | QIQLVQSGTEVKKPGASVKVSCKASGYSFTNYGMNWVRQAPGQGLEWMGWIN |
| | | TYTGEPTYADDFKGRFTFTTDTSTTTAYMELRSLRSDDTAVYFCARYDHAMD |
| | | YWGQGTLVTVSS |
| hVH-1.44 | 69 | QIQLVQSGAEVKKPGASVKVSCKASGYTFTNYGMNWVRQAPGQGLEWMGWIN |
| | | TYTGEATYADDFKGRFTFTTDTSTSTAYMELRSLRSDDTAVYFCARYDHVMD |
| | | YWGQGTLVTVSS |
| hVH-1.45 | 70 | QIQLVQSGSEVKKPGASVKVSCKASGYTFTNYGMNWVRQAPGQGLEWMGWIN |
| | | TYTGEPTYADDFKGRFTFTTDTSTSTAYMELRSLRSDDTAVYFCGRYDHAMD |
| | | YWGQGTLVTVSS |
| hVH-1.46 | 71 | QIQLVQSGPEVKKPGASVKVSCKASGYTFTNYGMNWVRQAPGQGLEWMGWIN |
| | | TYTGEPTYGDDFKGRFTFTTDTSTSTAYMELRSLRSDDTAVYFCGRYDHAMD |
| | | YWGQGTLVTVSS |
| hVH-1.47 | 72 | QIQLVQSGSEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHSMD |
| | | YWGQGTLVTVSS |
| hVH-1.48 | 73 | QIQLVQSGAEVKKPGASVKVSCKASGYTFTNYGMNWVRQAPGQGLEWMGWIN |
| | | TYTGEPTYADDFKGRFTFTTDTSTSTAYMELRSLRSDDTAVYFCARYDHAMD |
| | | YWGQGSLVTVSS |
| hVH-1.49 | 74 | QIQLVQSGAEVKKPGASVKVSCKASGYTFTNYGMNWVRQAPGQGLEWMGWIN |
| | | TYTGEPTYADDFKGRFTFTTDTSTTTGYMELRSLRSDDTAVYFCARYDHAMD |
| | | YWGQGTLVTVSS |
| hVH-1.50 | 75 | QIQLVQSGAEVKKPGASVKVSCKASGYTFTNYGMNWVRQAPGQGLEWMGWIN |
| | | TYTGESTYADDFKGRFTFTTDTSTSTAYMELRSLRSDDTAVYFCARYDHAMD |
| | | YWGQGTLVTVSS |
| hVH-1.51 | 76 | QIQLVQSGAEVKKPGASVKVSCKASGYTFTNYGMNWVRQAPGQGLEWMGWIN |
| | | TYTGEATYADDFKGRFTFTTDTSTSTAYMELRSLRSDDTAVYFCARYDHAMD |
| | | YWGQGSLVTVSS |
| hVH-1.52 | 77 | QIQLVQSGAEVKKPGASVKVSCKASGYIFTNYGMNWVRQAPGQGLEWMGWIN |
| | | TYTGESTYADDFKGRFTFTTDTSTSTVYMELRSLRSDDTAVYFCARYDHAMD |
| | | YWGQGTLVTVSS |
| hVH-1.53 | 78 | QIQLVQSGAEVKKPGASVKVSCKASGYTFTNYGMNWVRQAPGQGLEWMGWIN |
| | | TYTGEPTYADDFKGRFTFTTDTSTSTAYMDLRSLRSDDTAVYFCARYDHAMD |
| | | YWGQGTLVTVSS |
| hVH-1.54 | 79 | QIHLVQSGDEVKKPGASVKVSCKASGYTFTNYGMNWVRQAPGQGLEWMGWIN |
| | | TYTGEPTYTDDFKGRFTFTTDTSTSTAYMELRSLRSDDTAVYFCARYDHAMD |
| | | YWGQGTLVTVSS |
| hVH-1.55 | 80 | QIQLVQSGAEVKKPGASVKVSCKASGYTFTNYGMNWVRQAPGQGLEWMGWIN |
| | | TYTGETTYTDDFKGRFTFTTDTSTSTAYMELRSLRSDDTAVYFCARYDHAMD |
| | | YWGQGTLVTVSS |
| hVH-1.56 | 81 | QIQLVQSGSEVKKPGASVKVSCKASGYTFTNYGMNWVRQAPGQGLEWMGWIN |
| | | TYTGEPTYADDFKGRFTFTTDTSTSTAYMDLRSLRSDDTAVYFCARYDHAMD |
| | | YWGQGTLVTVSS |
| hVH-1.57 | 82 | QIQLVQSGPEVKKPGASVKVSCKASGYIFTNYGMNWVRQAPGQGLEWMGWIN |
| | | TYTGETTYADDFKGRFTFTTDTSTTTVYMELRSLRSDDTAVYFCARYDHAMD |
| | | YWGQGTLVTVSS |
| hVH-1.58 | 83 | QIHLVQSGDEVKKPGASVKVSCKASGYTFTNYGMNWVRQAPGQGLEWMGWIN |
| | | TYTGEPTYADDFKGRFTFTTDTSTSTGYMELRSLRSDDTAVYFCARYDHAMD |
| | | YWGQGSLVTVSS |
| hVH-1.59 | 84 | QIQLVQSGPEVKKPGASVKVSCKASGYTFTNYGMNWVRQAPGQGLEWMGWIN |
| | | TYTGEPTYADDFKGRFTFTTDTSTTTGYMELRSLRSDDTAVYFCARYDHAMD |
| | | YWGQGTLVTVSS |
| hVH-1.60 | 85 | QIQLVQSGVEVKKPGASVKVSCKASGYTFTNYGMNWVRQAPGQGLEWMGWIN |
| | | TYTGEPTYSDDFKGRFTFTTDTSTSTAYMEVRSLRSDDTAVYFCARYDHAMD |
| | | YWGQGTLVTVSS |
| hVH-1.61 | 86 | QIQLVQSGVEVKKPGASVKVSCKASGYTFTNYGMNWVRQAPGQGLEWMGWIN |
| | | TYTGETTYADDFKGRFTFTTDTSTSTAYMEVRSLRSDDTAVYFCARYDHAMD |
| | | YWGQGTLVTVSS |
| hVH-1.62 | 87 | QIQLVQSGPEVKKPGASVKVSCKASGYIFTNYGMNWVRQAPGQGLEWMGWIN |
| | | TYTGEPTYGDDFKGRFTFTTDTSTSTAYMELRSLRSDDTAVYFCGRYDHAMD |
| | | YWGQGTLVTVSS |
| hVH-1.63 | 88 | QIHLVQSGAEVKKPGASVKVSCKASGYTFTNYGMNWVRQAPGQGLEWMGWIN |
| | | TYTGEPKYADDFKGRFTFTTDTSTSTAYMELRSLRSDDTAVYFCARYDHNMD |
| | | YWGQGTLVTVSS |
| hVH-1.64 | 89 | QIQLVQSGAEVKKPGASVKVSCKASGYTFTKYGMNWVRQAPGQGLEWMGWIN |
| | | TYTGEPRYADDFKGRFTFTTDTSTSTAYMELRSLRSDDTAVYFCARYDHRMD |
| | | YWGQGTLVTVSS |
| hVH-1.65 | 90 | QIQLVQSGAEVKKPGASVKVSCKASGYTFTNYGMNWVRQAPGQGLEWMGWIN |
| | | TYTGEPKYADDFKGRFTFTTDTSTSTAYMELRSLRSDDTAVYFCARYDHVMD |
| | | YWGQGTLVTVSS |
| hVH-1.66 | 91 | QIHLVQSGAEVKKPGASVKVSCKASGYIFTNYGMNWVRQAPGQGLEWMGWIN |
| | | TYTGEPTYADDFKGRFTFTTDTSTSTVYMELRSLRSDDTAVYFCVRYDHAMD |
| | | YWGQGTLVTVSS |
| hVH-1.67 | 92 | QIQLVQSGTEVKKPGASVKVSCKASGYTFTNYGMNWVRQAPGQGLEWMGWIN |
| | | TYTGETTYADDFKGRFTFTTDTSTSTAYMDLRSLRSDDTAVYFCARYDHAMD |
| | | YWGQGTLVTVSS |
| hVH-1.68 | 93 | QIHLVQSGPEVKKPGASVKVSCKASGYTFTNYGMNWVRQAPGQGLEWMGWIN |
| | | TYTGEPTYADDFKGRFTFTTDTSTSTAYMELRSLRSDDTAVYFCGRYDHAMD |
| | | YWGQGTLVTVSS |
| hVH-1.69 | 94 | QIHLVQSGAEVKKPGASVKVSCKASGYSFTNYGMNWVRQAPGQGLEWMGWIN |
| | | TYTGEPTYADDFKGRFTFTTDTSTSTAYMELRSLRSDDTAVYFCVRYDHAMD |
| | | YWGQGTLVTVSS |
| hVH-1.70 | 95 | QIHLVQSGAEVKKPGASVKVSCKASGYIFTNYGMNWVRQAPGQGLEWMGWIN |
| | | TYTGEPTYADDFKGRFTFTTDTSTSTAYMELRSLRSDDTAVYFCVRYDHAMD |
| | | YWGQGTLVTVSS |
| hVH-1.71 | 96 | QIQLVQSGVEVKKPGASVKVSCKASGYTFTNYGMNWVRQAPGQGLEWMGWIN |
| | | TYTGETTYADDFKGRFTFTTDTSTSTAYMELRSLRSDDTAVYFCARYDHAMD |
| | | YWGQGTLVTVSS |
| hVH-1.72 | 97 | QIQLVQSGAEVKKPGASVKVSCKASGYTFTKYGMNWVRQAPGQGLEWMGWIN |
| | | TYTGEPRYVDDFKGRFTFTTDTSTSTAYMELRSLRSDDTAVYFCARYDHRMD |
| | | YWGQGTLVTVSS |
| hVH-1.73 | 98 | QIQLVQSGAEVKKPGASVKVSCKASGYTFTNYGMNWVRQAPGQGLEWMGWIN |
| | | TYTGEPKYADDFKGRFTFTTDTSTRTAYMELRSLRSDDTAVYFCARYDHTMD |
| | | YWGQGTLVTVSS |
| hVH-1.74 | 99 | QIQLVQSGAEVKKPGASVKVSCKASGYTFTNYGMNWVRQAPGQGLEWMGWIN |
| | | TYTGESTYADDFKGRFTFTTDTSTTTAYMELRSLRSDDTAVYFCARYDHAMD |
| | | YWGQGTLVTVSS |
| hVH-1.75 | 100 | QIQLVQSGAEVKKPGASVKVSCKASGYIFTNYGMNWVRQAPGQGLEWMGWIN |
| | | TYTGESTYADDFKGRFTFTTDTSTTTGYMELRSLRSDDTAVYFCARYDHAMD |
| | | YWGQGTLVTVSS |
| hVH-1.76 | 101 | QIQLVQSGAEVKKPGASVKVSCKASGYSFTNYGMNWVRQAPGQGLEWMGWIN |
| | | TYTGEPTYGDDFKGRFTFTTDTSTSTAYMELRSLRSDDTAVYFCARYDHAMD |
| | | YWGQGTLVTVSS |
| hVH-1.77 | 102 | QIQLVQSGAEVKKPGASVKVSCKASGYTFTNYGMNWVRQAPGQGLEWMGWIN |
| | | TYTGETTYTDDFKGRFTFTTDTSTSTAYMELRSLRSDDTAVYFCVRYDHAMD |
| | | YWGQGTLVTVSS |
| hVH-1.78 | 103 | QIQLVQSGAEVKKPGASVKVSCKASGYTFTNYGMNWVRQAPGQGLEWMGWIN |
| | | TYTGEPTYTDDFKGRFTFTTDTSTSTAYMELRSLRSDDTAVYFCGRYDHAMD |
| | | YWGQGTLVTVSS |
| hVH-1.79 | 17 | QIQLVQSGAEVKKPGASVKVSCKASGYTFTNYGMNWVRQAPGQGLEW |
| | | MGWINTYTGEPTYADDFKGRFTMTTDTSTSTAYMELRSLRSDDTAVY |
| | | FCARYDHAMDYWGQGTLVTVSS |
| hVL-1.1 | 105 | EIVLTQSPDSLAVSLGERATINCKSSQSLLNSGTRKNYLAWYQQKAGQSPKL |
| | | LIYWTSTRESGVPDRFSGSGSGTDFTLTIDSLQAEDVAVYYCKQSYTLYTFG |
| | | GGTKVEIK |
| hVL-1.2 | 106 | EIVLTQSPDSLAVSLGERATINCKSSQSLLNSGTRKNYLAWYQQKAGQSPKL |
| | | LIYWTSTRESGVPDRFSGSGSGTDFTLTIDSLQAEDVAVYYCKQSYNLYTFG |
| | | GGTKVEIK |
| hVL-1.3 | 107 | EIVLTQSPDSLAVSLGERATIKCKSSQSLLNSGTRKNYLAWYQQKAGQSPKL |
| | | LIYWTSTRESGVPDRFSGSGSGTDFTLTIDSLQAEDVAVYYCKQSYTLYTFG |
| | | GGTKVEIK |
| hVL-1.4 | 108 | EIVLTQSPDSLAVSLGERATINCKSSQSLLNSGTRKNYLAWYQQKPGQSPKL |
| | | LIYWTSTRESGVPDRFSGSGSGTDFTLTIDSLQAEDVAVYYCKQSYTLYTFG |
| | | GGTKVEIK |
| hVL-1.5 | 109 | EIVLTQSPDSLAVSLGERATINCKSSQSLFNSGTRKNYLAWYQQKAGQSPKL |
| | | LIYWTSTRESGVPDRFSGSGSGTDFTLTIDSLQAEDVAVYYCKQSYTLYTFG |
| | | GGTKVEIK |
| hVL-1.6 | 110 | EIVLTQSPDSLAVSLGERATINCKSSQSLLNSGTRKNYLAWYQQKAGQSPKL |
| | | VIYWTSTRESGVPDRFSGSGSGTDFTLTIDSLQAEDVAVYYCKQSYTLYTFG |
| | | GGTKVEIK |
| hVL-1.7 | 111 | EIVLTQSPDSLAVSLGERATINCKSSQSLLNSGTRKNYLAWYQQKAGQSPKL |
| | | LIYWTSTRESGVPDRFSGSGSGTDFTLSIDSLQAEDVAVYYCKQSYTLYTFG |
| | | GGTKVEIK |
| hVL-1.8 | 112 | EIVLTQSPDSLAVSLGERATINCKSSQSLLNSGTRKNYLSWYQQKAGQSPKL |
| | | LIYWTSTRESGVPDRFSGSGSGTDFTLTIDSLQAEDVAVYYCKQSYSLYTFG |
| | | GGTKVEIK |
| hVL-1.9 | 113 | EIVLTQSPDSLAVSLGERATINCKSSQSLLNSGTRKNYLTWYQQKAGQSPKL |
| | | LIYWTSTRESGVPDRFSGSGSGTDFTLTIDSLQAEDVAVYYCKQSYTLYTFG |
| | | GGTKVEIK |
| hVL-1.10 | 114 | EIVLTQSPDSLAVSLGERATINCKSSQSLLNSGTRKNYLAWYQQKAGQSPKL |
| | | LIYWTSTRESGVPDRFSGSGSGTDFTLTIDSLQAEDVAIYYCKQSYTLYTFG |
| | | GGTKVEIK |
| hVL-1.11 | 115 | EIVLTQSPDSLTVSLGERATINCKSSQSLLNSGTRKNYLAWYQQKAGQSPKL |
| | | LIYWTSTRESGVPDRFSGSGSGTDFTLTIDSLQAEDVAVYYCKQSYTLYTFG |
| | | GGTKVEIK |
| hVL-1.12 | 116 | EIVLTQSPDSLAVSLGERATINCKSSQSLLNSGTRKNYLAWYQQKAGQSPKL |
| | | IIYWTSTRESGVPDRFSGSGSGTDFTLTIDSLQAEDVAVYYCKQSYTLYTFG |
| | | GGTKVEIK |
| hVL-1.13 | 117 | EIVLTQSPDSLAVSLGERATINCKSSQSLLNSGTRKNYLAWYQQKAGQSPKL |
| | | LIYWTSTRESGVPDRFSGSGSGTDFTLTIDRLQAEDVAVYYCKQSYTLYTFG |
| | | GGTKVEIK |
| hVL-1.14 | 118 | EIVLTQSPDSLTVSLGERATINCKSSQSLLNSGTRKNYLAWYQQKAGQSPNL |
| | | LIYWTSTRESGVPDRFSGSGSGTDFTLTIDSLQAEDVAVYYCKQSYTLYTFG |
| | | GGTKVEIK |
| hVL-1.15 | 119 | EIVLTQSPDSLTVSLGERATINCKSSQSLLNSGTRKNYLAWYQQKAGQSPKL |
| | | LIYWTSTRESGVPDRFSGSGSGTDFTLTIDSLQAEDVAVYYCKQSYNLYTFG |
| | | GGTKVEIK |
| hVL-1.16 | 120 | EIVLTQSPDSLAVSLGERATINCKSSQSLLNSGTRKNYLAWYQQKAGQSPKL |
| | | LIYWTSTRESGVPDRFSGSGSGTDFTLTIDSLQAEDVAVYYCKQSYSLYTFG |
| | | GGTKVEIK |
| hVL-1.17 | 121 | EIVLTQSPDSLAVSLGERATIKCKSSQSLLNSGTRKNYLAWYQKKAGQSPKL |
| | | LIYWTSTRESGVPDRFSGSGSGTDFTLTIDSLQAEDVAVYYCKQSYTLYTFG |
| | | GGTKVEIK |
| hVL-1.18 | 122 | EIVLTQSPDSLAVSLGERATINCKSSQSLLNSGTRKNYLAWYQQKAGQSPKL |
| | | LIYWTSTRESGVPDRFSGSGSGTDFTLTIDSLQTEDVAVYYCKQSYTLYTFG |
| | | GGTKVEIK |
| hVL-1.19 | 123 | EIVLTQSPDSLAVSLGERATINCKSSQSLLNSGTRKNYLAWYQQKAGQSPKL |
| | | LIYWTSTRESGVPDRFSASGSGTDFTLTIDSLQAEDVAVYYCKQSYTLYTFG |
| | | GGTKVEIK |
| hVL-1.20 | 124 | EIVLTQSPDSLAVSLGERATINCKSSQSLLNSGTRKNYLTWYQQKAGQSPKL |
| | | LIYWTSTRESGVPDRFSGSGSGTDFTLTIDSLQAEDVALYYCKQSYTLYTFG |
| | | GGTKVEIK |
| hVL-1.21 | 125 | EIVLTQSPDSLSVSLGERATINCKSSQSLLNSGTRKNYLAWYQQKAGQSPKL |
| | | LIYWTSTRESGVPDRFSGSGSGTDFTLTIDSLQAEDVAVYYCKQSYTLYTFG |
| | | GGTKVEIK |
| hVL-1.22 | 126 | EIVLTQSPDSLAVSLGERATINCKSSQSLLNSGTRKNYLAWYQQKSGQSPKL |
| | | LIYWTSTRESGVPDRFSGSGSGTDFTLTIDSLQAEDVAVYYCKQSYTLYTFG |
| | | GGTKVEIK |
| hVL-1.23 | 127 | EIVLTQSPDSLAVSLGERATINCKSSQSLVNSGTRKNYLAWYQQKAGQSPKL |
| | | LIYWTSTRESGVPDRFSGSGSGTDFTLTIDSLQTEDVAVYYCKQSYTLYTFG |
| | | GGTKVEIK |
| hVL-1.24 | 128 | EIVLTQSPDSLAVSLGERATIKCKSSQSLLNSGTRKNYLAWYQQKAGQSPKL |
| | | LIYWTSTRESGVPDRFSGSGSGTDFTLTIDSLQAEDVALYYCKQSYTLYTFG |
| | | GGTKVEIK |
| hVL-1.25 | 129 | EIVLTQSPDSLAVSLGERATINCKSSQSLLNSGTRKNYLAWYQQKAGQSPKL |
| | | IIYWTSTRESGVPDRFSGSGSGTDFTLTIDSLQAEDVALYYCKQSYTLYTFG |
| | | GGTKVEIK |
| hVL-1.26 | 130 | EIVLTQSPDSLAVSLGERATINCKSSQSLLNSGTRKNYLAWYQKKSGQSPKL |
| | | LIYWTSTRESGVPDRFSGSGSGTDFTLSIDSLQAEDVAVYYCKQSYTLYTFG |
| | | GGTKVEIK |
| hVL-1.27 | 131 | EIVLTQSPDSLAVSLGERATIKCKSSQSLLNSGTRKNYLAWYQQKAGQSPKL |
| | | LIYWTSTRESGVPDRFSASGSGTDFTLTIDSLQAEDVAVYYCKQSYTLYTFG |
| | | GGTKVEIK |
| hVL-1.28 | 132 | EIVLTQSPDSLAVSLGERATINCKSSQSLLNSGTRKNYLTWYQQKSGQSPKL |
| | | LIYWTSTRESGVPDRFSGSGSGTDFTLTIDSLQAEDVAIYYCKQSYTLYTFG |
| | | GGTKVEIK |
| hVL-1.29 | 133 | EIVLTQSPDSLAVSLGERATINCKSSQSLLNSGTRKNYLAWYQKKAGQSPKL |
| | | LIYWTSTRESGVPDRFSGSGSGTDFTLSIDSLQAEDVAIYYCKQSYTLYTFG |
| | | GGTKVEIK |
| hVL-1.30 | 134 | EIVLTQSPDSLAVSLGERATINCKSSQSLLNSGTRKNYLAWYQQKSGQSPKL |
| | | LIYWTSTRESGVPDRFSGSGSGTDFTLTIDSLQAEDVAIYYCKQSYTLYTFG |
| | | GGTKVEIK |
| hVL-1.31 | 135 | EIVLTQSPDSLSVSLGERATINCKSSQSLLNSGTRKNYLAWYQQKAGQSPNL |
| | | LIYWTSTRESGVPDRFSGSGSGTDFTLSIDSLQAEDVAVYYCKQSYTLYTFG |
| | | GGTKVEIK |
| hVL-1.32 | 136 | EIVLTQSPDSLSVSLGERATINCKSSQSLLNSGTRKNYLAWYQQKAGQSPNL |
| | | LIYWTSTRESGVPDRFSGSGSGTDFTLTIDSLQAEDVAVYYCKQSYTLYTFG |
| | | GGTKVEIK |
| hVL-1.33 | 137 | EIVLTQSPDSLTVSLGERATINCKSSQSLLNSGTRKNYLAWYQQKAGQSPNL |
| | | LIYWTSTRESGVPDRFSGSGSGTDFTLTIDSLQAEDVALYYCKQSYTLYTFG |
| | | GGTKVEIK |
| hVL-1.34 | 138 | EIVLTQSPDSLSVSLGERATINCKSSQSLLNSGTRKNYLAWYQKKAGQSPKL |
| | | LIYWTSTRESGVPDRFSGSGSGTDFTLTIDRLQAEDVALYYCKQSYTLYTFG |
| | | GGTKVEIK |
| hVL-1.35 | 139 | EIVLTQSPDSLSVSLGERATIKCKSSQSLLNSGTRKNYLAWYQQKAGQSPKL |
| | | LIYWTSTRESGVPDRFSGSGSGTDFTLTIDSLQTEDVAVYYCKQSYTLYTFG |
| | | GGTKVEIK |
| hVL-1.36 | 140 | EIVLTQSPDSLAVSLGERATIKCKSSQSLLNSGTRKNYLAWYQQKAGQSPKL |
| | | LIYWTSTRESGVPDRFSGSGSGTDFTLTIDSLQAEDVAIYYCKQSYTLYTFG |
| | | GGTKVEIK |
| hVL-1.37 | 141 | EIVLTQSPDSLAVSLGERATINCKSSQSLFNSGTRKNYLAWYQQKAGQSPKL |
| | | LIYWTSTRESGVPDRFSGSGSGTDFTLSIDSLQAEDVAVYYCKQSYTLYTFG |
| | | GGTKVEIK |
| hVL-1.38 | 142 | EIVLTQSPDSLAVSLGERATINCKSSQSLLNSGTRKNYLAWYQQKAGQSPNL |
| | | LIYWTSTRESGVPDRFSGSGSGTDFTLTIDSLQAEDVAVYYCKQSYSLYTFG |
| | | GGTKVEIK |
| hVL-1.39 | 143 | EIVLTQSPDSLAVSLGERATINCKSSQSLLNSGTRKNYLAWYQQKPGQSPKL |
| | | LIYWTSTRESGVPDRFSGSGSGTDFTLTIDSLQAEDVAVYYCKQSYNLYTFG |
| | | GGTKVEIK |
| hVL-1.40 | 144 | EIVLTQSPDSLAVSLGERATINCKSSQSLLNSGTRKNYLSWYQQKPGQSPKL |
| | | IIYWTSTRESGVPDRFSGSGSGTDFTLTIDSLQAEDVAVYYCKQSYTLYTFG |
| | | GGTKVEIK |
| hVL-1.41 | 145 | EIVLTQSPDSLAVSLGERATINCKSSQSLLNSGTRKNYLAWYQQKSGQSPKL |
| | | LIYWTSTRESGVPDRFSGSGSGTDFTLTIDSLQTEDVAIYYCKQSYTLYTFG |
| | | GGTKVEIK |
| hVL-1.42 | 146 | EIVLTQSPDSLAVSLGERATINCKSSQSLLNSGTRKNYLSWYQQKAGQSPNL |
| | | VIYWTSTRESGVPDRFSGSGSGTDFTLTIDSLQAEDVAVYYCKQSYTLYTFG |
| | | GGTKVEIK |
| hVL-1.43 | 147 | EIVLTQSPDSLAVSLGERATINCKSSQSLLNSGTRKNYLAWYQKKSGQSPNL |
| | | LIYWTSTRESGVPDRFSGSGSGTDFTLTIDSLQAEDVAVYYCKQSYTLYTFG |
| | | GGTKVEIK |
| hVL-1.44 | 148 | EIVLTQSPDSLAVSLGERATINCKSSQSLLNSGTRKNYLSWYQKKSGQSPKL |
| | | LIYWTSTRESGVPDRFSGSGSGTDFTLTIDSLQAEDVAVYYCKQSYTLYTFG |
| | | GGTKVEIK |
| hVL-1.45 | 149 | EIVLTQSPDSLAVSLGERATINCKSSQSLFNSGTRKNYLAWYQKKAGQSPKL |
| | | LIYWTSTRESGVPDRFSGSGSGTDFTLSIDRLQAEDVAVYYCKQSYTLYTFG |
| | | GGTKVEIK |
| hVL-1.46 | 150 | EIVLTQSPDSLTVSLGERATINCKSSQSLLNSGTRKNYLAWYQQKPGQSPKL |
| | | LIYWTSTRESGVPDRFSGSGSGTDFTLTIDSLQAEDVAVYYCKQSYTLYTFG |
| | | GGTKVEIK |
| hVL-1.47 | 151 | EIVLTQSPDSLAVSLGERATIKCKSSQSLLNSGTRKNYLTWYQQKAGQSPNL |
| | | LIYWTSTRESGVPDRFSGSGSGTDFTLTIDSLQAEDVAVYYCKQSYTLYTFG |
| | | GGTKVEIK |
| hVL-1.48 | 152 | EIVLTQSPDSLSVSLGERATINCKSSQSLLNSGTRKNYLAWYQQKSGQSPNL |
| | | VIYWTSTRESGVPDRFSGSGSGTDFTLTIDSLQAEDVAVYYCKQSYTLYTFG |
| | | GGTKVEIK |
| hVL-1.49 | 153 | EIVLTQSPDSLSVSLGERATINCKSSQSLFNSGTRKNYLAWYQQKAGQSPKL |
| | | LIYWTSTRESGVPDRFSGSGSGTDFTLTIDSLQAEDVAVYYCKQSYTLYTFG |
| | | GGTKVEIK |
| hVL-1.50 | 154 | EIVLTQSPDSLTVSLGERATINCKSSQSLFNSGTRKNYLAWYQKKAGQSPKL |
| | | LIYWTSTRESGVPDRFSGSGSGTDFTLTIDSLQAEDVAVYYCKQSYNLYTFG |
| | | GGTKVEIK |
| hVL-1.51 | 155 | EIVLTQSPDSLAVSLGERATINCKSSQSLLNSGTRKNYLAWYQQKPGQSPKL |
| | | LIYWTSTRESGVPDRFSASGSGTDFTLTIDSLQAEDVAIYYCKQSYTLYTFG |
| | | GGTKVEIK |
| hVL-1.52 | 156 | EIVLTQSPDSLAVSLGERATINCKSSQSLFNSGTRKNYLAWYQQKAGQSPKL |
| | | IIYWTSTRESGVPDRFSASGSGTDFTLSIDRLQAEDVAVYYCKQSYTLYTFG |
| | | GGTKVEIK |
| hVL-1.53 | 157 | EIVLTQSPDSLSVSLGERATINCKSSQSLLNSGTRKNYLAWYQQKAGQSPKL |
| | | LIYWTSTRESGVPDRFSASGSGTDFTLTIDRLQAEDVALYYCKQSYTLYTFG |
| | | GGTKVEIK |
| hVL-1.54 | 24 | DIVMTQSPDSLAVSLGERATINCKSSQSLLNSGTRKNYLAWYQQKPG |
| | | QSPKLLIYWTSTRESGVPDRFSGSGSGTDFTLTISSLQAEDVAVYYC |
| | | KQSYTLYTFGQGTKLEIK |

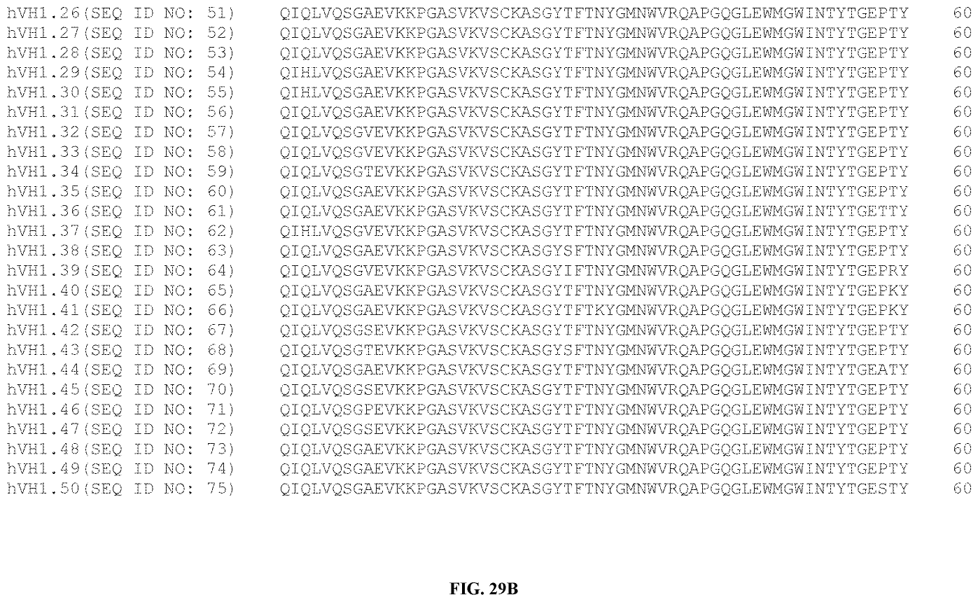

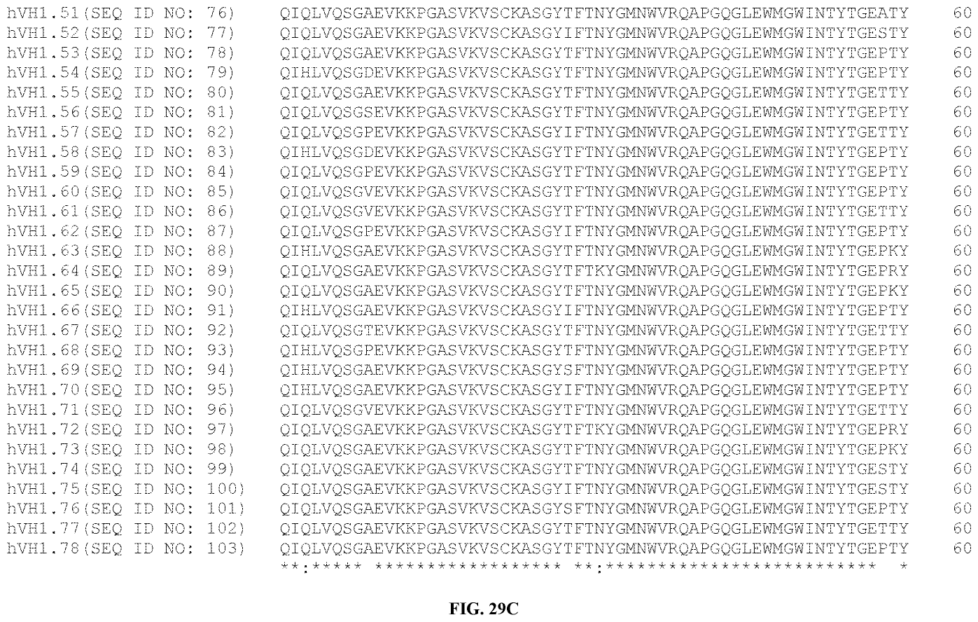

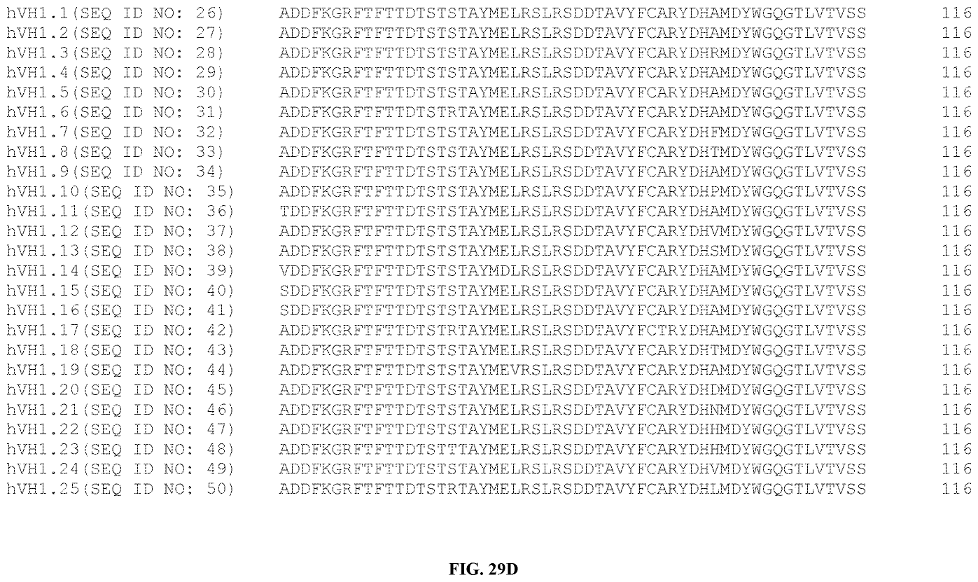

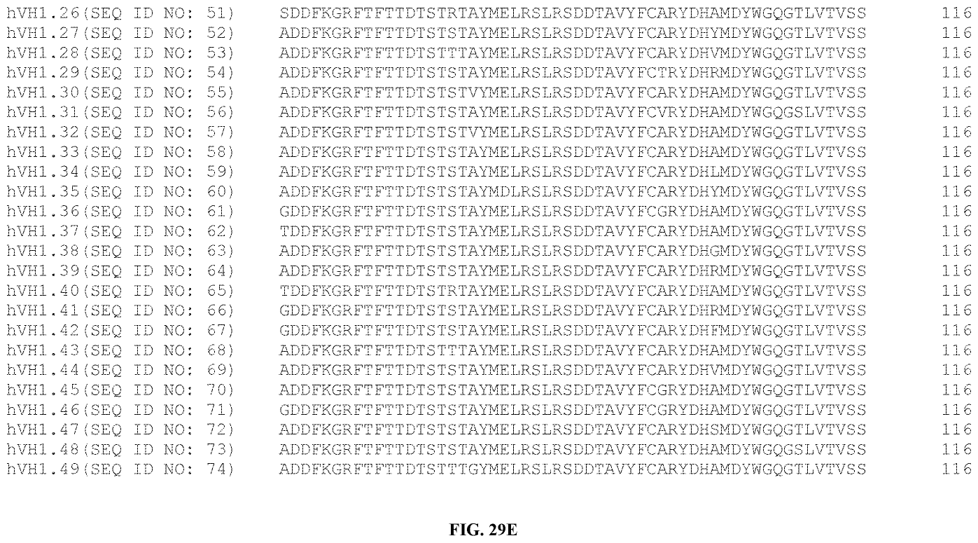

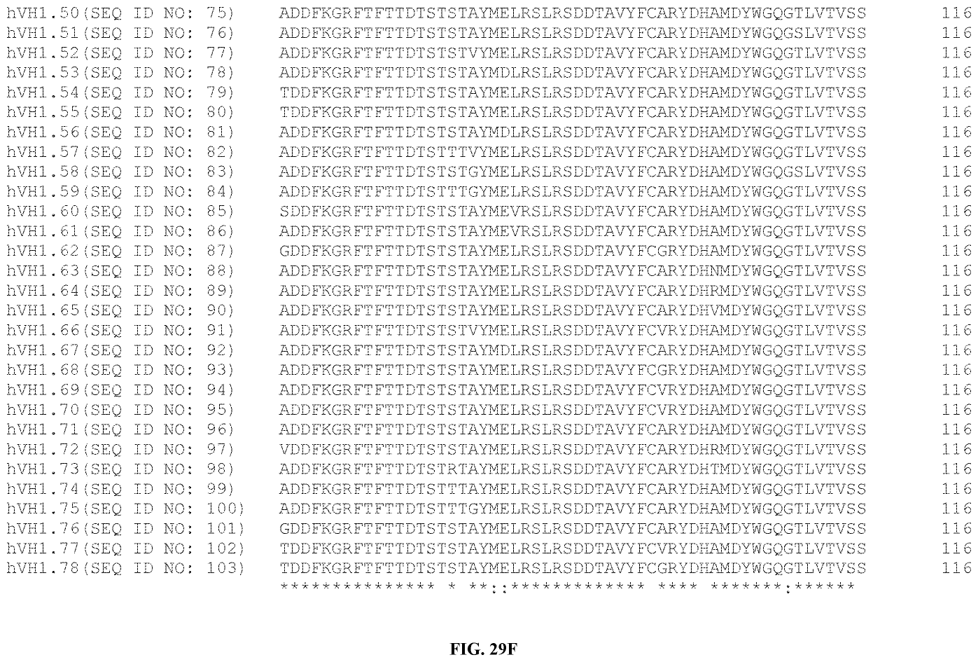

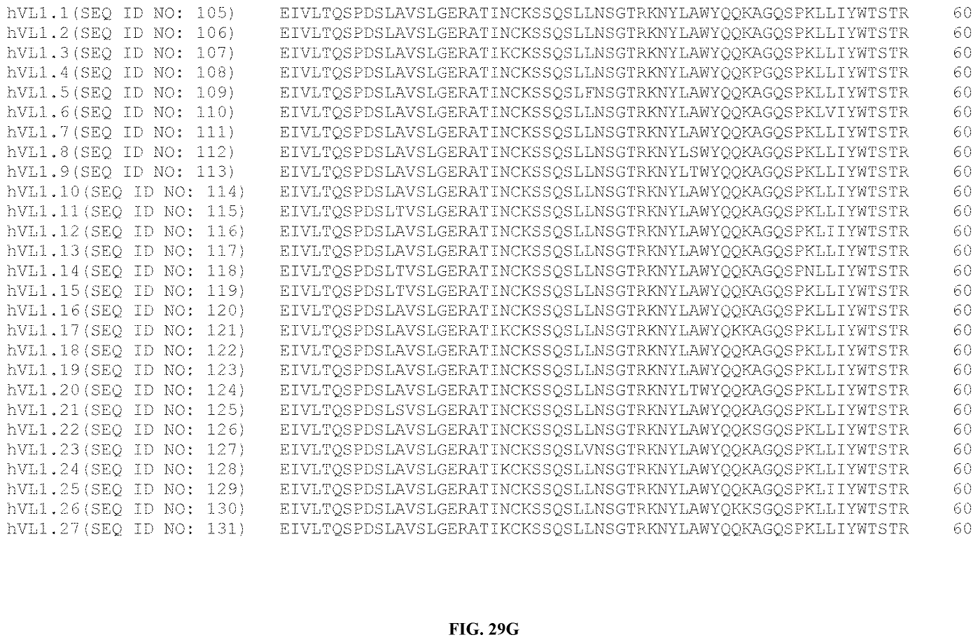

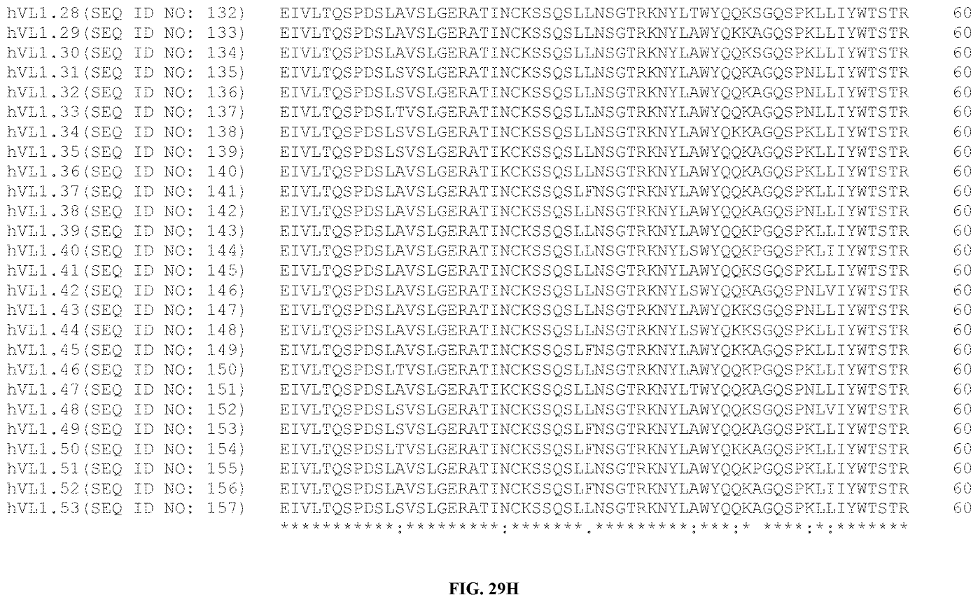

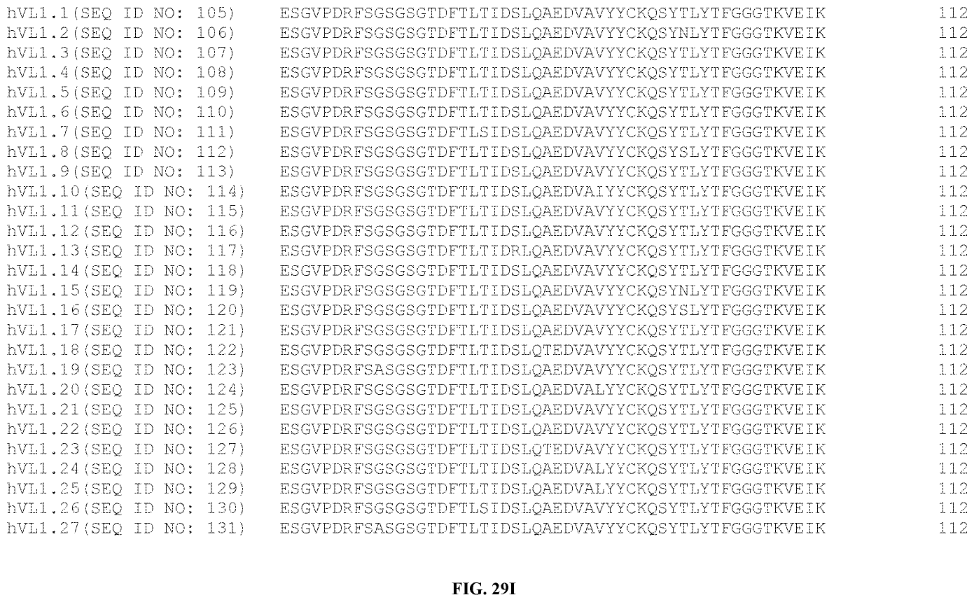

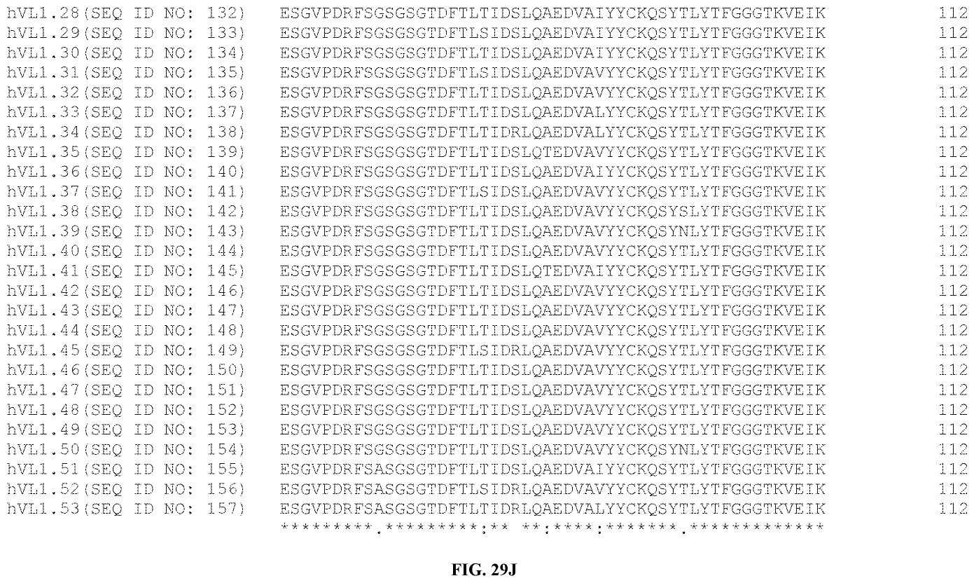

A sequence alignment of humanized variants hVH-1.1 through hVH-1.78 is depicted in FIGS. 29A-F, and hVL-1.1 through hVL-1.53 in FIGS. 29G-J.

DNA coding for the amino acid sequence of the variants were synthesized and cloned into a mammalian transient expression plasmid. Variants were expressed using a CHO based transient expression system and the resulting antibody containing cell culture supernatants were clarified by centrifugation and filtration. Variants were purified from cell culture supernatants via affinity chromatography. Purified antibodies were buffer exchanged into phosphate buffered saline solution. The purity of the resulting antibodies was determined to be >95%, as judged by reducing and denaturing SDS-PAGE gels. Antibody concentration was determined by measuring absorbance at 280 nm.

Binding assays were performed as follows. Antibody variants were immobilized using anti-human Fc onto the surface of a series of biosensors at 0.15 μg/ml. Antigen, at 100 nM, was passed over the surface to generate a binding response. Binding data for the antibody:antigen interactions were collected at 25° C. on the biosensors. Results for select antibody variants are shown in Table 14. In Table 14, X=610 represents captured antigen value (nM) at the beginning of the dissociation phase (measured at 610 seconds after the biosensor was initially contacted with antigen) and X=1495 represents captured antigen value (nM) at the end of the dissociation phase (measured at 1495 seconds after the biosensor was initially contacted with antigen). Antibodies with an X=610 value greater than 0.2 and a % dissociation less than 10% are shown in bold.

TABLE 14
Binding Assays

| Antibody | Capture Level | X = 610 | X = 1495 | % Diss |
|---|---|---|---|---|
| h77A-1 | 0.656 | 0.2740 | 0.2531 | 7.63 |
| h77A-1.1 | 0.645 | 0.2729 | 0.2623 | 3.88 |
| h77A-1.2 | 0.700 | 0.0496 | 0.0505 | −1.81 |
| h77A-1.3 | 0.735 | 0.2546 | 0.1816 | 28.67 |
| h77A-1.4 | 0.705 | 0.2583 | 0.1763 | 31.75 |
| h77A-1.5 | 0.706 | 0.2569 | 0.1919 | 25.30 |
| h77A-1.6 | 0.684 | 0.2901 | 0.2710 | 6.58 |
| h77A-1.7 | 0.710 | 0.2182 | 0.1524 | 30.16 |
| h77A-1.8 | 0.742 | 0.3331 | 0.3044 | 8.62 |
| h77A-1.9 | 0.692 | 0.2778 | 0.2269 | 18.32 |
| h77A-1.10 | 0.723 | 0.3162 | 0.2885 | 8.76 |
| h77A-1.11 | 0.713 | 0.3006 | 0.2684 | 10.71 |
| h77A-1.12 | 0.701 | 0.2997 | 0.2855 | 4.74 |
| h77A-1.13 | 0.721 | 0.1133 | 0.0981 | 13.42 |
| h77A-1.14 | 0.701 | 0.3123 | 0.2877 | 7.88 |
| h77A-1.15 | 0.721 | 0.2921 | 0.1956 | 33.04 |
| h77A-1.16 | 0.713 | 0.3048 | 0.2949 | 3.25 |
| h77A-1.17 | 0.741 | 0.3267 | 0.3037 | 7.04 |
| h77A-1.18 | 0.688 | 0.2841 | 0.2638 | 7.15 |
| h77A-1.19 | 0.705 | 0.3138 | 0.2864 | 8.73 |
| h77A-1.20 | 0.712 | 0.3111 | 0.2736 | 12.05 |
| h77A-1.21 | 0.691 | 0.2977 | 0.2679 | 10.01 |
| h77A-1.22 | 0.747 | 0.3309 | 0.3094 | 6.50 |
| h77A-1.23 | 0.758 | 0.3217 | 0.2444 | 24.03 |
| h77A-1.24 | 0.604 | 0.1601 | 0.1063 | 33.60 |
| h77A-1.25 | 0.726 | 0.2198 | 0.1687 | 23.25 |
| h77A-1.26 | 0.705 | 0.2961 | 0.261 | 11.85 |
| h77A-1.27 | 0.698 | 0.2922 | 0.2725 | 6.74 |
| h77A-1.28 | 0.696 | 0.273 | 0.2217 | 18.79 |
| h77A-1.29 | 1.032 | 0.3705 | 0.2656 | 28.31 |
| h77A-1.30 | 0.692 | 0.2843 | 0.2698 | 5.10 |
| h77A-1.31 | 0.688 | 0.1108 | 0.1147 | −3.52 |
| h77A-1.32 | 0.651 | 0.0256 | 0.0462 | −80.47 |
| h77A-1.33 | 0.700 | 0.2359 | 0.1873 | 20.60 |
| h77A-1.35 | 0.707 | 0.1665 | 0.1592 | 4.38 |
| h77A-1.36 | 0.702 | 0.1868 | 0.1738 | 6.96 |
| h77A-1.37 | 0.659 | 0.2385 | 0.1842 | 22.77 |
| h77A-1.38 | 0.546 | 0.0543 | 0.0486 | 10.50 |
| h77A-1.39 | 0.701 | 0.2269 | 0.1793 | 20.98 |
| h77A-1.40 | 0.679 | 0.0918 | 0.0809 | 11.87 |
| h77A-1.41 | 0.713 | 0.2754 | 0.1811 | 34.24 |
| h77A-1.42 | 0.721 | 0.2155 | 0.1433 | 33.50 |
| h77A-1.43 | 0.678 | 0.2466 | 0.1946 | 21.09 |
| h77A-1.45 | 0.698 | 0.2643 | 0.1748 | 33.86 |
| h77A-1.46 | 0.689 | 0.275 | 0.2579 | 6.22 |
| h77A-1.47 | 0.740 | 0.0917 | 0.0792 | 13.63 |
| h77A-1.48 | 0.666 | 0.1875 | 0.1229 | 34.45 |
| h77A-1.50 | 0.644 | 0.2639 | 0.2342 | 11.25 |
| h77A-1.52 | 0.681 | 0.0062 | 0.0133 | −114.52 |
| h77A-1.53 | 0.643 | 0.1222 | 0.1086 | 11.13 |
| h77A-1.54 | 0.665 | 0.0068 | 0.0096 | −41.18 |
| h77A-1.55 | 0.619 | 0.1545 | 0.1133 | 26.67 |
| h77A-1.57 | 0.646 | 0.1704 | 0.1262 | 25.94 |
| h77A-1.59 | 0.652 | 0.2509 | 0.2263 | 9.80 |
| h77A-1.61 | 0.639 | 0.2539 | 0.237 | 6.66 |
| h77A-1.62 | 0.579 | 0.2199 | 0.1928 | 12.32 |
| h77A-1.63 | 0.563 | 0.2257 | 0.1937 | 14.18 |
| h77A-1.64 | 0.572 | 0.2086 | 0.1648 | 21.00 |
| h77A-1.65 | 0.612 | 0.2513 | 0.1997 | 20.53 |
| h77A-1.66 | 0.665 | 0.23 | 0.1561 | 32.13 |
| h77A-1.70 | 0.674 | 0.2761 | 0.2441 | 11.59 |
| h77A-1.71 | 0.676 | 0.2899 | 0.2669 | 7.93 |
| h77A-1.72 | 0.636 | 0.1701 | 0.1165 | 31.51 |
| h77A-1.74 | 0.665 | 0.2934 | 0.2668 | 9.07 |
| h77A-1.75 | 0.682 | 0.3053 | 0.289 | 5.34 |
| h77A-1.76 | 0.670 | 0.2767 | 0.2431 | 12.14 |
| h77A-1.77 | 0.689 | 0.2285 | 0.1542 | 32.52 |
| h77A-1.79 | 0.683 | 0.0131 | 0.018 | −37.40 |
| h77A-1.80 | 0.630 | 0.0181 | 0.0197 | −8.84 |
| h77A-1.81 | 0.653 | 0.2644 | 0.1847 | 30.14 |
| h77A-1.85 | 0.616 | 0.2523 | 0.2117 | 16.09 |
| h77A-1.86 | 0.522 | 0.1908 | 0.1639 | 14.10 |
| h77A-1.91 | 0.637 | 0.2541 | 0.1762 | 30.66 |
| h77A-1.92 | 0.620 | 0.2346 | 0.1586 | 32.40 |
| h77A-1.94 | 0.660 | 0.2978 | 0.2822 | 5.24 |
| h77A-1.96 | 0.749 | 0.3288 | 0.2669 | 18.83 |
| h77A-1.97 | 0.645 | 0.1793 | 0.1305 | 27.22 |
| h77A-1.98 | 0.675 | 0.2325 | 0.1522 | 34.54 |

Further kinetics assays were conducted for select antibody variants. Assays were performed as follows. Antibody variants were immobilized using anti-human Fc onto the surface of a series of biosensors. Antigen was passed over the surface to generate a binding response. Binding data for the antibody:antigen interactions were collected at 25° C. on the biosensors. A dilution series of the antigen was used in the association step, in order to fit results globally and get the best values for ka, kd, and KD. The response data for the binding of antigen to the surface immobilized antibody were fitted to a 1:1 binding model. Kinetic parameters are summarized in Table 15.

TABLE 15
Binding kinetics

| | Capture | | | | | | | |
|---|---|---|---|---|---|---|---|---|
| | level | kₐ | kₐ | K_{D} | K_{D} | | | Rₘₐₓ at |
| Antibody | (nm) | (m⁻¹s⁻¹) | (s⁻¹) | (M) | (nM) | R² | X² | 33.3 nM |
| h77A-1 | 0.726 | 5.47E+04 | 1.31E−04 | 2.39 | 1.20E−09 | 0.9981 | 0.2056 | 0.2942 |
| h77A-1.1 | 0.68 | 8.85E+04 | 6.11E−05 | 0.69 | 3.20E−11 | 0.9969 | 0.2685 | 0.2271 |
| h77A-1.6 | 0.678 | 5.42E+04 | 1.14E−04 | 2.11 | 1.80E−09 | 0.9983 | 0.1792 | 0.2735 |
| h77A-1.8 | 0.7 | 5.48E+04 | 7.42E−05 | 1.35 | 3.10E−10 | 0.9984 | 0.188 | 0.2984 |
| h77A-1.10 | 0.677 | 5.84E+04 | 1.45E−04 | 2.48 | 2.60E−09 | 0.9981 | 0.1872 | 0.2679 |
| h77A-1.12 | 0.784 | 5.17E+04 | 7.37E−05 | 1.43 | 1.10E−09 | 0.9984 | 0.2097 | 0.3106 |
| h77A-1.14 | 0.704 | 6.31E+04 | 1.24E−04 | 1.97 | 7.70E−10 | 0.9978 | 0.1844 | 0.2516 |
| h77A-1.16 | 0.75 | 5.40E+04 | 6.84E−05 | 1.27 | 1.30E−09 | 0.9986 | 0.1422 | 0.2774 |
| h77A-1.17 | 0.686 | 6.14E+04 | 9.24E−05 | 1.51 | 1.30E−09 | 0.9972 | 0.3139 | 0.2752 |
| h77A-1.18 | 0.639 | 6.32E+04 | 9.10E−05 | 1.44 | 8.10E−10 | 0.9974 | 0.2789 | 0.2549 |
| h77A-1.19 | 0.71 | 8.85E+04 | 1.11E−04 | 1.26 | 1.20E−09 | 0.9983 | 0.2233 | 0.2722 |
| h77A-1.22 | 0.779 | 6.69E+04 | 7.64E−05 | 1.14 | 8.10E−10 | 0.9985 | 0.2631 | 0.3227 |
| h77A-1.27 | 0.746 | 7.64E+04 | 1.08E−04 | 1.41 | 8.90E−10 | 0.9986 | 0.2141 | 0.295 |
| h77A-1.30 | 0.744 | 5.67E+04 | 1.09E−04 | 1.93 | 8.60E−10 | 0.9984 | 0.1831 | 0.2812 |
| h77A-1.46 | 0.754 | 5.48E+04 | 1.23E−04 | 2.24 | 1.20E−09 | 0.9987 | 0.1571 | 0.2816 |
| h77A-1.59 | 0.708 | 4.08E+04 | 1.20E−04 | 2.93 | 7.40E−10 | 0.9987 | 0.0944 | 0.2583 |
| h77A-1.61 | 0.65 | 4.21E+04 | 1.23E−04 | 2.91 | 2.10E−09 | 0.9984 | 0.1083 | 0.2361 |
| h77A-1.71 | 0.747 | 4.90E+04 | 1.23E−04 | 2.51 | 1.30E−09 | 0.9988 | 0.1362 | 0.2911 |
| h77A-1.74 | 0.699 | 7.78E+04 | 1.25E−04 | 1.61 | 1.70E−09 | 0.9979 | 0.2514 | 0.2562 |
| h77A-1.75 | 0.748 | 8.81E+04 | 2.43E−05 | 0.28 | 2.30E−11 | 0.9972 | 0.4938 | 0.2906 |
| h77A-1.94 | 0.654 | 5.99E+04 | 1.11E−04 | 1.85 | 1.30E−09 | 0.9976 | 0.1953 | 0.2325 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Anderson D A, 3rd, Murphy K M, Briseno C G. Development, Diversity, and Function of Dendritic Cells in Mouse and Human. Cold Spring Harb Perspect Biol. 2018; 10(11).
Aprile et al., PLoS ONE, 8:e67962, 2013.
Bennett C L, van Rijn E, Jung S, et al. Inducible ablation of mouse Langerhans cells diminishes but fails to abrogate contact hypersensitivity. J Cell Biol. 2005; 169(4):569-576.
Berges C, Naujokat C, Tinapp S, et al. A cell line model for the differentiation of human dendritic cells. Biochem Biophys Res Commun. 2005; 333(3):896-907.
Binder R J. Heat-shock protein-based vaccines for cancer and infectious disease. Expert Rev Vaccines. 2008;7(3):383-393.
Bjorklund C C, Ma W, Wang Z Q, et al. Evidence of a role for activation of Wnt/beta-catenin signaling in the resistance of plasma cells to lenalidomide. J Biol Chem. 2011;286(13): 11009-11020.
Boudesco C, Cause S, Jego G, Garrido C. Hsp70: A Cancer Target Inside and Outside the Cell. Methods Mol Biol. 2018; 1709:371-396.
Bourque J, Hawiger D. Immunomodulatory Bonds of the Partnership between Dendritic Cells and T Cells. Crit Rev Immunol. 2018; 38(5):379-401.
Bruhns, Blood, 119:5640, 2012.
Casanova V, Naval-Macabuhay I, Massanella M, et al. Adenosine deaminase enhances the immunogenicity of human dendritic cells from healthy and HIV-infected individuals. PLoS One. 2012; 7(12):e51287.
Chacon J A, Sarnaik A A, Chen J Q, et al. Manipulating the tumor microenvironment ex vivo for enhanced expansion of tumor-infiltrating lymphocytes for adoptive cell therapy. Clin Cancer Res. 2015; 21(3):611-621.
Chatterjee M, Andrulis M, Stuhmer T, et al. The PI3K/Akt signaling pathway regulates the expression of Hsp70, which critically contributes to Hsp90-chaperone function and tumor cell survival in multiple myeloma. Haematologica. 2013; 98(7):1132-1141.
Chatterjee S, Burns T F. Targeting Heat Shock Proteins in Cancer: A Promising Therapeutic Approach. Int J Mol Sci. 2017; 18(9).
Chen N, Ritsma L M A, Vrisekoop N. In vivo characteristics of human and mouse breast tumor cell lines. Exp Cell Res. 2019.
Chen D S, Mellman I. Oncology meets immunology: the cancer-immunity cycle. Immunity. 2013; 39(1):1-10.
Craig E A, Marszalek J. How Do J-Proteins Get Hsp70 to Do So Many Different Things? Trends Biochem Sci. 2017; 42(5):355-368.
Dabaghian M, Latify A M, Tebianian M, et al. Vaccination with recombinant 4 x M2e.HSP70c fusion protein as a universal vaccine candidate enhances both humoral and cell-mediated immune responses and decreases viral shedding against experimental challenge of H9N2 influenza in chickens. Vet Microbiol. 2014; 174(1-2):116-126.
Daugaard M, Rohde M, Jaattela M. The heat shock protein 70 family: Highly homologous proteins with overlapping and distinct functions. FEBS Lett. 2007; 581(19):3702-3710.
Davidson E, Doranz B J. A high-throughput shotgun mutagenesis approach to mapping B-cell antibody epitopes. Immunology. 2014; 143(1):13-20.
Demaria O, De Gassart A, Coso S, et al. STING activation of tumor endothelial cells initiates spontaneous and therapeutic antitumor immunity. Proc Natl Acad Sci USA. 2015; 112(50):15408-15413.
Dodd et al., Oncogene, 34:1312, 2015.
Duffield J S, Forbes S J, Constandinou C M, et al. Selective depletion of macrophages reveals distinct, opposing roles during liver injury and repair. J Clin Invest. 2005; 115(1):56-65.
Fu C, Jiang A. Dendritic Cells and CD8 T Cell Immunity in Tumor Microenvironment. Front Immunol. 2018; 9:3059.
Fukaya T, Murakami R, Takagi H, et al. Conditional ablation of CD205+ conventional dendritic cells impacts the regulation of T-cell immunity and homeostasis in vivo. Proc Natl Acad Sci USA. 2012; 109(28):11288-11293.
Gornati L, Zanoni I, Granucci F. Dendritic Cells in the Cross Hair for the Generation of Tailored Vaccines. Front Immunol. 2018; 9:1484.
Greene L E, Zinner R, Naficy S, Eisenberg E. Effect of nucleotide on the binding of peptides to 70-kDa heat shock protein. J Biol Chem. 1995; 270(7):2967-2973.
Guzhova I V, Margulis B A. HSP70-based anti-cancer immunotherapy. Hum Vaccin Immunother. 2016; 12(10):2529-2535.
Hofgaard P O, Jodal H C, Bommert K, et al. A novel mouse model for multiple myeloma (MOPC315.BM) that allows noninvasive spatiotemporal detection of osteolytic disease. PLoS One. 2012; 7(12):e51892.
Hossain M K, Wall K A. Use of Dendritic Cell Receptors as Targets for Enhancing Anti-Cancer Immune Responses. Cancers (Basel). 2019; 11(3).
Huang A Y, Gulden P H, Woods A S, et al. The immunodominant major histocompatibility complex class I-restricted antigen of a murine colon tumor derives from an endogenous retroviral gene product. Proc Natl Acad Sci USA. 1996; 93(18):9730-9735.
Huarte E, Cubillos-Ruiz J R, Nesbeth Y C, et al. Depletion of dendritic cells delays ovarian cancer progression by boosting antitumor immunity. Cancer Res. 2008; 68(18):7684-7691.
Hunt C, Calderwood S. Characterization and sequence of a mouse hsp70 gene and its expression in mouse cell lines. Gene. 1990; 87(2):199-204.
Johnsen G, Elsayed S. Antigenic and allergenic determinants of ovalbumin-III. MHC Ia-binding peptide (OA 323-339) interacts with human and rabbit specific antibodies. Mol Immunol. 1990; 27(9):821-827.
Joly A L, Wettstein G, Mignot G, Ghiringhelli F, Garrido C. Dual role of heat shock proteins as regulators of apoptosis and innate immunity. J Innate Immun. 2010; 2(3):238-247.
Jung S, Unutmaz D, Wong P, et al. In vivo depletion of CD11c+ dendritic cells abrogates priming of CD8+ T cells by exogenous cell-associated antigens. Immunity. 2002; 17(2):211-220.
Kandasamy G, Andreasson C. Hsp70-Hsp110 chaperones deliver ubiquitin-dependent and -independent substrates to the 26S proteasome for proteolysis in yeast. J Cell Sci. 2018; 131(6).
Karttunen J, Sanderson S, Shastri N. Detection of rare antigen-presenting cells by the lacZ T-cell activation assay suggests an expression cloning strategy for T-cell antigens. Proc Natl Acad Sci USA. 1992; 89(13):6020-6024.
Kim S J, Schatzle S, Ahmed S S, et al. Increased cathepsin S in Prdm1(−/−) dendritic cells alters the TFH cell repertoire and contributes to lupus. Nat Immunol. 2017; 18(9):1016-1024.
Kim K, Skora A D, Li Z, et al. Eradication of metastatic mouse cancers resistant to immune checkpoint blockade by suppression of myeloid-derived cells. Proc Natl Acad Sci USA. 2014; 111(32):11774-11779.
Kissenpfennig A, Henri S, Dubois B, et al. Dynamics and function of Langerhans cells in vivo: dermal dendritic cells colonize lymph node areas distinct from slower migrating Langerhans cells. Immunity. 2005; 22(5):643-654.
Kodama A, Tanaka R, Saito M, Ansari A A, Tanaka Y. A novel and simple method for generation of human dendritic cells from unfractionated peripheral blood mononuclear cells within 2 days: its application for induction of HIV-1-reactive CD4(+) T cells in the hu-PBL SCID mice. Front Microbiol. 2013; 4:292.
Koelle D M, Magaret A, McClurkan C L, et al. Phase I dose-escalation study of a monovalent heat shock protein 70-herpes simplex virus type 2 (HSV-2) peptide-based vaccine designed to prime or boost CD8 T-cell responses in HSV-naive and HSV-2-infected subjects. Clin Vaccine Immunol. 2008; 15(5):773-782.
Kroemer G, Galluzzi L, Kepp O, Zitvogel L. Immunogenic cell death in cancer therapy. Annu Rev Immunol. 2013; 31:51-72.
Kuhn D J, Berkova Z, Jones R J, et al. Targeting the insulin-like growth factor-1 receptor to overcome bortezomib resistance in preclinical models of multiple myeloma. Blood. 2012; 120(16):3260-3270.
Kuhn D J, Chen Q, Voorhees P M, et al. Potent activity of carfilzomib, a novel, irreversible inhibitor of the ubiquitin-proteasome pathway, against preclinical models of multiple myeloma. Blood. 2007; 110(9):3281-3290.
Kuhn D J, Hunsucker S A, Chen Q, Voorhees P M, Orlowski M, Orlowski R Z. Targeted inhibition of the immunoproteasome is a potent strategy against models of multiple myeloma that overcomes resistance to conventional drugs and nonspecific proteasome inhibitors. Blood. 2009; 113(19):4667-4676.
Lao J, Madani J, Puertolas T, et al. Liposomal Doxorubicin in the treatment of breast cancer patients: a review. J Drug Deliv. 2013; 2013:456409.
Lee H C, Wang H, Baladandayuthapani V, et al. RNA polymerase I inhibition with CX-5461 as a novel therapeutic strategy to target MYC in multiple myeloma. Br J Haematol. 2016; In press.
Lee Y H, Martin-Orozco N, Zheng P, et al. Inhibition of the B7-H3 immune checkpoint limits tumor growth by enhancing cytotoxic lymphocyte function. Cell Res. 2017; 27(8):1034-1045.
Lewis D J, Wang Y, Huo Z, et al. Effect of vaginal immunization with HIVgp140 and HSP70 on HIV-1 replication and innate and T cell adaptive immunity in women. J Virol. 2014; 88(20):11648-11657.
Li H S, Liu C, Xiao Y, et al. Bypassing STAT3-mediated inhibition of the transcriptional regulator ID2 improves the antitumor efficacy of dendritic cells. Sci Signal. 2016; 9(447):ra94.
Li B, Fu J, Chen P, et al. The Nuclear Factor (Erythroid-derived 2)-like 2 and Proteasome Maturation Protein Axis Mediate Bortezomib Resistance in Multiple Myeloma. J Biol Chem. 2015; 290(50):29854-29868.
Lou Y, Liu C, Lizee G, et al. Antitumor activity mediated by CpG: the route of administration is critical. J Immunother. 2011; 34(3):279-288.
Lu X, Horner J W, Paul E, et al. Effective combinatorial immunotherapy for castration-resistant prostate cancer. Nature. 2017; 543(7647):728-732.
Lyseng-Williamson K A, Duggan S T, Keating G M. Pegylated liposomal doxorubicin: a guide to its use in various malignancies. BioDrugs. 2013; 27(5):533-540.
Ma W, Wang M, Wang Z Q, et al. Effect of long-term storage in TRIzol on microarray-based gene expression profiling. Cancer Epidemiol Biomarkers Prey. 2010; 19(10):2445-2452.
Malczyk A H, Kupke A, Prufer S, et al. A Highly Immunogenic and Protective Middle East Respiratory Syndrome Coronavirus Vaccine Based on a Recombinant Measles Virus Vaccine Platform. J Virol. 2015; 89(22):11654-11667.
Maleki Vareki S. High and low mutational burden tumors versus immunologically hot and cold tumors and response to immune checkpoint inhibitors. J Immunother Cancer. 2018; 6(1):157.
Malviya G, Anzola K L, Podesta E, et al. (99m)Tc-labeled rituximab for imaging B lymphocyte infiltration in inflammatory autoimmune disease patients. Mol Imaging Biol. 2012; 14(5):637-646.
McNulty S, Colaco C A, Blandford L E, Bailey C R, Baschieri S, Todryk S. Heat-shock proteins as dendritic cell-targeting vaccines—getting warmer. Immunology. 2013; 139(4):407-415.
Mitsiades N, Mitsiades C S, Poulaki V, et al. Molecular sequelae of proteasome inhibition in human multiple myeloma cells. Proc Natl Acad Sci USA. 2002; 99(22):14374-14379.
Moore M W, Carbone F R, Bevan M J. Introduction of soluble protein into the class I pathway of antigen processing and presentation. Cell. 1988; 54(6):777-785.
Murshid A, Gong J, Calderwood S K. The role of heat shock proteins in antigen cross presentation. Front Immunol. 2012; 3:63.
Niknam S, Barsoumian H B, Schoenhals J E, et al. Radiation Followed by OX40 Stimulation Drives Local and Abscopal Antitumor Effects in an Anti-PD1-Resistant Lung Tumor Model. Clin Cancer Res. 2018; 24(22):5735-5743.
Nollen E A, Brunsting J F, Song J, Kampinga H H, Morimoto R I. Bag1 functions in vivo as a negative regulator of Hsp70 chaperone activity. Mol Cell Biol. 2000; 20(3):1083-1088.
Park H J, Qin H, Cha S C, et al. Induction of TLR4-dependent CD8+ T cell immunity by murine betadefensin2 fusion protein vaccines. Vaccine. 2011; 29(18):3476-3482.
Peng P, Menoret A, Srivastava P K. Purification of immunogenic heat shock protein 70-peptide complexes by ADP-affinity chromatography. J Immunol Methods. 1997; 204(1):13-21.
Piva L, Tetlak P, Claser C, Karjalainen K, Renia L, Ruedl C. Cutting edge: Clec9A+ dendritic cells mediate the development of experimental cerebral malaria. J Immunol. 2012; 189(3):1128-1132.
Ralph P, Nakoinz I. Antibody-dependent killing of erythrocyte and tumor targets by macrophage-related cell lines: enhancement by PPD and LPS. J Immunol. 1977; 119(3):950-954.
Rampelt H, Mayer MP, Bukau B. Nucleotide Exchange Factors for Hsp70 Chaperones. Methods Mol Biol. 2018; 1709:179-188.
Raschke W C, Baird S, Ralph P, Nakoinz I. Functional macrophage cell lines transformed by Abelson leukemia virus. Cell. 1978; 15(1):261-267.
Rios-Doria J, Durham N, Wetzel L, et al. Doxil synergizes with cancer immunotherapies to enhance antitumor responses in syngeneic mouse models. Neoplasia. 2015; 17(8):661-670.
Robertson J D, Datta K, Biswal S S, Kehrer J P. Heat-shock protein 70 antisense oligomers enhance proteasome inhibitor-induced apoptosis. Biochem J. 1999; 344 Pt 2(1):477-485.
Rotzschke O, Falk K, Stevanovic S, Jung G, Walden P, Rammensee H G. Exact prediction of a natural T cell epitope. Eur J Immunol. 1991; 21(11):2891-2894.
Santema W, Rutten V, Segers R, et al. Postexposure subunit vaccination against chronic enteric mycobacterial infection in a natural host. Infect Immun. 2013; 81(6):1990-1995.
Sharpless N E, Depinho R A. The mighty mouse: genetically engineered mouse models in cancer drug development. Nat Rev Drug Discov. 2006; 5(9):741-754.
Shen Z, Reznikoff G, Dranoff G, Rock K L. Cloned dendritic cells can present exogenous antigens on both MHC class I and class II molecules. J Immunol. 1997; 158(6):2723-2730.
Shvetsov et al. Philos. Trans. R. Soc. Lond. B. Biol. Sci., 373:1738, 2018.
Shevtsov M, Multhoff G. Heat Shock Protein-Peptide and HSP-Based Immunotherapies for the Treatment of Cancer. Front Immunol. 2016; 7:171.
Shtivelman E, Davies M Q, Hwu P, et al. Pathways and therapeutic targets in melanoma. Oncotarget. 2014; 5(7):1701-1752.
Shulman M, Wilde C D, Kohler G. A better cell line for making hybridomas secreting specific antibodies. Nature. 1978; 276(5685):269-270.
Shultz L D, Schweitzer P A, Christianson S W, et al. Multiple defects in innate and adaptive immunologic function in NOD/LtSz-scid mice. J Immunol. 1995; 154(1):180-191.
Singh M, Khong H, Dai Z, et al. Effective innate and adaptive antimelanoma immunity through localized TLR7/8 activation. J Immunol. 2014; 193(9):4722-4731.
Stangl S, Gehrmann M, Riegger J, et al. Targeting membrane heat-shock protein 70 (Hsp70) on tumors by cmHsp70.1 antibody. Proc Natl Acad Sci USA. 2011; 108(2):733-738.
Swiecki M, Gilfillan S, Vermi W, Wang Y, Colonna M. Plasmacytoid dendritic cell ablation impacts early interferon responses and antiviral NK and CD8(+) T cell accrual. Immunity. 2010; 33(6):955-966.
Takagi H, Fukaya T, Eizumi K, et al. Plasmacytoid dendritic cells are crucial for the initiation of inflammation and T cell immunity in vivo. Immunity. 2011; 35(6):958-971.
Tseng W W, Malu S, Zhang M, et al. Analysis of the intratumoral adaptive immune response in well differentiated and dedifferentiated retroperitoneal liposarcoma. Sarcoma. 2015; 2015: 547460.
Turner M D, Nedjai B, Hurst T, Pennington DJ. Cytokines and chemokines: At the crossroads of cell signalling and inflammatory disease. Biochim Biophys Acta. 2014; 1843(11):2563-2582.
van de Donk N W, Janmaat M L, Mutis T, et al. Monoclonal antibodies targeting CD38 in hematological malignancies and beyond. Immunol Rev. 2016; 270(1):95-112.
van Rooijen N, Hendrikx E. Liposomes for specific depletion of macrophages from organs and tissues. Methods Mol Biol. 2010; 605:189-203.
Vega V L, Rodriguez-Silva M, Frey T, et al. Hsp70 translocates into the plasma membrane after stress and is released into the extracellular environment in a membrane-associated form that activates macrophages. J Immunol. 2008; 180(6):4299-4307.
Veglia F, Gabrilovich D I. Dendritic cells in cancer: the role revisited. Curr Opin Immunol. 2017; 45:43-51.
Wang R, Town T, Gokarn V, Flavell R A, Chandawarkar R Y. HSP70 enhances macrophage phagocytosis by interaction with lipid raft-associated TLR-7 and upregulating p38 MAPK and PI3K pathways. J Surg Res. 2006; 136(1):58-69.
Wang Y, Kelly C G, Singh M, et al. Stimulation of Th1-polarizing cytokines, C-C chemokines, maturation of dendritic cells, and adjuvant function by the peptide binding fragment of heat shock protein 70. J Immunol. 2002; 169(5):2422-2429.
Wesolowski R, Duggan M C, Stiff A, et al. Circulating myeloid-derived suppressor cells increase in patients undergoing neo-adjuvant chemotherapy for breast cancer. Cancer Immunol Immunother. 2017; 66(11):1437-1447.
Willert K, Brown J D, Danenberg E, et al. Wnt proteins are lipid-modified and can act as stem cell growth factors. Nature. 2003; 423(6938):448-452.
Wu et al., Trends Pharmacol. Sci., 38: 226, 2017.
Wu C. Systemic Therapy for Colon Cancer. Surg Oncol Clin N Am. 2018; 27(2):235-242.
Ying H, Kimmelman A C, Lyssiotis C A, et al. Oncogenic Kras maintains pancreatic tumors through regulation of anabolic glucose metabolism. Cell. 2012; 149(3):656-670.
Zaheed M, Wilcken N, Willson M L, O'Connell D L, Goodwin A. Sequencing of anthracyclines and taxanes in neoadjuvant and adjuvant therapy for early breast cancer. Cochrane Database Syst Rev. 2019; 2:CD012873.
Zhang X D, Baladandayuthapani V, Lin H, et al. Tight Junction Protein 1 Modulates Proteasome Capacity and Proteasome Inhibitor Sensitivity in Multiple Myeloma via EGFR/JAK1/STAT3 Signaling. Cancer Cell. 2016; 29(5):639-652.
Zhang X, Lee H C, Shirazi F, et al. Protein targeting chimeric molecules specific for bromodomain and extra-terminal motif family proteins are active against pre-clinical models of multiple myeloma. Leukemia. 2018.
Zhuang J, Shirazi F, Singh R K, et al. Ubiquitin-activating enzyme inhibition induces an unfolded protein response and overcomes drug resistance in myeloma. Blood. 2019; 133(14):1572-1584.
Zininga T, Ramatsui L, Shonhai A. Heat Shock Proteins as Immunomodulants. Molecules. 2018; 23(11): E2846.

What is claimed is:

1. A monoclonal antibody or antibody fragment, wherein the antibody or antibody fragment comprises a heavy chain variable region (VH) comprising a VHCDRI amino acid sequence of GYX₁FTX₂YG (SEQ ID NO: 214), wherein X₁ is T, S, or I, and X₂ is N or K, a VHCDR2 amino acid sequence of INTYTGEX₁ (SEQ ID NO: 215), wherein X₁ is P, S, T, or A, and a VHCDR3 amino acid sequence of X₁RYDHX₂MDY (SEQ ID NO: 216), wherein X₁ is A, T, V, or G, and X₂ is A, R, F, T, P, V, S, D, N, H, L, Y, or G; and a light chain variable region (VL) comprising a VLCDR1 amino acid sequence of QSLX₁NSGTRKNY (SEQ ID NO: 212), wherein X₁ is L, F, or V, a VLCDR2 amino acid sequence of SEQ ID NO: 5, and a VLCDR3 amino acid sequence of KQSYX₁LYT (SEQ ID NO: 213), wherein X₁ is T, N, or S.

2. The monoclonal antibody or antibody fragment of claim 1, wherein said antibody or antibody fragment comprises:
(i) a heavy chain variable region (VH) comprising a VHCDRI amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(ii) a heavy chain variable region (VH) comprising a VHCDRI amino acid sequence of SEQ ID NO: 164, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(iii) a heavy chain variable region (VH) comprising a VHCDRI amino acid sequence of SEQ ID NO: 1, a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(iv) a heavy chain variable region (VH) comprising a VHCDRI amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(v) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(vi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 172; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(vii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(viii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 173; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(ix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 174; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(x) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 175; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;
(xii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 176; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(xiv) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 177; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xv) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 178; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(xviii) a heavy chain variable region (VH) comprising a VHCDR1 amino acid sequence of SEQ ID NO: 164, a VHCDR2 amino acid sequence of SEQ ID NO: 167, and a VHCDR3 amino acid sequence of SEQ ID NO: 174; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 180; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(xxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 182; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxiv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 184; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 185; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxvi) a heavy chain variable region (VH) comprising a VHCDR1 amino acid sequence of SEQ ID NO: 166 a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvii) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxviii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxix) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 174; and a light chain variable region (VL) comprising a VLCDR1 amino acid sequence of SEQ ID NO: 160, a VLCDR2 amino acid sequence of SEQ ID NO: 5, and a VLCDR3 amino acid sequence of SEQ ID NO: 6;
(xxx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 184; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 175; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxii) a heavy chain variable region (VH) comprising a VHCDR1 amino acid sequence of SEQ ID NO: 1, a VHCDR2 amino acid sequence of SEQ ID NO: 167, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxiii) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxiv) a heavy chain variable region (VH) comprising a VHCDR1 amino acid sequence of SEQ ID NO: 166, a VHCDR2 amino acid sequence of SEQ ID NO: 167, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxvi) a heavy chain variable region (VH) comprising a VHCDR1 amino acid sequence of SEQ ID NO: 166, a VHCDR2 amino acid sequence of SEQ ID NO: 168, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xxxviii) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xxxix) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 184; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xl) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 178; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;
(xli) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(xlii) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xliii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xliv) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xlv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 162; or
(xlvi) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

3. The monoclonal antibody or antibody fragment of claim 1 or 2, wherein the antibody or antibody fragment comprises a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

4. The monoclonal antibody or antibody fragment of any one of claims 1-3, wherein said antibody or antibody fragment comprises:
(SEQ ID NO: 18)
X₁X₂QLX₃X₄SGX₅X₆X₇X₈KPGX₉SX₁₀X₁₁X₁₂SCKX₁₃SGYTFTNYG
MNWVRQAPGX₁₄GLX₁₅WX₁₆GWINTYTGEPTYADDFKGRX₁₇TX₁₈X₁₉
X₂₀DX₂₁SX₂₂X₂₃TX₂₄YX₂₅X₂₆X₂₇X₂₈X₂₉LX₃₀X₃₁X₃₂DTAVYF
CARYDHAMDYWGQGTX₃₃VTVSS,
wherein X₁ is Q or E, X₂ is I or V, X₃ is V or Q, X₄ is Q or E, X₅ is A, P, or G, X₆ is E or G, X₇ is V or L, X₈ is V or K, X₉ is A, E, G, or S, X₁₀ is V or L, X₁₁ is K or R, X₁₂ is V, L, or I, X₁₃ is A or T, X₁₄ is K or Q, X₁₅ is E or K, X₁₆ is M or V, X₁₇ is F or V, X₁₈ is F, M, or I, X₁₉ is T or S, X₂₀ is T, R, or A, X₂₁ is T, D, or E, X₂₂ is T, A, or K, X₂₃ is S or N, X₂₄ is L or A, X₂₅ is M or L, X₂₆ is E or Q, X₂₇ is L or M, X₂₈ is R, S, T, or N, X₂₉ is S or G, X₃₀ is R, K, or M, X₃₁ is S or T, X₃₂ is D or E, and X₃₃ is L, S, or T;
and a light chain variable sequence having a sequence of
(SEQ ID NO: 26)
X₁X₂X₃X₄TQSPX₅SLX₆X₇SX₈GX₉RX₁₀TIX₁₁CKSSQSLLNSGTRKN
YLAWYQQKX₁₂GX₁₃X₁₄PX₁₅LLIYWTSTRESGVPX₁₆RFSGSGSGTDF
TLTIX₁₇X₁₈LQX₁₉EDVAX₂₀YYCKQSYTLYTFGX₂₁GTKX₂₂EIK,
wherein X₁ is E or D, X₂ is I or V, X₃ is V or Q, X₄ is L or M, X₅ is D or S, X₆ is A or S, X₇ is V or A, X₈ is L or V, X₉ is E or D, X₁₀ is A or V, X₁₁ is N or T, X₁₂ is A or P, X₁₃ is Q or K, X₁₄ is S, V, or P, X₁₅ is K or R, X₁₆ is D or S, X₁₇ is S, D, or N, X₁₈ is S or T, X₁₉ is A or P, X₂₀ is V or T, X₂₁ is Q or G, and X₂₂ is L or V.

5. The monoclonal antibody or antibody fragment of claim 4, wherein said antibody or antibody fragment comprises:
(i) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(ii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(iii) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(iv) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(v) a heavy chain variable sequence having a sequence according to SEQ ID NO: 12, or a heavy chain variable sequence having at least 70%, 80%, 90%, 95%, 96%, 97%, 98%, or 99% identity to SEQ ID NO: 12; and a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(vi) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(vii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(viii) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(ix) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(x) a heavy chain variable sequence having a sequence according to SEQ ID NO: 13, or a heavy chain variable sequence having at least 70%, 80%, 90%, 95%, 96%, 97%, 98%, or 99% identity to SEQ ID NO: 13; and a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xi) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(xii) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(xiii) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xiv) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(xv) a heavy chain variable sequence having a sequence according to SEQ ID NO: 14, or a heavy chain variable sequence having at least 70%, 80%, 90%, 95%, 96%, 97%, 98%, or 99% identity to SEQ ID NO: 14; and a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xvi) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(xvii) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(xviii) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xix) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and a light chain variable sequence having a sequence according to SEQ ID NO: 22, or a light chain variable sequence having at least 70%, 80%, 90%, 95%, 96%, 97%, 98%, or 99% identity to SEQ ID NO: 22;
(xx) a heavy chain variable sequence having a sequence according to SEQ ID NO: 15, or a heavy chain variable sequence having at least 70%, 80%, 90%, 95%, 96%, 97%, 98%, or 99% identity to SEQ ID NO: 15; and a light chain variable sequence having a sequence according to SEQ ID NO: 23, or a light chain variable sequence having at least 70%, 80%, 90%, 95%, 96%, 97%, 98%, or 99% identity to SEQ ID NO: 23;
(xxi) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and a light chain variable sequence having a sequence according to SEQ ID NO: 19, or a light chain variable sequence having at least 70%, 80%, 90%, 95%, 96%, 97%, 98%, or 99% identity to SEQ ID NO: 19;
(xxii) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and a light chain variable sequence having a sequence according to SEQ ID NO: 20, or a light chain variable sequence having at least 70%, 80%, 90%, 95%, 96%, 97%, 98%, or 99% identity to SEQ ID NO: 20;
(xxiii) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and a light chain variable sequence having a sequence according to SEQ ID NO: 21, or a light chain variable sequence having at least 70%, 80%, 90%, 95%, 96%, 97%, 98%, or 99% identity to SEQ ID NO: 21;
(xxiv) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and a light chain variable sequence having a sequence according to SEQ ID NO: 22, or a light chain variable sequence having at least 70%, 80%, 90%, 95%, 96%, 97%, 98%, or 99% identity to SEQ ID NO: 22; or
(xxv) a heavy chain variable sequence having a sequence according to SEQ ID NO: 16, or a heavy chain variable sequence having at least 70%, 80%, 90%, 95%, 96%, 97%, 98%, or 99% identity to SEQ ID NO: 16; and a light chain variable sequence having a sequence according to SEQ ID NO: 23, or a light chain variable sequence having at least 70%, 80%, 90%, 95%, 96%, 97%, 98%, or 99% identity to SEQ ID NO: 23.

6. The monoclonal antibody or antibody fragment of any one of claims 1-3, wherein said antibody or antibody fragment comprises a heavy chain variable sequence having a sequence of
(SEQ ID NO: 104)
QIX₁LVQSGX₂EVKKPGASVKVSCKASGYX₃FTX₄YGMNWVRQAPGQGLE
WMGWINTYTGEX₅X₆YX₇DDFKGRFTFTTDTSTX₈TX₉YMX₁₀X₁₁RSLR
SDDTAVYFCX₁₂RYDHX₁₃MDYWGQGX₁₄LVTVSS
wherein X₁ is Q or H, X₂ is A, D, T, V, S, or P, X₃ is T, S, or I, X₄ is N or K, X₅ is P, S, T, or A, X₆ is T, R, K, or I, X₇ is A, T, V, S, or G, X₈ is S, R, or T, X₉ is A, V, or G, X₁₀ is E or D, X₁₁ is L or V, X₁₂ is A, T, V, or G, X₁₃ is A, R, F, T, P, V, S, D, N, H, L, Y, or G, and X₁₄ is T or S;
and a light chain variable sequence having a sequence of
(SEQ ID NO: 158)
EIVLTQSPDSLX₁VSLGERATIX₂CKSSQSLX₃NSGTRKNYLX₄WYQX₅K
X₆GQSPX₇LX₈IYWTSTRESGVPDRFSX₉SGSGTDFTLX₁₀IDX₁₁LQ
X₁₂EDVAX₁₃YYCKQSYX₁₄LYTFGGGTKVEIK,
wherein X₁ is A, T, or S, X₂ is N or K, X₃ is L, F, or V, X₄ is A, S, or T, X₅ is Q or K, X₆ is A, P, or S, X₇ is K or N, X₈ is L, V, or I, X₉ is G or A, X₁₀ is T or S, X₁₁ is S or R, X₁₂ is A or T, X₁₃ is V, I, or L, and X₁₄ is T, N, or S.

7. The monoclonal antibody or antibody fragment of claim 6, wherein said antibody or antibody fragment comprises:
(i) a heavy chain variable sequence having a sequence according to SEQ ID NO: 26, or a heavy chain variable sequence having at least 70%, 80%, 90%, 95%, 96%, 97%, 98%, or 99% identity to SEQ ID NO: 26; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(ii) a heavy chain variable sequence having a sequence according to SEQ ID NO: 27, or a heavy chain variable sequence having at least 70%, 80%, 90%, 95%, 96%, 97%, 98%, or 99% identity to SEQ ID NO: 27; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(iii) a heavy chain variable sequence having a sequence according to SEQ ID NO: 28, or a heavy chain variable sequence having at least 70%, 80%, 90%, 95%, 96%, 97%, 98%, or 99% identity to SEQ ID NO: 28; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(iv) a heavy chain variable sequence having a sequence according to SEQ ID NO: 29, or a heavy chain variable sequence having at least 70%, 80%, 90%, 95%, 96%, 97%, 98%, or 99% identity to SEQ ID NO: 29; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(v) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and a light chain variable sequence having a sequence according to SEQ ID NO: 106, or a light chain variable sequence having at least 70%, 80%, 90%, 95%, 96%, 97%, 98%, or 99% identity to SEQ ID NO: 106;
(vi) a heavy chain variable sequence having a sequence according to SEQ ID NO: 31, or a heavy chain variable sequence having at least 70%, 80%, 90%, 95%, 96%, 97%, 98%, or 99% identity to SEQ ID NO: 31; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(vii) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(viii) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and a light chain variable sequence having a sequence according to SEQ ID NO: 107, or a light chain variable sequence having at least 70%, 80%, 90%, 95%, 96%, 97%, 98%, or 99% identity to SEQ ID NO: 107;
(ix) a heavy chain variable sequence having a sequence according to SEQ ID NO: 33, or a heavy chain variable sequence having at least 70%, 80%, 90%, 95%, 96%, 97%, 98%, or 99% identity to SEQ ID NO: 33; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(x) a heavy chain variable sequence having a sequence according to SEQ ID NO: 34, or a heavy chain variable sequence having at least 70%, 80%, 90%, 95%, 96%, 97%, 98%, or 99% identity to SEQ ID NO: 34; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xi) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and a light chain variable sequence having a sequence according to SEQ ID NO: 108, or a light chain variable sequence having at least 70%, 80%, 90%, 95%, 96%, 97%, 98%, or 99% identity to SEQ ID NO: 108;
(xii) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(xiii) a heavy chain variable sequence having a sequence according to SEQ ID NO: 35, or a heavy chain variable sequence having at least 70%, 80%, 90%, 95%, 96%, 97%, 98%, or 99% identity to SEQ ID NO: 35; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xiv) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xv) a heavy chain variable sequence having a sequence according to SEQ ID NO: 37, or a heavy chain variable sequence having at least 70%, 80%, 90%, 95%, 96%, 97%, 98%, or 99% identity to SEQ ID NO: 37; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xvi) a heavy chain variable sequence having a sequence according to SEQ ID NO: 26, or a heavy chain variable sequence having at least 70%, 80%, 90%, 95%, 96%, 97%, 98%, or 99% identity to SEQ ID NO: 26; and a light chain variable sequence having a sequence according to SEQ ID NO: 107, or a light chain variable sequence having at least 70%, 80%, 90%, 95%, 96%, 97%, 98%, or 99% identity to SEQ ID NO: 107;
(xvii) a heavy chain variable sequence having a sequence according to SEQ ID NO: 38, or a heavy chain variable sequence having at least 70%, 80%, 90%, 95%, 96%, 97%, 98%, or 99% identity to SEQ ID NO: 38; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xviii) a heavy chain variable sequence having a sequence according to SEQ ID NO: 31, or a heavy chain variable sequence having at least 70%, 80%, 90%, 95%, 96%, 97%, 98%, or 99% identity to SEQ ID NO: 31; and a light chain variable sequence having a sequence according to SEQ ID NO: 110, or a light chain variable sequence having at least 70%, 80%, 90%, 95%, 96%, 97%, 98%, or 99% identity to SEQ ID NO: 110;
(xix) a heavy chain variable sequence having a sequence according to SEQ ID NO: 39, or a heavy chain variable sequence having at least 70%, 80%, 90%, 95%, 96%, 97%, 98%, or 99% identity to SEQ ID NO: 39; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xx) a heavy chain variable sequence having a sequence according to SEQ ID NO: 40, or a heavy chain variable sequence having at least 70%, 80%, 90%, 95%, 96%, 97%, 98%, or 99% identity to SEQ ID NO: 40; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxi) a heavy chain variable sequence having a sequence according to SEQ ID NO: 34, or a heavy chain variable sequence having at least 70%, 80%, 90%, 95%, 96%, 97%, 98%, or 99% identity to SEQ ID NO: 34; and a light chain variable sequence having a sequence according to SEQ ID NO: 111, or a light chain variable sequence having at least 70%, 80%, 90%, 95%, 96%, 97%, 98%, or 99% identity to SEQ ID NO: 111;
(xxii) a heavy chain variable sequence having a sequence according to SEQ ID NO: 41, or a heavy chain variable sequence having at least 70%, 80%, 90%, 95%, 96%, 97%, 98%, or 99% identity to SEQ ID NO: 41; and a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(xxiii) a heavy chain variable sequence having a sequence according to SEQ ID NO: 30, or a heavy chain variable sequence having at least 70%, 80%, 90%, 95%, 96%, 97%, 98%, or 99% identity to SEQ ID NO: 30; and a light chain variable sequence having a sequence according to SEQ ID NO: 112, or a light chain variable sequence having at least 70%, 80%, 90%, 95%, 96%, 97%, 98%, or 99% identity to SEQ ID NO: 112;
(xxiv) a heavy chain variable sequence having a sequence according to SEQ ID NO: 28, or a heavy chain variable sequence having at least 70%, 80%, 90%, 95%, 96%, 97%, 98%, or 99% identity to SEQ ID NO: 28; and a light chain variable sequence having a sequence according to SEQ ID NO: 113, or a light chain variable sequence having at least 70%, 80%, 90%, 95%, 96%, 97%, 98%, or 99% identity to SEQ ID NO: 113;
(xxv) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and a light chain variable sequence having a sequence according to SEQ ID NO: 114, or a light chain variable sequence having at least 70%, 80%, 90%, 95%, 96%, 97%, 98%, or 99% identity to SEQ ID NO: 114;
(xxvi) a heavy chain variable sequence having a sequence according to SEQ ID NO: 42, or a heavy chain variable sequence having at least 70%, 80%, 90%, 95%, 96%, 97%, 98%, or 99% identity to SEQ ID NO: 42; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxvii) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and a light chain variable sequence having a sequence according to SEQ ID NO: 115, or a light chain variable sequence having at least 70%, 80%, 90%, 95%, 96%, 97%, 98%, or 99% identity to SEQ ID NO: 115;
(xxviii) a heavy chain variable sequence having a sequence according to SEQ ID NO: 43, or a heavy chain variable sequence having at least 70%, 80%, 90%, 95%, 96%, 97%, 98%, or 99% identity to SEQ ID NO: 43; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxix) a heavy chain variable sequence having a sequence according to SEQ ID NO: 32, or a heavy chain variable sequence having at least 70%, 80%, 90%, 95%, 96%, 97%, 98%, or 99% identity to SEQ ID NO: 32; and a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(xxx) a heavy chain variable sequence having a sequence according to SEQ ID NO: 44, or a heavy chain variable sequence having at least 70%, 80%, 90%, 95%, 96%, 97%, 98%, or 99% identity to SEQ ID NO: 44; and a light chain variable sequence having a sequence according to SEQ ID NO: 116, or a light chain variable sequence having at least 70%, 80%, 90%, 95%, 96%, 97%, 98%, or 99% identity to SEQ ID NO: 116;
(xxi) a heavy chain variable sequence having a sequence according to SEQ ID NO: 35, or a heavy chain variable sequence having at least 70%, 80%, 90%, 95%, 96%, 97%, 98%, or 99% identity to SEQ ID NO: 35; and a light chain variable sequence having a sequence according to SEQ ID NO: 117, or a light chain variable sequence having at least 70%, 80%, 90%, 95%, 96%, 97%, 98%, or 99% identity to SEQ ID NO: 117;
(xxxii) a heavy chain variable sequence having a sequence according to SEQ ID NO: 45, or a heavy chain variable sequence having at least 70%, 80%, 90%, 95%, 96%, 97%, 98%, or 99% identity to SEQ ID NO: 45; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxxiii) a heavy chain variable sequence having a sequence according to SEQ ID NO: 46, or a heavy chain variable sequence having at least 70%, 80%, 90%, 95%, 96%, 97%, 98%, or 99% identity to SEQ ID NO: 46; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxxiv) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and a light chain variable sequence having a sequence according to SEQ ID NO: 118, or a light chain variable sequence having at least 70%, 80%, 90%, 95%, 96%, 97%, 98%, or 99% identity to SEQ ID NO: 118;
(xxxv) a heavy chain variable sequence having a sequence according to SEQ ID NO: 47, or a heavy chain variable sequence having at least 70%, 80%, 90%, 95%, 96%, 97%, 98%, or 99% identity to SEQ ID NO: 47; and a light chain variable sequence having a sequence according to SEQ ID NO: 115, or a light chain variable sequence having at least 70%, 80%, 90%, 95%, 96%, 97%, 98%, or 99% identity to SEQ ID NO: 115;
(xxxvi) a heavy chain variable sequence having a sequence according to SEQ ID NO: 48, or a heavy chain variable sequence having at least 70%, 80%, 90%, 95%, 96%, 97%, 98%, or 99% identity to SEQ ID NO: 48; and a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(xxxvii) a heavy chain variable sequence having a sequence according to SEQ ID NO: 49, or a heavy chain variable sequence having at least 70%, 80%, 90%, 95%, 96%, 97%, 98%, or 99% identity to SEQ ID NO: 49; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxxviii) a heavy chain variable sequence having a sequence according to SEQ ID NO: 50, or a heavy chain variable sequence having at least 70%, 80%, 90%, 95%, 96%, 97%, 98%, or 99% identity to SEQ ID NO: 50; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xxxix) a heavy chain variable sequence having a sequence according to SEQ ID NO: 51, or a heavy chain variable sequence having at least 70%, 80%, 90%, 95%, 96%, 97%, 98%, or 99% identity to SEQ ID NO: 51; and a light chain variable sequence having a sequence according to SEQ ID NO: 106, or a light chain variable sequence having at least 70%, 80%, 90%, 95%, 96%, 97%, 98%, or 99% identity to SEQ ID NO: 106;
(xl) a heavy chain variable sequence having a sequence according to SEQ ID NO: 52, or a heavy chain variable sequence having at least 70%, 80%, 90%, 95%, 96%, 97%, 98%, or 99% identity to SEQ ID NO: 52; and a light chain variable sequence having a sequence according to SEQ ID NO: 119, or a light chain variable sequence having at least 70%, 80%, 90%, 95%, 96%, 97%, 98%, or 99% identity to SEQ ID NO: 119;
(xli) a heavy chain variable sequence having a sequence according to SEQ ID NO: 53, or a heavy chain variable sequence having at least 70%, 80%, 90%, 95%, 96%, 97%, 98%, or 99% identity to SEQ ID NO: 53; and a light chain variable sequence having a sequence according to SEQ ID NO: 108, or a light chain variable sequence having at least 70%, 80%, 90%, 95%, 96%, 97%, 98%, or 99% identity to SEQ ID NO: 108;
(xlii) a heavy chain variable sequence having a sequence according to SEQ ID NO: 54, or a heavy chain variable sequence having at least 70%, 80%, 90%, 95%, 96%, 97%, 98%, or 99% identity to SEQ ID NO: 54; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(xliii) a heavy chain variable sequence having a sequence according to SEQ ID NO: 55, or a heavy chain variable sequence having at least 70%, 80%, 90%, 95%, 96%, 97%, 98%, or 99% identity to SEQ ID NO: 55; and a light chain variable sequence having a sequence according to SEQ ID NO: 116, or a light chain variable sequence having at least 70%, 80%, 90%, 95%, 96%, 97%, 98%, or 99% identity to SEQ ID NO: 116;
(xliv) a heavy chain variable sequence having a sequence according to SEQ ID NO: 56, or a heavy chain variable sequence having at least 70%, 80%, 90%, 95%, 96%, 97%, 98%, or 99% identity to SEQ ID NO: 56; and a light chain variable sequence having a sequence according to SEQ ID NO: 116, or a light chain variable sequence having at least 70%, 80%, 90%, 95%, 96%, 97%, 98%, or 99% identity to SEQ ID NO: 116;
(xlv) a heavy chain variable sequence having a sequence according to SEQ ID NO: 57, or a heavy chain variable sequence having at least 70%, 80%, 90%, 95%, 96%, 97%, 98%, or 99% identity to SEQ ID NO: 57; and a light chain variable sequence having a sequence according to SEQ ID NO: 120, or a light chain variable sequence having at least 70%, 80%, 90%, 95%, 96%, 97%, 98%, or 99% identity to SEQ ID NO: 120;
(xlvi) a heavy chain variable sequence having a sequence according to SEQ ID NO: 58, or a heavy chain variable sequence having at least 70%, 80%, 90%, 95%, 96%, 97%, 98%, or 99% identity to SEQ ID NO: 58; and a light chain variable sequence having a sequence according to SEQ ID NO: 121, or a light chain variable sequence having at least 70%, 80%, 90%, 95%, 96%, 97%, 98%, or 99% identity to SEQ ID NO: 121;
(xlvii) a heavy chain variable sequence having a sequence according to SEQ ID NO: 59, or a heavy chain variable sequence having at least 70%, 80%, 90%, 95%, 96%, 97%, 98%, or 99% identity to SEQ ID NO: 59; and a light chain variable sequence having a sequence according to SEQ ID NO: 122, or a light chain variable sequence having at least 70%, 80%, 90%, 95%, 96%, 97%, 98%, or 99% identity to SEQ ID NO: 122;
(xlviii) a heavy chain variable sequence having a sequence according to SEQ ID NO: 60, or a heavy chain variable sequence having at least 70%, 80%, 90%, 95%, 96%, 97%, 98%, or 99% identity to SEQ ID NO: 60; and a light chain variable sequence having a sequence according to SEQ ID NO: 108, or a light chain variable sequence having at least 70%, 80%, 90%, 95%, 96%, 97%, 98%, or 99% identity to SEQ ID NO: 108;
(xlix) a heavy chain variable sequence having a sequence according to SEQ ID NO: 61, or a heavy chain variable sequence having at least 70%, 80%, 90%, 95%, 96%, 97%, 98%, or 99% identity to SEQ ID NO: 61; and a light chain variable sequence having a sequence according to SEQ ID NO: 123, or a light chain variable sequence having at least 70%, 80%, 90%, 95%, 96%, 97%, 98%, or 99% identity to SEQ ID NO: 123;
(l) a heavy chain variable sequence having a sequence according to SEQ ID NO: 62, or a heavy chain variable sequence having at least 70%, 80%, 90%, 95%, 96%, 97%, 98%, or 99% identity to SEQ ID NO: 62; and a light chain variable sequence having a sequence according to SEQ ID NO: 114, or a light chain variable sequence having at least 70%, 80%, 90%, 95%, 96%, 97%, 98%, or 99% identity to SEQ ID NO: 114;
(li) a heavy chain variable sequence having a sequence according to SEQ ID NO: 63, or a heavy chain variable sequence having at least 70%, 80%, 90%, 95%, 96%, 97%, 98%, or 99% identity to SEQ ID NO: 63; and a light chain variable sequence having a sequence according to SEQ ID NO: 124, or a light chain variable sequence having at least 70%, 80%, 90%, 95%, 96%, 97%, 98%, or 99% identity to SEQ ID NO: 124;
(lii) a heavy chain variable sequence having a sequence according to SEQ ID NO: 64, or a heavy chain variable sequence having at least 70%, 80%, 90%, 95%, 96%, 97%, 98%, or 99% identity to SEQ ID NO: 64; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(liii) a heavy chain variable sequence having a sequence according to SEQ ID NO: 65, or a heavy chain variable sequence having at least 70%, 80%, 90%, 95%, 96%, 97%, 98%, or 99% identity to SEQ ID NO: 65; and a light chain variable sequence having a sequence according to SEQ ID NO: 125, or a light chain variable sequence having at least 70%, 80%, 90%, 95%, 96%, 97%, 98%, or 99% identity to SEQ ID NO: 125;
(liv) a heavy chain variable sequence having a sequence according to SEQ ID NO: 66, or a heavy chain variable sequence having at least 70%, 80%, 90%, 95%, 96%, 97%, 98%, or 99% identity to SEQ ID NO: 66; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lv) a heavy chain variable sequence having a sequence according to SEQ ID NO: 67, or a heavy chain variable sequence having at least 70%, 80%, 90%, 95%, 96%, 97%, 98%, or 99% identity to SEQ ID NO: 67; and a light chain variable sequence having a sequence according to SEQ ID NO: 125, or a light chain variable sequence having at least 70%, 80%, 90%, 95%, 96%, 97%, 98%, or 99% identity to SEQ ID NO: 125;
(lvi) a heavy chain variable sequence having a sequence according to SEQ ID NO: 68, or a heavy chain variable sequence having at least 70%, 80%, 90%, 95%, 96%, 97%, 98%, or 99% identity to SEQ ID NO: 68; and a light chain variable sequence having a sequence according to SEQ ID NO: 126, or a light chain variable sequence having at least 70%, 80%, 90%, 95%, 96%, 97%, 98%, or 99% identity to SEQ ID NO: 126;
(lvii) a heavy chain variable sequence having a sequence according to SEQ ID NO: 69, or a heavy chain variable sequence having at least 70%, 80%, 90%, 95%, 96%, 97%, 98%, or 99% identity to SEQ ID NO: 69; and a light chain variable sequence having a sequence according to SEQ ID NO: 127, or a light chain variable sequence having at least 70%, 80%, 90%, 95%, 96%, 97%, 98%, or 99% identity to SEQ ID NO: 127;
(lviii) a heavy chain variable sequence having a sequence according to SEQ ID NO: 70, or a heavy chain variable sequence having at least 70%, 80%, 90%, 95%, 96%, 97%, 98%, or 99% identity to SEQ ID NO: 70; and a light chain variable sequence having a sequence according to SEQ ID NO: 128, or a light chain variable sequence having at least 70%, 80%, 90%, 95%, 96%, 97%, 98%, or 99% identity to SEQ ID NO: 128;
(lix) a heavy chain variable sequence having a sequence according to SEQ ID NO: 71, or a heavy chain variable sequence having at least 70%, 80%, 90%, 95%, 96%, 97%, 98%, or 99% identity to SEQ ID NO: 71; and a light chain variable sequence having a sequence according to SEQ ID NO: 117, or a light chain variable sequence having at least 70%, 80%, 90%, 95%, 96%, 97%, 98%, or 99% identity to SEQ ID NO: 117;
(lx) a heavy chain variable sequence having a sequence according to SEQ ID NO: 72, or a heavy chain variable sequence having at least 70%, 80%, 90%, 95%, 96%, 97%, 98%, or 99% identity to SEQ ID NO: 72; and a light chain variable sequence having a sequence according to SEQ ID NO: 129, or a light chain variable sequence having at least 70%, 80%, 90%, 95%, 96%, 97%, 98%, or 99% identity to SEQ ID NO: 129;
(lxi) a heavy chain variable sequence having a sequence according to SEQ ID NO: 73, or a heavy chain variable sequence having at least 70%, 80%, 90%, 95%, 96%, 97%, 98%, or 99% identity to SEQ ID NO: 73; and a light chain variable sequence having a sequence according to SEQ ID NO: 130, or a light chain variable sequence having at least 70%, 80%, 90%, 95%, 96%, 97%, 98%, or 99% identity to SEQ ID NO: 130;
(lxii) a heavy chain variable sequence having a sequence according to SEQ ID NO: 74, or a heavy chain variable sequence having at least 70%, 80%, 90%, 95%, 96%, 97%, 98%, or 99% identity to SEQ ID NO: 74; and a light chain variable sequence having a sequence according to SEQ ID NO: 131, or a light chain variable sequence having at least 70%, 80%, 90%, 95%, 96%, 97%, 98%, or 99% identity to SEQ ID NO: 131;
(lxiii) a heavy chain variable sequence having a sequence according to SEQ ID NO: 73, or a heavy chain variable sequence having at least 70%, 80%, 90%, 95%, 96%, 97%, 98%, or 99% identity to SEQ ID NO: 73; and a light chain variable sequence having a sequence according to SEQ ID NO: 132, or a light chain variable sequence having at least 70%, 80%, 90%, 95%, 96%, 97%, 98%, or 99% identity to SEQ ID NO: 132;
(lxiv) a heavy chain variable sequence having a sequence according to SEQ ID NO: 75, or a heavy chain variable sequence having at least 70%, 80%, 90%, 95%, 96%, 97%, 98%, or 99% identity to SEQ ID NO: 75; and a light chain variable sequence having a sequence according to SEQ ID NO: 133, or a light chain variable sequence having at least 70%, 80%, 90%, 95%, 96%, 97%, 98%, or 99% identity to SEQ ID NO: 133;
(lxv) a heavy chain variable sequence having a sequence according to SEQ ID NO: 76, or a heavy chain variable sequence having at least 70%, 80%, 90%, 95%, 96%, 97%, 98%, or 99% identity to SEQ ID NO: 76; and a light chain variable sequence having a sequence according to SEQ ID NO: 134, or a light chain variable sequence having at least 70%, 80%, 90%, 95%, 96%, 97%, 98%, or 99% identity to SEQ ID NO: 134;
(lxvi) a heavy chain variable sequence having a sequence according to SEQ ID NO: 77, or a heavy chain variable sequence having at least 70%, 80%, 90%, 95%, 96%, 97%, 98%, or 99% identity to SEQ ID NO: 77; and a light chain variable sequence having a sequence according to SEQ ID NO: 107, or a light chain variable sequence having at least 70%, 80%, 90%, 95%, 96%, 97%, 98%, or 99% identity to SEQ ID NO: 107;
(lxvii) a heavy chain variable sequence having a sequence according to SEQ ID NO: 78, or a heavy chain variable sequence having at least 70%, 80%, 90%, 95%, 96%, 97%, 98%, or 99% identity to SEQ ID NO: 78; and a light chain variable sequence having a sequence according to SEQ ID NO: 135, or a light chain variable sequence having at least 70%, 80%, 90%, 95%, 96%, 97%, 98%, or 99% identity to SEQ ID NO: 135;
(lxviii) a heavy chain variable sequence having a sequence according to SEQ ID NO: 79, or a heavy chain variable sequence having at least 70%, 80%, 90%, 95%, 96%, 97%, 98%, or 99% identity to SEQ ID NO: 79; and a light chain variable sequence having a sequence according to SEQ ID NO: 136, or a light chain variable sequence having at least 70%, 80%, 90%, 95%, 96%, 97%, 98%, or 99% identity to SEQ ID NO: 136;
(lxix) a heavy chain variable sequence having a sequence according to SEQ ID NO: 80, or a heavy chain variable sequence having at least 70%, 80%, 90%, 95%, 96%, 97%, 98%, or 99% identity to SEQ ID NO: 80; and a light chain variable sequence having a sequence according to SEQ ID NO: 137, or a light chain variable sequence having at least 70%, 80%, 90%, 95%, 96%, 97%, 98%, or 99% identity to SEQ ID NO: 137;
(lxx) a heavy chain variable sequence having a sequence according to SEQ ID NO: 41, or a heavy chain variable sequence having at least 70%, 80%, 90%, 95%, 96%, 97%, 98%, or 99% identity to SEQ ID NO: 41; and a light chain variable sequence having a sequence according to SEQ ID NO: 138, or a light chain variable sequence having at least 70%, 80%, 90%, 95%, 96%, 97%, 98%, or 99% identity to SEQ ID NO: 138;
(lxxi) a heavy chain variable sequence having a sequence according to SEQ ID NO: 81, or a heavy chain variable sequence having at least 70%, 80%, 90%, 95%, 96%, 97%, 98%, or 99% identity to SEQ ID NO: 31; and a light chain variable sequence having a sequence according to SEQ ID NO: 139, or a light chain variable sequence having at least 70%, 80%, 90%, 95%, 96%, 97%, 98%, or 99% identity to SEQ ID NO: 139;
(lxxii) a heavy chain variable sequence having a sequence according to SEQ ID NO: 82, or a heavy chain variable sequence having at least 70%, 80%, 90%, 95%, 96%, 97%, 98%, or 99% identity to SEQ ID NO: 82; and a light chain variable sequence having a sequence according to SEQ ID NO: 105, or a light chain variable sequence having at least 70%, 80%, 90%, 95%, 96%, 97%, 98%, or 99% identity to SEQ ID NO: 105;
(lxxiii) a heavy chain variable sequence having a sequence according to SEQ ID NO: 83, or a heavy chain variable sequence having at least 70%, 80%, 90%, 95%, 96%, 97%, 98%, or 99% identity to SEQ ID NO: 83; and a light chain variable sequence having a sequence according to SEQ ID NO: 126, or a light chain variable sequence having at least 70%, 80%, 90%, 95%, 96%, 97%, 98%, or 99% identity to SEQ ID NO: 126;
(lxxiv) a heavy chain variable sequence having a sequence according to SEQ ID NO: 84, or a heavy chain variable sequence having at least 70%, 80%, 90%, 95%, 96%, 97%, 98%, or 99% identity to SEQ ID NO: 84; and a light chain variable sequence having a sequence according to SEQ ID NO: 140, or a light chain variable sequence having at least 70%, 80%, 90%, 95%, 96%, 97%, 98%, or 99% identity to SEQ ID NO: 140;
(lxxv) a heavy chain variable sequence having a sequence according to SEQ ID NO: 85, or a heavy chain variable sequence having at least 70%, 80%, 90%, 95%, 96%, 97%, 98%, or 99% identity to SEQ ID NO: 85; and a light chain variable sequence having a sequence according to SEQ ID NO: 141, or a light chain variable sequence having at least 70%, 80%, 90%, 95%, 96%, 97%, 98%, or 99% identity to SEQ ID NO: 141;
(lxxvi) a heavy chain variable sequence having a sequence according to SEQ ID NO: 86, or a heavy chain variable sequence having at least 70%, 80%, 90%, 95%, 96%, 97%, 98%, or 99% identity to SEQ ID NO: 86; and a light chain variable sequence having a sequence according to SEQ ID NO: 141, or a light chain variable sequence having at least 70%, 80%, 90%, 95%, 96%, 97%, 98%, or 99% identity to SEQ ID NO: 141;
(lxxvii) a heavy chain variable sequence having a sequence according to SEQ ID NO: 87, or a heavy chain variable sequence having at least 70%, 80%, 90%, 95%, 96%, 97%, 98%, or 99% identity to SEQ ID NO: 87; and a light chain variable sequence having a sequence according to SEQ ID NO: 117, or a light chain variable sequence having at least 70%, 80%, 90%, 95%, 96%, 97%, 98%, or 99% identity to SEQ ID NO: 117;
(lxxviii) a heavy chain variable sequence having a sequence according to SEQ ID NO: 88, or a heavy chain variable sequence having at least 70%, 80%, 90%, 95%, 96%, 97%, 98%, or 99% identity to SEQ ID NO: 88; and a light chain variable sequence having a sequence according to SEQ ID NO: 142, or a light chain variable sequence having at least 70%, 80%, 90%, 95%, 96%, 97%, 98%, or 99% identity to SEQ ID NO: 142;
(lxxix) a heavy chain variable sequence having a sequence according to SEQ ID NO: 89, or a heavy chain variable sequence having at least 70%, 80%, 90%, 95%, 96%, 97%, 98%, or 99% identity to SEQ ID NO: 89; and a light chain variable sequence having a sequence according to SEQ ID NO: 143, or a light chain variable sequence having at least 70%, 80%, 90%, 95%, 96%, 97%, 98%, or 99% identity to SEQ ID NO: 143;
(lxxx) a heavy chain variable sequence having a sequence according to SEQ ID NO: 90, or a heavy chain variable sequence having at least 70%, 80%, 90%, 95%, 96%, 97%, 98%, or 99% identity to SEQ ID NO: 90; and a light chain variable sequence having a sequence according to SEQ ID NO: 144, or a light chain variable sequence having at least 70%, 80%, 90%, 95%, 96%, 97%, 98%, or 99% identity to SEQ ID NO: 144;
(lxxxi) a heavy chain variable sequence having a sequence according to SEQ ID NO: 91, or a heavy chain variable sequence having at least 70%, 80%, 90%, 95%, 96%, 97%, 98%, or 99% identity to SEQ ID NO: 91; and a light chain variable sequence having a sequence according to SEQ ID NO: 109, or a light chain variable sequence having at least 70%, 80%, 90%, 95%, 96%, 97%, 98%, or 99% identity to SEQ ID NO: 109;
(lxxxii) a heavy chain variable sequence having a sequence according to SEQ ID NO: 92, or a heavy chain variable sequence having at least 70%, 80%, 90%, 95%, 96%, 97%, 98%, or 99% identity to SEQ ID NO: 92; and a light chain variable sequence having a sequence according to SEQ ID NO: 145, or a light chain variable sequence having at least 70%, 80%, 90%, 95%, 96%, 97%, 98%, or 99% identity to SEQ ID NO: 145;
(lxxxiii) a heavy chain variable sequence having a sequence according to SEQ ID NO: 93, or a heavy chain variable sequence having at least 70%, 80%, 90%, 95%, 96%, 97%, 98%, or 99% identity to SEQ ID NO: 93; and a light chain variable sequence having a sequence according to SEQ ID NO: 146, or a light chain variable sequence having at least 70%, 80%, 90%, 95%, 96%, 97%, 98%, or 99% identity to SEQ ID NO: 146;
(lxxxiv) a heavy chain variable sequence having a sequence according to SEQ ID NO: 94, or a heavy chain variable sequence having at least 70%, 80%, 90%, 95%, 96%, 97%, 98%, or 99% identity to SEQ ID NO: 94; and a light chain variable sequence having a sequence according to SEQ ID NO: 147, or a light chain variable sequence having at least 70%, 80%, 90%, 95%, 96%, 97%, 98%, or 99% identity to SEQ ID NO: 147;
(lxxxv) a heavy chain variable sequence having a sequence according to SEQ ID NO: 95, or a heavy chain variable sequence having at least 70%, 80%, 90%, 95%, 96%, 97%, 98%, or 99% identity to SEQ ID NO: 95; and a light chain variable sequence having a sequence according to SEQ ID NO: 148, or a light chain variable sequence having at least 70%, 80%, 90%, 95%, 96%, 97%, 98%, or 99% identity to SEQ ID NO: 148;
(lxxxvi) a heavy chain variable sequence having a sequence according to SEQ ID NO: 96, or a heavy chain variable sequence having at least 70%, 80%, 90%, 95%, 96%, 97%, 98%, or 99% identity to SEQ ID NO: 96; and a light chain variable sequence having a sequence according to SEQ ID NO: 149, or a light chain variable sequence having at least 70%, 80%, 90%, 95%, 96%, 97%, 98%, or 99% identity to SEQ ID NO: 149;
(lxxxvii) a heavy chain variable sequence having a sequence according to SEQ ID NO: 97, or a heavy chain variable sequence having at least 70%, 80%, 90%, 95%, 96%, 97%, 98%, or 99% identity to SEQ ID NO: 97; and a light chain variable sequence having a sequence according to SEQ ID NO: 150, or a light chain variable sequence having at least 70%, 80%, 90%, 95%, 96%, 97%, 98%, or 99% identity to SEQ ID NO: 150;
(lxxxviii) a heavy chain variable sequence having a sequence according to SEQ ID NO: 98, or a heavy chain variable sequence having at least 70%, 80%, 90%, 95%, 96%, 97%, 98%, or 99% identity to SEQ ID NO: 98; and a light chain variable sequence having a sequence according to SEQ ID NO: 151, or a light chain variable sequence having at least 70%, 80%, 90%, 95%, 96%, 97%, 98%, or 99% identity to SEQ ID NO: 151;
(lxxxix) a heavy chain variable sequence having a sequence according to SEQ ID NO: 99, or a heavy chain variable sequence having at least 70%, 80%, 90%, 95%, 96%, 97%, 98%, or 99% identity to SEQ ID NO: 99; and a light chain variable sequence having a sequence according to SEQ ID NO: 152, or a light chain variable sequence having at least 70%, 80%, 90%, 95%, 96%, 97%, 98%, or 99% identity to SEQ ID NO: 152;
(xc) a heavy chain variable sequence having a sequence according to SEQ ID NO: 100, or a heavy chain variable sequence having at least 70%, 80%, 90%, 95%, 96%, 97%, 98%, or 99% identity to SEQ ID NO: 100; and a light chain variable sequence having a sequence according to SEQ ID NO: 136, or a light chain variable sequence having at least 70%, 80%, 90%, 95%, 96%, 97%, 98%, or 99% identity to SEQ ID NO: 136;
(xci) a heavy chain variable sequence having a sequence according to SEQ ID NO: 91, or a heavy chain variable sequence having at least 70%, 80%, 90%, 95%, 96%, 97%, 98%, or 99% identity to SEQ ID NO: 91; and a light chain variable sequence having a sequence according to SEQ ID NO: 153, or a light chain variable sequence having at least 70%, 80%, 90%, 95%, 96%, 97%, 98%, or 99% identity to SEQ ID NO: 153;
(xcii) a heavy chain variable sequence having a sequence according to SEQ ID NO: 101, or a heavy chain variable sequence having at least 70%, 80%, 90%, 95%, 96%, 97%, 98%, or 99% identity to SEQ ID NO: 101; and a light chain variable sequence having a sequence according to SEQ ID NO: 154, or a light chain variable sequence having at least 70%, 80%, 90%, 95%, 96%, 97%, 98%, or 99% identity to SEQ ID NO: 154;
(xciii) a heavy chain variable sequence having a sequence according to SEQ ID NO: 102, or a heavy chain variable sequence having at least 70%, 80%, 90%, 95%, 96%, 97%, 98%, or 99% identity to SEQ ID NO: 102; and a light chain variable sequence having a sequence according to SEQ ID NO: 155, or a light chain variable sequence having at least 70%, 80%, 90%, 95%, 96%, 97%, 98%, or 99% identity to SEQ ID NO: 155;
(xciv) a heavy chain variable sequence having a sequence according to SEQ ID NO: 36, or a heavy chain variable sequence having at least 70%, 80%, 90%, 95%, 96%, 97%, 98%, or 99% identity to SEQ ID NO: 36; and a light chain variable sequence having a sequence according to SEQ ID NO: 156, or a light chain variable sequence having at least 70%, 80%, 90%, 95%, 96%, 97%, 98%, or 99% identity to SEQ ID NO: 156; or
(xc) a heavy chain variable sequence having a sequence according to SEQ ID NO: 103, or a heavy chain variable sequence having at least 70%, 80%, 90%, 95%, 96%, 97%, 98%, or 99% identity to SEQ ID NO: 103; and a light chain variable sequence having a sequence according to SEQ ID NO: 157, or a light chain variable sequence having at least 70%, 80%, 90%, 95%, 96%, 97%, 98%, or 99% identity to SEQ ID NO: 157.

8. The monoclonal antibody or antibody fragment of any one of claims 1-3, wherein said antibody or antibody fragment comprises a heavy chain variable sequence having at least 70%, 80%, or 90% identity to SEQ ID NO: 7 and a light chain variable sequence having at least 70%, 80%, or 90% identity to SEQ ID NO: 8.

9. The monoclonal antibody or antibody fragment of claim 8, wherein said antibody or antibody fragment comprises a heavy chain variable sequence having at least 95% identity to SEQ ID NO: 7 and a light chain variable sequence having at least 95% identity to SEQ ID NO: 8.

10. The monoclonal antibody or antibody fragment of claim 9, wherein said antibody or antibody fragment comprises a heavy chain variable sequence having a sequence according to SEQ ID NO: 7 and a light chain variable sequence having a sequence according to SEQ ID NO: 8.

11. The monoclonal antibody or antibody fragment of any one of claims 1-10, wherein said antibody or antibody fragment is encoded by a heavy chain variable sequence having at least 70%, 80%, or 90% identity to SEQ ID NO: 9 and a light chain variable sequence having at least 70%, 80%, or 90% identity to SEQ ID NO: 10.

12. The monoclonal antibody or antibody fragment of claim 11, wherein said antibody or antibody fragment is encoded by a heavy chain variable sequence having at least 95% identity to SEQ ID NO: 9 and a light chain variable sequence having at least 95% identity to SEQ ID NO: 10.

13. The monoclonal antibody or antibody fragment of claim 12, wherein said antibody or antibody fragment is encoded by a heavy chain variable sequence according to SEQ ID NO: 9 and a light chain variable sequence according to SEQ ID NO: 10.

14. The monoclonal antibody or antibody fragment of any one of claims 1-13, wherein the antibody or antibody fragment is capable of binding to HSP70.

15. The monoclonal antibody or antibody fragment of any one of claims 1-14, wherein said antibody or antibody fragment is a humanized antibody.

16. The monoclonal antibody or antibody fragment of any one of claims 1-15, wherein the antibody fragment is a monovalent scFv (single chain fragment variable) antibody, divalent scFv, Fab fragment, F(ab′)₂ fragment, F(ab′)₃ fragment, Fv fragment, or single chain antibody.

17. The monoclonal antibody or antibody fragment of any one of claims 1-16, wherein said antibody or antibody fragment is a chimeric antibody, bispecific antibody, or BiTE.

18. The monoclonal antibody or antibody fragment of any one of claims 1-17, wherein said antibody or antibody fragment is an IgG antibody or a recombinant IgG antibody or antibody fragment.

19. The monoclonal antibody or antibody fragment of any one of claims 1-18, wherein said antibody is an IgG1, IgG2, IgG3, or IgG4 antibody, or a recombinant IgG1, IgG2, IgG3, or IgG4 antibody or antibody fragment.

20. The monoclonal antibody or antibody fragment of any one of claims 1-19, wherein said antibody or antibody fragment comprises SEQ ID NOs: 217-221.

21. The monoclonal antibody or antibody fragment of any one of claims 1-20, wherein the antibody or antibody fragment is conjugated or fused to an imaging agent or a cytotoxic agent.

22. The monoclonal antibody or antibody fragment of claim any one of claims 1-21, wherein the antibody or antibody fragment is labeled.

23. The monoclonal antibody or antibody fragment of claim 22, wherein the label is a fluorescent label, an enzymatic label, or a radioactive label.

24. A monoclonal antibody or antibody fragment, which competes for binding to the same epitope as the monoclonal antibody or an antibody fragment according to any one of claims 1-23.

25. A monoclonal antibody or antibody fragment that binds to an epitope on HSP70 recognized by an antibody or antibody fragment of any one of claims 1-24.

26. A monoclonal antibody or antibody fragment, wherein the monoclonal antibody or antibody fragment binds to an epitope of HSP70 defined by a peptide corresponding to K573-Q601 of SEQ ID NO: 11.

27. The monoclonal antibody or antibody fragment of claim 26, wherein, when bound to HSP70, the monoclonal antibody or antibody fragment binds to one or two of the following residues: H594, K595, and Q601 of SEQ ID NO:11.

28. The monoclonal antibody or antibody fragment of claim 26 or 27, wherein, when bound to HSP70, the monoclonal antibody or antibody fragment binds to all of the following residues: H594, K595, and Q601 of SEQ ID NO:11.

29. The monoclonal antibody or antibody fragment of claim 27 or 28, wherein, when bound to HSP70, the monoclonal antibody or antibody fragment additionally binds to at least one of the following residues: K573, E576, W580, R596, and E598 of SEQ ID NO:11.

30. The monoclonal antibody or antibody fragment of claim 27 or 28, wherein, when bound to HSP70, the monoclonal antibody or antibody fragment binds to at least two, three, four, or five of the following residues: K573, E576, W580, R596, and E598 of SEQ ID NO:11.

31. The monoclonal antibody or antibody fragment of any one of claims 26-30, wherein, when bound to HSP70, the monoclonal antibody or antibody fragment binds to all of the following residues: K573, E576, W580, H594, K595, R596, E598, and Q601 of SEQ ID NO:11.

32. A monoclonal antibody or antibody fragment, wherein, when bound to HSP70, the antibody or antibody fragment enhances the uptake of tumor-derived ADP-HSP70-peptide antigen complexes by immune effector cells.

33. The monoclonal antibody or antibody fragment of any one of claims 24-43, wherein said antibody or antibody fragment is a humanized antibody.

34. The monoclonal antibody or antibody fragment of any one of claims 24-33, wherein the antibody fragment is a monovalent scFv (single chain fragment variable) antibody, divalent scFv, Fab fragment, F(ab′)₂ fragment, F(ab′)₃ fragment, Fv fragment, or single chain antibody.

35. The monoclonal antibody or antibody fragment of any one of claims 24-34, wherein said antibody or antibody fragment is a chimeric antibody or bispecific antibody.

36. The monoclonal antibody or antibody fragment of any one of claims 24-35, wherein said antibody is an IgG antibody or a recombinant IgG antibody or antibody fragment.

37. The monoclonal antibody or antibody fragment of any one of claims 24-36, wherein said antibody is an IgG1, IgG2, IgG3, or IgG4 antibody, or a recombinant IgG1, IgG2, IgG3, or IgG4 antibody or antibody fragment.

38. The monoclonal antibody or antibody fragment of any one of claims 24-37, wherein said antibody or antibody fragment comprises SEQ ID NOs: 217-221.

39. The monoclonal antibody or antibody fragment of any one of claims 24-38, wherein the antibody or antibody fragment is conjugated or fused to an imaging agent or a cytotoxic agent.

40. The monoclonal antibody or antibody fragment of any one of claims 24-39, wherein the monoclonal antibody or antibody fragment is the monoclonal antibody or antibody fragment of any one of claims 1-23.

41. An isolated nucleic acid encoding the antibody heavy and/or light chain variable region of the antibody or antibody fragment of any of claims 1-41.

42. The isolated nucleic acid of claim 41, comprising a nucleotide sequence that is at least 85% identical to SEQ ID NO: 9 or 10.

43. An expression vector comprising the nucleic acid of claim 41 or 42.

44. A hybridoma or engineered cell comprising a nucleic acid encoding an antibody or antibody fragment of any one of claims 1-40.

45. A hybridoma or engineered cell comprising a nucleic acid of claim 41 or 42.

46. A method of making the monoclonal antibody or antibody fragment of any one of claims 1-36, the method comprising culturing the hybridoma or engineered cell of claim 44 or 45 under conditions that allow expression of the antibody or antibody fragment and optionally isolating the antibody from the culture.

47. A pharmaceutical formulation comprising one or more antibody or antibody fragment of any one of claims 1-40.

48. A method of treating a patient having a cancer, the method comprising administering an effective amount of the antibody or antibody fragment of any one of claims 1-40.

49. The method of claim 48, wherein the method enhances uptake of HSP70 by antigen presenting cells.

50. The method of claim 49, wherein the uptake of HSP70 by antigen presenting cells is mediated by human FcγR2A and/or human FcγR2B.

51. The method of any one of claims 48-50, further defined as a method for enhancing cytotoxic T cell-mediated antitumor immunity.

52. The method of claim any one of claims 48-51, further defined as a method for increasing sensitivity to immunotherapy.

53. The method of any one of claims 48-52, further defined as a method of enhancing uptake of tumor-derived ADP-HSP70-peptide antigen complexes by immune effector cells.

54. The method of any one of claims 48-53, further defined as a method of enhancing antigen presentation by dendritic cells.

55. The method of any one of claims 48-54, further defined as a method of enhancing CD4+ and CD8+ T-cell responses to tumor antigens.

56. The method of any one of claims 48-55, wherein the cancer is a pancreatic cancer or a prostate cancer.

57. The method of any one of claims 48-56, further comprising administering at least a second anti-cancer therapy.

58. The method of claim 57, wherein the second anti-cancer therapy is a chemotherapy, immunotherapy, radiotherapy, gene therapy, surgery, hormonal therapy, anti-angiogenic therapy or cytokine therapy.

59. A chimeric antigen receptor (CAR) protein comprising an antigen binding domain that binds to human HSP70.

60. The CAR protein of claim 59, wherein the CAR protein comprises:
(i) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(ii) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(iii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(iv) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(v) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(vi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 172; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(vii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(viii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 173; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(ix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 174; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(x) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 175; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;
(xii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 176; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 171; and a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(xiv) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 177; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xv) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 178; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 179; and a light chain variable region (VL) comprising a VLCDR1 amino acid sequence of SEQ ID NO: 159, a VLCDR2 amino acid sequence of SEQ ID NO: 5, and a VLCDR3 amino acid sequence of SEQ ID NO: 6;
(xviii) a heavy chain variable region (VH) comprising a VHCDR1 amino acid sequence of SEQ ID NO: 164, a VHCDR2 amino acid sequence of SEQ ID NO: 167, and a VHCDR3 amino acid sequence of SEQ ID NO: 174; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xix) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 180; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(xxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 182; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxiii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 181; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxiv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 184; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 185; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxvi) a heavy chain variable region (VH) comprising a VHCDR1 amino acid sequence of SEQ ID NO: 166 a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xvii) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxviii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxix) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 174; and a light chain variable region (VL) comprising a VLCDR1 amino acid sequence of SEQ ID NO: 160, a VLCDR2 amino acid sequence of SEQ ID NO: 5, and a VLCDR3 amino acid sequence of SEQ ID NO: 6;
(xxx) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 184; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxi) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 175; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxii) a heavy chain variable region (VH) comprising a VHCDR1 amino acid sequence of SEQ ID NO: 1, a VHCDR2 amino acid sequence of SEQ ID NO: 167, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxiii) a heavy chain variable region (VH) comprising a VHCDR1 amino acid sequence of SEQ ID NO: 1, a VHCDR2 amino acid sequence of SEQ ID NO: 169, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxiv) a heavy chain variable region (VH) comprising a VHCDR1 amino acid sequence of SEQ ID NO: 166, a VHCDR2 amino acid sequence of SEQ ID NO: 167, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxv) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxvi) a heavy chain variable region (VH) comprising a VHCDR1 amino acid sequence of SEQ ID NO: 166, a VHCDR2 amino acid sequence of SEQ ID NO: 168, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xxxvii) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xxxviii) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 3; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xxxix) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 184; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xl) a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 178; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 163;
(xli) a heavy chain variable region (VH) comprising a VHCDR1 amino acid sequence of SEQ ID NO: 164, a VHCDR2 amino acid sequence of SEQ ID NO: 2, and a VHCDR3 amino acid sequence of SEQ ID NO: 170; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 162;
(xlii) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 6;
(xliii) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xliv) a heavy chain variable region (VH) comprising a VHCDR1 amino acid sequence of SEQ ID NO: 166, a VHCDR2 amino acid sequence of SEQ ID NO: 2, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6;
(xlv) a heavy chain variable region (VH) comprising a VHCDR1 amino acid sequence of SEQ ID NO: 165, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 161, a VLCDR2 amino acid sequence of SEQ ID NO: 5, and a VLCDR3 amino acid sequence of SEQ ID NO: 162; or
(xlvi) a heavy chain variable region (VH) comprising a VHCDR1 amino acid sequence of SEQ ID NO: 1, a VHCDR2 amino acid sequence of SEQ ID NO: 168, and a VHCDR3 amino acid sequence of SEQ ID NO: 183; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

61. The CAR protein of claim 59 or 60, wherein the antigen binding domain comprises a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 4, a VLCDR2 amino acid sequence of SEQ ID NO: 5, and a VLCDR3 amino acid sequence of SEQ ID NO: 6.

62. The CAR of any one of claims 59-61, wherein the antigen binding domain comprises a heavy chain variable sequence having at least 70%, 80%, or 90% identity to SEQ ID NO: 7 and a light chain variable sequence having at least 70%, 80%, or 90% identity to SEQ ID NO: 8.

63. The CAR of any one of claims 59-62, wherein the antigen binding domain comprises a heavy chain variable sequence having at least 95% identity to SEQ ID NO: 7 and a light chain variable sequence having at least 95% identity to SEQ ID NO: 8.

64. The CAR of any one of claims 59-63, wherein the antigen binding domain comprises a heavy chain variable sequence having a sequence according to SEQ ID NO: 7 and a light chain variable sequence having a sequence according to SEQ ID NO: 8.

65. The CAR of any one of claims 59-64, wherein the antigen binding domain is encoded by a heavy chain variable sequence having at least 70%, 80%, or 90% identity to SEQ ID NO: 9 and a light chain variable sequence having at least 70%, 80%, or 90% identity to SEQ ID NO: 10.

66. The CAR of any one of claims 59-65, wherein the antigen binding domain is encoded by a heavy chain variable sequence having at least 95% identity to SEQ ID NO: 9 and a light chain variable sequence having at least 95% identity to SEQ ID NO: 10.

67. The CAR of any one of claims 59-66, wherein the antigen binding domain is encoded by a heavy chain variable sequence according to SEQ ID NO: 9 and a light chain variable sequence according to SEQ ID NO: 10.

68. The CAR of any one of claims 59-67, wherein the CAR is capable of binding to HSP70.

69. The CAR of any one of claims 59-68, wherein the antigen binding domain is a humanized antigen-binding domain.

70. The CAR of any one of claims 59-69, further comprising a hinge domain, a transmembrane domain, and an intracellular signaling domain.

71. The CAR of claim 70, wherein the hinge domain is a CD8a hinge domain or an IgG4 hinge domain.

72. The CAR of claim 70 or 71, wherein the transmembrane domain is a CD8a transmembrane domain or a CD28 transmembrane domain.

73. The CAR of any one of claims 70-72, wherein the intracellular signaling domain comprises a CD3z intracellular signaling domain.

74. A nucleic acid molecule encoding a CAR of any one of claims 59-73.

75. The nucleic acid molecule of claim 74, wherein the sequence encoding the CAR is operatively linked to expression control sequences.

76. The nucleic acid molecule of claim 74 or 75, further defined as an expression vector.

77. An engineered cell comprising a nucleic acid molecule encoding a chimeric antigen receptor (CAR) comprising an antigen binding domain that binds to human HSP70.

78. The cell of claim 77, wherein the nucleic acid molecule encodes a CAR of any one of claims 59-73.

79. The cell of claim 77 or 78, wherein the cell is a T cell.

80. The cell of claim 77 or 78, wherein the cell is an NK cell.

81. The cell of any one of claims 77-80, wherein the nucleic acid is integrated into the genome of the cell.

82. The cell of any one of claims 77-81, wherein the cell is a human cell.

83. A pharmaceutical composition comprising a population of cells in accordance with any one of claims 77-82 in a pharmaceutically acceptable carrier.

84. A method of treating cancer in a human patient in need thereof comprising administering to the patient an anti-tumor effective amount of a cell therapy comprising one or more cells in accordance with any one of claims 78-82.

85. The method of claim 84, wherein the cells are allogeneic cells.

86. The method of claim 84, wherein the cells are autologous cells.

87. The method of any one of claims 84-86, wherein the cells are HLA matched to the subj ect.

88. The method of any one of claims 84-87, wherein the cancer is a pancreatic cancer or a prostate cancer.

89. The method of any one of claims 84-88, further comprising administering at least a second anti-cancer therapy.

90. The method of claim 89, wherein the second anti-cancer therapy is a chemotherapy, immunotherapy, radiotherapy, gene therapy, surgery, hormonal therapy, anti-angiogenic therapy or cytokine therapy.

91. A method of detecting HSP70 in an in vitro sample, the method comprising contacting the in vitro sample with an antibody or antibody fragment of any one of claims 1-36 and detecting the binding of the antibody or antibody fragment to the sample.

92. The method of claim 91, wherein the detecting is by flow cytometry, mass spectrometry, western blot, immunohistochemistry, ELISA, or RIA.

93. An antibody or antibody fragment of any of claims 1-40, a pharmaceutical composition of claim 47, a cell of any of claims 77-82, or a pharmaceutical composition of claim 83 for use in treating a cancer in a subject.

94. Use of an antibody or antibody fragment of any of claims 1-40, a pharmaceutical composition of claim 47, a cell of any of claims 77-82, or a pharmaceutical composition of claim 83, in the manufacture of a medicament for treating a cancer in a subject.


SEQUENCE LISTING (234 sequences)

SEQ ID NO: 1 - protein, 8 aa, Artificial Sequence
  Engineered polypeptide
GYTFTNYG

SEQ ID NO: 2 - protein, 8 aa, Artificial Sequence
  Engineered polypeptide
INTYTGEP

SEQ ID NO: 3 - protein, 9 aa, Artificial Sequence
  Engineered polypeptide
ARYDHAMDY

SEQ ID NO: 4 - protein, 12 aa, Artificial Sequence
  Engineered polypeptide
QSLLNSGTRKNY

SEQ ID NO: 5 - protein, 3 aa, Artificial Sequence
  Engineered polypeptide
WTS

SEQ ID NO: 6 - protein, 8 aa, Artificial Sequence
  Engineered polypeptide
KQSYTLYT

SEQ ID NO: 7 - protein, 116 aa, Artificial Sequence
  Engineered polypeptide
QIQLVQSGPELKKPGETVKISCKASGYTFTNYGMNWVKQAPGKGLKWMGWINTYTGEPTYADDFKGRFAFSLETSATTAYLQINNLKNEDTATYFCARYDHAMDYWGQGTSVTVSS

SEQ ID NO: 8 - protein, 112 aa, Artificial Sequence
  Engineered polypeptide
DIVMSQSPSSLAVSAGEKVTMSCKSSQSLLNSGTRKNYLAWYQQKPGQSPKLLIYWTSTRESGVPDRFTGSGSGTDFTLTINSVQAEDLAVYYCKQSYTLYTFGGGTKLEIK

SEQ ID NO: 9 - DNA, 349 bp, Artificial Sequence
  Engineered polynucleotide
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccaccactgcctatttgcagatcaacaacctcaaaaatgaggacacggctacatatttctgtgcaaggtacgaccatgctatggactactggggtcaaggaacctcagtcaccgtctcctcag

SEQ ID NO: 10 - DNA, 337 bp, Artificial Sequence
  Engineered polynucleotide
gacattgtgatgtcacagtctccatcctccctggctgtgtcagctggagagaaggtcactatgagctgcaaatccagtcagagtctgctcaacagtggaacccgaaagaactacttggcttggtaccagcagaaaccagggcagtctcctaaactgctgatctactggacatccactagggaatctggggtccctgatcgcttcacaggcagtggatctgggacagatttcactctcaccatcaacagtgtgcaggctgaagacctggcagtttattactgcaagcaatcttatactctgtacacgttcggaggggggaccaagctggaaataaaac

SEQ ID NO: 11 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 12 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 13 - protein, 116 aa, Artificial Sequence
  Engineered polypeptide
QIQLVQSGAEVKKPGSSVKVSCKASGYTFTNYGMNWVRQAPGQGLEWMGWINTYTGEPTYADDFKGRFTFTADESTSTAYMELSSLRSEDTAVYFCARYDHAMDYWGQGTLVTVSS

SEQ ID NO: 14 - protein, 116 aa, Artificial Sequence
  Engineered polypeptide
QVQLQQSGPEVKKPGASVKISCKTSGYTFTNYGMNWVRQAPGQGLEWMGWINTYTGEPTYADDFKGRVTMTTDTSTSTAYLELTGLMSDDTAVYFCARYDHAMDYWGQGTTVTVSS

SEQ ID NO: 15 - protein, 116 aa, Artificial Sequence
  Engineered polypeptide
EVQLVESGGGLVKPGGSLRLSCKASGYTFTNYGMNWVRQAPGKGLKWVGWINTYTGEPTYADDFKGRFTFSRDDSKNTLYLQMNSLKTEDTAVYFCARYDHAMDYWGQGTSVTVSS

SEQ ID NO: 16 - protein, 116 aa, Artificial Sequence
  Engineered polypeptide
QIQLVQSGPEVKKPGESVKVSCKASGYTFTNYGMNWVRQAPGKGLEWMGWINTYTGEPTYADDFKGRVTITRDTSASTAYMELSSLRSEDTAVYFCARYDHAMDYWGQGTSVTVSS

SEQ ID NO: 17 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTMTTDTSTSTAYMELRSLRSDDTAVYFCARYDHAMDYWGQGTLVTVSS

SEQ ID NO: 18 - protein, 116 aa, Artificial Sequence
  Engineered polypeptide
  MISC_FEATURE (1)..(1): Xaa is Gln or Glu
  MISC_FEATURE (2)..(2): Xaa is Ile or Val
  MISC_FEATURE (5)..(5): Xaa is Val or Gln
  MISC_FEATURE (6)..(6): Xaa is Gln or Glu
  MISC_FEATURE (9)..(9): Xaa is Ala, Pro, or Gly
  MISC_FEATURE (10)..(10): Xaa is Glu or Gly
  MISC_FEATURE (11)..(11): Xaa is Val or Leu
  MISC_FEATURE (12)..(12): Xaa is Val or Lys
  MISC_FEATURE (16)..(16): Xaa is Ala, Glu, Gly, or Ser
  MISC_FEATURE (18)..(18): Xaa is Val or Leu
  MISC_FEATURE (19)..(19): Xaa is Lys or Arg
  MISC_FEATURE (20)..(20): Xaa is Val, Leu, or Ile
  MISC_FEATURE (24)..(24): Xaa is Ala or Thr
  MISC_FEATURE (43)..(43): Xaa is Lys or Gln
  MISC_FEATURE (46)..(46): Xaa is Glu or Lys
  MISC_FEATURE (48)..(48): Xaa is Met or Val
  MISC_FEATURE (68)..(68): Xaa is Phe or Val
  MISC_FEATURE (70)..(70): Xaa is Phe, Met, or Ile
  MISC_FEATURE (71)..(71): Xaa is Thr or Ser
  MISC_FEATURE (72)..(72): Xaa is Thr, Arg, or Ala
  MISC_FEATURE (74)..(74): Xaa is Thr, Asp, or Glu
  MISC_FEATURE (76)..(76): Xaa is Thr, Ala, or Lys
  MISC_FEATURE (77)..(77): Xaa is Ser or Asn
  MISC_FEATURE (79)..(79): Xaa is Leu or Ala
  MISC_FEATURE (81)..(81): Xaa is Met or Leu
  MISC_FEATURE (82)..(82): Xaa is Glu or Gln
  MISC_FEATURE (83)..(83): Xaa is Leu or Met
  MISC_FEATURE (84)..(84): Xaa is Arg, Ser, Thr, or Asn
  MISC_FEATURE (85)..(85): Xaa is Ser or Gly
  MISC_FEATURE (87)..(87): Xaa is Arg, Lys, or Met
  MISC_FEATURE (88)..(88): Xaa is Ser or Thr
  MISC_FEATURE (89)..(89): Xaa is Asp or Glu
  MISC_FEATURE (111)..(111): Xaa is Leu, Ser, or Thr
XXQLXXSGXXXXKPGXSXXXSCKXSGYTFTNYGMNWVRQAPGXGLXWXGWINTYTGEPTYADDFKGRXTXXXDXSXXTXYXXXXXLXXXDTAVYFCARYDHAMDYWGQGTXVTVSS

SEQ ID NO: 19 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 20 - protein, 112 aa, Artificial Sequence
  Engineered polypeptide
DIVMTQSPDSLSVSLGERATINCKSSQSLLNSGTRKNYLAWYQQKPGQPPRLLIYWTSTRESGVPDRFSGSGSGTDFTLTINTLQAEDVAVYYCKQSYTLYTFGQGTKLEIK

SEQ ID NO: 21 - protein, 112 aa, Artificial Sequence
  Engineered polypeptide
DVVMTQSPDSLAVSLGERVTINCKSSQSLLNSGTRKNYLAWYQQKPGQSPKLLIYWTSTRESGVPDRFSGSGSGTDFTLTISSLQAEDVAVYYCKQSYTLYTFGQGTKLEIK

SEQ ID NO: 22 - protein, 112 aa, Artificial Sequence
  Engineered polypeptide
DIQMTQSPSSLSASVGDRVTITCKSSQSLLNSGTRKNYLAWYQQKPGKVPKLLIYWTSTRESGVPSRFSGSGSGTDFTLTISSLQPEDVATYYCKQSYTLYTFGGGTKLEIK

SEQ ID NO: 23 - protein, 112 aa, Artificial Sequence
  Engineered polypeptide
DIVMTQSPDSLAVSLGERATINCKSSQSLLNSGTRKNYLAWYQQKPGQSPKLLIYWTSTRESGVPDRFSGSGSGTDFTLTISSLQAEDVAVYYCKQSYTLYTFGGGTKLEIK

SEQ ID NO: 24 - protein, 112 aa, Artificial Sequence
  Engineered polypeptide
DIVMTQSPDSLAVSLGERATINCKSSQSLLNSGTRKNYLAWYQQKPGQSPKLLIYWTSTRESGVPDRFSGSGSGTDFTLTISSLQAEDVAVYYCKQSYTLYTFGQGTKLEIK

SEQ ID NO: 25 - protein, 112 aa, Artificial Sequence
  Engineered polypeptide
  MISC_FEATURE (1)..(1): Xaa is Glu or Asp
  MISC_FEATURE (2)..(2): Xaa is Ile or Val
  MISC_FEATURE (3)..(3): Xaa is Val or Gln
  MISC_FEATURE (4)..(4): Xaa is Leu or Met
  MISC_FEATURE (9)..(9): Xaa is Asp or Ser
  MISC_FEATURE (12)..(12): Xaa is Ala or Ser
  MISC_FEATURE (13)..(13): Xaa is Val or Ala
  MISC_FEATURE (15)..(15): Xaa is Leu or Val
  MISC_FEATURE (17)..(17): Xaa is Glu or Asp
  MISC_FEATURE (19)..(19): Xaa is Ala or Val
  MISC_FEATURE (22)..(22): Xaa is Asn or Thr
  MISC_FEATURE (46)..(46): Xaa is Ala or Pro
  MISC_FEATURE (48)..(48): Xaa is Gln or Lys
  MISC_FEATURE (49)..(49): Xaa is Ser, Val, or Pro
  MISC_FEATURE (51)..(51): Xaa is Lys or Arg
  MISC_FEATURE (66)..(66): Xaa is Asp or Ser
  MISC_FEATURE (82)..(82): Xaa is Ser, Asp, or Asn
  MISC_FEATURE (83)..(83): Xaa is Ser or Thr
  MISC_FEATURE (86)..(86): Xaa is Ala or Pro
  MISC_FEATURE (91)..(91): Xaa is Val or Thr
  MISC_FEATURE (105)..(105): Xaa is Gln or Gly
  MISC_FEATURE (109)..(109): Xaa is Leu or Val
XXXXTQSPXSLXXSXGXRXTIXCKSSQSLLNSGTRKNYLAWYQQKXGXXPXLLIYWTSTRESGVPXRFSGSGSGTDFTLTIXXLQXEDVAXYYCKQSYTLYTFGXGTKXEIK

SEQ ID NO: 26 - protein, 116 aa, Artificial Sequence
  Engineered polypeptide
QIQLVQSGAEVKKPGASVKVSCKASGYTFTKYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 27 - protein, 116 aa, Artificial Sequence
  Engineered polypeptide
QIQLVQSGAEVKKPGASVKVSCKASGYTFTNYGMNWVRQAPGQGLEWMGWINTYTGEPRYADDFKGRFTFTTDTSTSTAYMELRSLRSDDTAVYFCARYDHAMDYWGQGTLVTVSS

SEQ ID NO: 28 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHRMDYWGQGTLVTVSS

SEQ ID NO: 29 - protein, 116 aa, Artificial Sequence
  Engineered polypeptide
QIQLVQSGAEVKKPGASVKVSCKASGYTFTNYGMNWVRQAPGQGLEWMGWINTYTGEPIYADDFKGRFTFTTDTSTSTAYMELRSLRSDDTAVYFCARYDHAMDYWGQGTLVTVSS

SEQ ID NO: 30 - protein, 116 aa, Artificial Sequence; identical to SEQ ID NO: 12
  Engineered polypeptide

SEQ ID NO: 31 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTRTAYMELRSLRSDDTAVYFCARYDHAMDYWGQGTLVTVSS

SEQ ID NO: 32 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHFMDYWGQGTLVTVSS

SEQ ID NO: 33 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHTMDYWGQGTLVTVSS

SEQ ID NO: 34 - protein, 116 aa, Artificial Sequence
  Engineered polypeptide
QIQLVQSGDEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 35 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHPMDYWGQGTLVTVSS

SEQ ID NO: 36 - protein, 116 aa, Artificial Sequence
  Engineered polypeptide
QIQLVQSGAEVKKPGASVKVSCKASGYTFTNYGMNWVRQAPGQGLEWMGWINTYTGEPTYTDDFKGRFTFTTDTSTSTAYMELRSLRSDDTAVYFCARYDHAMDYWGQGTLVTVSS

SEQ ID NO: 37 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHVMDYWGQGTLVTVSS

SEQ ID NO: 38 - protein, 116 aa, Artificial Sequence
  Engineered polypeptide
QIQLVQSGAEVKKPGASVKVSCKASGYTFTKYGMNWVRQAPGQGLEWMGWINTYTGEPTYADDFKGRFTFTTDTSTSTAYMELRSLRSDDTAVYFCARYDHSMDYWGQGTLVTVSS

SEQ ID NO: 39 - protein, 116 aa, Artificial Sequence
  Engineered polypeptide
QIQLVQSGAEVKKPGASVKVSCKASGYTFTNYGMNWVRQAPGQGLEWMGWINTYTGEPTYVDDFKGRFTFTTDTSTSTAYMDLRSLRSDDTAVYFCARYDHAMDYWGQGTLVTVSS

SEQ ID NO: 40 - protein, 116 aa, Artificial Sequence
  Engineered polypeptide
QIQLVQSGDEVKKPGASVKVSCKASGYTFTNYGMNWVRQAPGQGLEWMGWINTYTGEPTYSDDFKGRFTFTTDTSTSTAYMELRSLRSDDTAVYFCARYDHAMDYWGQGTLVTVSS

SEQ ID NO: 41 - protein, 116 aa, Artificial Sequence
  Engineered polypeptide
QIQLVQSGAEVKKPGASVKVSCKASGYTFTNYGMNWVRQAPGQGLEWMGWINTYTGEPTYSDDFKGRFTFTTDTSTSTAYMELRSLRSDDTAVYFCARYDHAMDYWGQGTLVTVSS

SEQ ID NO: 42 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTRTAYMELRSLRSDDTAVYFCTRYDHAMDYWGQGTLVTVSS

SEQ ID NO: 43 - protein, 116 aa, Artificial Sequence
  Engineered polypeptide
QIQLVQSGTEVKKPGASVKVSCKASGYTFTNYGMNWVRQAPGQGLEWMGWINTYTGEPTYADDFKGRFTFTTDTSTSTAYMELRSLRSDDTAVYFCARYDHTMDYWGQGTLVTVSS

SEQ ID NO: 44 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMEVRSLRSDDTAVYFCARYDHAMDYWGQGTLVTVSS

SEQ ID NO: 45 - protein, 116 aa, Artificial Sequence
  Engineered polypeptide
QIQLVQSGAEVKKPGASVKVSCKASGYTFTNYGMNWVRQAPGQGLEWMGWINTYTGEPRYADDFKGRFTFTTDTSTSTAYMELRSLRSDDTAVYFCARYDHDMDYWGQGTLVTVSS

SEQ ID NO: 46 - protein, 116 aa, Artificial Sequence
  Engineered polypeptide
QIQLVQSGAEVKKPGASVKVSCKASGYTFTKYGMNWVRQAPGQGLEWMGWINTYTGEPTYADDFKGRFTFTTDTSTSTAYMELRSLRSDDTAVYFCARYDHNMDYWGQGTLVTVSS

SEQ ID NO: 47 - protein, 116 aa, Artificial Sequence
  Engineered polypeptide
QIQLVQSGTEVKKPGASVKVSCKASGYTFTNYGMNWVRQAPGQGLEWMGWINTYTGEPTYADDFKGRFTFTTDTSTSTAYMELRSLRSDDTAVYFCARYDHHMDYWGQGTLVTVSS

SEQ ID NO: 48 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTTTAYMELRSLRSDDTAVYFCARYDHHMDYWGQGTLVTVSS

SEQ ID NO: 49 - protein, 116 aa, Artificial Sequence
  Engineered polypeptide
QIQLVQSGAEVKKPGASVKVSCKASGYTFTKYGMNWVRQAPGQGLEWMGWINTYTGESTYADDFKGRFTFTTDTSTSTAYMELRSLRSDDTAVYFCARYDHVMDYWGQGTLVTVSS

SEQ ID NO: 50 - protein, 116 aa, Artificial Sequence
  Engineered polypeptide
QIQLVQSGTEVKKPGASVKVSCKASGYTFTNYGMNWVRQAPGQGLEWMGWINTYTGEPTYADDFKGRFTFTTDTSTRTAYMELRSLRSDDTAVYFCARYDHLMDYWGQGTLVTVSS

SEQ ID NO: 51 - protein, 116 aa, Artificial Sequence
  Engineered polypeptide
QIQLVQSGAEVKKPGASVKVSCKASGYTFTNYGMNWVRQAPGQGLEWMGWINTYTGEPTYSDDFKGRFTFTTDTSTRTAYMELRSLRSDDTAVYFCARYDHAMDYWGQGTLVTVSS

SEQ ID NO: 52 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHYMDYWGQGTLVTVSS

SEQ ID NO: 53 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTTTAYMELRSLRSDDTAVYFCARYDHVMDYWGQGTLVTVSS

SEQ ID NO: 54 - protein, 116 aa, Artificial Sequence
  Engineered polypeptide
QIHLVQSGAEVKKPGASVKVSCKASGYTFTNYGMNWVRQAPGQGLEWMGWINTYTGEPTYADDFKGRFTFTTDTSTSTAYMELRSLRSDDTAVYFCTRYDHRMDYWGQGTLVTVSS

SEQ ID NO: 55 - protein, 116 aa, Artificial Sequence
  Engineered polypeptide
QIHLVQSGAEVKKPGASVKVSCKASGYTFTNYGMNWVRQAPGQGLEWMGWINTYTGEPTYADDFKGRFTFTTDTSTSTVYMELRSLRSDDTAVYFCARYDHAMDYWGQGTLVTVSS

SEQ ID NO: 56 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCVRYDHAMDYWGQGSLVTVSS

SEQ ID NO: 57 - protein, 116 aa, Artificial Sequence
  Engineered polypeptide
QIQLVQSGVEVKKPGASVKVSCKASGYTFTNYGMNWVRQAPGQGLEWMGWINTYTGEPTYADDFKGRFTFTTDTSTSTVYMELRSLRSDDTAVYFCARYDHAMDYWGQGTLVTVSS

SEQ ID NO: 58 - protein, 116 aa, Artificial Sequence
  Engineered polypeptide
QIQLVQSGVEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGTLVTVSS

SEQ ID NO: 59 - protein, 116 aa, Artificial Sequence
  Engineered polypeptide
QIQLVQSGTEVKKPGASVKVSCKASGYTFTNYGMNWVRQAPGQGLEWMGWINTYTGEPTYADDFKGRFTFTTDTSTSTAYMELRSLRSDDTAVYFCARYDHLMDYWGQGTLVTVSS

SEQ ID NO: 60 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMDLRSLRSDDTAVYFCARYDHYMDYWGQGTLVTVSS

SEQ ID NO: 61 - protein, 116 aa, Artificial Sequence
  Engineered polypeptide
QIQLVQSGAEVKKPGASVKVSCKASGYTFTNYGMNWVRQAPGQGLEWMGWINTYTGETTYGDDFKGRFTFTTDTSTSTAYMELRSLRSDDTAVYFCGRYDHAMDYWGQGTLVTVSS

SEQ ID NO: 62 - protein, 116 aa, Artificial Sequence
  Engineered polypeptide
QIHLVQSGVEVKKPGASVKVSCKASGYTFTNYGMNWVRQAPGQGLEWMGWINTYTGEPTYTDDFKGRFTFTTDTSTSTAYMELRSLRSDDTAVYFCARYDHAMDYWGQGTLVTVSS

SEQ ID NO: 63 - protein, 116 aa, Artificial Sequence
  Engineered polypeptide
QIQLVQSGAEVKKPGASVKVSCKASGYSFTNYGMNWVRQAPGQGLEWMGWINTYTGEPTYADDFKGRFTFTTDTSTSTAYMELRSLRSDDTAVYFCARYDHGMDYWGQGTLVTVSS

SEQ ID NO: 64 - protein, 116 aa, Artificial Sequence
  Engineered polypeptide
QIQLVQSGVEVKKPGASVKVSCKASGYIFTNYGMNWVRQAPGQGLEWMGWINTYTGEPRYADDFKGRFTFTTDTSTSTAYMELRSLRSDDTAVYFCARYDHRMDYWGQGTLVTVSS

SEQ ID NO: 65 - protein, 116 aa, Artificial Sequence
  Engineered polypeptide
QIQLVQSGAEVKKPGASVKVSCKASGYTFTNYGMNWVRQAPGQGLEWMGWINTYTGEPKYTDDFKGRFTFTTDTSTRTAYMELRSLRSDDTAVYFCARYDHAMDYWGQGTLVTVSS

SEQ ID NO: 66 - protein, 116 aa, Artificial Sequence
  Engineered polypeptide
QIQLVQSGAEVKKPGASVKVSCKASGYTFTKYGMNWVRQAPGQGLEWMGWINTYTGEPKYGDDFKGRFTFTTDTSTSTAYMELRSLRSDDTAVYFCARYDHRMDYWGQGTLVTVSS

SEQ ID NO: 67 - protein, 116 aa, Artificial Sequence
  Engineered polypeptide
QIQLVQSGSEVKKPGASVKVSCKASGYTFTNYGMNWVRQAPGQGLEWMGWINTYTGEPTYGDDFKGRFTFTTDTSTSTAYMELRSLRSDDTAVYFCARYDHFMDYWGQGTLVTVSS

SEQ ID NO: 68 - protein, 116 aa, Artificial Sequence
  Engineered polypeptide
QIQLVQSGTEVKKPGASVKVSCKASGYSFTNYGMNWVRQAPGQGLEWMGWINTYTGEPTYADDFKGRFTFTTDTSTTTAYMELRSLRSDDTAVYFCARYDHAMDYWGQGTLVTVSS

SEQ ID NO: 69 - protein, 116 aa, Artificial Sequence
  Engineered polypeptide
QIQLVQSGAEVKKPGASVKVSCKASGYTFTNYGMNWVRQAPGQGLEWMGWINTYTGEATYADDFKGRFTFTTDTSTSTAYMELRSLRSDDTAVYFCARYDHVMDYWGQGTLVTVSS

SEQ ID NO: 70 - protein, 116 aa, Artificial Sequence
  Engineered polypeptide
QIQLVQSGSEVKKPGASVKVSCKASGYTFTNYGMNWVRQAPGQGLEWMGWINTYTGEPTYADDFKGRFTFTTDTSTSTAYMELRSLRSDDTAVYFCGRYDHAMDYWGQGTLVTVSS

SEQ ID NO: 71 - protein, 116 aa, Artificial Sequence
  Engineered polypeptide
QIQLVQSGPEVKKPGASVKVSCKASGYTFTNYGMNWVRQAPGQGLEWMGWINTYTGEPTYGDDFKGRFTFTTDTSTSTAYMELRSLRSDDTAVYFCGRYDHAMDYWGQGTLVTVSS

SEQ ID NO: 72 - protein, 116 aa, Artificial Sequence
  Engineered polypeptide
QIQLVQSGSEVKKPGASVKVSCKASGYTFTNYGMNWVRQAPGQGLEWMGWINTYTGEPTYADDFKGRFTFTTDTSTSTAYMELRSLRSDDTAVYFCARYDHSMDYWGQGTLVTVSS

SEQ ID NO: 73 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMELRSLRSDDTAVYFCARYDHAMDYWGQGSLVTVSS

SEQ ID NO: 74 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTTTGYMELRSLRSDDTAVYFCARYDHAMDYWGQGTLVTVSS

SEQ ID NO: 75 - protein, 116 aa, Artificial Sequence
  Engineered polypeptide
QIQLVQSGAEVKKPGASVKVSCKASGYTFTNYGMNWVRQAPGQGLEWMGWINTYTGESTYADDFKGRFTFTTDTSTSTAYMELRSLRSDDTAVYFCARYDHAMDYWGQGTLVTVSS

SEQ ID NO: 76 - protein, 116 aa, Artificial Sequence
  Engineered polypeptide
QIQLVQSGAEVKKPGASVKVSCKASGYTFTNYGMNWVRQAPGQGLEWMGWINTYTGEATYADDFKGRFTFTTDTSTSTAYMELRSLRSDDTAVYFCARYDHAMDYWGQGSLVTVSS

SEQ ID NO: 77 - protein, 116 aa, Artificial Sequence
  Engineered polypeptide
QIQLVQSGAEVKKPGASVKVSCKASGYIFTNYGMNWVRQAPGQGLEWMGWINTYTGESTYADDFKGRFTFTTDTSTSTVYMELRSLRSDDTAVYFCARYDHAMDYWGQGTLVTVSS

SEQ ID NO: 78 - protein, 116 aa, Artificial Sequence
  Engineered polypeptide
QIQLVQSGAEVKKPGASVKVSCKASGYTFTNYGMNWVRQAPGQGLEWMGWINTYTGEPTYADDFKGRFTFTTDTSTSTAYMDLRSLRSDDTAVYFCARYDHAMDYWGQGTLVTVSS

SEQ ID NO: 79 - protein, 116 aa, Artificial Sequence
  Engineered polypeptide
QIHLVQSGDEVKKPGASVKVSCKASGYTFTNYGMNWVRQAPGQGLEWMGWINTYTGEPTYTDDFKGRFTFTTDTSTSTAYMELRSLRSDDTAVYFCARYDHAMDYWGQGTLVTVSS

SEQ ID NO: 80 - protein, 116 aa, Artificial Sequence
  Engineered polypeptide
QIQLVQSGAEVKKPGASVKVSCKASGYTFTNYGMNWVRQAPGQGLEWMGWINTYTGETTYTDDFKGRFTFTTDTSTSTAYMELRSLRSDDTAVYFCARYDHAMDYWGQGTLVTVSS

SEQ ID NO: 81 - protein, 116 aa, Artificial Sequence
  Engineered polypeptide
QIQLVQSGSEVKKPGASVKVSCKASGYTFTNYGMNWVRQAPGQGLEWMGWINTYTGEPTYADDFKGRFTFTTDTSTSTAYMDLRSLRSDDTAVYFCARYDHAMDYWGQGTLVTVSS

SEQ ID NO: 82 - protein, 116 aa, Artificial Sequence
  Engineered polypeptide
QIQLVQSGPEVKKPGASVKVSCKASGYIFTNYGMNWVRQAPGQGLEWMGWINTYTGETTYADDFKGRFTFTTDTSTTTVYMELRSLRSDDTAVYFCARYDHAMDYWGQGTLVTVSS

SEQ ID NO: 83 - protein, 116 aa, Artificial Sequence
  Engineered polypeptide
QIHLVQSGDEVKKPGASVKVSCKASGYTFTNYGMNWVRQAPGQGLEWMGWINTYTGEPTYADDFKGRFTFTTDTSTSTGYMELRSLRSDDTAVYFCARYDHAMDYWGQGSLVTVSS

SEQ ID NO: 84 - protein, 116 aa, Artificial Sequence
  Engineered polypeptide
QIQLVQSGPEVKKPGASVKVSCKASGYTFTNYGMNWVRQAPGQGLEWMGWINTYTGEPTYADDFKGRFTFTTDTSTTTGYMELRSLRSDDTAVYFCARYDHAMDYWGQGTLVTVSS

SEQ ID NO: 85 - protein, 116 aa, Artificial Sequence
  Engineered polypeptide
QIQLVQSGVEVKKPGASVKVSCKASGYTFTNYGMNWVRQAPGQGLEWMGWINTYTGEPTYSDDFKGRFTFTTDTSTSTAYMEVRSLRSDDTAVYFCARYDHAMDYWGQGTLVTVSS

SEQ ID NO: 86 - protein, 116 aa, Artificial Sequence
  Engineered polypeptide
QIQLVQSGVEVKKPGASVKVSCKASGYTFTNYGMNWVRQAPGQGLEWMGWINTYTGETTYADDFKGRFTFTTDTSTSTAYMEVRSLRSDDTAVYFCARYDHAMDYWGQGTLVTVSS

SEQ ID NO: 87 - protein, 116 aa, Artificial Sequence
  Engineered polypeptide
QIQLVQSGPEVKKPGASVKVSCKASGYIFTNYGMNWVRQAPGQGLEWMGWINTYTGEPTYGDDFKGRFTFTTDTSTSTAYMELRSLRSDDTAVYFCGRYDHAMDYWGQGTLVTVSS

SEQ ID NO: 88 - protein, 116 aa, Artificial Sequence
  Engineered polypeptide
QIHLVQSGAEVKKPGASVKVSCKASGYTFTNYGMNWVRQAPGQGLEWMGWINTYTGEPKYADDFKGRFTFTTDTSTSTAYMELRSLRSDDTAVYFCARYDHNMDYWGQGTLVTVSS

SEQ ID NO: 89 - protein, 116 aa, Artificial Sequence
  Engineered polypeptide
QIQLVQSGAEVKKPGASVKVSCKASGYTFTKYGMNWVRQAPGQGLEWMGWINTYTGEPRYADDFKGRFTFTTDTSTSTAYMELRSLRSDDTAVYFCARYDHRMDYWGQGTLVTVSS

SEQ ID NO: 90 - protein, 116 aa, Artificial Sequence
  Engineered polypeptide
QIQLVQSGAEVKKPGASVKVSCKASGYTFTNYGMNWVRQAPGQGLEWMGWINTYTGEPKYADDFKGRFTFTTDTSTSTAYMELRSLRSDDTAVYFCARYDHVMDYWGQGTLVTVSS

SEQ ID NO: 91 - protein, 116 aa, Artificial Sequence
  Engineered polypeptide
QIHLVQSGAEVKKPGASVKVSCKASGYIFTNYGMNWVRQAPGQGLEWMGWINTYTGEPTYADDFKGRFTFTTDTSTSTVYMELRSLRSDDTAVYFCVRYDHAMDYWGQGTLVTVSS

SEQ ID NO: 92 - protein, 116 aa, Artificial Sequence
  Engineered polypeptide
QIQLVQSGTEVKKPGASVKVSCKASGYTFTNYGMNWVRQAPGQGLEWMGWINTYTGETTYADDFKGRFTFTTDTSTSTAYMDLRSLRSDDTAVYFCARYDHAMDYWGQGTLVTVSS

SEQ ID NO: 93 - protein, 116 aa, Artificial Sequence
  Engineered polypeptide
QIHLVQSGPEVKKPGASVKVSCKASGYTFTNYGMNWVRQAPGQGLEWMGWINTYTGEPTYADDFKGRFTFTTDTSTSTAYMELRSLRSDDTAVYFCGRYDHAMDYWGQGTLVTVSS

SEQ ID NO: 94 - protein, 116 aa, Artificial Sequence
  Engineered polypeptide
QIHLVQSGAEVKKPGASVKVSCKASGYSFTNYGMNWVRQAPGQGLEWMGWINTYTGEPTYADDFKGRFTFTTDTSTSTAYMELRSLRSDDTAVYFCVRYDHAMDYWGQGTLVTVSS

SEQ ID NO: 95 - protein, 116 aa, Artificial Sequence
  Engineered polypeptide
QIHLVQSGAEVKKPGASVKVSCKASGYIFTNYGMNWVRQAPGQGLEWMGWINTYTGEPTYADDFKGRFTFTTDTSTSTAYMELRSLRSDDTAVYFCVRYDHAMDYWGQGTLVTVSS

SEQ ID NO: 96 - protein, 116 aa, Artificial Sequence
  Engineered polypeptide
QIQLVQSGVEVKKPGASVKVSCKASGYTFTNYGMNWVRQAPGQGLEWMGWINTYTGETTYADDFKGRFTFTTDTSTSTAYMELRSLRSDDTAVYFCARYDHAMDYWGQGTLVTVSS

SEQ ID NO: 97 - protein, 116 aa, Artificial Sequence
  Engineered polypeptide
QIQLVQSGAEVKKPGASVKVSCKASGYTFTKYGMNWVRQAPGQGLEWMGWINTYTGEPRYVDDFKGRFTFTTDTSTSTAYMELRSLRSDDTAVYFCARYDHRMDYWGQGTLVTVSS

SEQ ID NO: 98 - protein, 116 aa, Artificial Sequence
  Engineered polypeptide
QIQLVQSGAEVKKPGASVKVSCKASGYTFTNYGMNWVRQAPGQGLEWMGWINTYTGEPKYADDFKGRFTFTTDTSTRTAYMELRSLRSDDTAVYFCARYDHTMDYWGQGTLVTVSS

SEQ ID NO: 99 - protein, 116 aa, Artificial Sequence
  Engineered polypeptide
QIQLVQSGAEVKKPGASVKVSCKASGYTFTNYGMNWVRQAPGQGLEWMGWINTYTGESTYADDFKGRFTFTTDTSTTTAYMELRSLRSDDTAVYFCARYDHAMDYWGQGTLVTVSS

SEQ ID NO: 100 - protein, 116 aa, Artificial Sequence
  Engineered polypeptide
QIQLVQSGAEVKKPGASVKVSCKASGYIFTNYGMNWVRQAPGQGLEWMGWINTYTGESTYADDFKGRFTFTTDTSTTTGYMELRSLRSDDTAVYFCARYDHAMDYWGQGTLVTVSS

SEQ ID NO: 101 - protein, 116 aa, Artificial Sequence
  Engineered polypeptide
QIQLVQSGAEVKKPGASVKVSCKASGYSFTNYGMNWVRQAPGQGLEWMGWINTYTGEPTYGDDFKGRFTFTTDTSTSTAYMELRSLRSDDTAVYFCARYDHAMDYWGQGTLVTVSS

SEQ ID NO: 102 - protein, 116 aa, Artificial Sequence
  Engineered polypeptide
QIQLVQSGAEVKKPGASVKVSCKASGYTFTNYGMNWVRQAPGQGLEWMGWINTYTGETTYTDDFKGRFTFTTDTSTSTAYMELRSLRSDDTAVYFCVRYDHAMDYWGQGTLVTVSS

SEQ ID NO: 103 - protein, 116 aa, Artificial Sequence
  Engineered polypeptide
QIQLVQSGAEVKKPGASVKVSCKASGYTFTNYGMNWVRQAPGQGLEWMGWINTYTGEPTYTDDFKGRFTFTTDTSTSTAYMELRSLRSDDTAVYFCGRYDHAMDYWGQGTLVTVSS

SEQ ID NO: 104 - protein, 116 aa, Artificial Sequence
  Engineered polypeptide
  MISC_FEATURE (3)..(3): Xaa is Gln or His
  MISC_FEATURE (9)..(9): Xaa is Ala, Asp, Thr, Val, Ser, or Pro
  MISC_FEATURE (28)..(28): Xaa is Thr, Ser, or Ile
  MISC_FEATURE (31)..(31): Xaa is Asn or Lys
  MISC_FEATURE (58)..(58): Xaa is Pro, Ser, Thr, or Ala
  MISC_FEATURE (59)..(59): Xaa is Thr, Arg, Lys, or Ile
  MISC_FEATURE (61)..(61): Xaa is Ala, Thr, Val, Ser, or Gly
  MISC_FEATURE (77)..(77): Xaa is Ser, Arg, or Thr
  MISC_FEATURE (79)..(79): Xaa is Ala, Val, or Gly
  MISC_FEATURE (82)..(82): Xaa is Glu or Asp
  MISC_FEATURE (83)..(83): Xaa is Leu or Val
  MISC_FEATURE (97)..(97): Xaa is Ala, Thr, Val, or Gly
  MISC_FEATURE (102)..(102): Xaa is Ala, Arg, Phe, Thr, Pro, Val, Ser, Asp, Asn, His, Leu, Tyr, or Gly
  MISC_FEATURE (110)..(110): Xaa is Thr or Ser
QIXLVQSGXEVKKPGASVKVSCKASGYXFTXYGMNWVRQAPGQGLEWMGWINTYTGEXXYXDDFKGRFTFTTDTSTXTXYMXXRSLRSDDTAVYFCXRYDHXMDYWGQGXLVTVSS

SEQ ID NO: 105 - protein, 112 aa, Artificial Sequence; identical to SEQ ID NO: 19
  Engineered polypeptide

SEQ ID NO: 106 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSGSGSGTDFTLTIDSLQAEDVAVYYCKQSYNLYTFGGGTKVEIK

SEQ ID NO: 107 - protein, 112 aa, Artificial Sequence
  Engineered polypeptide
EIVLTQSPDSLAVSLGERATIKCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 108 - protein, 112 aa, Artificial Sequence
  Engineered polypeptide
EIVLTQSPDSLAVSLGERATINCKSSQSLLNSGTRKNYLAWYQQKPGQSPKLLIYWTSTRESGVPDRFSGSGSGTDFTLTIDSLQAEDVAVYYCKQSYTLYTFGGGTKVEIK

SEQ ID NO: 109 - protein, 112 aa, Artificial Sequence
  Engineered polypeptide
EIVLTQSPDSLAVSLGERATINCKSSQSLFNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 110 - protein, 112 aa, Artificial Sequence
  Engineered polypeptide
EIVLTQSPDSLAVSLGERATINCKSSQSLLNSGTRKNYLAWYQQKAGQSPKLVIYWTSTRESGVPDRFSGSGSGTDFTLTIDSLQAEDVAVYYCKQSYTLYTFGGGTKVEIK

SEQ ID NO: 111 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSGSGSGTDFTLSIDSLQAEDVAVYYCKQSYTLYTFGGGTKVEIK

SEQ ID NO: 112 - protein, 112 aa, Artificial Sequence
  Engineered polypeptide
EIVLTQSPDSLAVSLGERATINCKSSQSLLNSGTRKNYLSWYQQKAGQSPKLLIYWTSTRESGVPDRFSGSGSGTDFTLTIDSLQAEDVAVYYCKQSYSLYTFGGGTKVEIK

SEQ ID NO: 113 - protein, 112 aa, Artificial Sequence
  Engineered polypeptide
EIVLTQSPDSLAVSLGERATINCKSSQSLLNSGTRKNYLTWYQQKAGQSPKLLIYWTSTRESGVPDRFSGSGSGTDFTLTIDSLQAEDVAVYYCKQSYTLYTFGGGTKVEIK

SEQ ID NO: 114 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSGSGSGTDFTLTIDSLQAEDVAIYYCKQSYTLYTFGGGTKVEIK

SEQ ID NO: 115 - protein, 112 aa, Artificial Sequence
  Engineered polypeptide
EIVLTQSPDSLTVSLGERATINCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 116 - protein, 112 aa, Artificial Sequence
  Engineered polypeptide
EIVLTQSPDSLAVSLGERATINCKSSQSLLNSGTRKNYLAWYQQKAGQSPKLIIYWTSTRESGVPDRFSGSGSGTDFTLTIDSLQAEDVAVYYCKQSYTLYTFGGGTKVEIK

SEQ ID NO: 117 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSGSGSGTDFTLTIDRLQAEDVAVYYCKQSYTLYTFGGGTKVEIK

SEQ ID NO: 118 - protein, 112 aa, Artificial Sequence
  Engineered polypeptide
EIVLTQSPDSLTVSLGERATINCKSSQSLLNSGTRKNYLAWYQQKAGQSPNLLIYWTSTRESGVPDRFSGSGSGTDFTLTIDSLQAEDVAVYYCKQSYTLYTFGGGTKVEIK

SEQ ID NO: 119 - protein, 112 aa, Artificial Sequence
  Engineered polypeptide
EIVLTQSPDSLTVSLGERATINCKSSQSLLNSGTRKNYLAWYQQKAGQSPKLLIYWTSTRESGVPDRFSGSGSGTDFTLTIDSLQAEDVAVYYCKQSYNLYTFGGGTKVEIK

SEQ ID NO: 120 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSGSGSGTDFTLTIDSLQAEDVAVYYCKQSYSLYTFGGGTKVEIK

SEQ ID NO: 121 - protein, 112 aa, Artificial Sequence
  Engineered polypeptide
EIVLTQSPDSLAVSLGERATIKCKSSQSLLNSGTRKNYLAWYQKKAGQSPKLLIYWTSTRESGVPDRFSGSGSGTDFTLTIDSLQAEDVAVYYCKQSYTLYTFGGGTKVEIK

SEQ ID NO: 122 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSGSGSGTDFTLTIDSLQTEDVAVYYCKQSYTLYTFGGGTKVEIK

SEQ ID NO: 123 - protein, 112 aa, Artificial Sequence
  Engineered polypeptide
EIVLTQSPDSLAVSLGERATINCKSSQSLLNSGTRKNYLAWYQQKAGQSPKLLIYWTSTRESGVPDRFSASGSGTDFTLTIDSLQAEDVAVYYCKQSYTLYTFGGGTKVEIK

SEQ ID NO: 124 - protein, 112 aa, Artificial Sequence
  Engineered polypeptide
EIVLTQSPDSLAVSLGERATINCKSSQSLLNSGTRKNYLTWYQQKAGQSPKLLIYWTSTRESGVPDRFSGSGSGTDFTLTIDSLQAEDVALYYCKQSYTLYTFGGGTKVEIK

SEQ ID NO: 125 - protein, 112 aa, Artificial Sequence
  Engineered polypeptide
EIVLTQSPDSLSVSLGERATINCKSSQSLLNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 126 - protein, 112 aa, Artificial Sequence
  Engineered polypeptide
EIVLTQSPDSLAVSLGERATINCKSSQSLLNSGTRKNYLAWYQQKSGQSPKLLIYWTSTRESGVPDRFSGSGSGTDFTLTIDSLQAEDVAVYYCKQSYTLYTFGGGTKVEIK

SEQ ID NO: 127 - protein, 112 aa, Artificial Sequence
  Engineered polypeptide
EIVLTQSPDSLAVSLGERATINCKSSQSLVNSGTRKNYLAWYQQKAGQSPKLLIYWTSTRESGVPDRFSGSGSGTDFTLTIDSLQTEDVAVYYCKQSYTLYTFGGGTKVEIK

SEQ ID NO: 128 - protein, 112 aa, Artificial Sequence
  Engineered polypeptide
EIVLTQSPDSLAVSLGERATIKCKSSQSLLNSGTRKNYLAWYQQKAGQSPKLLIYWTSTRESGVPDRFSGSGSGTDFTLTIDSLQAEDVALYYCKQSYTLYTFGGGTKVEIK

SEQ ID NO: 129 - protein, 112 aa, Artificial Sequence
  Engineered polypeptide
EIVLTQSPDSLAVSLGERATINCKSSQSLLNSGTRKNYLAWYQQKAGQSPKLIIYWTSTRESGVPDRFSGSGSGTDFTLTIDSLQAEDVALYYCKQSYTLYTFGGGTKVEIK

SEQ ID NO: 130 - protein, 112 aa, Artificial Sequence
  Engineered polypeptide
EIVLTQSPDSLAVSLGERATINCKSSQSLLNSGTRKNYLAWYQKKSGQSPKLLIYWTSTRESGVPDRFSGSGSGTDFTLSIDSLQAEDVAVYYCKQSYTLYTFGGGTKVEIK

SEQ ID NO: 131 - protein, 112 aa, Artificial Sequence
  Engineered polypeptide
EIVLTQSPDSLAVSLGERATIKCKSSQSLLNSGTRKNYLAWYQQKAGQSPKLLIYWTSTRESGVPDRFSASGSGTDFTLTIDSLQAEDVAVYYCKQSYTLYTFGGGTKVEIK

SEQ ID NO: 132 - protein, 112 aa, Artificial Sequence
  Engineered polypeptide
EIVLTQSPDSLAVSLGERATINCKSSQSLLNSGTRKNYLTWYQQKSGQSPKLLIYWTSTRESGVPDRFSGSGSGTDFTLTIDSLQAEDVAIYYCKQSYTLYTFGGGTKVEIK

SEQ ID NO: 133 - protein, 112 aa, Artificial Sequence
  Engineered polypeptide
EIVLTQSPDSLAVSLGERATINCKSSQSLLNSGTRKNYLAWYQKKAGQSPKLLIYWTSTRESGVPDRFSGSGSGTDFTLSIDSLQAEDVAIYYCKQSYTLYTFGGGTKVEIK

SEQ ID NO: 134 - protein, 112 aa, Artificial Sequence
  Engineered polypeptide
EIVLTQSPDSLAVSLGERATINCKSSQSLLNSGTRKNYLAWYQQKSGQSPKLLIYWTSTRESGVPDRFSGSGSGTDFTLTIDSLQAEDVAIYYCKQSYTLYTFGGGTKVEIK

SEQ ID NO: 135 - protein, 112 aa, Artificial Sequence
  Engineered polypeptide
EIVLTQSPDSLSVSLGERATINCKSSQSLLNSGTRKNYLAWYQQKAGQSPNLLIYWTSTRESGVPDRFSGSGSGTDFTLSIDSLQAEDVAVYYCKQSYTLYTFGGGTKVEIK

SEQ ID NO: 136 - protein, 112 aa, Artificial Sequence
  Engineered polypeptide
EIVLTQSPDSLSVSLGERATINCKSSQSLLNSGTRKNYLAWYQQKAGQSPNLLIYWTSTRESGVPDRFSGSGSGTDFTLTIDSLQAEDVAVYYCKQSYTLYTFGGGTKVEIK

SEQ ID NO: 137 - protein, 112 aa, Artificial Sequence
  Engineered polypeptide
EIVLTQSPDSLTVSLGERATINCKSSQSLLNSGTRKNYLAWYQQKAGQSPNLLIYWTSTRESGVPDRFSGSGSGTDFTLTIDSLQAEDVALYYCKQSYTLYTFGGGTKVEIK

SEQ ID NO: 138 - protein, 112 aa, Artificial Sequence
  Engineered polypeptide
EIVLTQSPDSLSVSLGERATINCKSSQSLLNSGTRKNYLAWYQKKAGQSPKLLIYWTSTRESGVPDRFSGSGSGTDFTLTIDRLQAEDVALYYCKQSYTLYTFGGGTKVEIK

SEQ ID NO: 139 - protein, 112 aa, Artificial Sequence
  Engineered polypeptide
EIVLTQSPDSLSVSLGERATIKCKSSQSLLNSGTRKNYLAWYQQKAGQSPKLLIYWTSTRESGVPDRFSGSGSGTDFTLTIDSLQTEDVAVYYCKQSYTLYTFGGGTKVEIK

SEQ ID NO: 140 - protein, 112 aa, Artificial Sequence
  Engineered polypeptide
EIVLTQSPDSLAVSLGERATIKCKSSQSLLNSGTRKNYLAWYQQKAGQSPKLLIYWTSTRESGVPDRFSGSGSGTDFTLTIDSLQAEDVAIYYCKQSYTLYTFGGGTKVEIK

SEQ ID NO: 141 - protein, 112 aa, Artificial Sequence
  Engineered polypeptide
EIVLTQSPDSLAVSLGERATINCKSSQSLFNSGTRKNYLAWYQQKAGQSPKLLIYWTSTRESGVPDRFSGSGSGTDFTLSIDSLQAEDVAVYYCKQSYTLYTFGGGTKVEIK

SEQ ID NO: 142 - protein, 112 aa, Artificial Sequence
  Engineered polypeptide
EIVLTQSPDSLAVSLGERATINCKSSQSLLNSGTRKNYLAWYQQKAGQSPNLLIYWTSTRESGVPDRFSGSGSGTDFTLTIDSLQAEDVAVYYCKQSYSLYTFGGGTKVEIK

SEQ ID NO: 143 - protein, 112 aa, Artificial Sequence
  Engineered polypeptide
EIVLTQSPDSLAVSLGERATINCKSSQSLLNSGTRKNYLAWYQQKPGQSPKLLIYWTSTRESGVPDRFSGSGSGTDFTLTIDSLQAEDVAVYYCKQSYNLYTFGGGTKVEIK

SEQ ID NO: 144 - protein, 112 aa, Artificial Sequence
  Engineered polypeptide
EIVLTQSPDSLAVSLGERATINCKSSQSLLNSGTRKNYLSWYQQKPGQSPKLIIYWTSTRESGVPDRFSGSGSGTDFTLTIDSLQAEDVAVYYCKQSYTLYTFGGGTKVEIK

SEQ ID NO: 145 - protein, 112 aa, Artificial Sequence
  Engineered polypeptide
EIVLTQSPDSLAVSLGERATINCKSSQSLLNSGTRKNYLAWYQQKSGQSPKLLIYWTSTRESGVPDRFSGSGSGTDFTLTIDSLQTEDVAIYYCKQSYTLYTFGGGTKVEIK

SEQ ID NO: 146 - protein, 112 aa, Artificial Sequence
  Engineered polypeptide
EIVLTQSPDSLAVSLGERATINCKSSQSLLNSGTRKNYLSWYQQKAGQSPNLVIYWTSTRESGVPDRFSGSGSGTDFTLTIDSLQAEDVAVYYCKQSYTLYTFGGGTKVEIK

SEQ ID NO: 147 - protein, 112 aa, Artificial Sequence
  Engineered polypeptide
EIVLTQSPDSLAVSLGERATINCKSSQSLLNSGTRKNYLAWYQKKSGQSPNLLIYWTSTRESGVPDRFSGSGSGTDFTLTIDSLQAEDVAVYYCKQSYTLYTFGGGTKVEIK

SEQ ID NO: 148 - protein, 112 aa, Artificial Sequence
  Engineered polypeptide
EIVLTQSPDSLAVSLGERATINCKSSQSLLNSGTRKNYLSWYQKKSGQSPKLLIYWTSTRESGVPDRFSGSGSGTDFTLTIDSLQAEDVAVYYCKQSYTLYTFGGGTKVEIK

SEQ ID NO: 149 - protein, 112 aa, Artificial Sequence
  Engineered polypeptide
EIVLTQSPDSLAVSLGERATINCKSSQSLFNSGTRKNYLAWYQKKAGQSPKLLIYWTSTRESGVPDRFSGSGSGTDFTLSIDRLQAEDVAVYYCKQSYTLYTFGGGTKVEIK

SEQ ID NO: 150 - protein, 112 aa, Artificial Sequence
  Engineered polypeptide
EIVLTQSPDSLTVSLGERATINCKSSQSLLNSGTRKNYLAWYQQKPGQSPKLLIYWTSTRESGVPDRFSGSGSGTDFTLTIDSLQAEDVAVYYCKQSYTLYTFGGGTKVEIK

SEQ ID NO: 151 - protein, 112 aa, Artificial Sequence
  Engineered polypeptide
EIVLTQSPDSLAVSLGERATIKCKSSQSLLNSGTRKNYLTWYQQKAGQSPNLLIYWTSTRESGVPDRFSGSGSGTDFTLTIDSLQAEDVAVYYCKQSYTLYTFGGGTKVEIK

SEQ ID NO: 152 - protein, 112 aa, Artificial Sequence
  Engineered polypeptide
EIVLTQSPDSLSVSLGERATINCKSSQSLLNSGTRKNYLAWYQQKSGQSPNLVIYWTSTRESGVPDRFSGSGSGTDFTLTIDSLQAEDVAVYYCKQSYTLYTFGGGTKVEIK

SEQ ID NO: 153 - protein, 112 aa, Artificial Sequence
  Engineered polypeptide
EIVLTQSPDSLSVSLGERATINCKSSQSLFNSGTRKNYLAWYQQKAGQSPKLLIYWTSTRESGVPDRFSGSGSGTDFTLTIDSLQAEDVAVYYCKQSYTLYTFGGGTKVEIK

SEQ ID NO: 154 - protein, 112 aa, Artificial Sequence
  Engineered polypeptide
EIVLTQSPDSLTVSLGERATINCKSSQSLFNSGTRKNYLAWYQKKAGQSPKLLIYWTSTRESGVPDRFSGSGSGTDFTLTIDSLQAEDVAVYYCKQSYNLYTFGGGTKVEIK

SEQ ID NO: 155 - protein, 112 aa, Artificial Sequence
  Engineered polypeptide
EIVLTQSPDSLAVSLGERATINCKSSQSLLNSGTRKNYLAWYQQKPGQSPKLLIYWTSTRESGVPDRFSASGSGTDFTLTIDSLQAEDVAIYYCKQSYTLYTFGGGTKVEIK

SEQ ID NO: 156 - protein, 112 aa, Artificial Sequence
  Engineered polypeptide
EIVLTQSPDSLAVSLGERATINCKSSQSLFNSGTRKNYLAWYQQKAGQSPKLIIYWTSTRESGVPDRFSASGSGTDFTLSIDRLQAEDVAVYYCKQSYTLYTFGGGTKVEIK

SEQ ID NO: 157 - protein, 112 aa, Artificial Sequence
  Engineered polypeptide
EIVLTQSPDSLSVSLGERATINCKSSQSLLNSGTRKNYLAWYQQKAGQSPKLLIYWTSTRESGVPDRFSASGSGTDFTLTIDRLQAEDVALYYCKQSYTLYTFGGGTKVEIK

SEQ ID NO: 158 - protein, 112 aa, Artificial Sequence
  Engineered polypeptide
  MISC_FEATURE (12)..(12): Xaa is Ala, Thr, or Ser
  MISC_FEATURE (22)..(22): Xaa is Asn or Lys
  MISC_FEATURE (30)..(30): Xaa is Leu, Phe, or Val
  MISC_FEATURE (40)..(40): Xaa is Ala, Ser, or Thr
  MISC_FEATURE (44)..(44): Xaa is Gln or Lys
  MISC_FEATURE (46)..(46): Xaa is Ala, Pro, or Ser
  MISC_FEATURE (51)..(51): Xaa is Lys or Asn
  MISC_FEATURE (53)..(53): Xaa is Leu, Val, or Ile
  MISC_FEATURE (70)..(70): Xaa is Gly or Ala
  MISC_FEATURE (80)..(80): Xaa is Thr or Ser
  MISC_FEATURE (83)..(83): Xaa is Ser or Arg
  MISC_FEATURE (86)..(86): Xaa is Ala or Thr
  MISC_FEATURE (91)..(91): Xaa is Val, Ile, or Leu
  MISC_FEATURE (99)..(99): Xaa is Thr, Asn, or Ser
EIVLTQSPDSLXVSLGERATIXCKSSQSLXNSGTRKNYLXWYQXKXGQSPXLXIYWTSTRESGVPDRFSXSGSGTDFTLXIDXLQXEDVAXYYCKQSYXLYTFGGGTKVEIK

SEQ ID NO: 159 - protein, 12 aa, Artificial Sequence
  Engineered polypeptide
QSLFNSGTRKNY

SEQ ID NO: 160 - protein, 12 aa, Artificial Sequence
  Engineered polypeptide
QSLVNSGTRKNY

SEQ ID NO: 161 - protein, 12 aa, Artificial Sequence
  Engineered polypeptide
QSLFNSGTRKNY

SEQ ID NO: 162 - protein, 8 aa, Artificial Sequence
  Engineered polypeptide
KQSYNLYT

SEQ ID NO: 163 - protein, 8 aa, Artificial Sequence
  Engineered polypeptide
KQSYSLYT

SEQ ID NO: 164 - protein, 8 aa, Artificial Sequence
  Engineered polypeptide
GYTFTKYG

SEQ ID NO: 165 - protein, 8 aa, Artificial Sequence
  Engineered polypeptide
GYSFTNYG

SEQ ID NO: 166 - protein, 8 aa, Artificial Sequence
  Engineered polypeptide
GYIFTNYG

SEQ ID NO: 167 - protein, 8 aa, Artificial Sequence
  Engineered polypeptide
INTYTGES

SEQ ID NO: 168 - protein, 8 aa, Artificial Sequence
  Engineered polypeptide
INTYTGET

SEQ ID NO: 169 - protein, 8 aa, Artificial Sequence
  Engineered polypeptide
INTYTGEA

SEQ ID NO: 170 - protein, 9 aa, Artificial Sequence
  Engineered polypeptide
ARYDHRMDY

SEQ ID NO: 171 - protein, 9 aa, Artificial Sequence
  Engineered polypeptide
ARYDHFMDY

SEQ ID NO: 172 - protein, 9 aa, Artificial Sequence
  Engineered polypeptide
ARYDHTMDY

SEQ ID NO: 173 - protein, 9 aa, Artificial Sequence
  Engineered polypeptide
ARYDHPMDY

SEQ ID NO: 174 - protein, 9 aa, Artificial Sequence
  Engineered polypeptide
ARYDHVMDY

SEQ ID NO: 175 - protein, 9 aa, Artificial Sequence
  Engineered polypeptide
ARYDHSMDY

SEQ ID NO: 176 - protein, 9 aa, Artificial Sequence
  Engineered polypeptide
TRYDHAMDY

SEQ ID NO: 177 - protein, 9 aa, Artificial Sequence
  Engineered polypeptide
ARYDHDMDY

SEQ ID NO: 178 - protein, 9 aa, Artificial Sequence
  Engineered polypeptide
ARYDHNMDY

SEQ ID NO: 179 - protein, 9 aa, Artificial Sequence
  Engineered polypeptide
ARYDHHMDY

SEQ ID NO: 180 - protein, 9 aa, Artificial Sequence
  Engineered polypeptide
ARYDHLMDY

SEQ ID NO: 181 - protein, 9 aa, Artificial Sequence
  Engineered polypeptide
ARYDHYMDY

SEQ ID NO: 182 - protein, 9 aa, Artificial Sequence
  Engineered polypeptide
TRYDHRMDY

SEQ ID NO: 183 - protein, 9 aa, Artificial Sequence
  Engineered polypeptide
VRYDHAMDY

SEQ ID NO: 184 - protein, 9 aa, Artificial Sequence
  Engineered polypeptide
GRYDHAMDY

SEQ ID NO: 185 - protein, 9 aa, Artificial Sequence
  Engineered polypeptide
ARYDHGMDY

SEQ ID NO: 186 - protein, 17 aa, Artificial Sequence
  Engineered polypeptide
KSSQSLLNSGTRKNYLA

SEQ ID NO: 187 - protein, 17 aa, Artificial Sequence
  Engineered polypeptide
KSSQSLFNSGTRKNYLA

SEQ ID NO: 188 - protein, 17 aa, Artificial Sequence
  Engineered polypeptide
KSSQSLLNSGTRKNYLS

SEQ ID NO: 189 - protein, 17 aa, Artificial Sequence
  Engineered polypeptide
KSSQSLLNSGTRKNYLT

SEQ ID NO: 190 - protein, 17 aa, Artificial Sequence
  Engineered polypeptide
KSSQSLVNSGTRKNYLA

SEQ ID NO: 191 - protein, 7 aa, Artificial Sequence
  Engineered polypeptide
WTSTRES

SEQ ID NO: 192 - protein, 10 aa, Artificial Sequence
  Engineered polypeptide
GYTFTKYGMN

SEQ ID NO: 193 - protein, 10 aa, Artificial Sequence
  Engineered polypeptide
GYTFTNYGMN

SEQ ID NO: 194 - protein, 10 aa, Artificial Sequence
  Engineered polypeptide
GYSFTNYGMN

SEQ ID NO: 195 - protein, 10 aa, Artificial Sequence
  Engineered polypeptide
GYIFTNYGMN

SEQ ID NO: 196 - protein, 17 aa, Artificial Sequence
  Engineered polypeptide
WINTYTGEPTYADDFKG

SEQ ID NO: 197 - protein, 17 aa, Artificial Sequence
  Engineered polypeptide
WINTYTGEPRYADDFKG

SEQ ID NO: 198 - protein, 17 aa, Artificial Sequence
  Engineered polypeptide
WINTYTGEPIYADDFKG

SEQ ID NO: 199 - protein, 17 aa, Artificial Sequence
  Engineered polypeptide
WINTYTGEPTYTDDFKG

SEQ ID NO: 200 - protein, 17 aa, Artificial Sequence
  Engineered polypeptide
WINTYTGEPTYVDDFKG

SEQ ID NO: 201 - protein, 17 aa, Artificial Sequence
  Engineered polypeptide
WINTYTGEPTYSDDFKG

SEQ ID NO: 202 - protein, 17 aa, Artificial Sequence
  Engineered polypeptide
WINTYTGESTYADDFKG

SEQ ID NO: 203 - protein, 17 aa, Artificial Sequence
  Engineered polypeptide
WINTYTGETTYGDDFKG

SEQ ID NO: 204 - protein, 17 aa, Artificial Sequence
  Engineered polypeptide
WINTYTGEPKYTDDFKG

SEQ ID NO: 205 - protein, 17 aa, Artificial Sequence
  Engineered polypeptide
WINTYTGEPKYGDDFKG

SEQ ID NO: 206 - protein, 17 aa, Artificial Sequence
  Engineered polypeptide
WINTYTGEPTYGDDFKG

SEQ ID NO: 207 - protein, 17 aa, Artificial Sequence
  Engineered polypeptide
WINTYTGEATYADDFKG

SEQ ID NO: 208 - protein, 17 aa, Artificial Sequence
  Engineered polypeptide
WINTYTGETTYTDDFKG

SEQ ID NO: 209 - protein, 17 aa, Artificial Sequence
  Engineered polypeptide
WINTYTGETTYADDFKG

SEQ ID NO: 210 - protein, 17 aa, Artificial Sequence
  Engineered polypeptide
WINTYTGEPKYADDFKG

SEQ ID NO: 211 - protein, 17 aa, Artificial Sequence
  Engineered polypeptide
WINTYTGEPRYVDDFKG

SEQ ID NO: 212 - protein, 12 aa, Artificial Sequence
  Engineered polypeptide
  MISC_FEATURE (4)..(4): Xaa is Leu, Phe, or Val
QSLXNSGTRKNY

SEQ ID NO: 213 - protein, 8 aa, Artificial Sequence
  Engineered polypeptide
  MISC_FEATURE (5)..(5): Xaa is Thr, Asn, Ser
KQSYXLYT

SEQ ID NO: 214 - protein, 8 aa, Artificial Sequence
  Engineered polypeptide
  MISC_FEATURE (3)..(3): Xaa is Thr, Ser, or Ile
  MISC_FEATURE (6)..(6): Xaa is Asn or Lys
GYXFTXYG

SEQ ID NO: 215 - protein, 8 aa, Artificial Sequence
  Engineered polypeptide
  MISC_FEATURE (8)..(8): Xaa is Pro, Ser, Thr, or Ala
INTYTGEX

SEQ ID NO: 216 - protein, 9 aa, Artificial Sequence
  Engineered polypeptide
  MISC_FEATURE (1)..(1): Xaa is Ala, Thr, Val, or Gly
  MISC_FEATURE (6)..(6): Xaa is Ala, Arg, Phe, Thr, Pro, Val, Ser, Asp, Asn, His, Leu, Tyr, or Gly
XRYDHXMDY

SEQ ID NO: 217 - protein, 330 aa, Homo sapiens [sequence omitted]

SEQ ID NO: 218 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 220 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 221 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 222 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 107 aa, Homo sapiens [sequence omitted]

SEQ ID NO: 224 - protein, 105 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 225 - protein, 116 aa, Artificial Sequence
  Engineered polypeptide
QVQLQQSGSELKKPGASVKVSCKASGYTFTNYGMNWVRQAPGQGLEWMGWINTYTGEPTYADDFKGRFVFSLDTSVSTAYLQISSLKAEDTAVYYCARYDHAMDYWGQGTMVTVSS

SEQ ID NO: 226 - protein, 116 aa, Artificial Sequence
  Engineered polypeptide
QVQLVQSGSELKKPGASVKISCKASGYTFTNYGMNWMRRAPGQGLKWMGWINTYTGEPTYADDFKGRFVFSLDTSVNTAFLQISSLQAEDTAVYYCARYDHAMDYWGQGTTVTVSS

SEQ ID NO: 227 - protein, 116 aa, Artificial Sequence; identical to SEQ ID NO: 14
  Engineered polypeptide

SEQ ID NO: 228 - protein, 116 aa, Artificial Sequence
  Engineered polypeptide
QVHLVQSGSEVKKPGASVKVSCQASGYTFTNYGMNWVRQAPGQGLEWMGWINTYTGEPTYADDFKGRFVFSLDTSVSTAYLQINRLKAEDTAVYFCARYDHAMDYWGQGTLVTVSS

SEQ ID NO: 229 - protein, 116 aa, Artificial Sequence
  Engineered polypeptide
QVQLVQSGPELKKPGESVKVSCKASGYTFTNYGMNWVRQAPGRGLKWMGWINTYTGEPTYADDFKGRFVFSLDTSVSTAYLQISSLKAEDTAVYFCARYDHAMDYWGQGTSVTVSS

SEQ ID NO: 230 - protein, 112 aa, Artificial Sequence; identical to SEQ ID NO: 19
  Engineered polypeptide

SEQ ID NO: 231 - protein, 112 aa, Artificial Sequence; identical to SEQ ID NO: 20
  Engineered polypeptide

SEQ ID NO: 232 - protein, 112 aa, Artificial Sequence; identical to SEQ ID NO: 21
  Engineered polypeptide

SEQ ID NO: 233 - protein, 112 aa, Artificial Sequence
  Engineered polypeptide
DIVMTQSPVSLAVSLGERATINCKSSQSLLNSGTRKNYLAWYQQKPGQPPKLLIYWTSTRESGVPDRFSGSGSGTDFTLTISSLQAEDVAVYYCKQSYTLYTFGPGTKVDIK

SEQ ID NO: 234 - protein, 112 aa, Artificial Sequence
  Engineered polypeptide
DIVMTQSPDSLAVSLGERATINCKSSQSLLNSGTRKNYLAWYQQKPGQPPKLLIYWTSTRESGVPDRFSGSGSGTDFTLTISSLQAEDVAVYYCKQSYTLYTFGGGTKLEIK